(19) United States
(12) Patent Application Publication
(10) Pub. No.: US 2025/0319093 A1
(43) Pub. Date: Oct. 16, 2025

(54) COMPOSITIONS FOR PREVENTING OR TREATING IDIOPATHIC PULMONARY FIBROSIS (IPF)

(71) Applicant: CHONG KUN DANG PHARMACEUTICAL CORP., Seoul (KR)

(72) Inventors: Nina Ha, Gyeonggi-do (KR); Min Cheol Kim, Gyeonggi-do (KR); Suk Jin Lee, Gyeonggi-do (KR); Jiyeon Baek, Gyeonggi-do (KR); Dong-hyeon Suh, Gyeonggi-do (KR)

(21) Appl. No.: 18/865,914

(22) PCT Filed: May 15, 2023
(86) PCT No.: PCT/IB2023/054969

§ 371 Date: Nov. 14, 2024

(30) Foreign Application Priority Data
May 16, 2022 (KR) 10-2022-0059861

(51) Int. Cl. A61K 31/5377; A61K 31/4245; A61K 31/438; A61K 31/4439; A61K 31/454; A61K 31/496; A61K 31/541; A61K 31/551; A61K 31/553; A61P 11/00

(52) U.S. Cl. CPC A61K 31/5377; A61K 31/4245; A61K 31/438; A61K 31/4439; A61K 31/454; A61K 31/496; A61K 31/541; A61K 31/551; A61K 31/553; A61P 11/00

(57) ABSTRACT

The present disclosure relates to a pharmaceutical composition for preventing or treating idiopathic pulmonary fibrosis, comprising a compound represented by formula I, optical isomers thereof or pharmaceutically acceptable salts thereof as an effective ingredient, a method for preventing or treating idiopathic pulmonary fibrosis using the compound, a use of the compound for preventing or treating idiopathic pulmonary fibrosis, and a use of the compound in preparing a medicament for preventing or treating idiopathic pulmonary fibrosis.

### TECHNICAL FIELD

The present disclosure relates to a pharmaceutical composition for preventing or treating idiopathic pulmonary fibrosis, comprising a compound represented by formula I, optical isomers thereof or pharmaceutically acceptable salts thereof as an effective ingredient, a method for preventing or treating idiopathic pulmonary fibrosis using the compound, a use of the compound for preventing or treating idiopathic pulmonary fibrosis, and a use of the compound in preparing a medicament for preventing or treating idiopathic pulmonary fibrosis.

### BACKGROUND

Idiopathic pulmonary fibrosis (IPF) is a disease in which the lung parenchyma becomes fibrous due to an abnormal tissue repair mechanism after damage to alveolar epithelial cells for unknown reasons. The IPF shows symptoms such as chronic dry cough, shortness of breath, etc., and is a disease with a poor prognosis, which usually has a survival period of about 3-5 years only after being diagnosed due to the occurrence of the symptoms. It is known that an incidence rate is higher in men over the age of 50.

Currently, two drugs, nintedanib and pirfenidone, are mainly used for the treatment of idiopathic pulmonary fibrosis, but the drugs slow only a progression of the disease without improving a survival rate. Accordingly, there is an urgent need for developing a drug capable of effectively treating idiopathic pulmonary fibrosis.

### RELATED ART REFERENCE

### Patent Documents

(Patent Document 1) Korean Unexamined Patent Application Publication No. 10-2017-0017792

### DETAILED DESCRIPTION OF THE INVENTION

### Technical Problem

The present disclosure provides a pharmaceutical composition for preventing or treating idiopathic pulmonary fibrosis, including a compound represented by formula I, optical isomers thereof or pharmaceutically acceptable salts thereof as an effective ingredient.

The present disclosure provides a method for preventing or treating idiopathic pulmonary fibrosis, including administering a compound represented by formula I, optical isomers thereof or pharmaceutically acceptable salts thereof into an individual.

The present disclosure provides a use of a compound represented by formula I, optical isomers thereof or pharmaceutically acceptable salts thereof for preventing or treating idiopathic pulmonary fibrosis.

The present disclosure provides a use of a compound represented by formula I, optical isomers thereof or pharmaceutically acceptable salts thereof in preparing a medicament for preventing or treating idiopathic pulmonary fibrosis.

### Technical Solution

This is described in detail as follows. Meanwhile, each description and embodiment disclosed in the present disclosure may be also applied to other descriptions and embodiments thereof, respectively. In other words, all the combinations of various elements disclosed in the present disclosure fall within the scope of the present disclosure. Also, it cannot be seen that the scope of the present disclosure is limited to the specific description described below.

The present disclosure provides a pharmaceutical composition for preventing or treating idiopathic pulmonary fibrosis, including a compound represented by a following formula I, optical isomers thereof or pharmaceutically acceptable salts thereof as an effective ingredient.

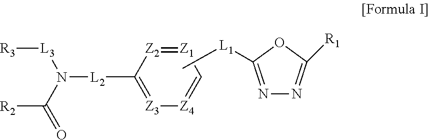

in formula I,
wherein L₁, L₂ and L₃ are each independently a bond or —(C₁-C₂ alkylene)-;
R₁ is —CX₂H or —CX₃;
R₂ is —NR^{A}R^{B}, —OR^{C},

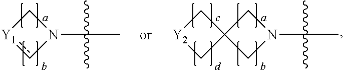

{wherein at least one H of

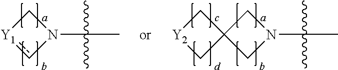

may be each independently substituted with —X, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be each independently substituted with —X, —OH, —CF₃ or —CF₂H]};
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl,

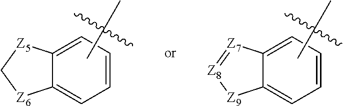

{wherein, at least one H of —(C₁-C₄ alkyl) may be each independently substituted with —X or —OH,
at least one H of -aryl or -heteroaryl may be each independently substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), -aryl, heteroaryl,

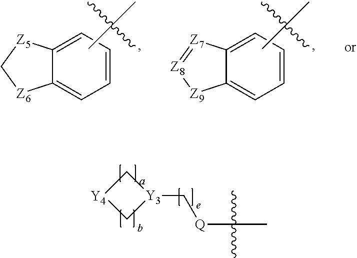

[wherein, at least one H of

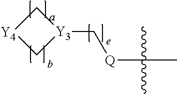

may be each independently substituted with —X, —(C₁-C₄ alkyl), —NR^{D}R^{E}, —CF₃ or —CF₂H],
at least one H of —(C₃-C₇ cycloalkyl), —(C₂-C₆ cycloheteroalkyl), adamantyl,

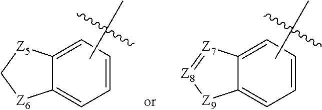

may be each independently substituted with —X, —OH or (C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ are each independently N or CR^{Z}, {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ alkyl)-heteroaryl, —(C₃-C₇ cycloalkyl), —(C₂-C₆ heterocycloalkyl) or

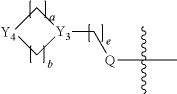

{wherein, at least one H of the —(C₁-C₄ alkyl), —(C₁-C₄ alkyl)-OH or —(C₁-C₄ alkyl)-NR^{D}R^{E} may be each independently substituted with —X,
at least one H of the -aryl, —(C₁-C₄ alkyl)-aryl, -heteroaryl, —(C₁-C₄ alkyl)-heteroaryl, (C₃-C₇ cycloalkyl) or —(C₂-C₆ heterocycloalkyl) may be each independently substituted with —X, —OH, —O(C₁-C₄ alkyl), —(C₁-C₄ alkyl), —CF₃, —CF₂H or —CN,
at least one H of

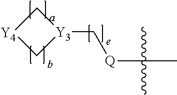

may be each independently substituted with —X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl)};
R^{C} is —(C₁-C₄ alkyl), -aryl, —(C₁-C₄ alkyl)-aryl, -heteroaryl or —(C₁-C₄ alkyl)-heteroaryl {wherein, at least one H of —(C₁-C₄ alkyl) may be each independently substituted with —X or —OH, at least one H of -aryl, —(C₁-C₄ alkyl)-aryl, -heteroaryl or —(C₁-C₄ alkyl)-heteroaryl may be each independently substituted with —X, —OH, —CF₃ or —CF₂H};
R^{D} and R^{E} are each independently —H, —(C₁-C₄ alkyl), -aryl or —(C₁-C₄ alkyl)-aryl {wherein, at least one H of —(C₁-C₄ alkyl) may be each independently substituted with —X or —OH, at least one H of -aryl or —(C₁-C₄ alkyl)-aryl may be each independently substituted with —X, —OH, —CF₃ or —CF₂H};
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein, at least one H of —(C₁-C₄ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), (C₁-C₄ alkyl)-NR^{D}R^{E} or —S(═O)₂—(C₁-C₄ alkyl) may be each independently substituted with —X,
at least one H of -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —C₂-C₆ heterocycloalkyl or —(C₁-C₄ alkyl)-C(═O)—(C₂-C₆ heterocycloalkyl) may be each independently substituted with —X, —OH, —CF₃ or —CF₂H};
R^{G} is —H or —(C₁-C₄ alkyl);
Q is —O— or a bond;
[symbol] is a single bond or double bond {provided that, when [symbol] is a double bond, then Y₁ is ═CH—};
a to e are each independently an integer of 0, 1, 2, 3 or 4 {provided that, a and b may not be simultaneously 0, and c and d may not be simultaneously 0};
X is each independently F, Cl, Br or I.

In the present disclosure, in the compound represented by the above formula I, wherein
L₁, L₂ and L₃ are each independently a bond or —(C₁-C₂ alkylene)-;
R₁ is —CX₂H or —CX₃;
R₂ is —NR^{A}R^{B}, —OR^{C},

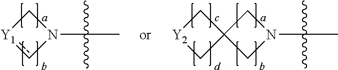

{wherein at least one of H of

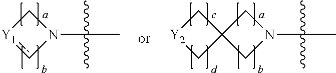

may be each independently substituted with —X, —OH, —NR^{D}R^{E} or —(C₁-C₄ alkyl)};
R₃ is —(C₁-C₄ alkyl), —(C₃-C₇ cycloalkyl), -aryl, -heteroaryl, -adamantyl,

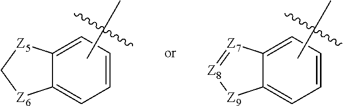

{wherein at least one H of -aryl or -heteroaryl may be each independently substituted with —X, —O(C₁-C₄ alkyl), —OCF₃, —O-aryl, —NR^{D}R^{E}, —(C₁-C₄ alkyl), —CF₃, —S(═O)₂—(C₁-C₄ alkyl), -aryl, -heteroaryl,

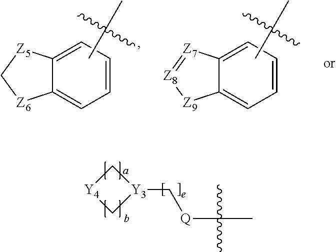

[wherein at least one H of

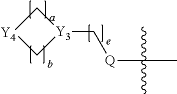

may be each independently substituted with —NR^{D}R^{E} or —(C₁-C₄ alkyl)],

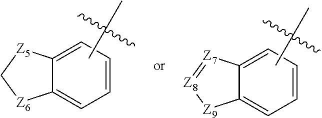

at least one H of may be each independently substituted with —(C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ is each independently N or CR^{Z} {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, —(C₃-C₇ cycloalkyl) or

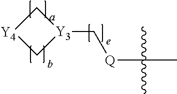

{wherein at least one H of

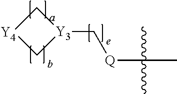

may be each independently substituted with —X, —(C₁-C₄ alkyl), —CF₃, —(C₂-C₆ heterocycloalkyl), —(C₁-C₄ alkyl)-aryl, -heteroaryl or -heteroaryl-(C₁-C₄ alkyl)};
R^{C} is —(C₁-C₄ alkyl) or -aryl;
R^{D} and R^{E} are each independently —H, —(C₁-C₄ alkyl) or —(C₁-C₄ alkyl)-aryl;
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein at least one H of —(C₁-C₄ alkyl) or —C(═O)—O(C₁-C₄ alkyl) may be each independently substituted with —X,
at least one H of -aryl may be substituted with —X};
R^{G} is —(C₁-C₄ alkyl);
Q is —O— or a bond;
[symbol] is a single bond or a double bond {provided that, when [symbol] is a double bond, then Y₁ is —CH—};
a to e are each independently an integer of 0, 1, 2, 3 or 4 {provided that, a and b may not be simultaneously 0, and c and d may not be simultaneously 0};
X is each independently F, Cl, Br or I.

In the present disclosure, the compound represented by the above formula I may be the compound represented by a following formula Ia:

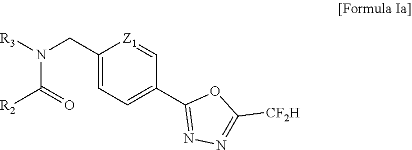

in formula Ia,
R₂ is

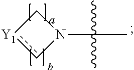

R₃ is -aryl {wherein at least one H of -aryl may be each independently substituted with —X};
Y₁ is —S(═O)₂—;
Z₁ is N or CR^{Z} {wherein R^{Z} is —X};
a and b are each independently an integer of 0, 1, 2, 3 or 4 {wherein a and b may not be simultaneously 0};
X is each independently F, Cl, Br or I.

In the present disclosure, in the compound represented by formula Ia, wherein
R₂ is

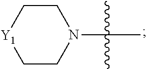

R₃ is -phenyl {wherein at least one H of -phenyl may be each independently substituted with —F or —Cl};
Y₁ is —S(═O)₂—;
Z₁ is N or CF.

In the present disclosure, the compound represented by the above formula I may be a compound described in a following table.

| Compound | Structure | Compound | Structure |
|---|---|---|---|
| 1 | 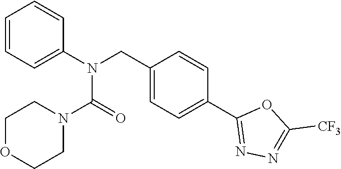 [SMILES: O=C(N1CCOCC1)N(Cc1ccc(-c2nnc(C(F)(F)F)o2)cc1)c1ccccc1] | 2 | 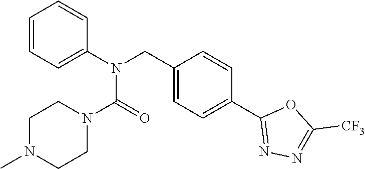 [SMILES: CN1CCN(C(=O)N(Cc2ccc(-c3nnc(C(F)(F)F)o3)cc2)c2ccccc2)CC1] |
| 3 | 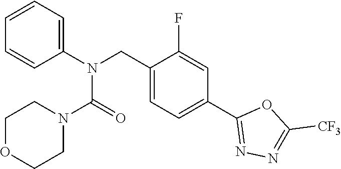 [SMILES: O=C(N1CCOCC1)N(Cc1ccc(-c2nnc(C(F)(F)F)o2)cc1F)c1ccccc1] | 4 | 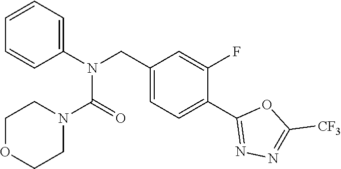 [SMILES: O=C(N1CCOCC1)N(Cc1ccc(-c2nnc(C(F)(F)F)o2)c(F)c1)c1ccccc1] |
| 5 | 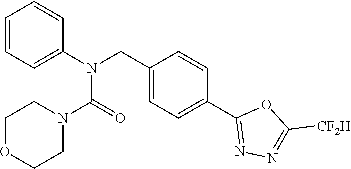 [SMILES: O=C(N1CCOCC1)N(Cc1ccc(-c2nnc(C(F)F)o2)cc1)c1ccccc1] | 6 | 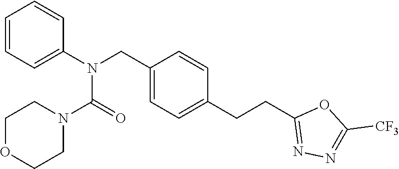 [SMILES: O=C(N1CCOCC1)N(Cc1ccc(CCc2nnc(C(F)(F)F)o2)cc1)c1ccccc1] |
| 7 | 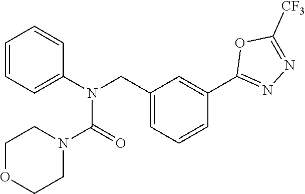 [SMILES: O=C(N1CCOCC1)N(Cc1cccc(-c2nnc(C(F)(F)F)o2)c1)c1ccccc1] | 8 | 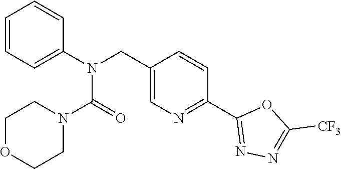 [SMILES: O=C(N1CCOCC1)N(Cc1ccc(-c2nnc(C(F)(F)F)o2)nc1)c1ccccc1] |
| 9 | 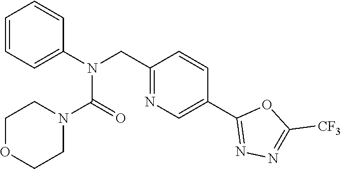 [SMILES: O=C(N1CCOCC1)N(Cc1ccc(-c2nnc(C(F)(F)F)o2)cn1)c1ccccc1] | 10 | 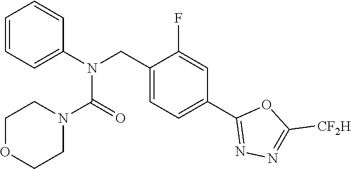 [SMILES: O=C(N1CCOCC1)N(Cc1ccc(-c2nnc(C(F)F)o2)cc1F)c1ccccc1] |
| 11 | 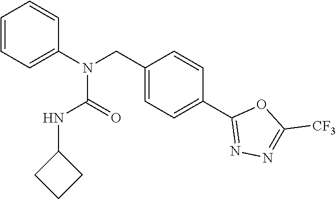 [SMILES: O=C(NC1CCC1)N(Cc1ccc(-c2nnc(C(F)(F)F)o2)cc1)c1ccccc1] | 12 | 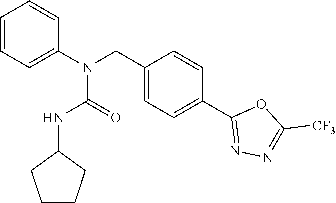 [SMILES: O=C(NC1CCCC1)N(Cc1ccc(-c2nnc(C(F)(F)F)o2)cc1)c1ccccc1] |
| 13 | 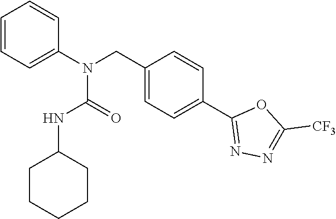 [SMILES: O=C(NC1CCCCC1)N(Cc1ccc(-c2nnc(C(F)(F)F)o2)cc1)c1ccccc1] | 14 | 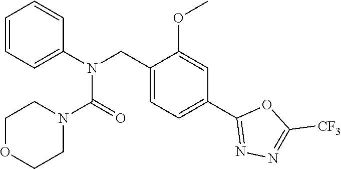 [SMILES: COc1cc(-c2nnc(C(F)(F)F)o2)ccc1CN(C(=O)N1CCOCC1)c1ccccc1] |
| 15 | 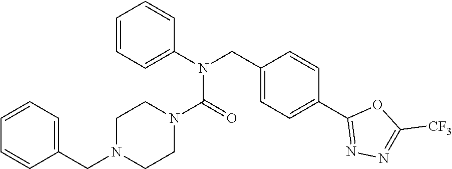 [SMILES: O=C(N1CCN(Cc2ccccc2)CC1)N(Cc1ccc(-c2nnc(C(F)(F)F)o2)cc1)c1ccccc1] | 16 | 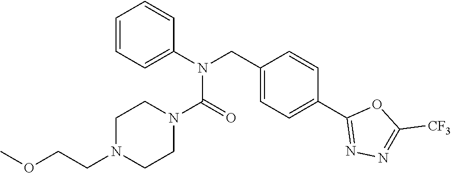 [SMILES: COCCN1CCN(C(=O)N(Cc2ccc(-c3nnc(C(F)(F)F)o3)cc2)c2ccccc2)CC1] |
| 17 | 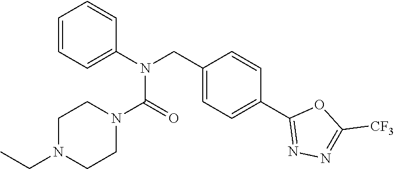 [SMILES: CCN1CCN(C(=O)N(Cc2ccc(-c3nnc(C(F)(F)F)o3)cc2)c2ccccc2)CC1] | 18 | 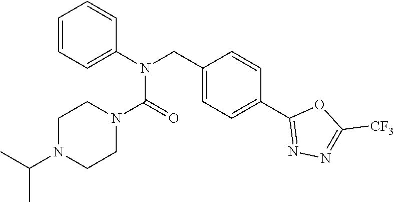 [SMILES: CC(C)N1CCN(C(=O)N(Cc2ccc(-c3nnc(C(F)(F)F)o3)cc2)c2ccccc2)CC1] |
| 19 | 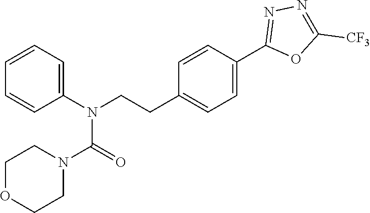 [SMILES: O=C(N1CCOCC1)N(CCc1ccc(-c2nnc(C(F)(F)F)o2)cc1)c1ccccc1] | 20 | 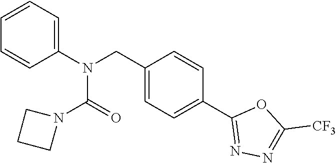 [SMILES: O=C(N1CCC1)N(Cc1ccc(-c2nnc(C(F)(F)F)o2)cc1)c1ccccc1] |
| 21 | 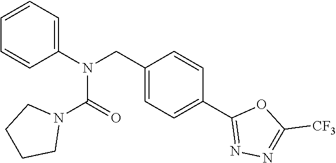 [SMILES: O=C(N1CCCC1)N(Cc1ccc(-c2nnc(C(F)(F)F)o2)cc1)c1ccccc1] | 22 | 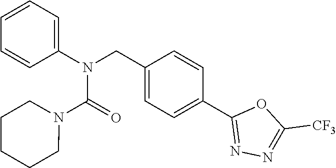 [SMILES: O=C(N1CCCCC1)N(Cc1ccc(-c2nnc(C(F)(F)F)o2)cc1)c1ccccc1] |
| 23 | 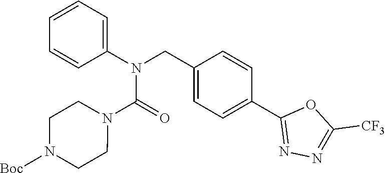 [SMILES: CC(C)(C)OC(=O)N1CCN(C(=O)N(Cc2ccc(-c3nnc(C(F)(F)F)o3)cc2)c2ccccc2)CC1] | 24 | 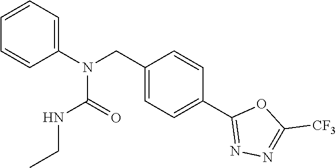 [SMILES: CCNC(=O)N(Cc1ccc(-c2nnc(C(F)(F)F)o2)cc1)c1ccccc1] |
| 25 | 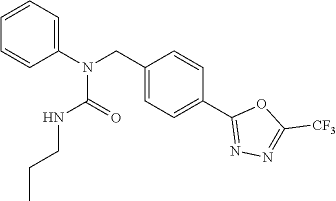 [SMILES: CCCNC(=O)N(Cc1ccc(-c2nnc(C(F)(F)F)o2)cc1)c1ccccc1] | 26 | 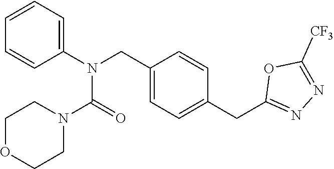 [SMILES: O=C(N1CCOCC1)N(Cc1ccc(Cc2nnc(C(F)(F)F)o2)cc1)c1ccccc1] |
| 27 | 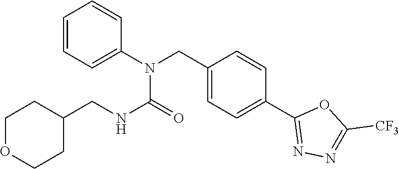 [SMILES: O=C(NCC1CCOCC1)N(Cc1ccc(-c2nnc(C(F)(F)F)o2)cc1)c1ccccc1] | 28 | 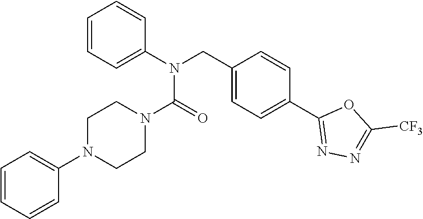 [SMILES: O=C(N1CCN(c2ccccc2)CC1)N(Cc1ccc(-c2nnc(C(F)(F)F)o2)cc1)c1ccccc1] |
| 29 | 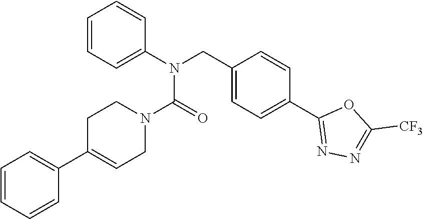 [SMILES: O=C(N1CC=C(c2ccccc2)CC1)N(Cc1ccc(-c2nnc(C(F)(F)F)o2)cc1)c1ccccc1] | 30 | 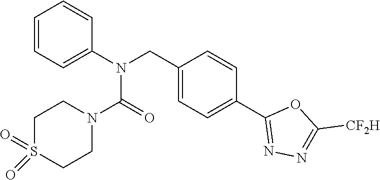 [SMILES: O=C(N1CCS(=O)(=O)CC1)N(Cc1ccc(-c2nnc(C(F)F)o2)cc1)c1ccccc1] |
| 31 | 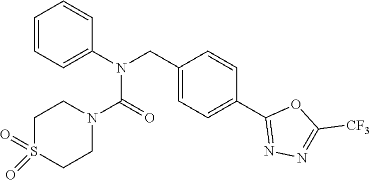 [SMILES: O=C(N1CCS(=O)(=O)CC1)N(Cc1ccc(-c2nnc(C(F)(F)F)o2)cc1)c1ccccc1] | 32 | 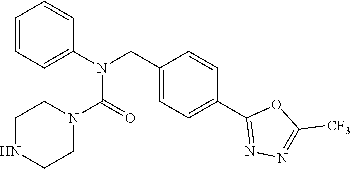 [SMILES: O=C(N1CCNCC1)N(Cc1ccc(-c2nnc(C(F)(F)F)o2)cc1)c1ccccc1] |
| 33 | 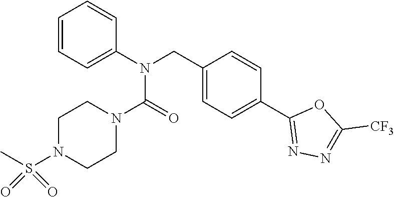 [SMILES: CS(=O)(=O)N1CCN(C(=O)N(Cc2ccc(-c3nnc(C(F)(F)F)o3)cc2)c2ccccc2)CC1] | 34 | 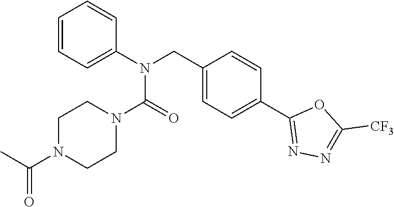 [SMILES: CC(=O)N1CCN(C(=O)N(Cc2ccc(-c3nnc(C(F)(F)F)o3)cc2)c2ccccc2)CC1] |
| 35 | 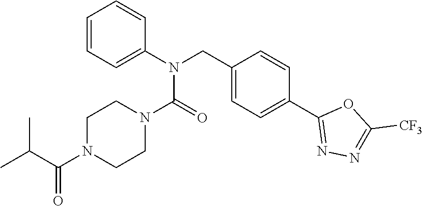 [SMILES: CC(C)C(=O)N1CCN(C(=O)N(Cc2ccc(-c3nnc(C(F)(F)F)o3)cc2)c2ccccc2)CC1] | 36 | 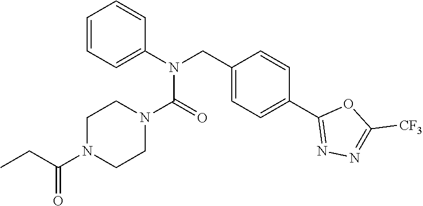 [SMILES: CCC(=O)N1CCN(C(=O)N(Cc2ccc(-c3nnc(C(F)(F)F)o3)cc2)c2ccccc2)CC1] |
| 37 | 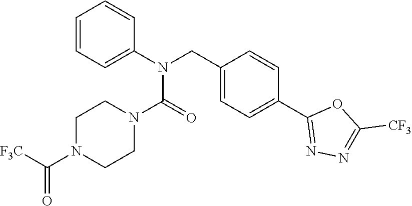 [SMILES: O=C(N1CCN(C(=O)C(F)(F)F)CC1)N(Cc1ccc(-c2nnc(C(F)(F)F)o2)cc1)c1ccccc1] | 38 | 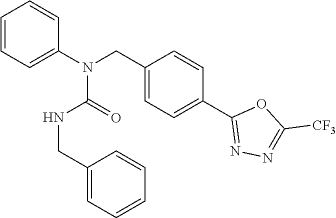 [SMILES: O=C(NCc1ccccc1)N(Cc1ccc(-c2nnc(C(F)(F)F)o2)cc1)c1ccccc1] |
| 39 | 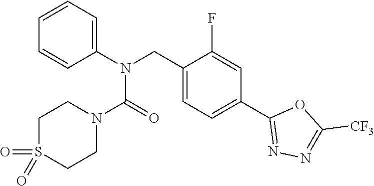 [SMILES: O=C(N1CCS(=O)(=O)CC1)N(Cc1ccc(-c2nnc(C(F)(F)F)o2)cc1F)c1ccccc1] | 40 | 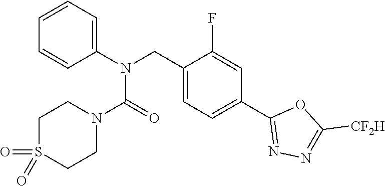 [SMILES: O=C(N1CCS(=O)(=O)CC1)N(Cc1ccc(-c2nnc(C(F)F)o2)cc1F)c1ccccc1] |
| 41 | 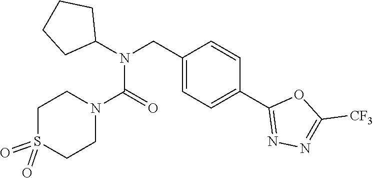 [SMILES: O=C(N1CCS(=O)(=O)CC1)N(Cc1ccc(-c2nnc(C(F)(F)F)o2)cc1)C1CCCC1] | 42 | 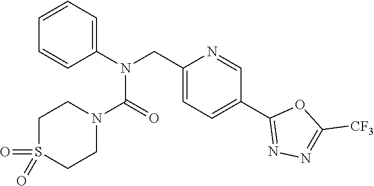 [SMILES: O=C(N1CCS(=O)(=O)CC1)N(Cc1ccc(-c2nnc(C(F)(F)F)o2)cn1)c1ccccc1] |
| 43 | 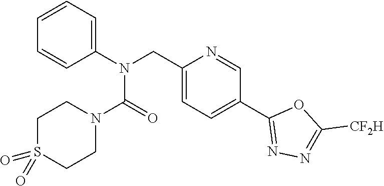 [SMILES: O=C(N1CCS(=O)(=O)CC1)N(Cc1ccc(-c2nnc(C(F)F)o2)cn1)c1ccccc1] | 44 | 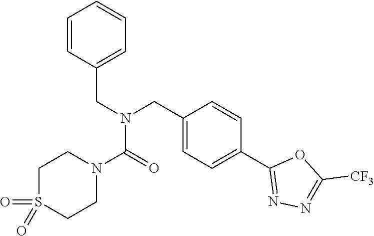 [SMILES: O=C(N1CCS(=O)(=O)CC1)N(Cc1ccccc1)Cc1ccc(-c2nnc(C(F)(F)F)o2)cc1] |
| 45 | 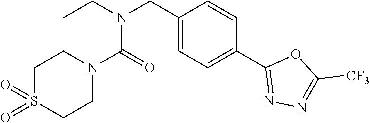 [SMILES: CCN(Cc1ccc(-c2nnc(C(F)(F)F)o2)cc1)C(=O)N1CCS(=O)(=O)CC1] | 46 | 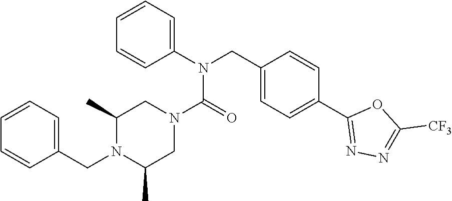 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 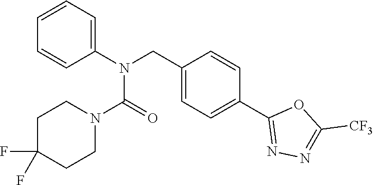 [SMILES: O=C(N1CCC(F)(F)CC1)N(Cc1ccc(-c2nnc(C(F)(F)F)o2)cc1)c1ccccc1] | 48 | 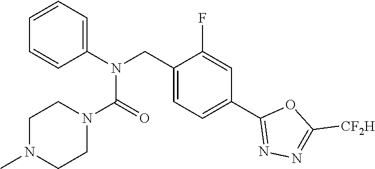 [SMILES: CN1CCN(C(=O)N(Cc2ccc(-c3nnc(C(F)F)o3)cc2F)c2ccccc2)CC1] |
| 49 | 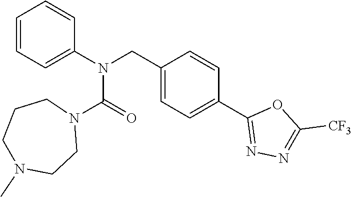 [SMILES: CN1CCCN(C(=O)N(Cc2ccc(-c3nnc(C(F)(F)F)o3)cc2)c2ccccc2)CC1] | 50 | 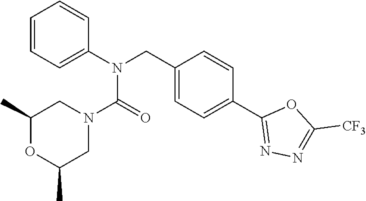 [SMILES: C[C@@H]1CN(C(=O)N(Cc2ccc(-c3nnc(C(F)(F)F)o3)cc2)c2ccccc2)C[C@H](C)O1] |
| 51 | 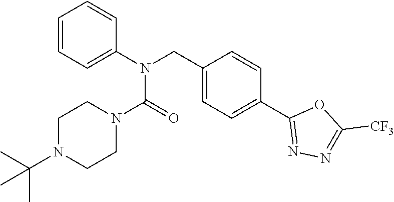 [SMILES: CC(C)(C)N1CCN(C(=O)N(Cc2ccc(-c3nnc(C(F)(F)F)o3)cc2)c2ccccc2)CC1] | 52 | 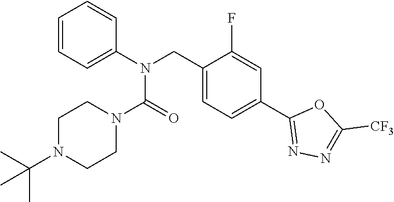 [SMILES: CC(C)(C)N1CCN(C(=O)N(Cc2ccc(-c3nnc(C(F)(F)F)o3)cc2F)c2ccccc2)CC1] |
| 53 | 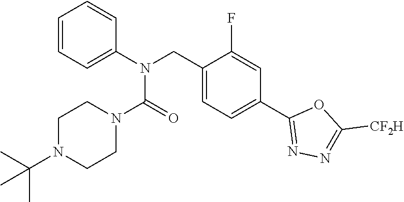 [SMILES: CC(C)(C)N1CCN(C(=O)N(Cc2ccc(-c3nnc(C(F)F)o3)cc2F)c2ccccc2)CC1] | 54 | 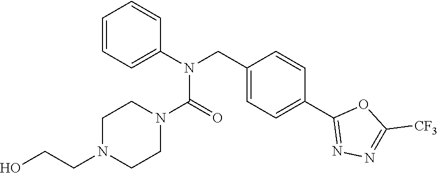 [SMILES: O=C(N1CCN(CCO)CC1)N(Cc1ccc(-c2nnc(C(F)(F)F)o2)cc1)c1ccccc1] |
| 55 | 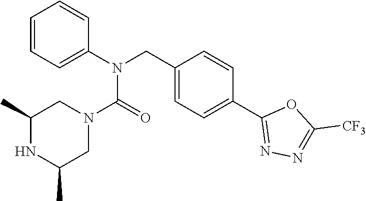 [SMILES: C[C@@H]1CN(C(=O)N(Cc2ccc(-c3nnc(C(F)(F)F)o3)cc2)c2ccccc2)C[C@H](C)N1] | 56 | 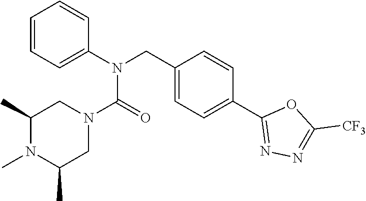 [SMILES: C[C@@H]1CN(C(=O)N(Cc2ccc(-c3nnc(C(F)(F)F)o3)cc2)c2ccccc2)C[C@H](C)N1C] |
| 57 | 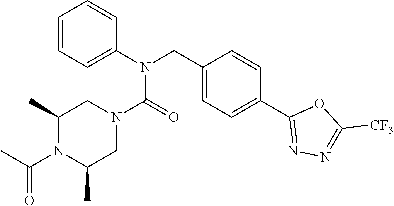 [SMILES: CC(=O)N1[C@H](C)CN(C(=O)N(Cc2ccc(-c3nnc(C(F)(F)F)o3)cc2)c2ccccc2)C[C@@H]1C] | 58 | 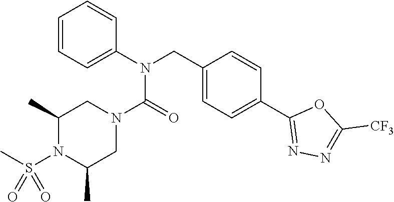 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 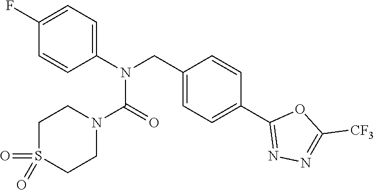 [SMILES: O=C(N1CCS(=O)(=O)CC1)N(Cc1ccc(-c2nnc(C(F)(F)F)o2)cc1)c1ccc(F)cc1] | 60 | 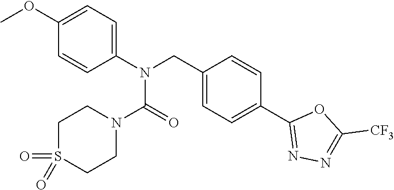 [SMILES: COc1ccc(N(Cc2ccc(-c3nnc(C(F)(F)F)o3)cc2)C(=O)N2CCS(=O)(=O)CC2)cc1] |
| 61 | 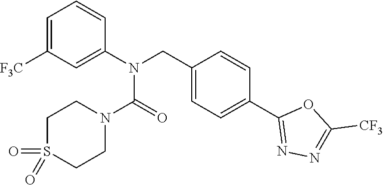 [SMILES: O=C(N1CCS(=O)(=O)CC1)N(Cc1ccc(-c2nnc(C(F)(F)F)o2)cc1)c1cccc(C(F)(F)F)c1] | 62 | 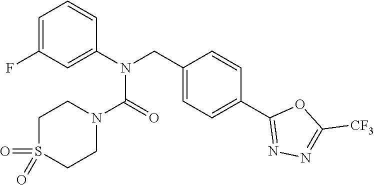 [SMILES: O=C(N1CCS(=O)(=O)CC1)N(Cc1ccc(-c2nnc(C(F)(F)F)o2)cc1)c1cccc(F)c1] |
| 63 | 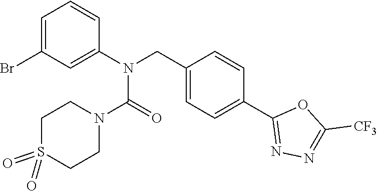 [SMILES: O=C(N1CCS(=O)(=O)CC1)N(Cc1ccc(-c2nnc(C(F)(F)F)o2)cc1)c1cccc(Br)c1] | 64 | 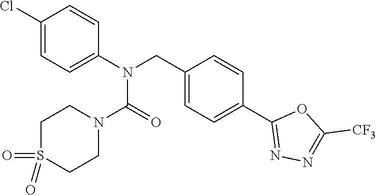 [SMILES: O=C(N1CCS(=O)(=O)CC1)N(Cc1ccc(-c2nnc(C(F)(F)F)o2)cc1)c1ccc(Cl)cc1] |
| 65 | 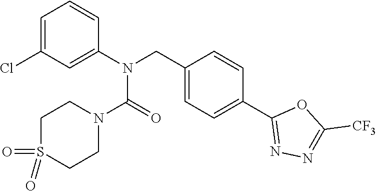 [SMILES: O=C(N1CCS(=O)(=O)CC1)N(Cc1ccc(-c2nnc(C(F)(F)F)o2)cc1)c1cccc(Cl)c1] | 66 | 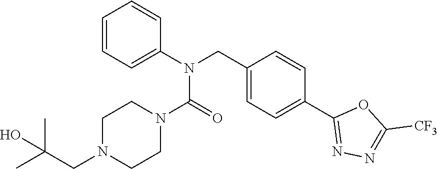 [SMILES: CC(C)(O)CN1CCN(C(=O)N(Cc2ccc(-c3nnc(C(F)(F)F)o3)cc2)c2ccccc2)CC1] |
| 67 | 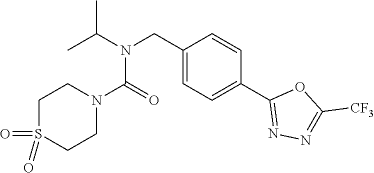 [SMILES: CC(C)N(Cc1ccc(-c2nnc(C(F)(F)F)o2)cc1)C(=O)N1CCS(=O)(=O)CC1] | 68 | 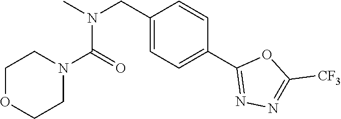 [SMILES: CN(Cc1ccc(-c2nnc(C(F)(F)F)o2)cc1)C(=O)N1CCOCC1] |
| 69 | 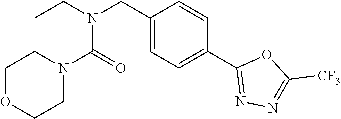 [SMILES: CCN(Cc1ccc(-c2nnc(C(F)(F)F)o2)cc1)C(=O)N1CCOCC1] | 70 | 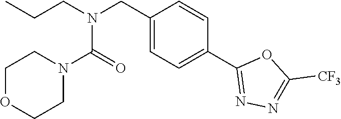 [SMILES: CCCN(Cc1ccc(-c2nnc(C(F)(F)F)o2)cc1)C(=O)N1CCOCC1] |
| 71 | 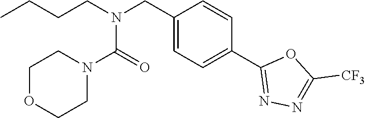 [SMILES: CCCCN(Cc1ccc(-c2nnc(C(F)(F)F)o2)cc1)C(=O)N1CCOCC1] | 72 | 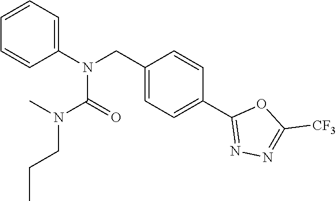 [SMILES: CCCN(C)C(=O)N(Cc1ccc(-c2nnc(C(F)(F)F)o2)cc1)c1ccccc1] |
| 73 | 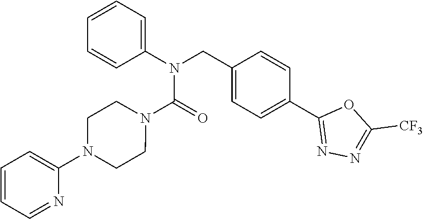 [SMILES: O=C(N1CCN(c2ccccn2)CC1)N(Cc1ccc(-c2nnc(C(F)(F)F)o2)cc1)c1ccccc1] | 74 | 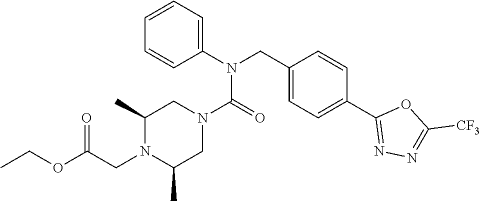 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 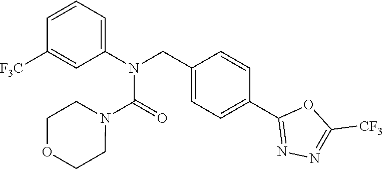 [SMILES: O=C(N1CCOCC1)N(Cc1ccc(-c2nnc(C(F)(F)F)o2)cc1)c1cccc(C(F)(F)F)c1] | 76 | 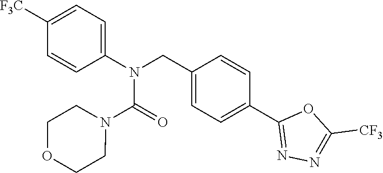 [SMILES: O=C(N1CCOCC1)N(Cc1ccc(-c2nnc(C(F)(F)F)o2)cc1)c1ccc(C(F)(F)F)cc1] |
| 77 | 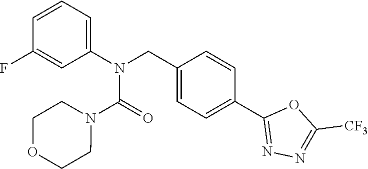 [SMILES: O=C(N1CCOCC1)N(Cc1ccc(-c2nnc(C(F)(F)F)o2)cc1)c1cccc(F)c1] | 78 | 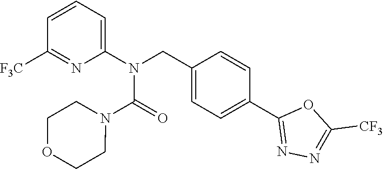 [SMILES: O=C(N1CCOCC1)N(Cc1ccc(-c2nnc(C(F)(F)F)o2)cc1)c1cccc(C(F)(F)F)n1] |
| 79 | 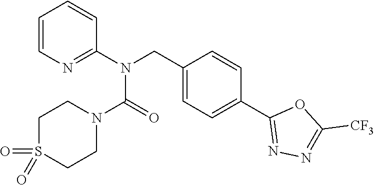 [SMILES: O=C(N1CCS(=O)(=O)CC1)N(Cc1ccc(-c2nnc(C(F)(F)F)o2)cc1)c1ccccn1] | 80 | 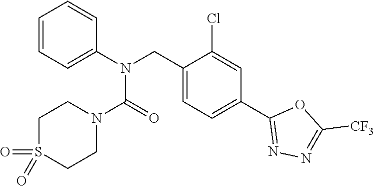 [SMILES: O=C(N1CCS(=O)(=O)CC1)N(Cc1ccc(-c2nnc(C(F)(F)F)o2)cc1Cl)c1ccccc1] |
| 81 | 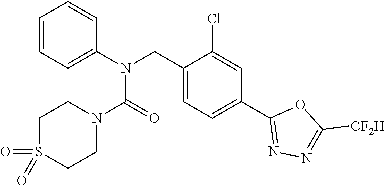 [SMILES: O=C(N1CCS(=O)(=O)CC1)N(Cc1ccc(-c2nnc(C(F)F)o2)cc1Cl)c1ccccc1] | 82 | 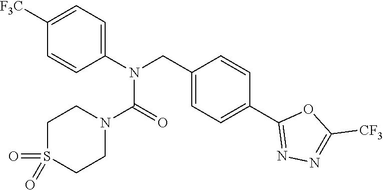 [SMILES: O=C(N1CCS(=O)(=O)CC1)N(Cc1ccc(-c2nnc(C(F)(F)F)o2)cc1)c1ccc(C(F)(F)F)cc1] |
| 83 | 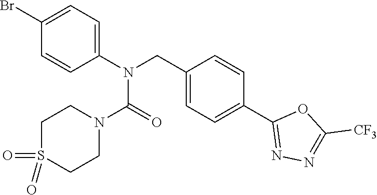 [SMILES: O=C(N1CCS(=O)(=O)CC1)N(Cc1ccc(-c2nnc(C(F)(F)F)o2)cc1)c1ccc(Br)cc1] | 84 | 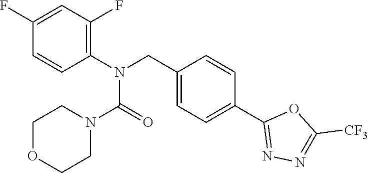 [SMILES: O=C(N1CCOCC1)N(Cc1ccc(-c2nnc(C(F)(F)F)o2)cc1)c1ccc(F)cc1F] |
| 85 | 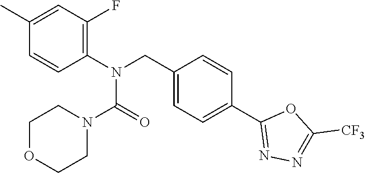 [SMILES: Cc1ccc(N(Cc2ccc(-c3nnc(C(F)(F)F)o3)cc2)C(=O)N2CCOCC2)c(F)c1] | 86 | 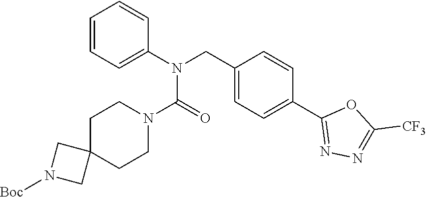 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 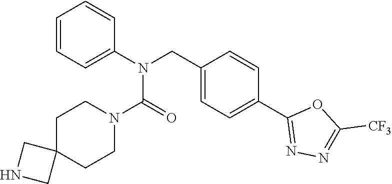 [SMILES: O=C(N1CCC2(CC1)CNC2)N(Cc1ccc(-c2nnc(C(F)(F)F)o2)cc1)c1ccccc1] | 88 | 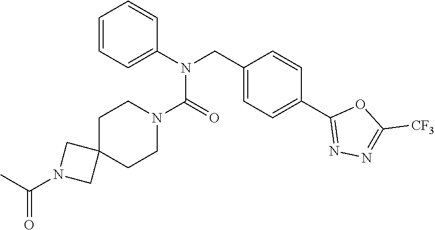 [SMILES: CC(=O)N1CC2(CCN(C(=O)N(Cc3ccc(-c4nnc(C(F)(F)F)o4)cc3)c3ccccc3)CC2)C1] |
| 89 | 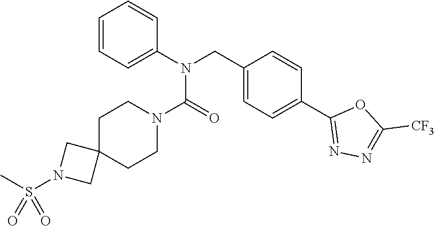 [SMILES: CS(=O)(=O)N1CC2(CCN(C(=O)N(Cc3ccc(-c4nnc(C(F)(F)F)o4)cc3)c3ccccc3)CC2)C1] | 90 | 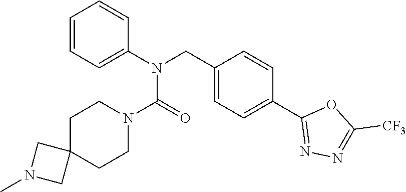 [SMILES: CN1CC2(CCN(C(=O)N(Cc3ccc(-c4nnc(C(F)(F)F)o4)cc3)c3ccccc3)CC2)C1] |
| 91 | 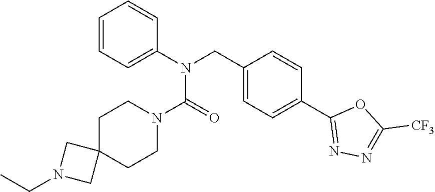 [SMILES: CCN1CC2(CCN(C(=O)N(Cc3ccc(-c4nnc(C(F)(F)F)o4)cc3)c3ccccc3)CC2)C1] | 92 | 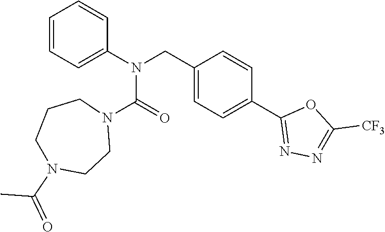 [SMILES: CC(=O)N1CCCN(C(=O)N(Cc2ccc(-c3nnc(C(F)(F)F)o3)cc2)c2ccccc2)CC1] |
| 93 | 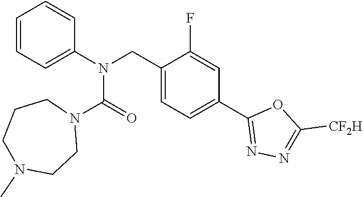 [SMILES: CN1CCCN(C(=O)N(Cc2ccc(-c3nnc(C(F)F)o3)cc2F)c2ccccc2)CC1] | 94 | 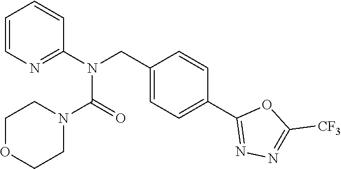 [SMILES: O=C(N1CCOCC1)N(Cc1ccc(-c2nnc(C(F)(F)F)o2)cc1)c1ccccn1] |
| 95 | 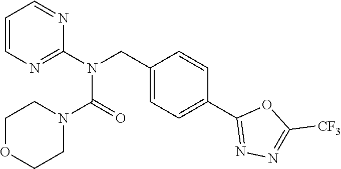 [SMILES: O=C(N1CCOCC1)N(Cc1ccc(-c2nnc(C(F)(F)F)o2)cc1)c1ncccn1] | 96 | 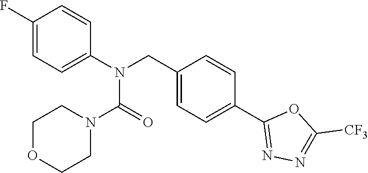 [SMILES: O=C(N1CCOCC1)N(Cc1ccc(-c2nnc(C(F)(F)F)o2)cc1)c1ccc(F)cc1] |
| 97 | 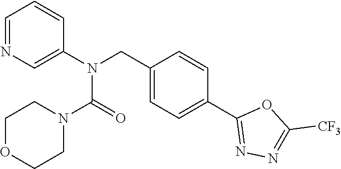 [SMILES: O=C(N1CCOCC1)N(Cc1ccc(-c2nnc(C(F)(F)F)o2)cc1)c1cccnc1] | 98 | 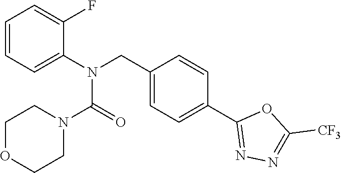 [SMILES: O=C(N1CCOCC1)N(Cc1ccc(-c2nnc(C(F)(F)F)o2)cc1)c1ccccc1F] |
| 99 | 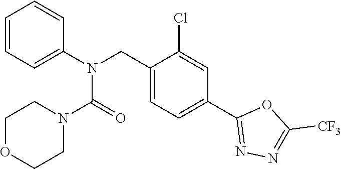 [SMILES: O=C(N1CCOCC1)N(Cc1ccc(-c2nnc(C(F)(F)F)o2)cc1Cl)c1ccccc1] | 100 | 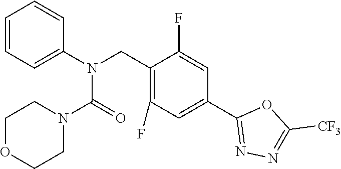 [SMILES: O=C(N1CCOCC1)N(Cc1c(F)cc(-c2nnc(C(F)(F)F)o2)cc1F)c1ccccc1] |
| 101 | 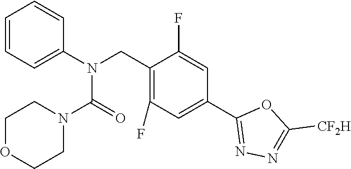 [SMILES: O=C(N1CCOCC1)N(Cc1c(F)cc(-c2nnc(C(F)F)o2)cc1F)c1ccccc1] | 102 | 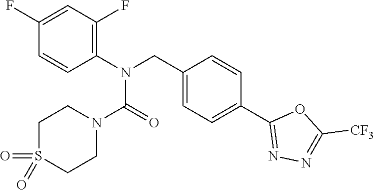 [SMILES: O=C(N1CCS(=O)(=O)CC1)N(Cc1ccc(-c2nnc(C(F)(F)F)o2)cc1)c1ccc(F)cc1F] |
| 103 | 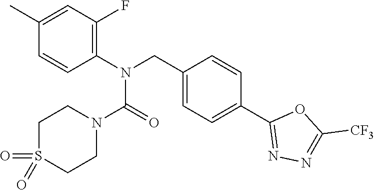 [SMILES: Cc1ccc(N(Cc2ccc(-c3nnc(C(F)(F)F)o3)cc2)C(=O)N2CCS(=O)(=O)CC2)c(F)c1] | 104 | 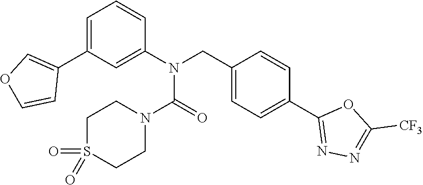 [SMILES: O=C(N1CCS(=O)(=O)CC1)N(Cc1ccc(-c2nnc(C(F)(F)F)o2)cc1)c1cccc(-c2ccoc2)c1] |
| 105 | 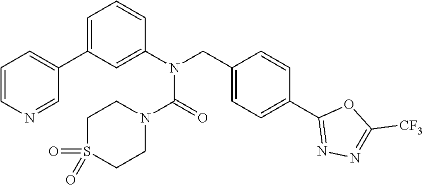 [SMILES: O=C(N1CCS(=O)(=O)CC1)N(Cc1ccc(-c2nnc(C(F)(F)F)o2)cc1)c1cccc(-c2cccnc2)c1] | 106 | 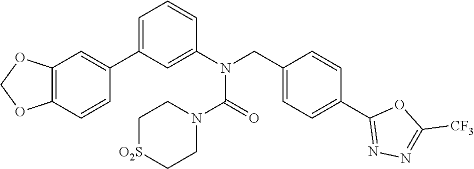 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 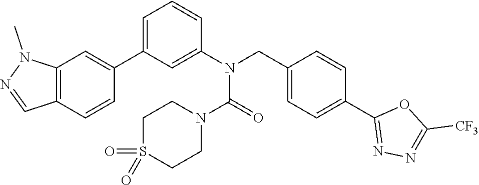 [SMILES: Cn1ncc2ccc(-c3cccc(N(Cc4ccc(-c5nnc(C(F)(F)F)o5)cc4)C(=O)N4CCS(=O)(=O)CC4)c3)cc21] | 108 | 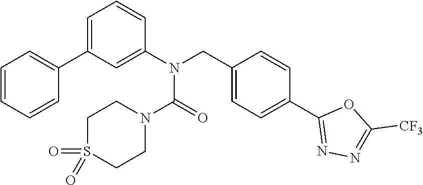 [SMILES: O=C(N1CCS(=O)(=O)CC1)N(Cc1ccc(-c2nnc(C(F)(F)F)o2)cc1)c1cccc(-c2ccccc2)c1] |
| 109 | 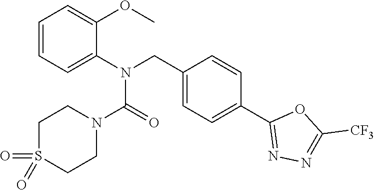 [SMILES: COc1ccccc1N(Cc1ccc(-c2nnc(C(F)(F)F)o2)cc1)C(=O)N1CCS(=O)(=O)CC1] | 110 | 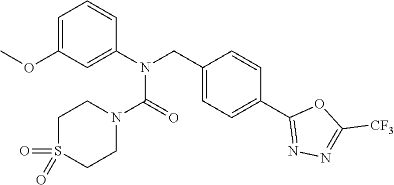 [SMILES: COc1cccc(N(Cc2ccc(-c3nnc(C(F)(F)F)o3)cc2)C(=O)N2CCS(=O)(=O)CC2)c1] |
| 111 | 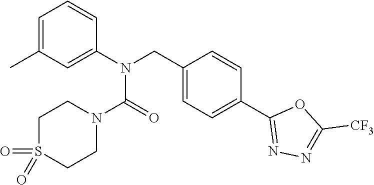 [SMILES: Cc1cccc(N(Cc2ccc(-c3nnc(C(F)(F)F)o3)cc2)C(=O)N2CCS(=O)(=O)CC2)c1] | 112 | 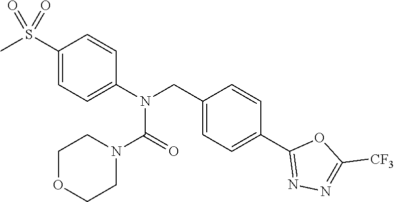 [SMILES: CS(=O)(=O)c1ccc(N(Cc2ccc(-c3nnc(C(F)(F)F)o3)cc2)C(=O)N2CCOCC2)cc1] |
| 113 | 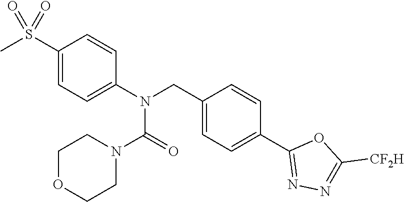 [SMILES: CS(=O)(=O)c1ccc(N(Cc2ccc(-c3nnc(C(F)F)o3)cc2)C(=O)N2CCOCC2)cc1] | 114 | 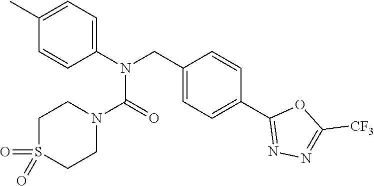 [SMILES: Cc1ccc(N(Cc2ccc(-c3nnc(C(F)(F)F)o3)cc2)C(=O)N2CCS(=O)(=O)CC2)cc1] |
| 115 | 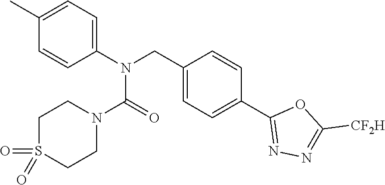 [SMILES: Cc1ccc(N(Cc2ccc(-c3nnc(C(F)F)o3)cc2)C(=O)N2CCS(=O)(=O)CC2)cc1] | 116 | 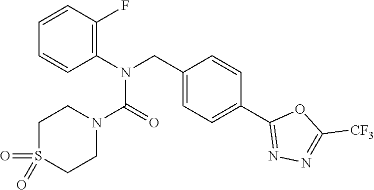 [SMILES: O=C(N1CCS(=O)(=O)CC1)N(Cc1ccc(-c2nnc(C(F)(F)F)o2)cc1)c1ccccc1F] |
| 117 | 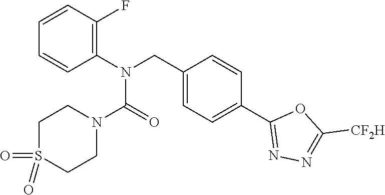 [SMILES: O=C(N1CCS(=O)(=O)CC1)N(Cc1ccc(-c2nnc(C(F)F)o2)cc1)c1ccccc1F] | 118 | 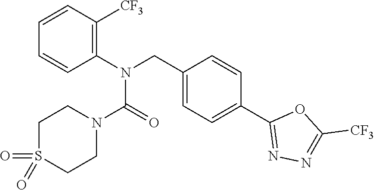 [SMILES: O=C(N1CCS(=O)(=O)CC1)N(Cc1ccc(-c2nnc(C(F)(F)F)o2)cc1)c1ccccc1C(F)(F)F] |
| 119 | 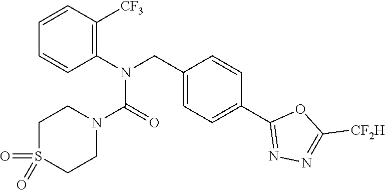 [SMILES: O=C(N1CCS(=O)(=O)CC1)N(Cc1ccc(-c2nnc(C(F)F)o2)cc1)c1ccccc1C(F)(F)F] | 120 | 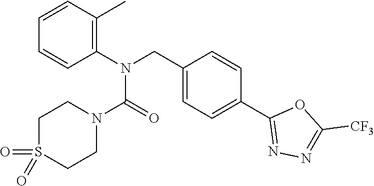 [SMILES: Cc1ccccc1N(Cc1ccc(-c2nnc(C(F)(F)F)o2)cc1)C(=O)N1CCS(=O)(=O)CC1] |
| 121 | 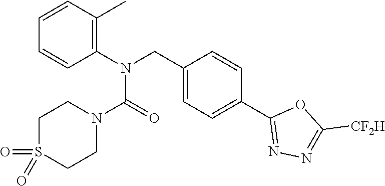 [SMILES: Cc1ccccc1N(Cc1ccc(-c2nnc(C(F)F)o2)cc1)C(=O)N1CCS(=O)(=O)CC1] | 122 | 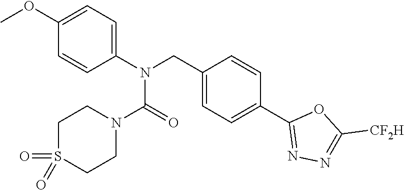 [SMILES: COc1ccc(N(Cc2ccc(-c3nnc(C(F)F)o3)cc2)C(=O)N2CCS(=O)(=O)CC2)cc1] |
| 123 | 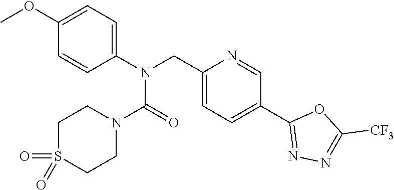 [SMILES: COc1ccc(N(Cc2ccc(-c3nnc(C(F)(F)F)o3)cn2)C(=O)N2CCS(=O)(=O)CC2)cc1] | 124 | 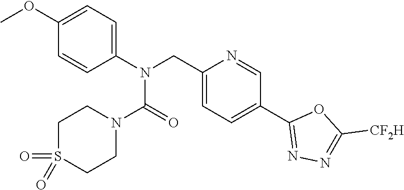 [SMILES: COc1ccc(N(Cc2ccc(-c3nnc(C(F)F)o3)cn2)C(=O)N2CCS(=O)(=O)CC2)cc1] |
| 125 | 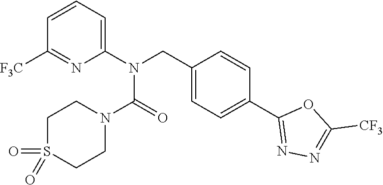 [SMILES: O=C(N1CCS(=O)(=O)CC1)N(Cc1ccc(-c2nnc(C(F)(F)F)o2)cc1)c1cccc(C(F)(F)F)n1] | 126 | 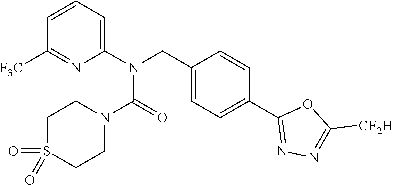 [SMILES: O=C(N1CCS(=O)(=O)CC1)N(Cc1ccc(-c2nnc(C(F)F)o2)cc1)c1cccc(C(F)(F)F)n1] |
| 127 | 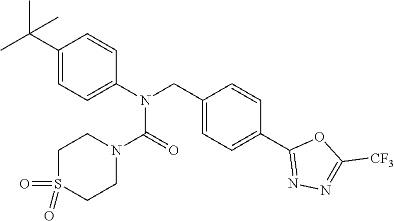 [SMILES: CC(C)(C)c1ccc(N(Cc2ccc(-c3nnc(C(F)(F)F)o3)cc2)C(=O)N2CCS(=O)(=O)CC2)cc1] | 128 | 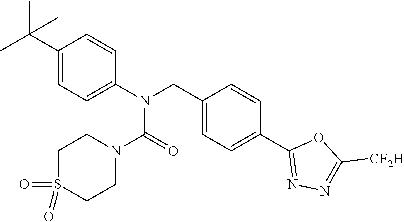 [SMILES: CC(C)(C)c1ccc(N(Cc2ccc(-c3nnc(C(F)F)o3)cc2)C(=O)N2CCS(=O)(=O)CC2)cc1] |
| 129 | 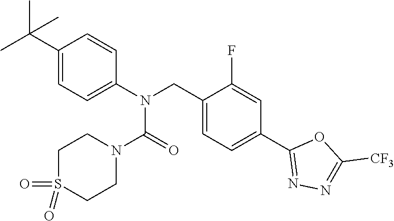 [SMILES: CC(C)(C)c1ccc(N(Cc2ccc(-c3nnc(C(F)(F)F)o3)cc2F)C(=O)N2CCS(=O)(=O)CC2)cc1] | 130 | 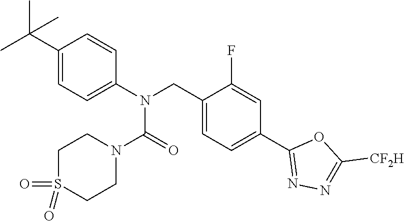 [SMILES: CC(C)(C)c1ccc(N(Cc2ccc(-c3nnc(C(F)F)o3)cc2F)C(=O)N2CCS(=O)(=O)CC2)cc1] |
| 131 | 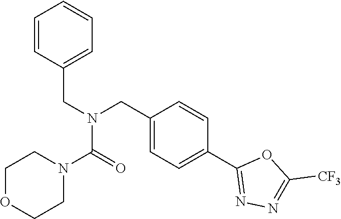 [SMILES: O=C(N1CCOCC1)N(Cc1ccccc1)Cc1ccc(-c2nnc(C(F)(F)F)o2)cc1] | 132 | 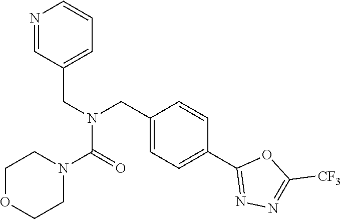 [SMILES: O=C(N1CCOCC1)N(Cc1ccc(-c2nnc(C(F)(F)F)o2)cc1)Cc1cccnc1] |
| 133 | 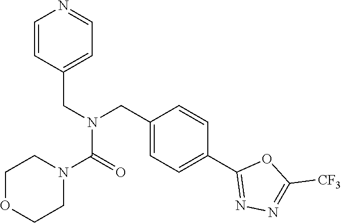 [SMILES: O=C(N1CCOCC1)N(Cc1ccncc1)Cc1ccc(-c2nnc(C(F)(F)F)o2)cc1] | 134 | 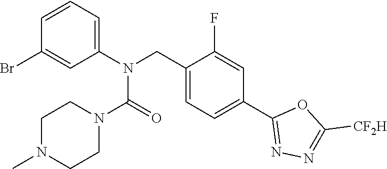 [SMILES: CN1CCN(C(=O)N(Cc2ccc(-c3nnc(C(F)F)o3)cc2F)c2cccc(Br)c2)CC1] |
| 135 | 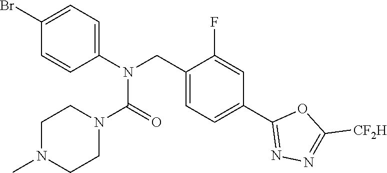 [SMILES: CN1CCN(C(=O)N(Cc2ccc(-c3nnc(C(F)F)o3)cc2F)c2ccc(Br)cc2)CC1] | 136 | 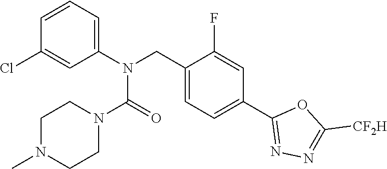 [SMILES: CN1CCN(C(=O)N(Cc2ccc(-c3nnc(C(F)F)o3)cc2F)c2cccc(Cl)c2)CC1] |
| 137 | 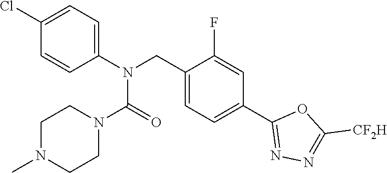 [SMILES: CN1CCN(C(=O)N(Cc2ccc(-c3nnc(C(F)F)o3)cc2F)c2ccc(Cl)cc2)CC1] | 138 | 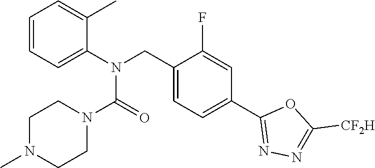 [SMILES: Cc1ccccc1N(Cc1ccc(-c2nnc(C(F)F)o2)cc1F)C(=O)N1CCN(C)CC1] |
| 139 | 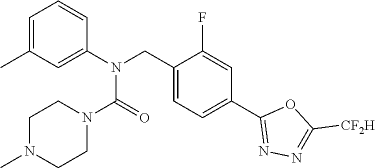 [SMILES: Cc1cccc(N(Cc2ccc(-c3nnc(C(F)F)o3)cc2F)C(=O)N2CCN(C)CC2)c1] | 140 | 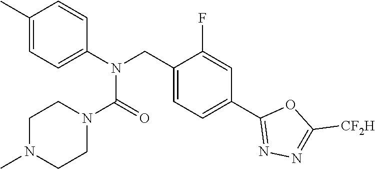 [SMILES: Cc1ccc(N(Cc2ccc(-c3nnc(C(F)F)o3)cc2F)C(=O)N2CCN(C)CC2)cc1] |
| 141 | 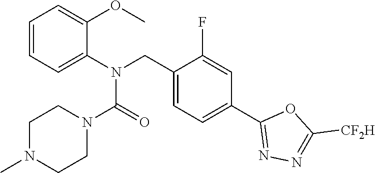 [SMILES: COc1ccccc1N(Cc1ccc(-c2nnc(C(F)F)o2)cc1F)C(=O)N1CCN(C)CC1] | 142 | 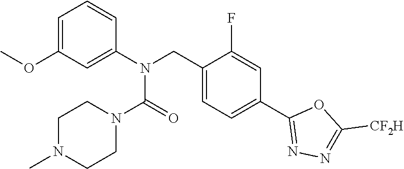 [SMILES: COc1cccc(N(Cc2ccc(-c3nnc(C(F)F)o3)cc2F)C(=O)N2CCN(C)CC2)c1] |
| 143 | 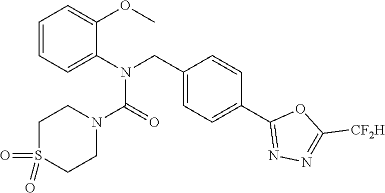 [SMILES: COc1ccccc1N(Cc1ccc(-c2nnc(C(F)F)o2)cc1)C(=O)N1CCS(=O)(=O)CC1] | 144 | 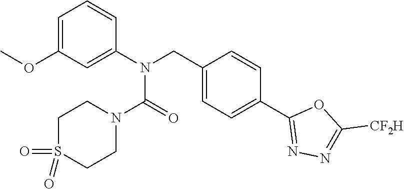 [SMILES: COc1cccc(N(Cc2ccc(-c3nnc(C(F)F)o3)cc2)C(=O)N2CCS(=O)(=O)CC2)c1] |
| 145 | 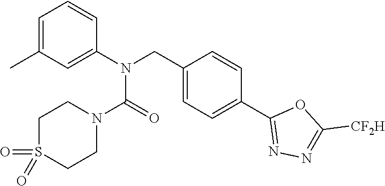 [SMILES: Cc1cccc(N(Cc2ccc(-c3nnc(C(F)F)o3)cc2)C(=O)N2CCS(=O)(=O)CC2)c1] | 146 | 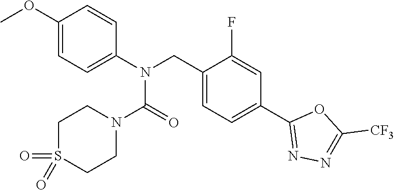 [SMILES: COc1ccc(N(Cc2ccc(-c3nnc(C(F)(F)F)o3)cc2F)C(=O)N2CCS(=O)(=O)CC2)cc1] |
| 147 | 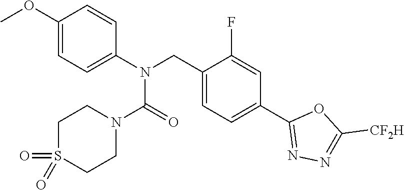 [SMILES: COc1ccc(N(Cc2ccc(-c3nnc(C(F)F)o3)cc2F)C(=O)N2CCS(=O)(=O)CC2)cc1] | 148 | 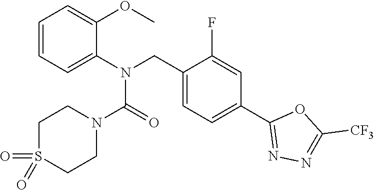 [SMILES: COc1ccccc1N(Cc1ccc(-c2nnc(C(F)(F)F)o2)cc1F)C(=O)N1CCS(=O)(=O)CC1] |
| 149 | 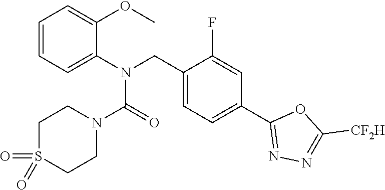 [SMILES: COc1ccccc1N(Cc1ccc(-c2nnc(C(F)F)o2)cc1F)C(=O)N1CCS(=O)(=O)CC1] | 150 | 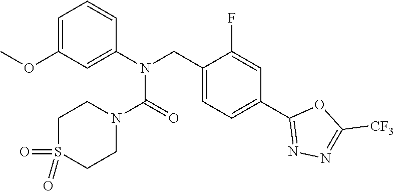 [SMILES: COc1cccc(N(Cc2ccc(-c3nnc(C(F)(F)F)o3)cc2F)C(=O)N2CCS(=O)(=O)CC2)c1] |
| 151 | 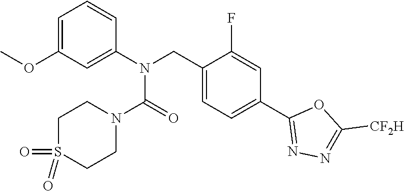 [SMILES: COc1cccc(N(Cc2ccc(-c3nnc(C(F)F)o3)cc2F)C(=O)N2CCS(=O)(=O)CC2)c1] | 152 | 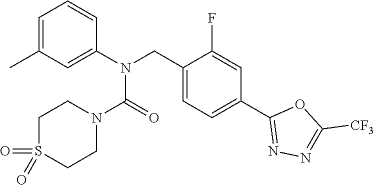 [SMILES: Cc1cccc(N(Cc2ccc(-c3nnc(C(F)(F)F)o3)cc2F)C(=O)N2CCS(=O)(=O)CC2)c1] |
| 153 | 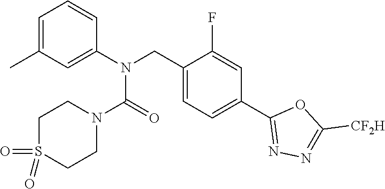 [SMILES: Cc1cccc(N(Cc2ccc(-c3nnc(C(F)F)o3)cc2F)C(=O)N2CCS(=O)(=O)CC2)c1] | 154 | 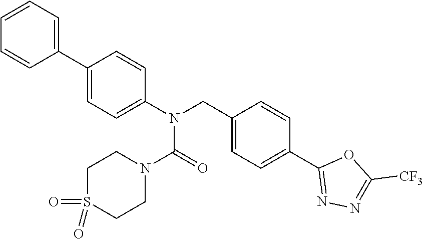 [SMILES: O=C(N1CCS(=O)(=O)CC1)N(Cc1ccc(-c2nnc(C(F)(F)F)o2)cc1)c1ccc(-c2ccccc2)cc1] |
| 155 | 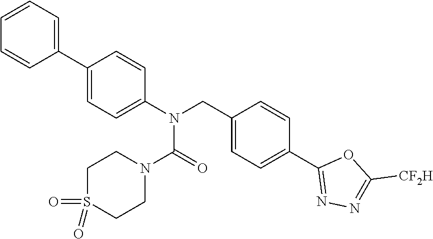 [SMILES: O=C(N1CCS(=O)(=O)CC1)N(Cc1ccc(-c2nnc(C(F)F)o2)cc1)c1ccc(-c2ccccc2)cc1] | 156 | 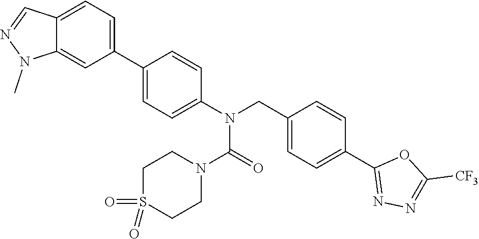 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 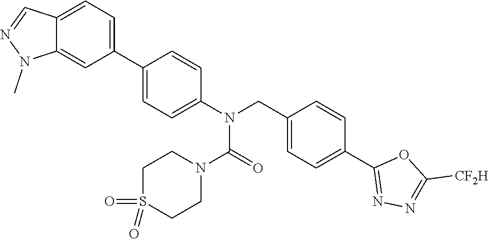 [SMILES: Cn1ncc2ccc(-c3ccc(N(Cc4ccc(-c5nnc(C(F)F)o5)cc4)C(=O)N4CCS(=O)(=O)CC4)cc3)cc21] | 158 | 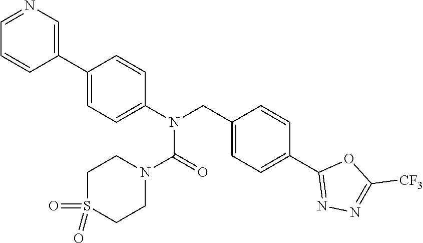 [SMILES: O=C(N1CCS(=O)(=O)CC1)N(Cc1ccc(-c2nnc(C(F)(F)F)o2)cc1)c1ccc(-c2cccnc2)cc1] |
| 159 | 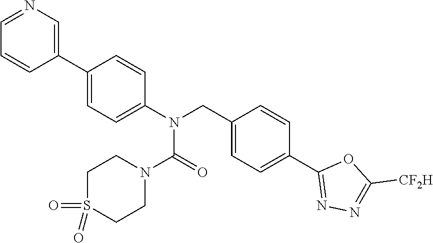 [SMILES: O=C(N1CCS(=O)(=O)CC1)N(Cc1ccc(-c2nnc(C(F)F)o2)cc1)c1ccc(-c2cccnc2)cc1] | 160 | 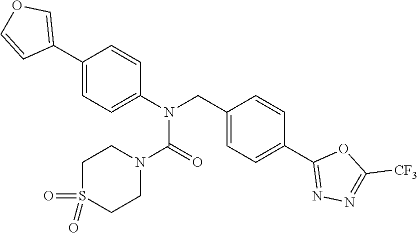 [SMILES: O=C(N1CCS(=O)(=O)CC1)N(Cc1ccc(-c2nnc(C(F)(F)F)o2)cc1)c1ccc(-c2ccoc2)cc1] |
| 161 | 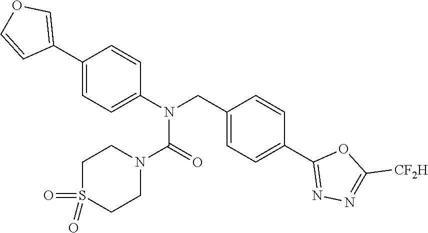 [SMILES: O=C(N1CCS(=O)(=O)CC1)N(Cc1ccc(-c2nnc(C(F)F)o2)cc1)c1ccc(-c2ccoc2)cc1] | 162 | 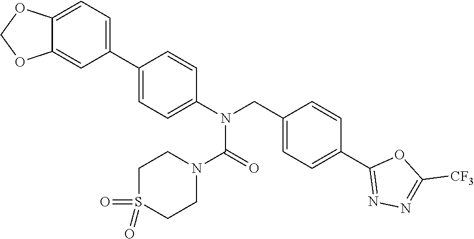 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 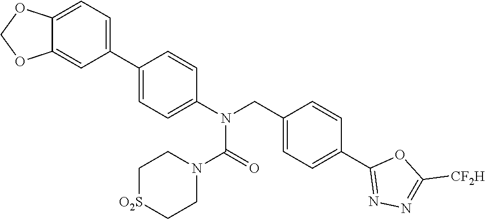 [SMILES: O=C(N1CCS(=O)(=O)CC1)N(Cc1ccc(-c2nnc(C(F)F)o2)cc1)c1ccc(-c2ccc3c(c2)OCO3)cc1] | 164 | 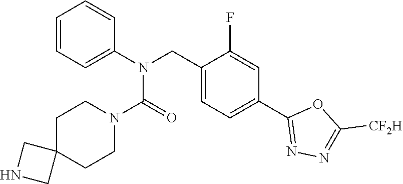 [SMILES: O=C(N1CCC2(CC1)CNC2)N(Cc1ccc(-c2nnc(C(F)F)o2)cc1F)c1ccccc1] |
| 165 | 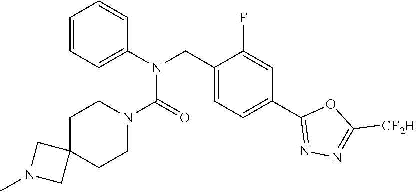 [SMILES: CN1CC2(CCN(C(=O)N(Cc3ccc(-c4nnc(C(F)F)o4)cc3F)c3ccccc3)CC2)C1] | 166 | 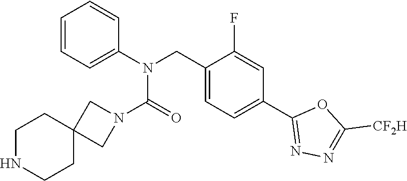 [SMILES: O=C(N1CC2(CCNCC2)C1)N(Cc1ccc(-c2nnc(C(F)F)o2)cc1F)c1ccccc1] |
| 167 | 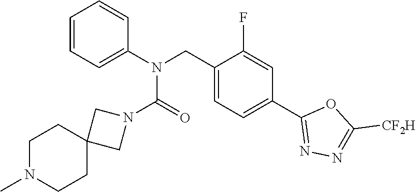 [SMILES: CN1CCC2(CC1)CN(C(=O)N(Cc1ccc(-c3nnc(C(F)F)o3)cc1F)c1ccccc1)C2] | 168 | 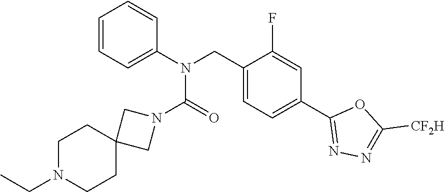 [SMILES: CCN1CCC2(CC1)CN(C(=O)N(Cc1ccc(-c3nnc(C(F)F)o3)cc1F)c1ccccc1)C2] |
| 169 | 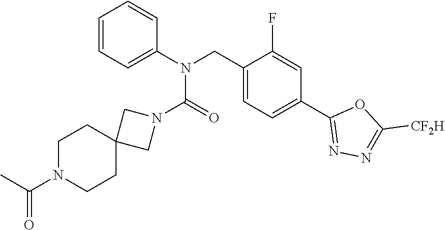 [SMILES: CC(=O)N1CCC2(CC1)CN(C(=O)N(Cc1ccc(-c3nnc(C(F)F)o3)cc1F)c1ccccc1)C2] | 170 | 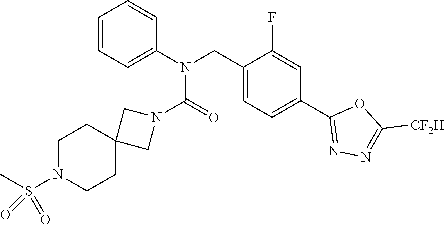 [SMILES: CS(=O)(=O)N1CCC2(CC1)CN(C(=O)N(Cc1ccc(-c3nnc(C(F)F)o3)cc1F)c1ccccc1)C2] |
| 171 | 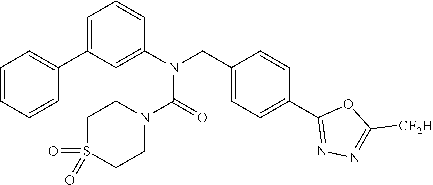 [SMILES: O=C(N1CCS(=O)(=O)CC1)N(Cc1ccc(-c2nnc(C(F)F)o2)cc1)c1cccc(-c2ccccc2)c1] | 172 | 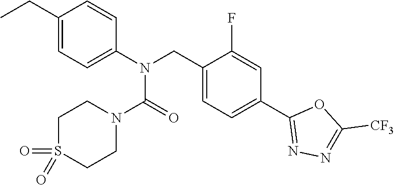 [SMILES: CCc1ccc(N(Cc2ccc(-c3nnc(C(F)(F)F)o3)cc2F)C(=O)N2CCS(=O)(=O)CC2)cc1] |
| 173 | 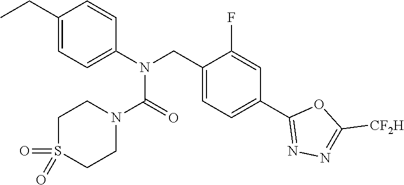 [SMILES: CCc1ccc(N(Cc2ccc(-c3nnc(C(F)F)o3)cc2F)C(=O)N2CCS(=O)(=O)CC2)cc1] | 174 | 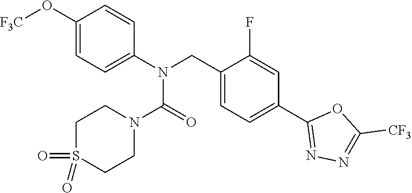 [SMILES: O=C(N1CCS(=O)(=O)CC1)N(Cc1ccc(-c2nnc(C(F)(F)F)o2)cc1F)c1ccc(OC(F)(F)F)cc1] |
| 175 | 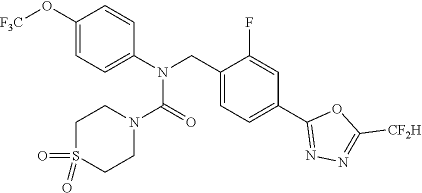 [SMILES: O=C(N1CCS(=O)(=O)CC1)N(Cc1ccc(-c2nnc(C(F)F)o2)cc1F)c1ccc(OC(F)(F)F)cc1] | 176 | 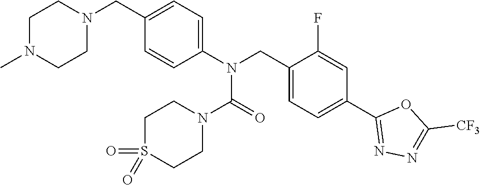 [SMILES: CN1CCN(Cc2ccc(N(Cc3ccc(-c4nnc(C(F)(F)F)o4)cc3F)C(=O)N3CCS(=O)(=O)CC3)cc2)CC1] |
| 177 | 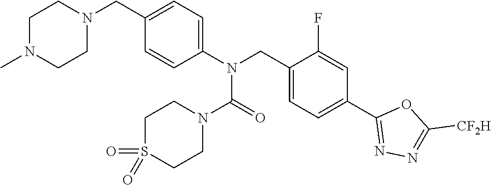 [SMILES: CN1CCN(Cc2ccc(N(Cc3ccc(-c4nnc(C(F)F)o4)cc3F)C(=O)N3CCS(=O)(=O)CC3)cc2)CC1] | 178 | 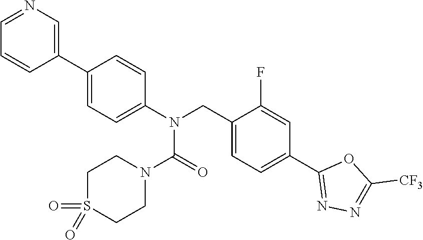 [SMILES: O=C(N1CCS(=O)(=O)CC1)N(Cc1ccc(-c2nnc(C(F)(F)F)o2)cc1F)c1ccc(-c2cccnc2)cc1] |
| 179 | 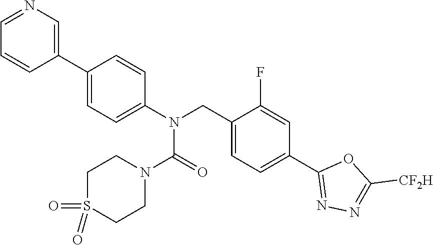 [SMILES: O=C(N1CCS(=O)(=O)CC1)N(Cc1ccc(-c2nnc(C(F)F)o2)cc1F)c1ccc(-c2cccnc2)cc1] | 180 | 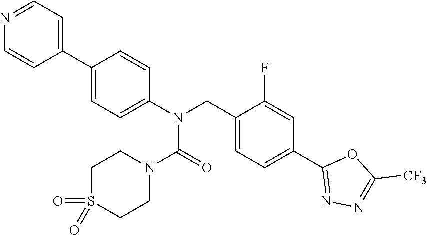 [SMILES: O=C(N1CCS(=O)(=O)CC1)N(Cc1ccc(-c2nnc(C(F)(F)F)o2)cc1F)c1ccc(-c2ccncc2)cc1] |
| 181 | 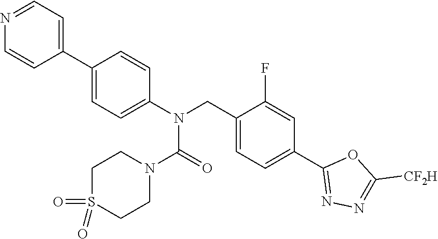 [SMILES: O=C(N1CCS(=O)(=O)CC1)N(Cc1ccc(-c2nnc(C(F)F)o2)cc1F)c1ccc(-c2ccncc2)cc1] | 182 | 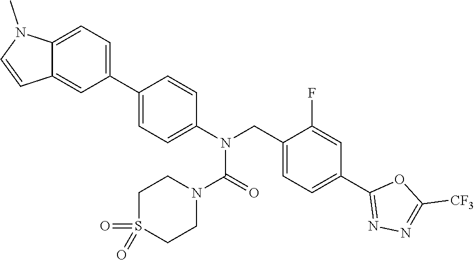 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 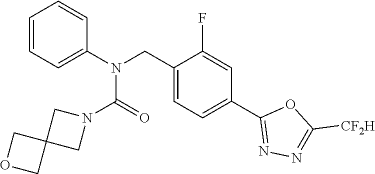 [SMILES: O=C(N1CC2(COC2)C1)N(Cc1ccc(-c2nnc(C(F)F)o2)cc1F)c1ccccc1] | 184 | 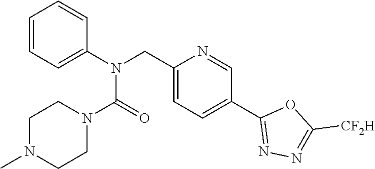 [SMILES: CN1CCN(C(=O)N(Cc2ccc(-c3nnc(C(F)F)o3)cn2)c2ccccc2)CC1] |
| 185 | 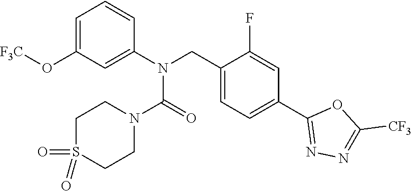 [SMILES: O=C(N1CCS(=O)(=O)CC1)N(Cc1ccc(-c2nnc(C(F)(F)F)o2)cc1F)c1cccc(OC(F)(F)F)c1] | 186 | 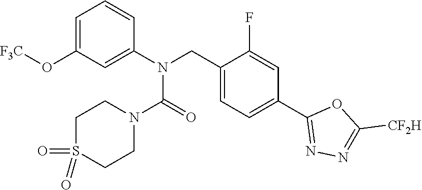 [SMILES: O=C(N1CCS(=O)(=O)CC1)N(Cc1ccc(-c2nnc(C(F)F)o2)cc1F)c1cccc(OC(F)(F)F)c1] |
| 187 | 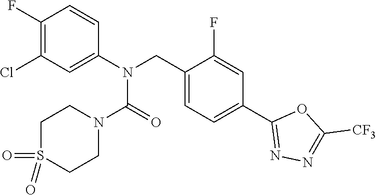 [SMILES: O=C(N1CCS(=O)(=O)CC1)N(Cc1ccc(-c2nnc(C(F)(F)F)o2)cc1F)c1ccc(F)c(Cl)c1] | 188 | 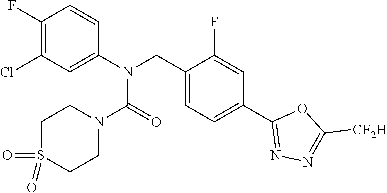 [SMILES: O=C(N1CCS(=O)(=O)CC1)N(Cc1ccc(-c2nnc(C(F)F)o2)cc1F)c1ccc(F)c(Cl)c1] |
| 189 | 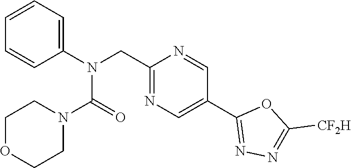 [SMILES: O=C(N1CCOCC1)N(Cc1ncc(-c2nnc(C(F)F)o2)cn1)c1ccccc1] | 190 | 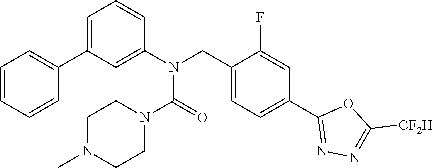 [SMILES: CN1CCN(C(=O)N(Cc2ccc(-c3nnc(C(F)F)o3)cc2F)c2cccc(-c3ccccc3)c2)CC1] |
| 191 | 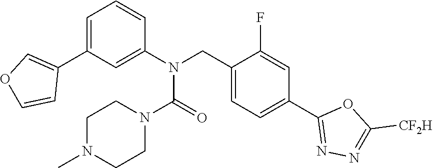 [SMILES: CN1CCN(C(=O)N(Cc2ccc(-c3nnc(C(F)F)o3)cc2F)c2cccc(-c3ccoc3)c2)CC1] | 192 | 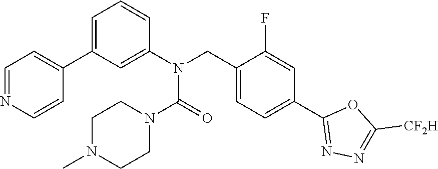 [SMILES: CN1CCN(C(=O)N(Cc2ccc(-c3nnc(C(F)F)o3)cc2F)c2cccc(-c3ccncc3)c2)CC1] |
| 193 | 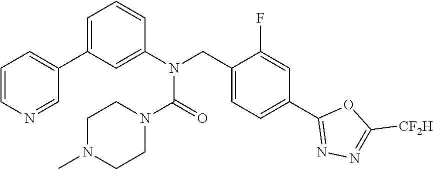 [SMILES: CN1CCN(C(=O)N(Cc2ccc(-c3nnc(C(F)F)o3)cc2F)c2cccc(-c3cccnc3)c2)CC1] | 194 | 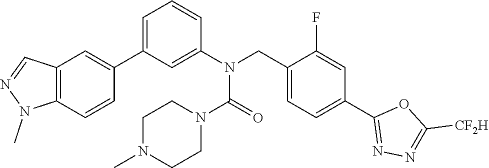 [SMILES: CN1CCN(C(=O)N(Cc2ccc(-c3nnc(C(F)F)o3)cc2F)c2cccc(-c3ccc4c(cnn4C)c3)c2)CC1] |
| 195 | 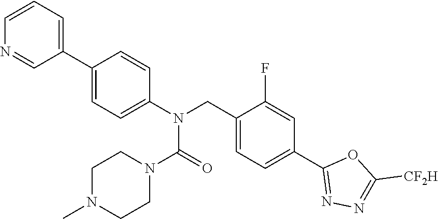 [SMILES: CN1CCN(C(=O)N(Cc2ccc(-c3nnc(C(F)F)o3)cc2F)c2ccc(-c3cccnc3)cc2)CC1] | 196 | 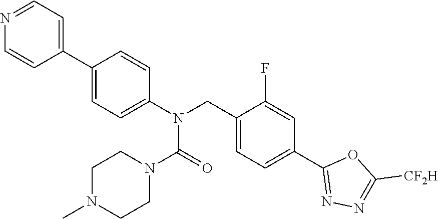 [SMILES: CN1CCN(C(=O)N(Cc2ccc(-c3nnc(C(F)F)o3)cc2F)c2ccc(-c3ccncc3)cc2)CC1] |
| 197 | 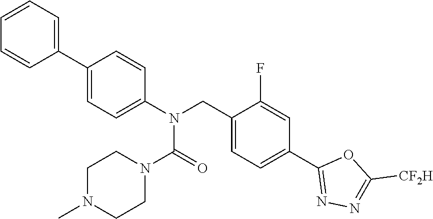 [SMILES: CN1CCN(C(=O)N(Cc2ccc(-c3nnc(C(F)F)o3)cc2F)c2ccc(-c3ccccc3)cc2)CC1] | 198 | 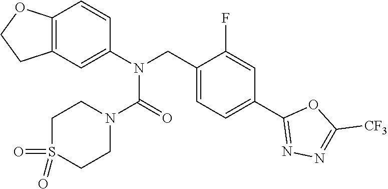 [SMILES: O=C(N1CCS(=O)(=O)CC1)N(Cc1ccc(-c2nnc(C(F)(F)F)o2)cc1F)c1ccc2c(c1)CCO2] |
| 199 | 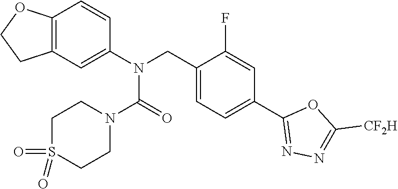 [SMILES: O=C(N1CCS(=O)(=O)CC1)N(Cc1ccc(-c2nnc(C(F)F)o2)cc1F)c1ccc2c(c1)CCO2] | 200 | 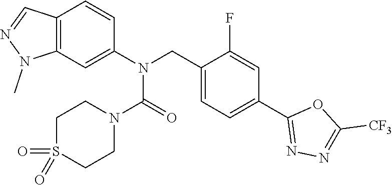 [SMILES: Cn1ncc2ccc(N(Cc3ccc(-c4nnc(C(F)(F)F)o4)cc3F)C(=O)N3CCS(=O)(=O)CC3)cc21] |
| 201 | 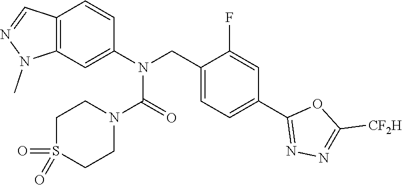 [SMILES: Cn1ncc2ccc(N(Cc3ccc(-c4nnc(C(F)F)o4)cc3F)C(=O)N3CCS(=O)(=O)CC3)cc21] | 202 | 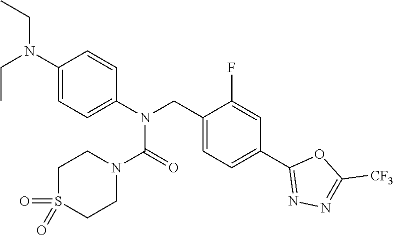 [SMILES: CCN(CC)c1ccc(N(Cc2ccc(-c3nnc(C(F)(F)F)o3)cc2F)C(=O)N2CCS(=O)(=O)CC2)cc1] |
| 203 | 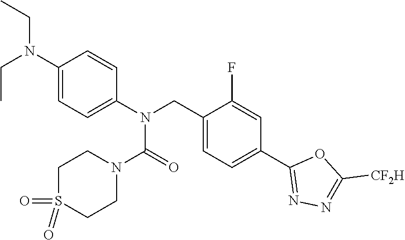 [SMILES: CCN(CC)c1ccc(N(Cc2ccc(-c3nnc(C(F)F)o3)cc2F)C(=O)N2CCS(=O)(=O)CC2)cc1] | 204 | 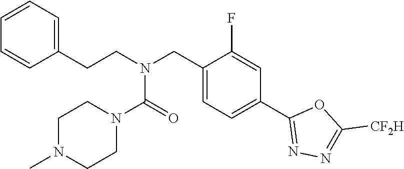 [SMILES: CN1CCN(C(=O)N(CCc2ccccc2)Cc2ccc(-c3nnc(C(F)F)o3)cc2F)CC1] |
| 205 | 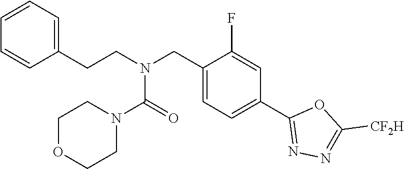 [SMILES: O=C(N1CCOCC1)N(CCc1ccccc1)Cc1ccc(-c2nnc(C(F)F)o2)cc1F] | 206 | 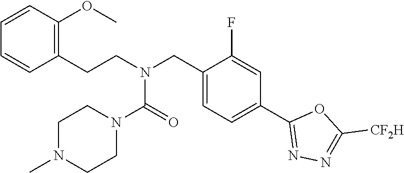 [SMILES: COc1ccccc1CCN(Cc1ccc(-c2nnc(C(F)F)o2)cc1F)C(=O)N1CCN(C)CC1] |
| 207 | 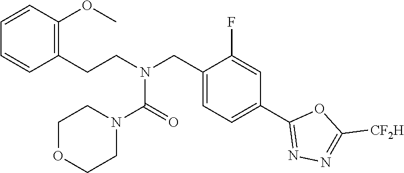 [SMILES: COc1ccccc1CCN(Cc1ccc(-c2nnc(C(F)F)o2)cc1F)C(=O)N1CCOCC1] | 208 | 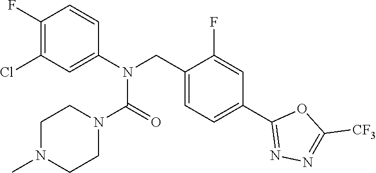 [SMILES: CN1CCN(C(=O)N(Cc2ccc(-c3nnc(C(F)(F)F)o3)cc2F)c2ccc(F)c(Cl)c2)CC1] |
| 209 | 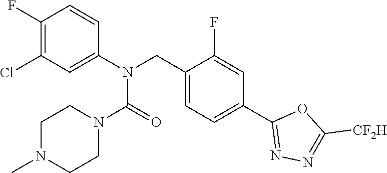 [SMILES: CN1CCN(C(=O)N(Cc2ccc(-c3nnc(C(F)F)o3)cc2F)c2ccc(F)c(Cl)c2)CC1] | 210 | 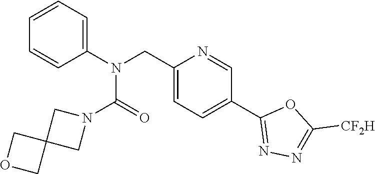 [SMILES: O=C(N1CC2(COC2)C1)N(Cc1ccc(-c2nnc(C(F)F)o2)cn1)c1ccccc1] |
| 211 | 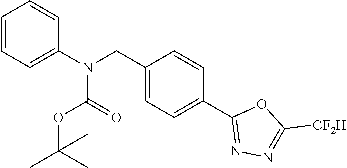 [SMILES: CC(C)(C)OC(=O)N(Cc1ccc(-c2nnc(C(F)F)o2)cc1)c1ccccc1] | 212 | 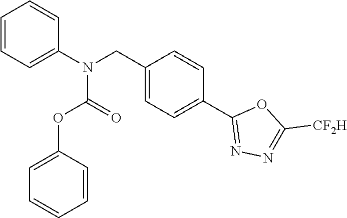 [SMILES: O=C(Oc1ccccc1)N(Cc1ccc(-c2nnc(C(F)F)o2)cc1)c1ccccc1] |
| 213 | 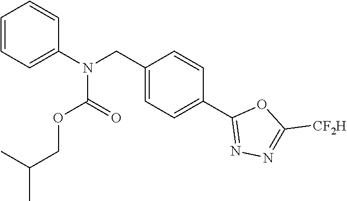 [SMILES: CC(C)COC(=O)N(Cc1ccc(-c2nnc(C(F)F)o2)cc1)c1ccccc1] | 214 | 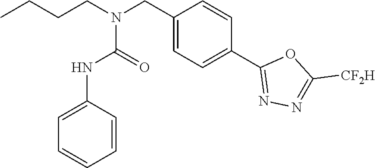 [SMILES: CCCCN(Cc1ccc(-c2nnc(C(F)F)o2)cc1)C(=O)Nc1ccccc1] |
| 215 | 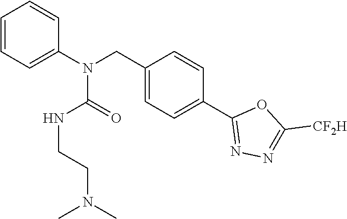 [SMILES: CN(C)CCNC(=O)N(Cc1ccc(-c2nnc(C(F)F)o2)cc1)c1ccccc1] | 216 | 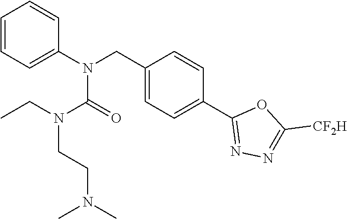 [SMILES: CCN(CCN(C)C)C(=O)N(Cc1ccc(-c2nnc(C(F)F)o2)cc1)c1ccccc1] |
| 217 | 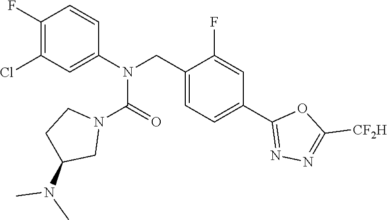 [SMILES: CN(C)[C@H]1CCN(C(=O)N(Cc2ccc(-c3nnc(C(F)F)o3)cc2F)c2ccc(F)c(Cl)c2)C1] | 218 | 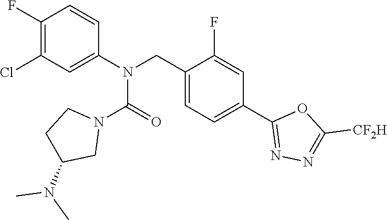 [SMILES: CN(C)[C@@H]1CCN(C(=O)N(Cc2ccc(-c3nnc(C(F)F)o3)cc2F)c2ccc(F)c(Cl)c2)C1] |
| 219 | 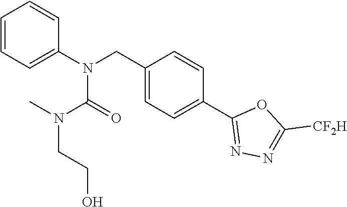 [SMILES: CN(CCO)C(=O)N(Cc1ccc(-c2nnc(C(F)F)o2)cc1)c1ccccc1] | 220 | 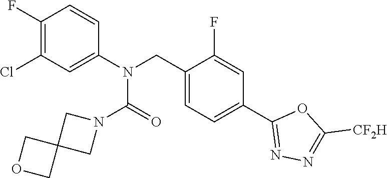 [SMILES: O=C(N1CC2(COC2)C1)N(Cc1ccc(-c2nnc(C(F)F)o2)cc1F)c1ccc(F)c(Cl)c1] |
| 221 | 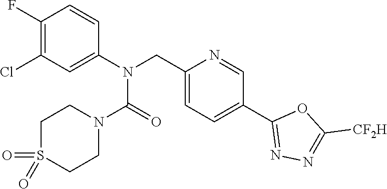 [SMILES: O=C(N1CCS(=O)(=O)CC1)N(Cc1ccc(-c2nnc(C(F)F)o2)cn1)c1ccc(F)c(Cl)c1] | 222 | 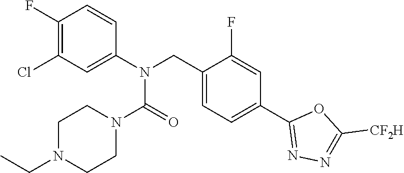 [SMILES: CCN1CCN(C(=O)N(Cc2ccc(-c3nnc(C(F)F)o3)cc2F)c2ccc(F)c(Cl)c2)CC1] |
| 223 | 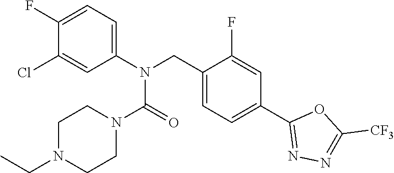 [SMILES: CCN1CCN(C(=O)N(Cc2ccc(-c3nnc(C(F)(F)F)o3)cc2F)c2ccc(F)c(Cl)c2)CC1] | 224 | 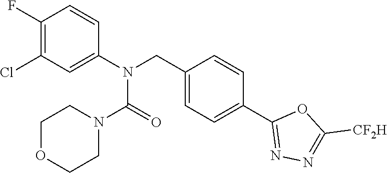 [SMILES: O=C(N1CCOCC1)N(Cc1ccc(-c2nnc(C(F)F)o2)cc1)c1ccc(F)c(Cl)c1] |
| 225 | 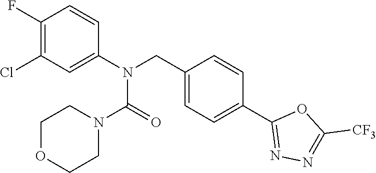 [SMILES: O=C(N1CCOCC1)N(Cc1ccc(-c2nnc(C(F)(F)F)o2)cc1)c1ccc(F)c(Cl)c1] | 226 | 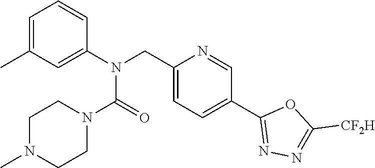 [SMILES: Cc1cccc(N(Cc2ccc(-c3nnc(C(F)F)o3)cn2)C(=O)N2CCN(C)CC2)c1] |
| 227 | 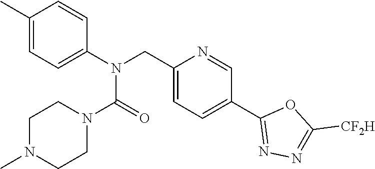 [SMILES: Cc1ccc(N(Cc2ccc(-c3nnc(C(F)F)o3)cn2)C(=O)N2CCN(C)CC2)cc1] | 228 | 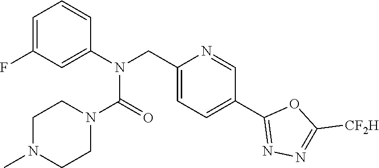 [SMILES: CN1CCN(C(=O)N(Cc2ccc(-c3nnc(C(F)F)o3)cn2)c2cccc(F)c2)CC1] |
| 229 | 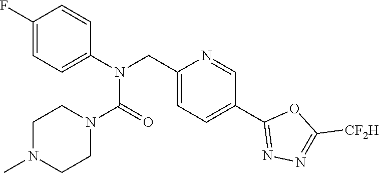 [SMILES: CN1CCN(C(=O)N(Cc2ccc(-c3nnc(C(F)F)o3)cn2)c2ccc(F)cc2)CC1] | 230 | 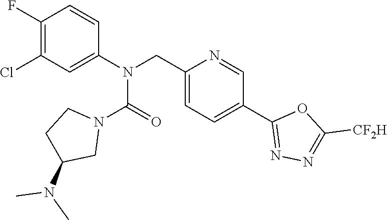 [SMILES: CN(C)[C@H]1CCN(C(=O)N(Cc2ccc(-c3nnc(C(F)F)o3)cn2)c2ccc(F)c(Cl)c2)C1] |
| 231 | 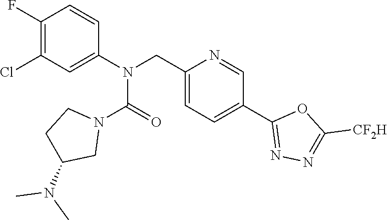 [SMILES: CN(C)[C@@H]1CCN(C(=O)N(Cc2ccc(-c3nnc(C(F)F)o3)cn2)c2ccc(F)c(Cl)c2)C1] | 232 | 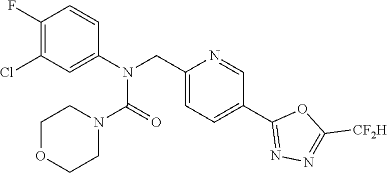 [SMILES: O=C(N1CCOCC1)N(Cc1ccc(-c2nnc(C(F)F)o2)cn1)c1ccc(F)c(Cl)c1] |
| 233 | 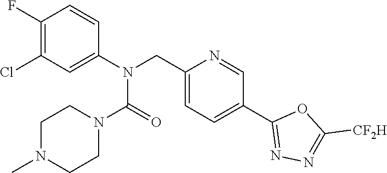 [SMILES: CN1CCN(C(=O)N(Cc2ccc(-c3nnc(C(F)F)o3)cn2)c2ccc(F)c(Cl)c2)CC1] | 234 | 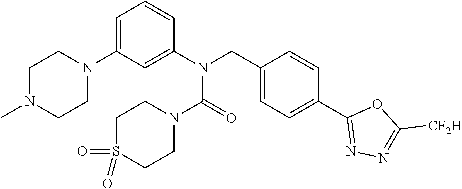 [SMILES: CN1CCN(c2cccc(N(Cc3ccc(-c4nnc(C(F)F)o4)cc3)C(=O)N3CCS(=O)(=O)CC3)c2)CC1] |
| 235 | 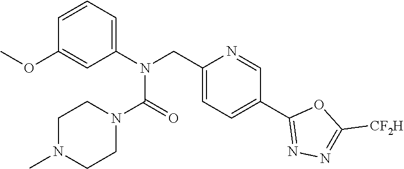 [SMILES: COc1cccc(N(Cc2ccc(-c3nnc(C(F)F)o3)cn2)C(=O)N2CCN(C)CC2)c1] | 236 | 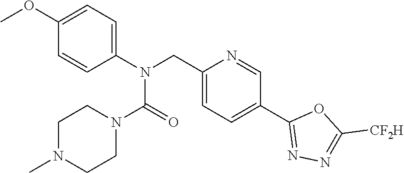 [SMILES: COc1ccc(N(Cc2ccc(-c3nnc(C(F)F)o3)cn2)C(=O)N2CCN(C)CC2)cc1] |
| 237 | 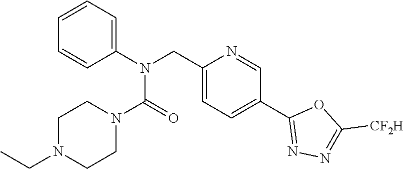 [SMILES: CCN1CCN(C(=O)N(Cc2ccc(-c3nnc(C(F)F)o3)cn2)c2ccccc2)CC1] | 238 | 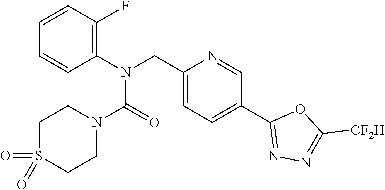 [SMILES: O=C(N1CCS(=O)(=O)CC1)N(Cc1ccc(-c2nnc(C(F)F)o2)cn1)c1ccccc1F] |
| 239 | 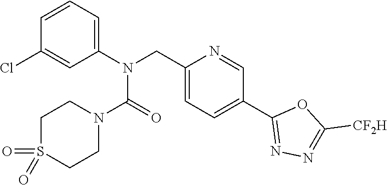 [SMILES: O=C(N1CCS(=O)(=O)CC1)N(Cc1ccc(-c2nnc(C(F)F)o2)cn1)c1cccc(Cl)c1] | 240 | 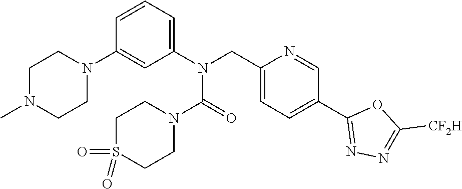 [SMILES: CN1CCN(c2cccc(N(Cc3ccc(-c4nnc(C(F)F)o4)cn3)C(=O)N3CCS(=O)(=O)CC3)c2)CC1] |
| 241 | 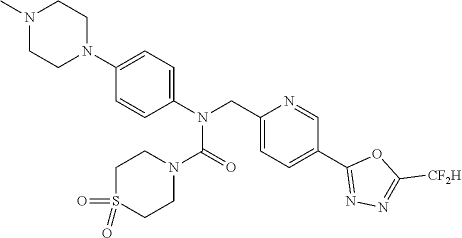 [SMILES: CN1CCN(c2ccc(N(Cc3ccc(-c4nnc(C(F)F)o4)cn3)C(=O)N3CCS(=O)(=O)CC3)cc2)CC1] | 242 | 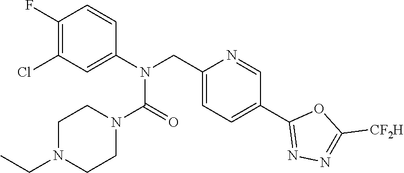 [SMILES: CCN1CCN(C(=O)N(Cc2ccc(-c3nnc(C(F)F)o3)cn2)c2ccc(F)c(Cl)c2)CC1] |
| 243 | 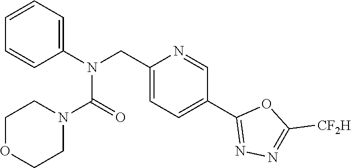 [SMILES: O=C(N1CCOCC1)N(Cc1ccc(-c2nnc(C(F)F)o2)cn1)c1ccccc1] | 244 | 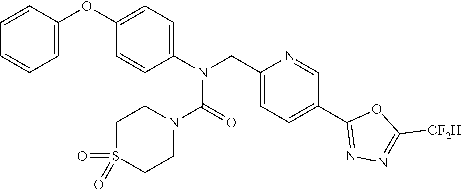 [SMILES: O=C(N1CCS(=O)(=O)CC1)N(Cc1ccc(-c2nnc(C(F)F)o2)cn1)c1ccc(Oc2ccccc2)cc1] |
| 245 | 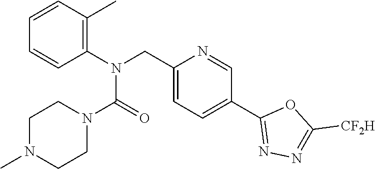 [SMILES: Cc1ccccc1N(Cc1ccc(-c2nnc(C(F)F)o2)cn1)C(=O)N1CCN(C)CC1] | 246 | 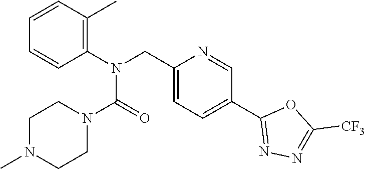 [SMILES: Cc1ccccc1N(Cc1ccc(-c2nnc(C(F)(F)F)o2)cn1)C(=O)N1CCN(C)CC1] |
| 247 | 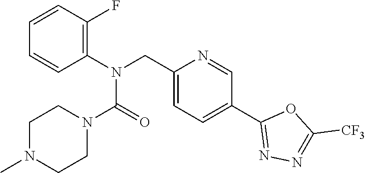 [SMILES: CN1CCN(C(=O)N(Cc2ccc(-c3nnc(C(F)(F)F)o3)cn2)c2ccccc2F)CC1] | 248 | 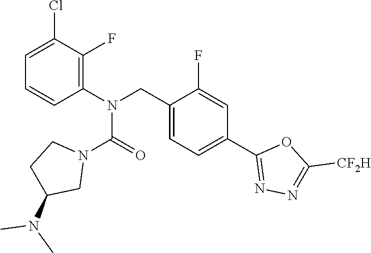 [SMILES: CN(C)[C@H]1CCN(C(=O)N(Cc2ccc(-c3nnc(C(F)F)o3)cc2F)c2cccc(Cl)c2F)C1] |
| 249 | 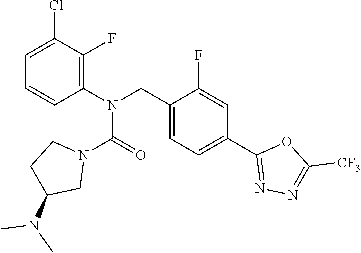 [SMILES: CN(C)[C@H]1CCN(C(=O)N(Cc2ccc(-c3nnc(C(F)(F)F)o3)cc2F)c2cccc(Cl)c2F)C1] | 250 | 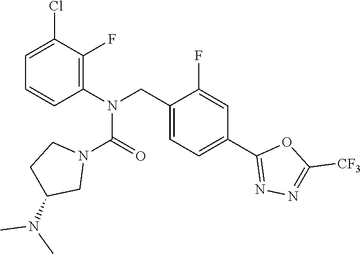 [SMILES: CN(C)[C@@H]1CCN(C(=O)N(Cc2ccc(-c3nnc(C(F)(F)F)o3)cc2F)c2cccc(Cl)c2F)C1] |
| 251 | 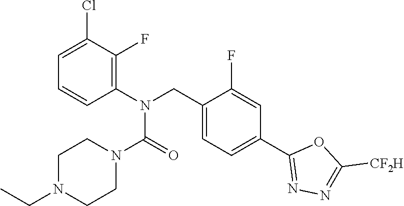 [SMILES: CCN1CCN(C(=O)N(Cc2ccc(-c3nnc(C(F)F)o3)cc2F)c2cccc(Cl)c2F)CC1] | 252 | 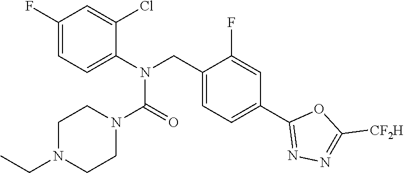 [SMILES: CCN1CCN(C(=O)N(Cc2ccc(-c3nnc(C(F)F)o3)cc2F)c2ccc(F)cc2Cl)CC1] |
| 253 | 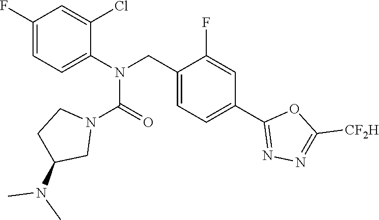 [SMILES: CN(C)[C@H]1CCN(C(=O)N(Cc2ccc(-c3nnc(C(F)F)o3)cc2F)c2ccc(F)cc2Cl)C1] | 254 | 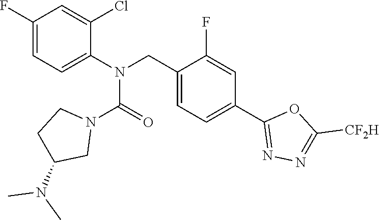 [SMILES: CN(C)[C@@H]1CCN(C(=O)N(Cc2ccc(-c3nnc(C(F)F)o3)cc2F)c2ccc(F)cc2Cl)C1] |
| 255 | 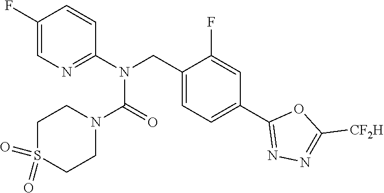 [SMILES: O=C(N1CCS(=O)(=O)CC1)N(Cc1ccc(-c2nnc(C(F)F)o2)cc1F)c1ccc(F)cn1] | 256 | 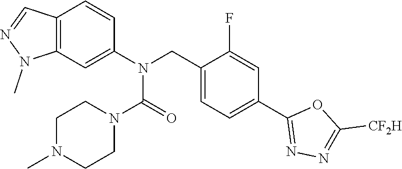 [SMILES: CN1CCN(C(=O)N(Cc2ccc(-c3nnc(C(F)F)o3)cc2F)c2ccc3cnn(C)c3c2)CC1] |
| 257 | 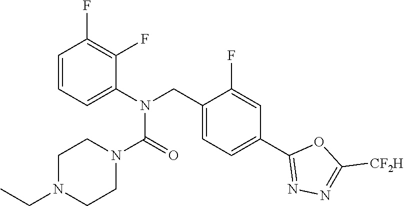 [SMILES: CCN1CCN(C(=O)N(Cc2ccc(-c3nnc(C(F)F)o3)cc2F)c2cccc(F)c2F)CC1] | 258 | 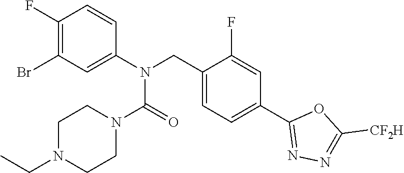 [SMILES: CCN1CCN(C(=O)N(Cc2ccc(-c3nnc(C(F)F)o3)cc2F)c2ccc(F)c(Br)c2)CC1] |
| 259 | 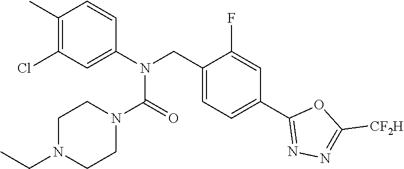 [SMILES: CCN1CCN(C(=O)N(Cc2ccc(-c3nnc(C(F)F)o3)cc2F)c2ccc(C)c(Cl)c2)CC1] | 260 | 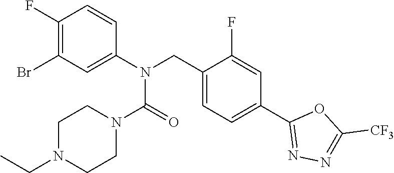 [SMILES: CCN1CCN(C(=O)N(Cc2ccc(-c3nnc(C(F)(F)F)o3)cc2F)c2ccc(F)c(Br)c2)CC1] |
| 261 | 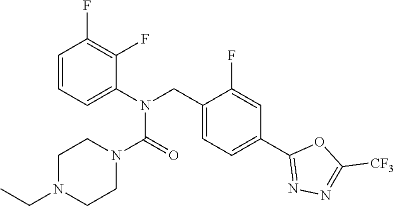 [SMILES: CCN1CCN(C(=O)N(Cc2ccc(-c3nnc(C(F)(F)F)o3)cc2F)c2cccc(F)c2F)CC1] | 262 | 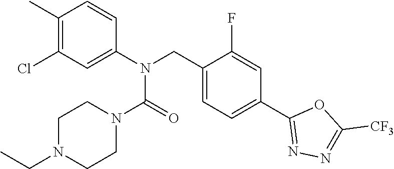 [SMILES: CCN1CCN(C(=O)N(Cc2ccc(-c3nnc(C(F)(F)F)o3)cc2F)c2ccc(C)c(Cl)c2)CC1] |
| 263 | 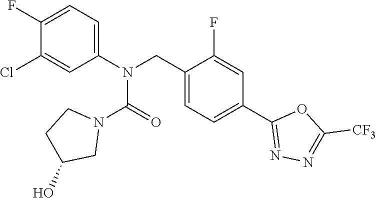 [SMILES: O=C(N1CC[C@@H](O)C1)N(Cc1ccc(-c2nnc(C(F)(F)F)o2)cc1F)c1ccc(F)c(Cl)c1] | 264 | 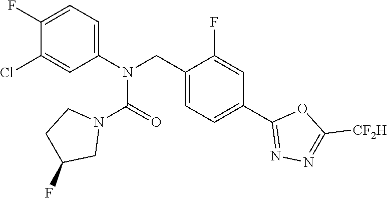 [SMILES: O=C(N1CC[C@H](F)C1)N(Cc1ccc(-c2nnc(C(F)F)o2)cc1F)c1ccc(F)c(Cl)c1] |
| 265 | 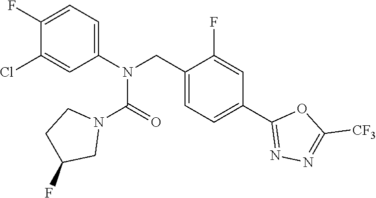 [SMILES: O=C(N1CC[C@H](F)C1)N(Cc1ccc(-c2nnc(C(F)(F)F)o2)cc1F)c1ccc(F)c(Cl)c1] | 266 | 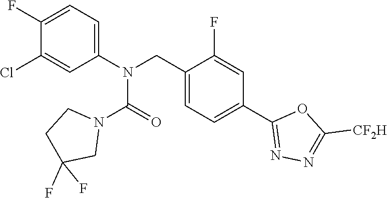 [SMILES: O=C(N1CCC(F)(F)C1)N(Cc1ccc(-c2nnc(C(F)F)o2)cc1F)c1ccc(F)c(Cl)c1] |
| 267 | 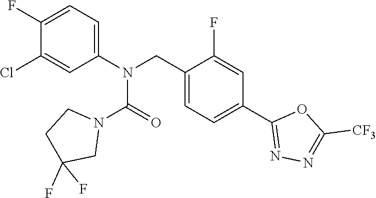 [SMILES: O=C(N1CCC(F)(F)C1)N(Cc1ccc(-c2nnc(C(F)(F)F)o2)cc1F)c1ccc(F)c(Cl)c1] | 268 | 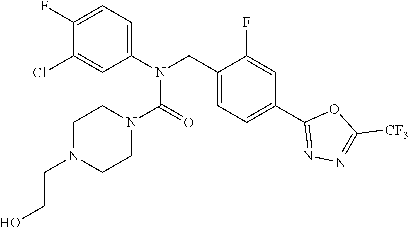 [SMILES: O=C(N1CCN(CCO)CC1)N(Cc1ccc(-c2nnc(C(F)(F)F)o2)cc1F)c1ccc(F)c(Cl)c1] |
| 269 | 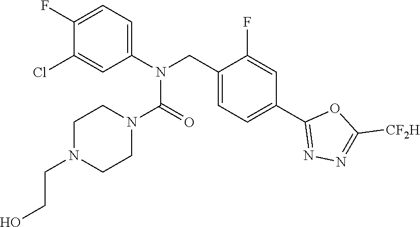 [SMILES: O=C(N1CCN(CCO)CC1)N(Cc1ccc(-c2nnc(C(F)F)o2)cc1F)c1ccc(F)c(Cl)c1] | 270 | 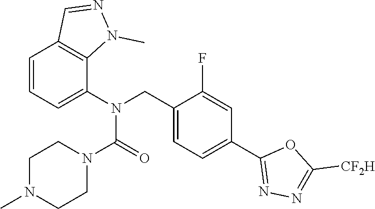 [SMILES: CN1CCN(C(=O)N(Cc2ccc(-c3nnc(C(F)F)o3)cc2F)c2cccc3cnn(C)c23)CC1] |
| 271 | 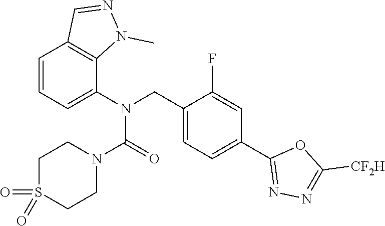 [SMILES: Cn1ncc2cccc(N(Cc3ccc(-c4nnc(C(F)F)o4)cc3F)C(=O)N3CCS(=O)(=O)CC3)c21] | 272 | 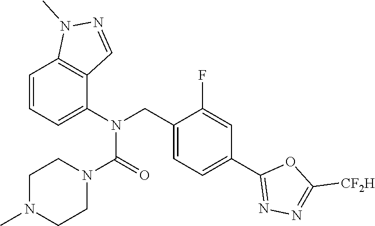 [SMILES: CN1CCN(C(=O)N(Cc2ccc(-c3nnc(C(F)F)o3)cc2F)c2cccc3c2cnn3C)CC1] |
| 273 | 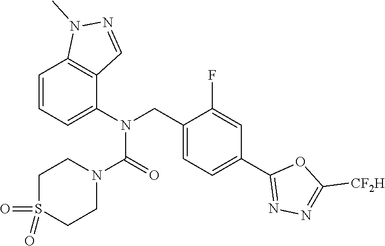 [SMILES: Cn1ncc2c(N(Cc3ccc(-c4nnc(C(F)F)o4)cc3F)C(=O)N3CCS(=O)(=O)CC3)cccc21] | 274 | 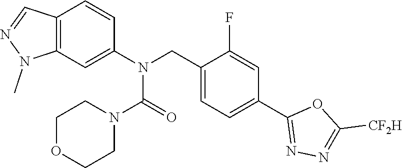 |
| 275 | 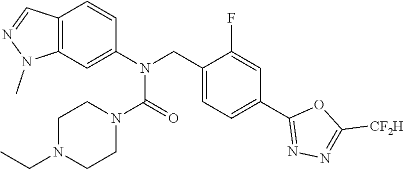 [SMILES: CCN1CCN(C(=O)N(Cc2ccc(-c3nnc(C(F)F)o3)cc2F)c2ccc3cnn(C)c3c2)CC1] | 276 | 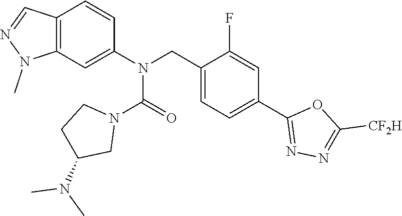 [SMILES: CN(C)[C@@H]1CCN(C(=O)N(Cc2ccc(-c3nnc(C(F)F)o3)cc2F)c2ccc3cnn(C)c3c2)C1] |
| 277 | 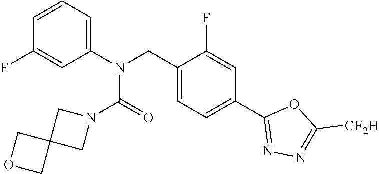 [SMILES: O=C(N1CC2(COC2)C1)N(Cc1ccc(-c2nnc(C(F)F)o2)cc1F)c1cccc(F)c1] | 278 | 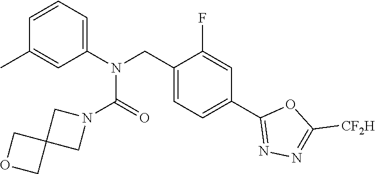 [SMILES: Cc1cccc(N(Cc2ccc(-c3nnc(C(F)F)o3)cc2F)C(=O)N2CC3(COC3)C2)c1] |
| 279 | 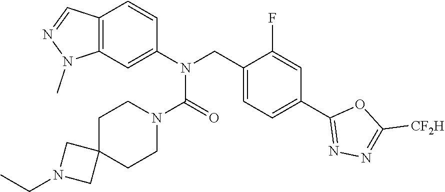 [SMILES: CCN1CC2(CCN(C(=O)N(Cc3ccc(-c4nnc(C(F)F)o4)cc3F)c3ccc4cnn(C)c4c3)CC2)C1] | 280 | 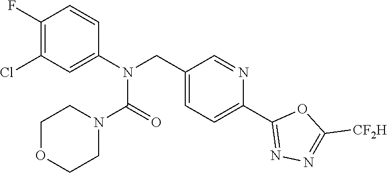 [SMILES: O=C(N1CCOCC1)N(Cc1ccc(-c2nnc(C(F)F)o2)nc1)c1ccc(F)c(Cl)c1] |
| 281 | 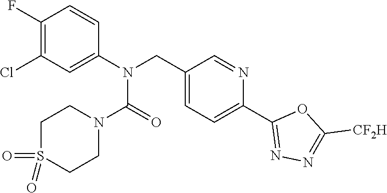 [SMILES: O=C(N1CCS(=O)(=O)CC1)N(Cc1ccc(-c2nnc(C(F)F)o2)nc1)c1ccc(F)c(Cl)c1] | 282 | 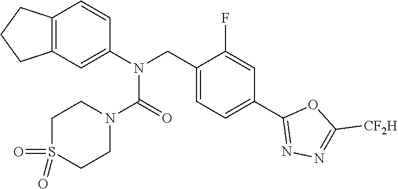 [SMILES: O=C(N1CCS(=O)(=O)CC1)N(Cc1ccc(-c2nnc(C(F)F)o2)cc1F)c1ccc2c(c1)CCC2] |
| 283 | 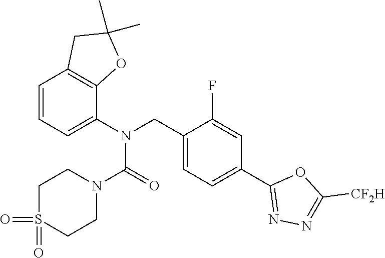 [SMILES: CC1(C)Cc2cccc(N(Cc3ccc(-c4nnc(C(F)F)o4)cc3F)C(=O)N3CCS(=O)(=O)CC3)c2O1] | 284 | 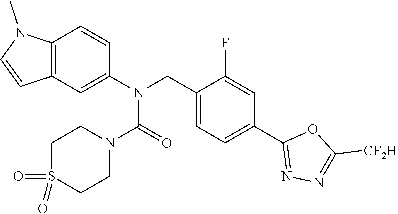 [SMILES: Cn1ccc2cc(N(Cc3ccc(-c4nnc(C(F)F)o4)cc3F)C(=O)N3CCS(=O)(=O)CC3)ccc21] |
| 285 | 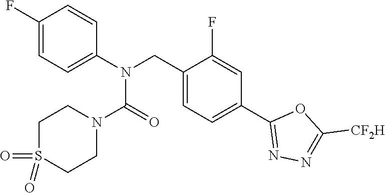 [SMILES: O=C(N1CCS(=O)(=O)CC1)N(Cc1ccc(-c2nnc(C(F)F)o2)cc1F)c1ccc(F)cc1] | 286 | 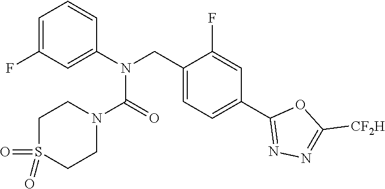 [SMILES: O=C(N1CCS(=O)(=O)CC1)N(Cc1ccc(-c2nnc(C(F)F)o2)cc1F)c1cccc(F)c1] |
| 287 | 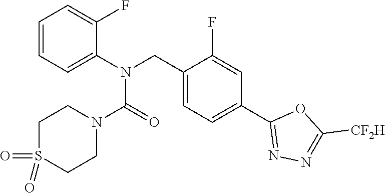 [SMILES: O=C(N1CCS(=O)(=O)CC1)N(Cc1ccc(-c2nnc(C(F)F)o2)cc1F)c1ccccc1F] | 288 | 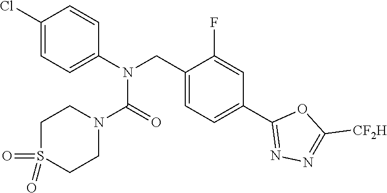 [SMILES: O=C(N1CCS(=O)(=O)CC1)N(Cc1ccc(-c2nnc(C(F)F)o2)cc1F)c1ccc(Cl)cc1] |
| 289 | 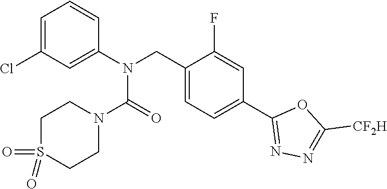 [SMILES: O=C(N1CCS(=O)(=O)CC1)N(Cc1ccc(-c2nnc(C(F)F)o2)cc1F)c1cccc(Cl)c1] | 290 | 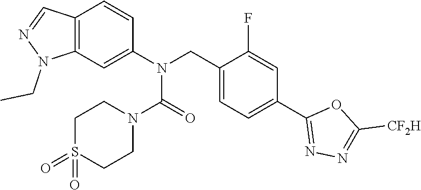 [SMILES: CCn1ncc2ccc(N(Cc3ccc(-c4nnc(C(F)F)o4)cc3F)C(=O)N3CCS(=O)(=O)CC3)cc21] |
| 291 | 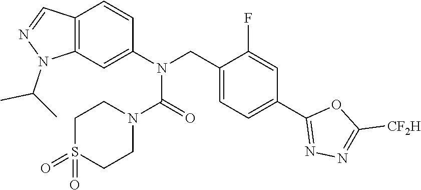 [SMILES: CC(C)n1ncc2ccc(N(Cc3ccc(-c4nnc(C(F)F)o4)cc3F)C(=O)N3CCS(=O)(=O)CC3)cc21] | 292 | 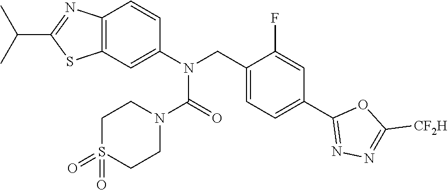 [SMILES: CC(C)c1nc2ccc(N(Cc3ccc(-c4nnc(C(F)F)o4)cc3F)C(=O)N3CCS(=O)(=O)CC3)cc2s1] |
| 293 | 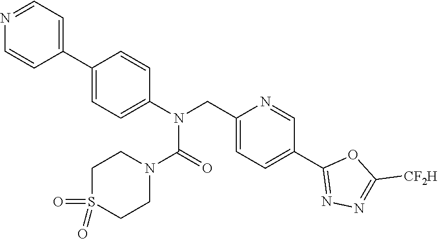 [SMILES: O=C(N1CCS(=O)(=O)CC1)N(Cc1ccc(-c2nnc(C(F)F)o2)cn1)c1ccc(-c2ccncc2)cc1] | 294 | 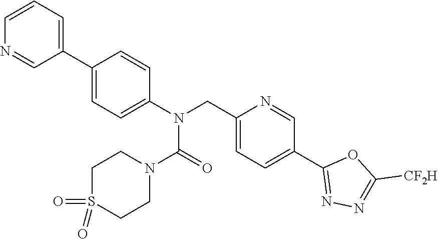 [SMILES: O=C(N1CCS(=O)(=O)CC1)N(Cc1ccc(-c2nnc(C(F)F)o2)cn1)c1ccc(-c2cccnc2)cc1] |
| 295 | 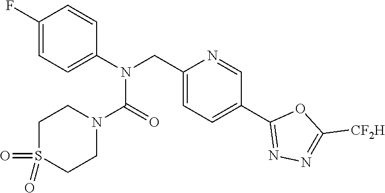 [SMILES: O=C(N1CCS(=O)(=O)CC1)N(Cc1ccc(-c2nnc(C(F)F)o2)cn1)c1ccc(F)cc1] | 296 | 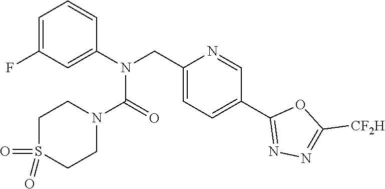 [SMILES: O=C(N1CCS(=O)(=O)CC1)N(Cc1ccc(-c2nnc(C(F)F)o2)cn1)c1cccc(F)c1] |
| 297 | 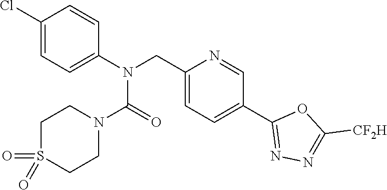 [SMILES: O=C(N1CCS(=O)(=O)CC1)N(Cc1ccc(-c2nnc(C(F)F)o2)cn1)c1ccc(Cl)cc1] | 298 | 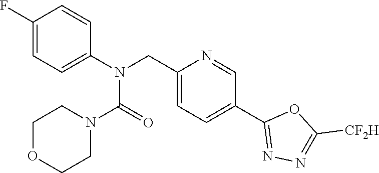 [SMILES: O=C(N1CCOCC1)N(Cc1ccc(-c2nnc(C(F)F)o2)cn1)c1ccc(F)cc1] |
| 299 | 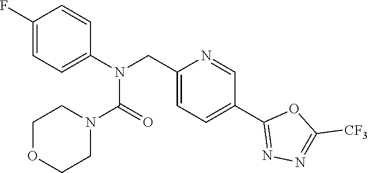 [SMILES: O=C(N1CCOCC1)N(Cc1ccc(-c2nnc(C(F)(F)F)o2)cn1)c1ccc(F)cc1] | 300 | 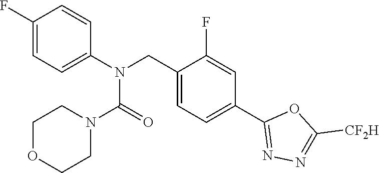 [SMILES: O=C(N1CCOCC1)N(Cc1ccc(-c2nnc(C(F)F)o2)cc1F)c1ccc(F)cc1] |
| 301 | 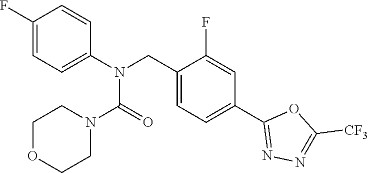 [SMILES: O=C(N1CCOCC1)N(Cc1ccc(-c2nnc(C(F)(F)F)o2)cc1F)c1ccc(F)cc1] | 302 | 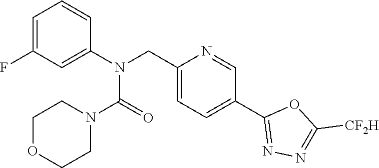 [SMILES: O=C(N1CCOCC1)N(Cc1ccc(-c2nnc(C(F)F)o2)cn1)c1cccc(F)c1] |
| 303 | 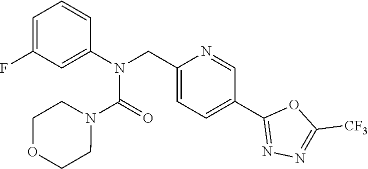 [SMILES: O=C(N1CCOCC1)N(Cc1ccc(-c2nnc(C(F)(F)F)o2)cn1)c1cccc(F)c1] | 304 | 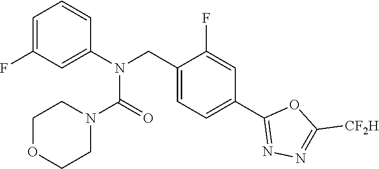 [SMILES: O=C(N1CCOCC1)N(Cc1ccc(-c2nnc(C(F)F)o2)cc1F)c1cccc(F)c1] |
| 305 | 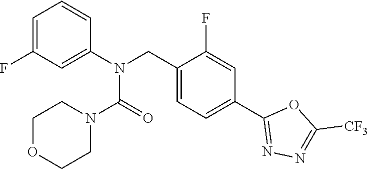 [SMILES: O=C(N1CCOCC1)N(Cc1ccc(-c2nnc(C(F)(F)F)o2)cc1F)c1cccc(F)c1] | 306 | 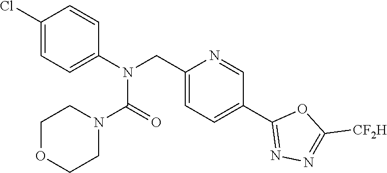 [SMILES: O=C(N1CCOCC1)N(Cc1ccc(-c2nnc(C(F)F)o2)cn1)c1ccc(Cl)cc1] |
| 307 | 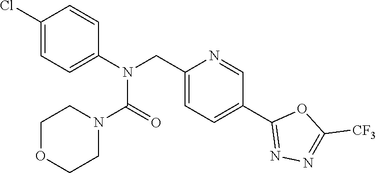 [SMILES: O=C(N1CCOCC1)N(Cc1ccc(-c2nnc(C(F)(F)F)o2)cn1)c1ccc(Cl)cc1] | 308 | 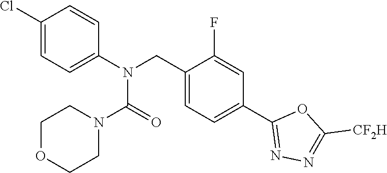 [SMILES: O=C(N1CCOCC1)N(Cc1ccc(-c2nnc(C(F)F)o2)cc1F)c1ccc(Cl)cc1] |
| 309 | 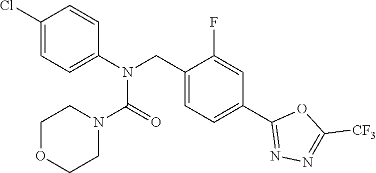 [SMILES: O=C(N1CCOCC1)N(Cc1ccc(-c2nnc(C(F)(F)F)o2)cc1F)c1ccc(Cl)cc1] | 310 | 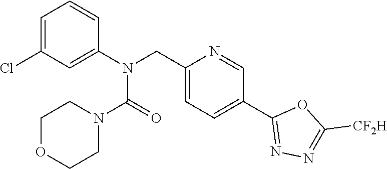 [SMILES: O=C(N1CCOCC1)N(Cc1ccc(-c2nnc(C(F)F)o2)cn1)c1cccc(Cl)c1] |
| 311 | 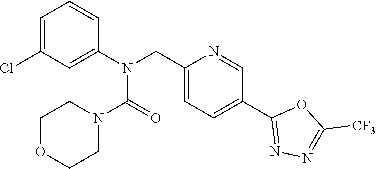 [SMILES: O=C(N1CCOCC1)N(Cc1ccc(-c2nnc(C(F)(F)F)o2)cn1)c1cccc(Cl)c1] | 312 | 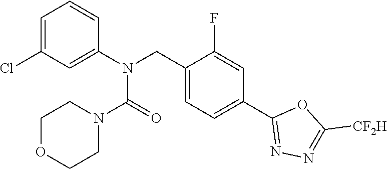 [SMILES: O=C(N1CCOCC1)N(Cc1ccc(-c2nnc(C(F)F)o2)cc1F)c1cccc(Cl)c1] |
| 313 | 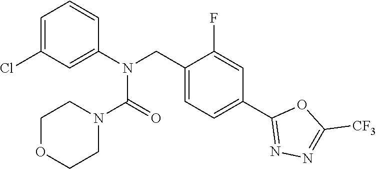 [SMILES: O=C(N1CCOCC1)N(Cc1ccc(-c2nnc(C(F)(F)F)o2)cc1F)c1cccc(Cl)c1] | 314 | 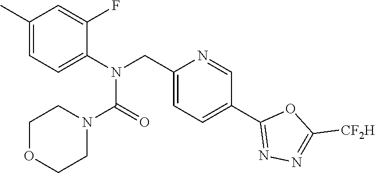 [SMILES: Cc1ccc(N(Cc2ccc(-c3nnc(C(F)F)o3)cn2)C(=O)N2CCOCC2)c(F)c1] |
| 315 | 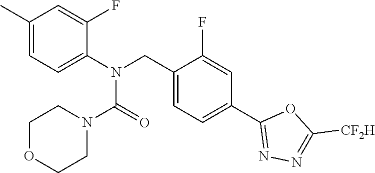 [SMILES: Cc1ccc(N(Cc2ccc(-c3nnc(C(F)F)o3)cc2F)C(=O)N2CCOCC2)c(F)c1] | 316 | 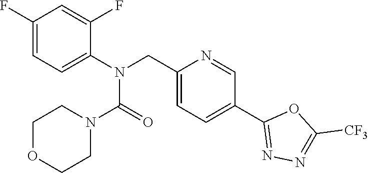 [SMILES: O=C(N1CCOCC1)N(Cc1ccc(-c2nnc(C(F)(F)F)o2)cn1)c1ccc(F)cc1F] |
| 317 | 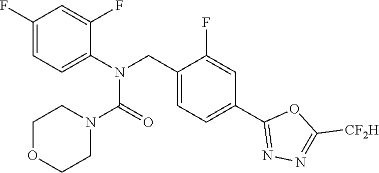 [SMILES: O=C(N1CCOCC1)N(Cc1ccc(-c2nnc(C(F)F)o2)cc1F)c1ccc(F)cc1F] | 318 | 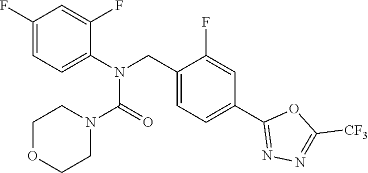 [SMILES: O=C(N1CCOCC1)N(Cc1ccc(-c2nnc(C(F)(F)F)o2)cc1F)c1ccc(F)cc1F] |
| 319 | 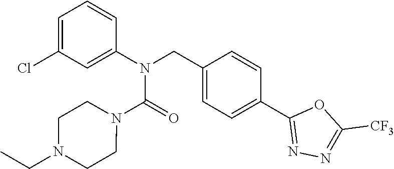 [SMILES: CCN1CCN(C(=O)N(Cc2ccc(-c3nnc(C(F)(F)F)o3)cc2)c2cccc(Cl)c2)CC1] | 320 | 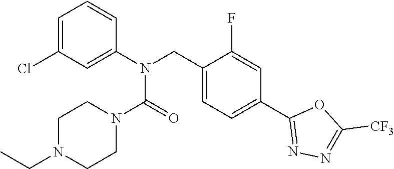 [SMILES: CCN1CCN(C(=O)N(Cc2ccc(-c3nnc(C(F)(F)F)o3)cc2F)c2cccc(Cl)c2)CC1] |
| 321 | 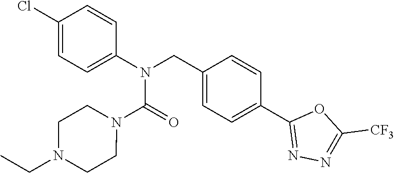 [SMILES: CCN1CCN(C(=O)N(Cc2ccc(-c3nnc(C(F)(F)F)o3)cc2)c2ccc(Cl)cc2)CC1] | 322 | 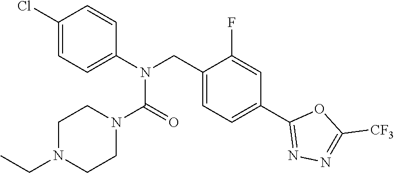 [SMILES: CCN1CCN(C(=O)N(Cc2ccc(-c3nnc(C(F)(F)F)o3)cc2F)c2ccc(Cl)cc2)CC1] |
| 323 | 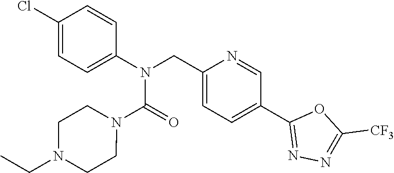 [SMILES: CCN1CCN(C(=O)N(Cc2ccc(-c3nnc(C(F)(F)F)o3)cn2)c2ccc(Cl)cc2)CC1] | 324 | 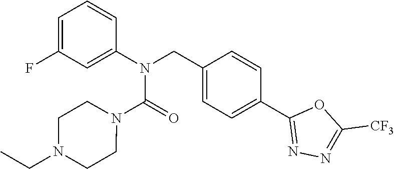 [SMILES: CCN1CCN(C(=O)N(Cc2ccc(-c3nnc(C(F)(F)F)o3)cc2)c2cccc(F)c2)CC1] |
| 325 | 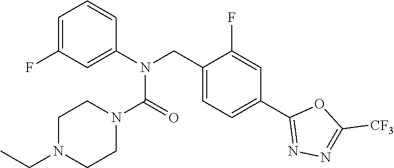 [SMILES: CCN1CCN(C(=O)N(Cc2ccc(-c3nnc(C(F)(F)F)o3)cc2F)c2cccc(F)c2)CC1] | 326 | 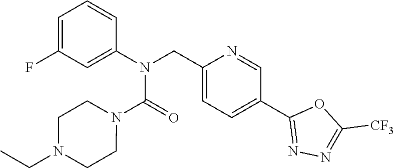 [SMILES: CCN1CCN(C(=O)N(Cc2ccc(-c3nnc(C(F)(F)F)o3)cn2)c2cccc(F)c2)CC1] |
| 327 | 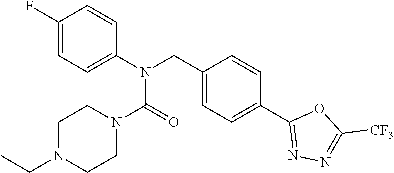 [SMILES: CCN1CCN(C(=O)N(Cc2ccc(-c3nnc(C(F)(F)F)o3)cc2)c2ccc(F)cc2)CC1] | 328 | 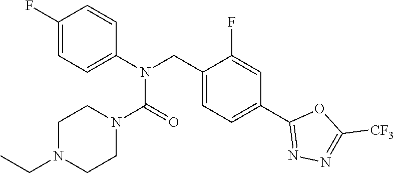 [SMILES: CCN1CCN(C(=O)N(Cc2ccc(-c3nnc(C(F)(F)F)o3)cc2F)c2ccc(F)cc2)CC1] |
| 329 | 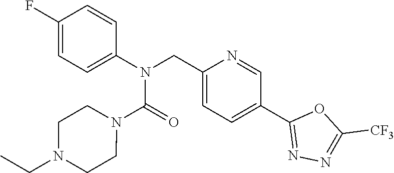 [SMILES: CCN1CCN(C(=O)N(Cc2ccc(-c3nnc(C(F)(F)F)o3)cn2)c2ccc(F)cc2)CC1] | 330 | 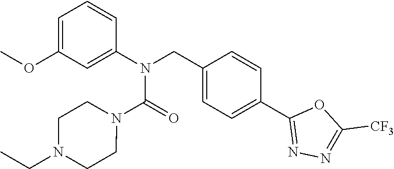 [SMILES: CCN1CCN(C(=O)N(Cc2ccc(-c3nnc(C(F)(F)F)o3)cc2)c2cccc(OC)c2)CC1] |
| 331 | 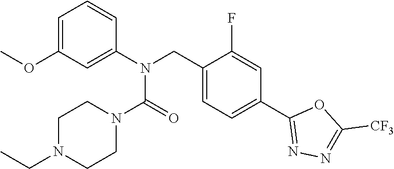 [SMILES: CCN1CCN(C(=O)N(Cc2ccc(-c3nnc(C(F)(F)F)o3)cc2F)c2cccc(OC)c2)CC1] | 332 | 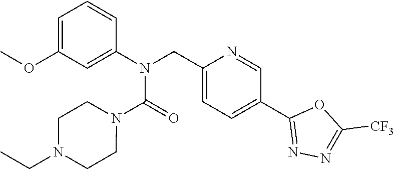 [SMILES: CCN1CCN(C(=O)N(Cc2ccc(-c3nnc(C(F)(F)F)o3)cn2)c2cccc(OC)c2)CC1] |
| 333 | 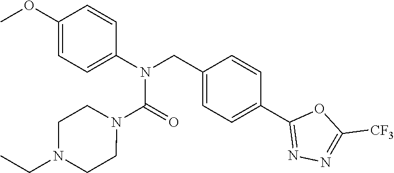 [SMILES: CCN1CCN(C(=O)N(Cc2ccc(-c3nnc(C(F)(F)F)o3)cc2)c2ccc(OC)cc2)CC1] | 334 | 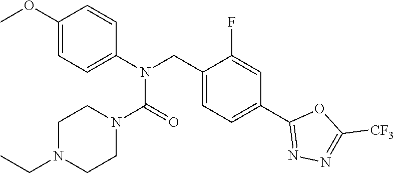 [SMILES: CCN1CCN(C(=O)N(Cc2ccc(-c3nnc(C(F)(F)F)o3)cc2F)c2ccc(OC)cc2)CC1] |
| 335 | 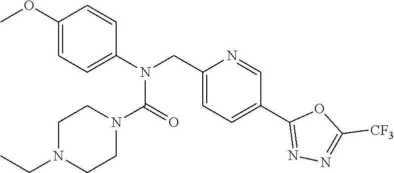 [SMILES: CCN1CCN(C(=O)N(Cc2ccc(-c3nnc(C(F)(F)F)o3)cn2)c2ccc(OC)cc2)CC1] | 336 | 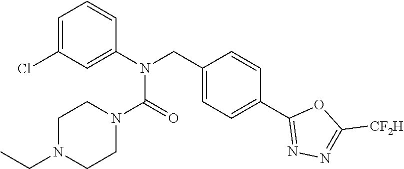 [SMILES: CCN1CCN(C(=O)N(Cc2ccc(-c3nnc(C(F)F)o3)cc2)c2cccc(Cl)c2)CC1] |
| 337 | 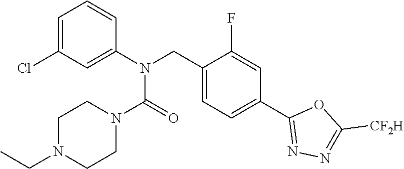 [SMILES: CCN1CCN(C(=O)N(Cc2ccc(-c3nnc(C(F)F)o3)cc2F)c2cccc(Cl)c2)CC1] | 338 | 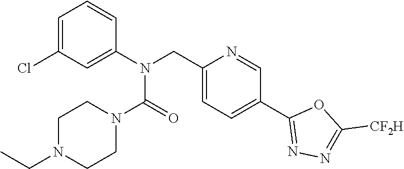 [SMILES: CCN1CCN(C(=O)N(Cc2ccc(-c3nnc(C(F)F)o3)cn2)c2cccc(Cl)c2)CC1] |
| 339 | 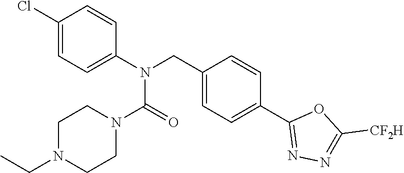 [SMILES: CCN1CCN(C(=O)N(Cc2ccc(-c3nnc(C(F)F)o3)cc2)c2ccc(Cl)cc2)CC1] | 340 | 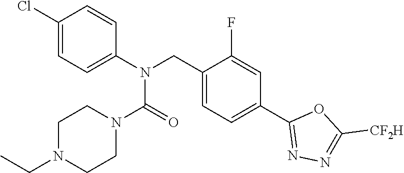 [SMILES: CCN1CCN(C(=O)N(Cc2ccc(-c3nnc(C(F)F)o3)cc2F)c2ccc(Cl)cc2)CC1] |
| 341 | 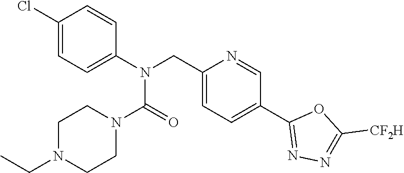 [SMILES: CCN1CCN(C(=O)N(Cc2ccc(-c3nnc(C(F)F)o3)cn2)c2ccc(Cl)cc2)CC1] | 342 | 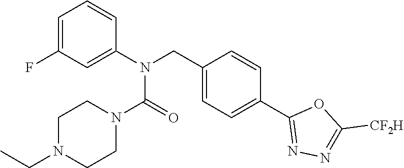 [SMILES: CCN1CCN(C(=O)N(Cc2ccc(-c3nnc(C(F)F)o3)cc2)c2cccc(F)c2)CC1] |
| 343 | 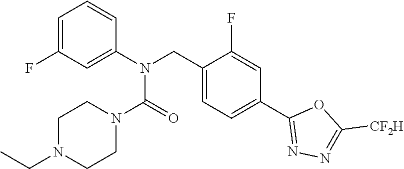 [SMILES: CCN1CCN(C(=O)N(Cc2ccc(-c3nnc(C(F)F)o3)cc2F)c2cccc(F)c2)CC1] | 344 | 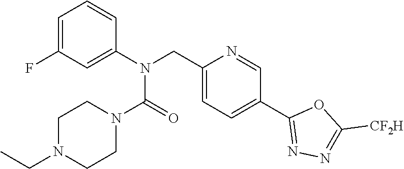 [SMILES: CCN1CCN(C(=O)N(Cc2ccc(-c3nnc(C(F)F)o3)cn2)c2cccc(F)c2)CC1] |
| 345 | 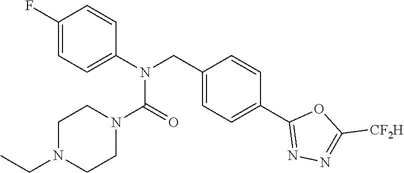 [SMILES: CCN1CCN(C(=O)N(Cc2ccc(-c3nnc(C(F)F)o3)cc2)c2ccc(F)cc2)CC1] | 346 | 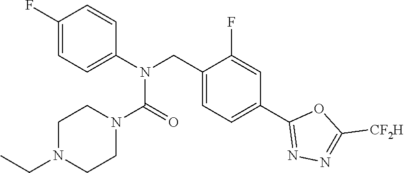 [SMILES: CCN1CCN(C(=O)N(Cc2ccc(-c3nnc(C(F)F)o3)cc2F)c2ccc(F)cc2)CC1] |
| 347 | 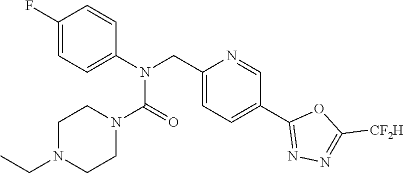 [SMILES: CCN1CCN(C(=O)N(Cc2ccc(-c3nnc(C(F)F)o3)cn2)c2ccc(F)cc2)CC1] | 348 | 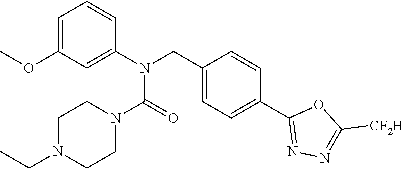 [SMILES: CCN1CCN(C(=O)N(Cc2ccc(-c3nnc(C(F)F)o3)cc2)c2cccc(OC)c2)CC1] |
| 349 | 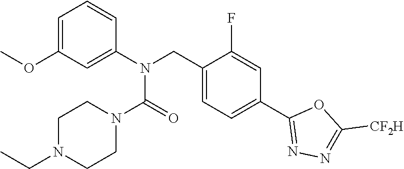 [SMILES: CCN1CCN(C(=O)N(Cc2ccc(-c3nnc(C(F)F)o3)cc2F)c2cccc(OC)c2)CC1] | 350 | 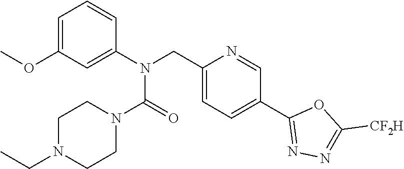 [SMILES: CCN1CCN(C(=O)N(Cc2ccc(-c3nnc(C(F)F)o3)cn2)c2cccc(OC)c2)CC1] |
| 351 | 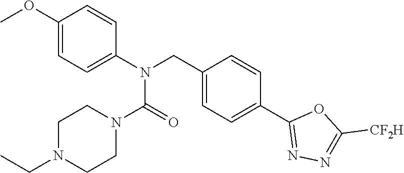 [SMILES: CCN1CCN(C(=O)N(Cc2ccc(-c3nnc(C(F)F)o3)cc2)c2ccc(OC)cc2)CC1] | 352 | 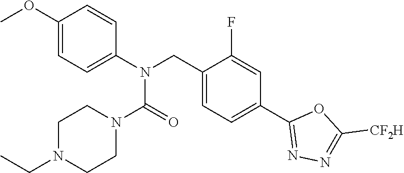 [SMILES: CCN1CCN(C(=O)N(Cc2ccc(-c3nnc(C(F)F)o3)cc2F)c2ccc(OC)cc2)CC1] |
| 353 | 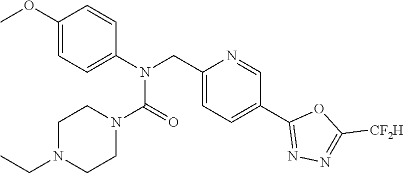 [SMILES: CCN1CCN(C(=O)N(Cc2ccc(-c3nnc(C(F)F)o3)cn2)c2ccc(OC)cc2)CC1] | 354 | 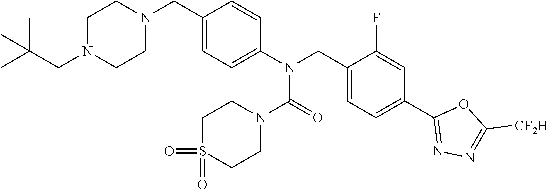 [SMILES: CC(C)(C)CN1CCN(Cc2ccc(N(Cc3ccc(-c4nnc(C(F)F)o4)cc3F)C(=O)N3CCS(=O)(=O)CC3)cc2)CC1] |
| 355 | 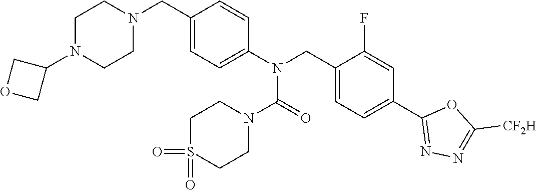 [SMILES: O=C(N1CCS(=O)(=O)CC1)N(Cc1ccc(-c2nnc(C(F)F)o2)cc1F)c1ccc(CN2CCN(C3COC3)CC2)cc1] | 356 | 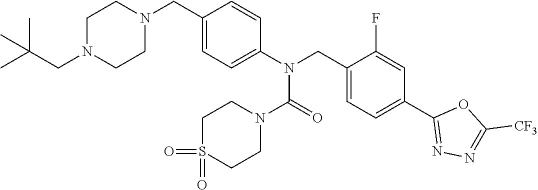 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 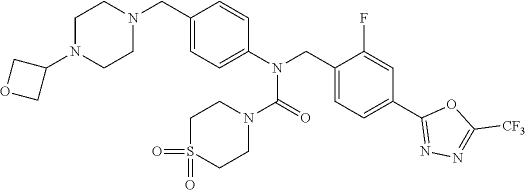 [SMILES: O=C(N1CCS(=O)(=O)CC1)N(Cc1ccc(-c2nnc(C(F)(F)F)o2)cc1F)c1ccc(CN2CCN(C3COC3)CC2)cc1] | 358 | 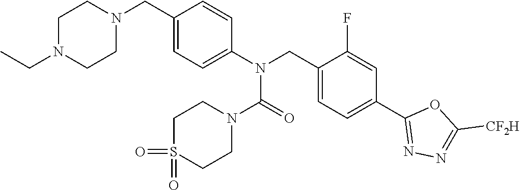 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 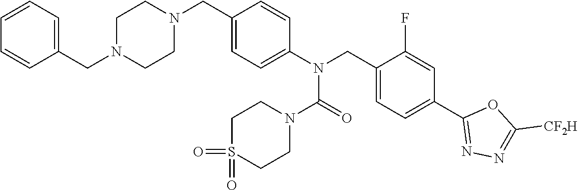 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] | 360 | 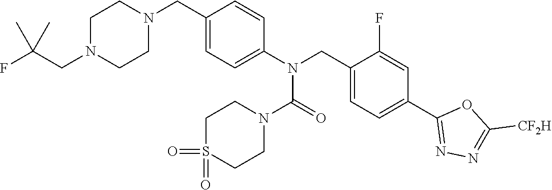 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 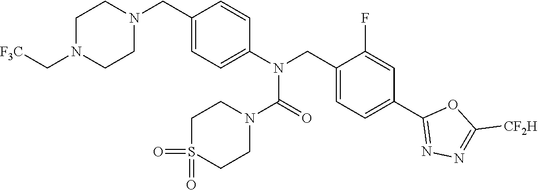 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] | 362 | 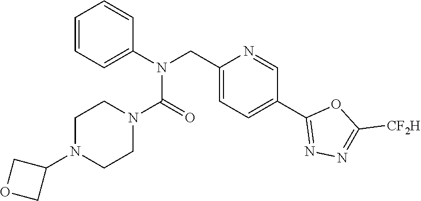 [SMILES: O=C(N1CCN(C2COC2)CC1)N(Cc1ccc(-c2nnc(C(F)F)o2)cn1)c1ccccc1] |
| 363 | 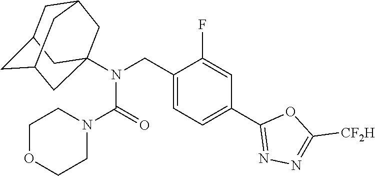 [SMILES: O=C(N1CCOCC1)N(Cc1ccc(-c2nnc(C(F)F)o2)cc1F)C12CC3CC(CC(C3)C1)C2] | 364 | 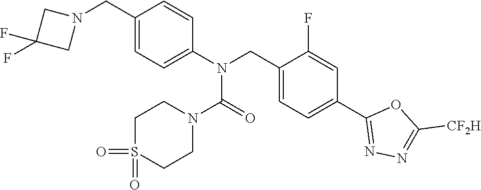 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 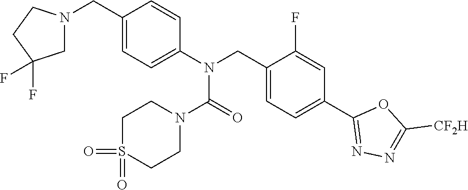 [SMILES: O=C(N1CCS(=O)(=O)CC1)N(Cc1ccc(-c2nnc(C(F)F)o2)cc1F)c1ccc(CN2CCC(F)(F)C2)cc1] | 366 | 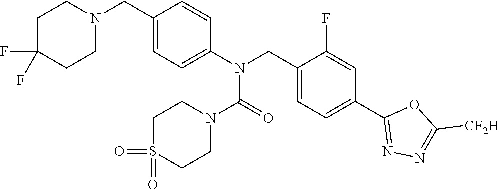 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 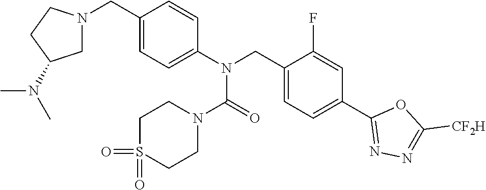 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] | 368 | 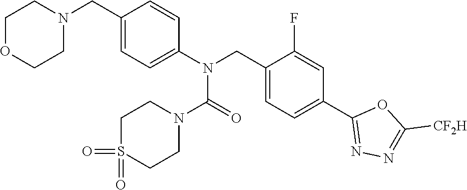 [SMILES: O=C(N1CCS(=O)(=O)CC1)N(Cc1ccc(-c2nnc(C(F)F)o2)cc1F)c1ccc(CN2CCOCC2)cc1] |
| 369 | 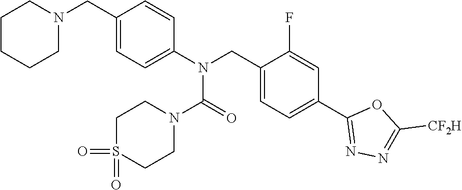 [SMILES: O=C(N1CCS(=O)(=O)CC1)N(Cc1ccc(-c2nnc(C(F)F)o2)cc1F)c1ccc(CN2CCCCC2)cc1] | 370 | 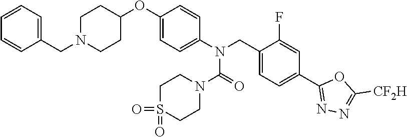 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 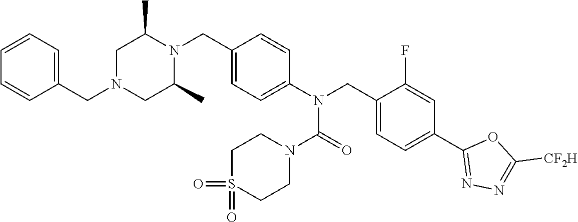 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] | 372 | 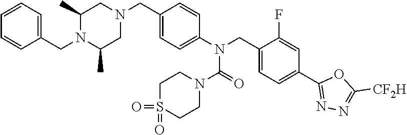 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 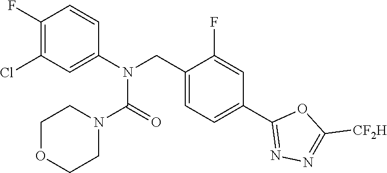 [SMILES: O=C(N1CCOCC1)N(Cc1ccc(-c2nnc(C(F)F)o2)cc1F)c1ccc(F)c(Cl)c1] | 374 | 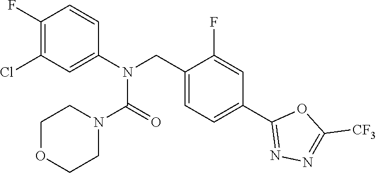 [SMILES: O=C(N1CCOCC1)N(Cc1ccc(-c2nnc(C(F)(F)F)o2)cc1F)c1ccc(F)c(Cl)c1] |
| 375 | 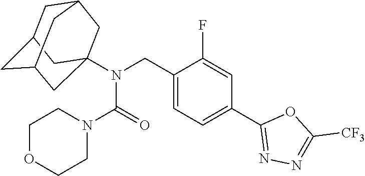 [SMILES: O=C(N1CCOCC1)N(Cc1ccc(-c2nnc(C(F)(F)F)o2)cc1F)C12CC3CC(CC(C3)C1)C2] | 376 | 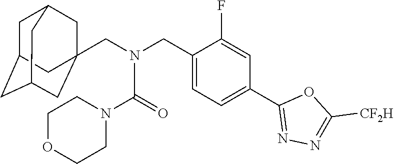 [SMILES: O=C(N1CCOCC1)N(Cc1ccc(-c2nnc(C(F)F)o2)cc1F)CC12CC3CC(CC(C3)C1)C2] |
| 377 | 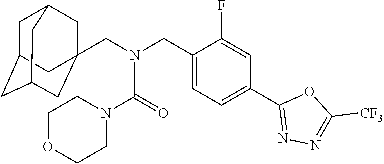 [SMILES: O=C(N1CCOCC1)N(Cc1ccc(-c2nnc(C(F)(F)F)o2)cc1F)CC12CC3CC(CC(C3)C1)C2] | 378 | 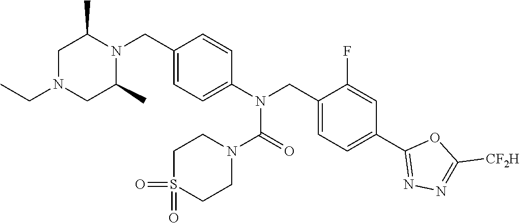 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 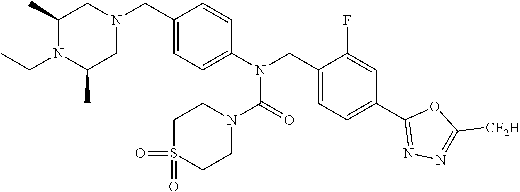 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] | 380 | 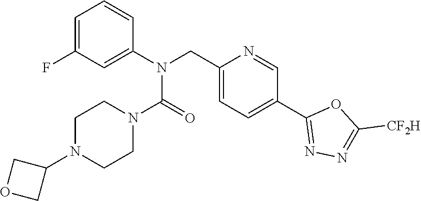 [SMILES: O=C(N1CCN(C2COC2)CC1)N(Cc1ccc(-c2nnc(C(F)F)o2)cn1)c1cccc(F)c1] |
| 381 | 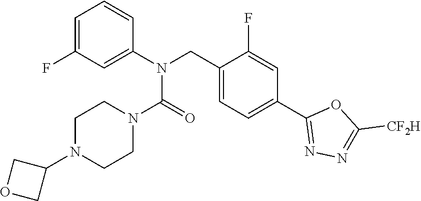 [SMILES: O=C(N1CCN(C2COC2)CC1)N(Cc1ccc(-c2nnc(C(F)F)o2)cc1F)c1cccc(F)c1] | 382 | 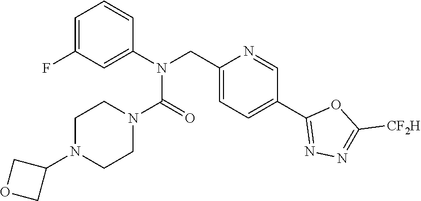 [SMILES: O=C(N1CCN(C2COC2)CC1)N(Cc1ccc(-c2nnc(C(F)F)o2)cn1)c1cccc(F)c1] |
| 383 | 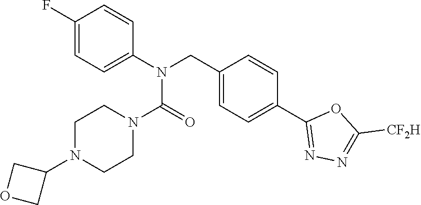 [SMILES: O=C(N1CCN(C2COC2)CC1)N(Cc1ccc(-c2nnc(C(F)F)o2)cc1)c1ccc(F)cc1] | 384 | 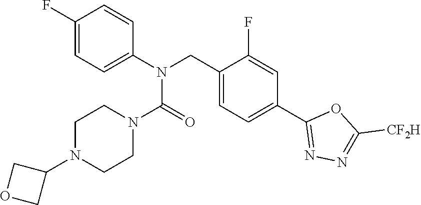 [SMILES: O=C(N1CCN(C2COC2)CC1)N(Cc1ccc(-c2nnc(C(F)F)o2)cc1F)c1ccc(F)cc1] |
| 385 | 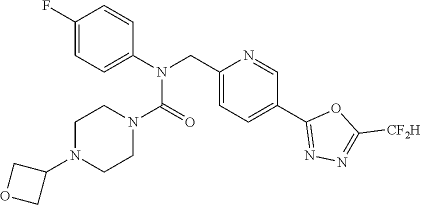 [SMILES: O=C(N1CCN(C2COC2)CC1)N(Cc1ccc(-c2nnc(C(F)F)o2)cn1)c1ccc(F)cc1] | 386 | 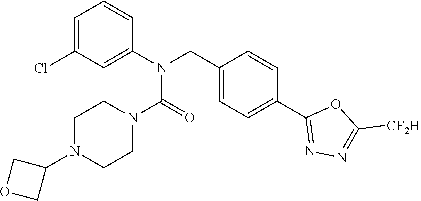 [SMILES: O=C(N1CCN(C2COC2)CC1)N(Cc1ccc(-c2nnc(C(F)F)o2)cc1)c1cccc(Cl)c1] |
| 387 | 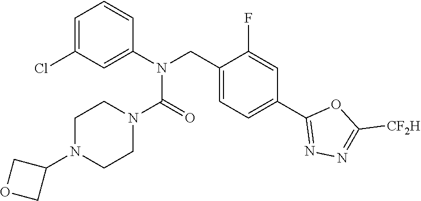 [SMILES: O=C(N1CCN(C2COC2)CC1)N(Cc1ccc(-c2nnc(C(F)F)o2)cc1F)c1cccc(Cl)c1] | 388 | 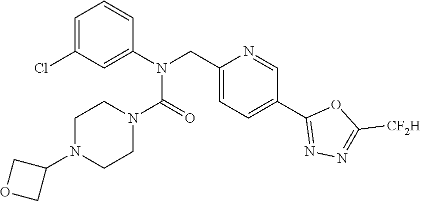 [SMILES: O=C(N1CCN(C2COC2)CC1)N(Cc1ccc(-c2nnc(C(F)F)o2)cn1)c1cccc(Cl)c1] |
| 389 | 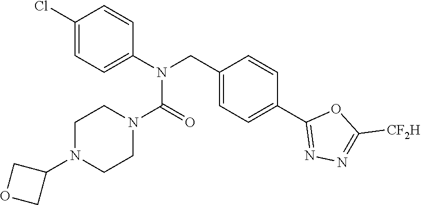 [SMILES: O=C(N1CCN(C2COC2)CC1)N(Cc1ccc(-c2nnc(C(F)F)o2)cc1)c1ccc(Cl)cc1] | 390 | 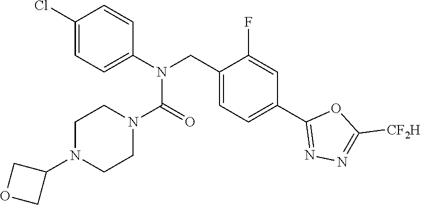 [SMILES: O=C(N1CCN(C2COC2)CC1)N(Cc1ccc(-c2nnc(C(F)F)o2)cc1F)c1ccc(Cl)cc1] |
| 391 | 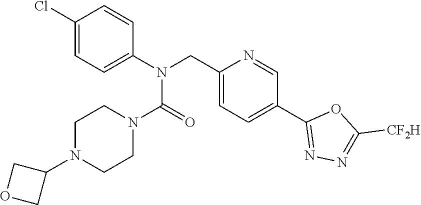 [SMILES: O=C(N1CCN(C2COC2)CC1)N(Cc1ccc(-c2nnc(C(F)F)o2)cn1)c1ccc(Cl)cc1] | 392 | 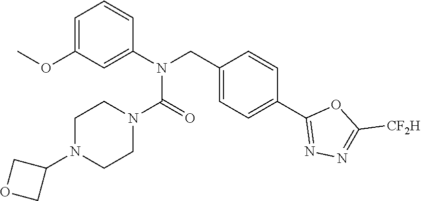 [SMILES: COc1cccc(N(Cc2ccc(-c3nnc(C(F)F)o3)cc2)C(=O)N2CCN(C3COC3)CC2)c1] |
| 393 | 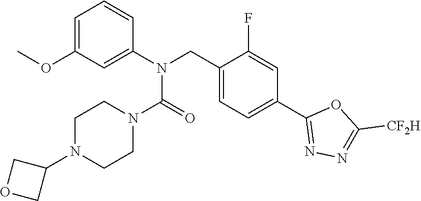 [SMILES: COc1cccc(N(Cc2ccc(-c3nnc(C(F)F)o3)cc2F)C(=O)N2CCN(C3COC3)CC2)c1] | 394 | 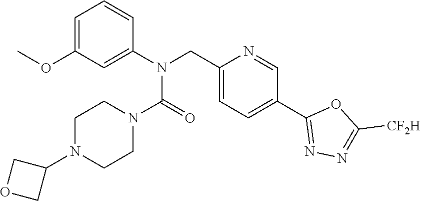 [SMILES: COc1cccc(N(Cc2ccc(-c3nnc(C(F)F)o3)cn2)C(=O)N2CCN(C3COC3)CC2)c1] |
| 395 | 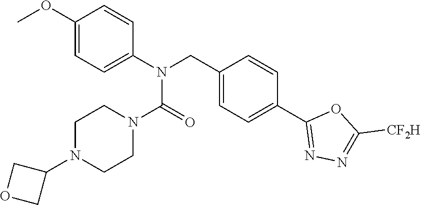 [SMILES: COc1ccc(N(Cc2ccc(-c3nnc(C(F)F)o3)cc2)C(=O)N2CCN(C3COC3)CC2)cc1] | 396 | 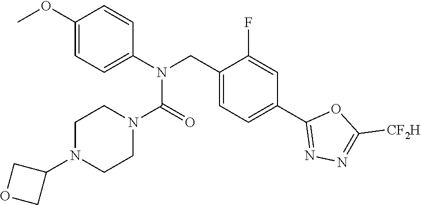 [SMILES: COc1ccc(N(Cc2ccc(-c3nnc(C(F)F)o3)cc2F)C(=O)N2CCN(C3COC3)CC2)cc1] |
| 397 | 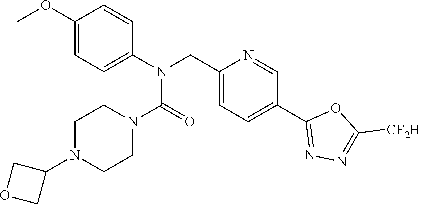 [SMILES: COc1ccc(N(Cc2ccc(-c3nnc(C(F)F)o3)cn2)C(=O)N2CCN(C3COC3)CC2)cc1] | 398 | 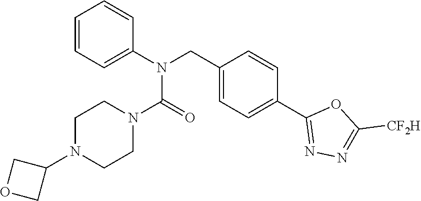 [SMILES: O=C(N1CCN(C2COC2)CC1)N(Cc1ccc(-c2nnc(C(F)F)o2)cc1)c1ccccc1] |
| 399 | 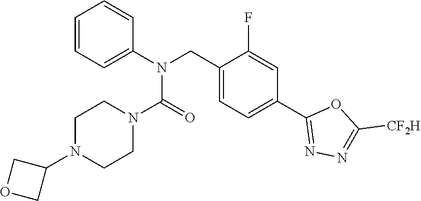 [SMILES: O=C(N1CCN(C2COC2)CC1)N(Cc1ccc(-c2nnc(C(F)F)o2)cc1F)c1ccccc1] | 400 | 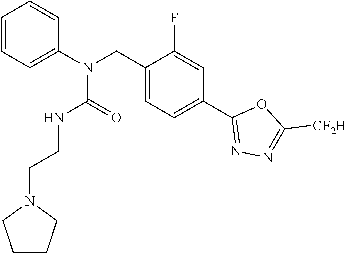 [SMILES: O=C(NCCN1CCCC1)N(Cc1ccc(-c2nnc(C(F)F)o2)cc1F)c1ccccc1] |
| 401 | 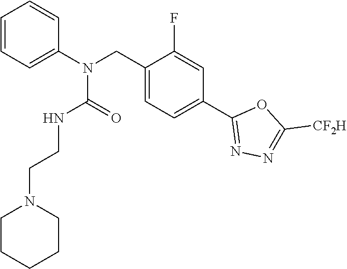 [SMILES: O=C(NCCN1CCCCC1)N(Cc1ccc(-c2nnc(C(F)F)o2)cc1F)c1ccccc1] | 402 | 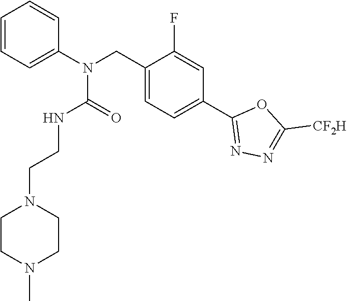 [SMILES: CN1CCN(CCNC(=O)N(Cc2ccc(-c3nnc(C(F)F)o3)cc2F)c2ccccc2)CC1] |
| 403 | 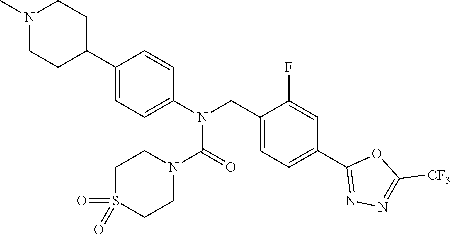 [SMILES: CN1CCC(c2ccc(N(Cc3ccc(-c4nnc(C(F)(F)F)o4)cc3F)C(=O)N3CCS(=O)(=O)CC3)cc2)CC1] | 404 | 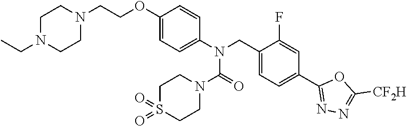 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 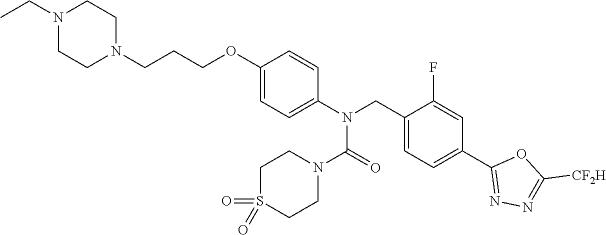 [SMILES: CCN1CCN(CCCOc2ccc(N(Cc3ccc(-c4nnc(C(F)F)o4)cc3F)C(=O)N3CCS(=O)(=O)CC3)cc2)CC1] | 406 | 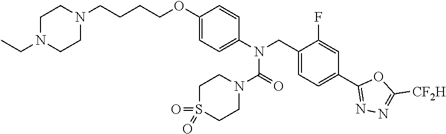 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 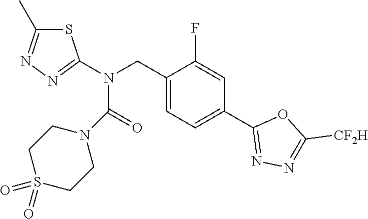 [SMILES: Cc1nnc(N(Cc2ccc(-c3nnc(C(F)F)o3)cc2F)C(=O)N2CCS(=O)(=O)CC2)s1] | 408 | 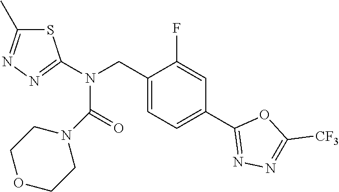 [SMILES: Cc1nnc(N(Cc2ccc(-c3nnc(C(F)(F)F)o3)cc2F)C(=O)N2CCOCC2)s1] |
| 409 | 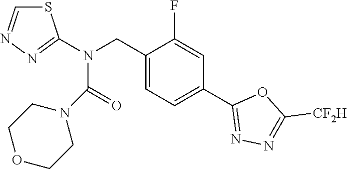 [SMILES: O=C(N1CCOCC1)N(Cc1ccc(-c2nnc(C(F)F)o2)cc1F)c1nncs1] | 410 | 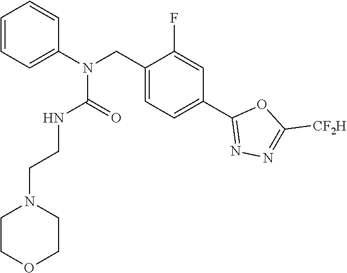 [SMILES: O=C(NCCN1CCOCC1)N(Cc1ccc(-c2nnc(C(F)F)o2)cc1F)c1ccccc1] |
| 411 | 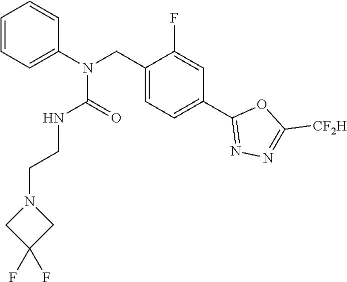 [SMILES: O=C(NCCN1CC(F)(F)C1)N(Cc1ccc(-c2nnc(C(F)F)o2)cc1F)c1ccccc1] | 412 | 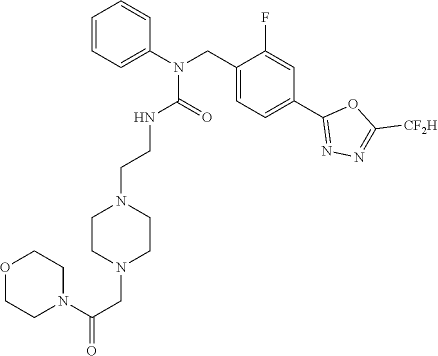 [SMILES: O=C(CN1CCN(CCNC(=O)N(Cc2ccc(-c3nnc(C(F)F)o3)cc2F)c2ccccc2)CC1)N1CCOCC1] |
| 413 | 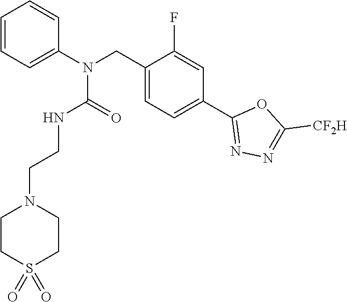 [SMILES: O=C(NCCN1CCS(=O)(=O)CC1)N(Cc1ccc(-c2nnc(C(F)F)o2)cc1F)c1ccccc1] | 414 | 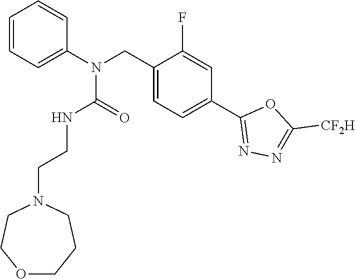 [SMILES: O=C(NCCN1CCCOCC1)N(Cc1ccc(-c2nnc(C(F)F)o2)cc1F)c1ccccc1] |
| 415 | 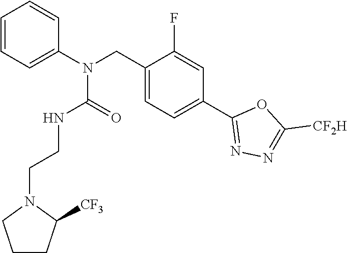 [SMILES: O=C(NCCN1CCC[C@@H]1C(F)(F)F)N(Cc1ccc(-c2nnc(C(F)F)o2)cc1F)c1ccccc1] | 416 | 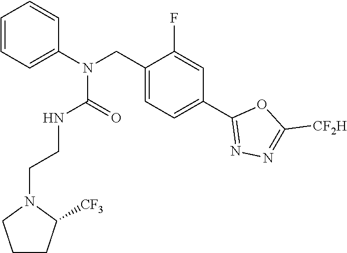 [SMILES: O=C(NCCN1CCC[C@H]1C(F)(F)F)N(Cc1ccc(-c2nnc(C(F)F)o2)cc1F)c1ccccc1] |
| 417 | 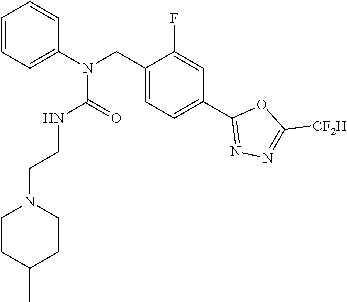 [SMILES: CC1CCN(CCNC(=O)N(Cc2ccc(-c3nnc(C(F)F)o3)cc2F)c2ccccc2)CC1] | 418 | 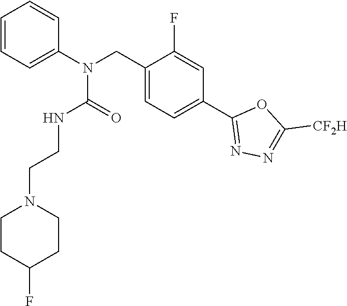 [SMILES: O=C(NCCN1CCC(F)CC1)N(Cc1ccc(-c2nnc(C(F)F)o2)cc1F)c1ccccc1] |
| 419 | 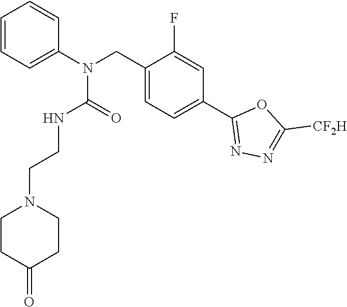 [SMILES: O=C1CCN(CCNC(=O)N(Cc2ccc(-c3nnc(C(F)F)o3)cc2F)c2ccccc2)CC1] | 420 | 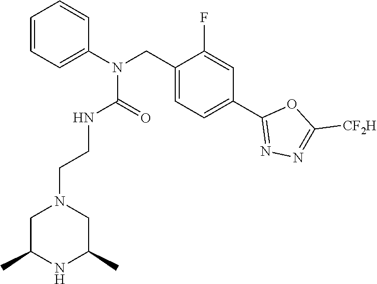 [SMILES: C[C@@H]1CN(CCNC(=O)N(Cc2ccc(-c3nnc(C(F)F)o3)cc2F)c2ccccc2)C[C@H](C)N1] |
| 421 | 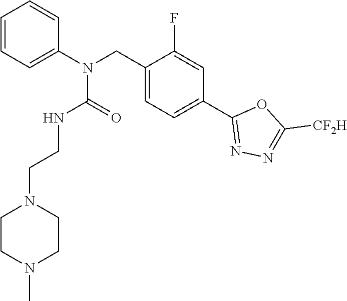 [SMILES: CN1CCN(CCNC(=O)N(Cc2ccc(-c3nnc(C(F)F)o3)cc2F)c2ccccc2)CC1] | 422 | 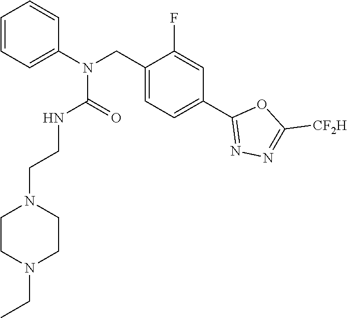 [SMILES: CCN1CCN(CCNC(=O)N(Cc2ccc(-c3nnc(C(F)F)o3)cc2F)c2ccccc2)CC1] |
| 423 | 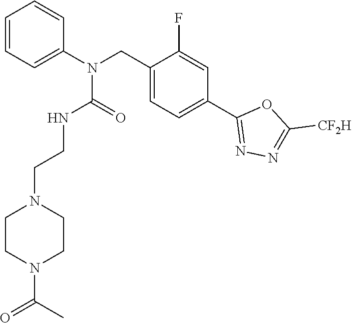 [SMILES: CC(=O)N1CCN(CCNC(=O)N(Cc2ccc(-c3nnc(C(F)F)o3)cc2F)c2ccccc2)CC1] | 424 | 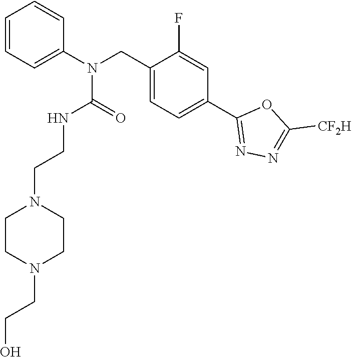 [SMILES: O=C(NCCN1CCN(CCO)CC1)N(Cc1ccc(-c2nnc(C(F)F)o2)cc1F)c1ccccc1] |
| 425 | 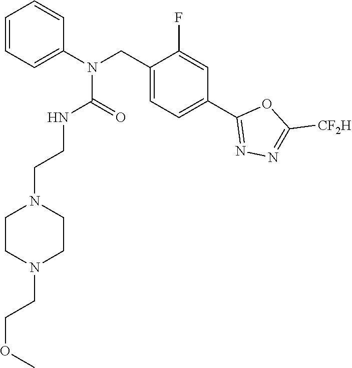 [SMILES: COCCN1CCN(CCNC(=O)N(Cc2ccc(-c3nnc(C(F)F)o3)cc2F)c2ccccc2)CC1] | 426 | 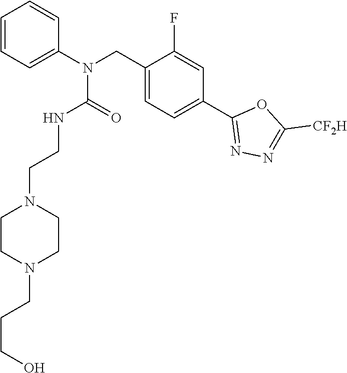 [SMILES: O=C(NCCN1CCN(CCCO)CC1)N(Cc1ccc(-c2nnc(C(F)F)o2)cc1F)c1ccccc1] |
| 427 | 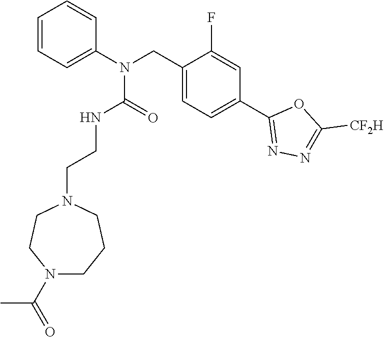 [SMILES: CC(=O)N1CCCN(CCNC(=O)N(Cc2ccc(-c3nnc(C(F)F)o3)cc2F)c2ccccc2)CC1] | 428 | 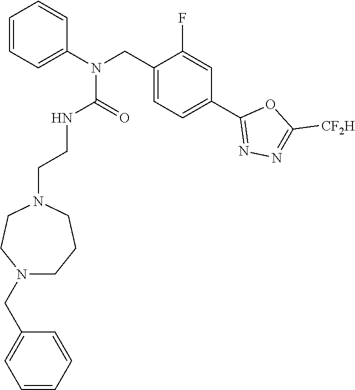 [SMILES: O=C(NCCN1CCCN(Cc2ccccc2)CC1)N(Cc1ccc(-c2nnc(C(F)F)o2)cc1F)c1ccccc1] |
| 429 | 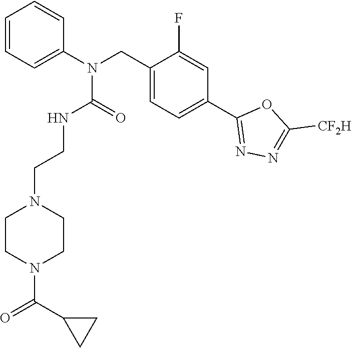 [SMILES: O=C(C1CC1)N1CCN(CCNC(=O)N(Cc2ccc(-c3nnc(C(F)F)o3)cc2F)c2ccccc2)CC1] | 430 | 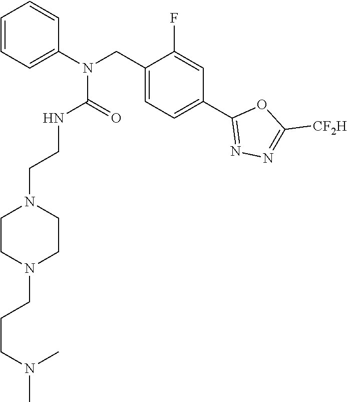 [SMILES: CN(C)CCCN1CCN(CCNC(=O)N(Cc2ccc(-c3nnc(C(F)F)o3)cc2F)c2ccccc2)CC1] |
| 431 | 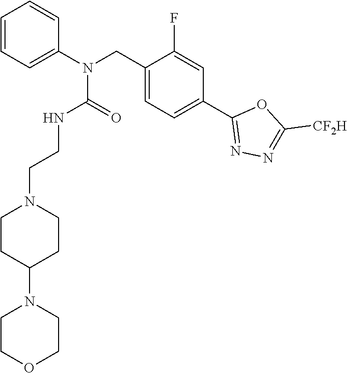 [SMILES: O=C(NCCN1CCC(N2CCOCC2)CC1)N(Cc1ccc(-c2nnc(C(F)F)o2)cc1F)c1ccccc1] | 432 | 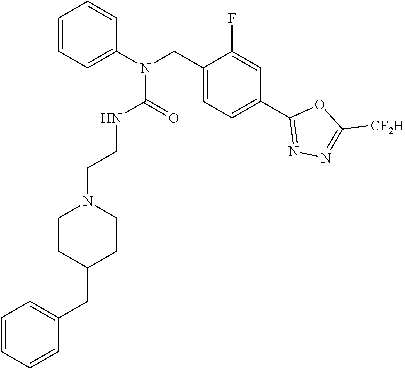 [SMILES: O=C(NCCN1CCC(Cc2ccccc2)CC1)N(Cc1ccc(-c2nnc(C(F)F)o2)cc1F)c1ccccc1] |
| 433 | 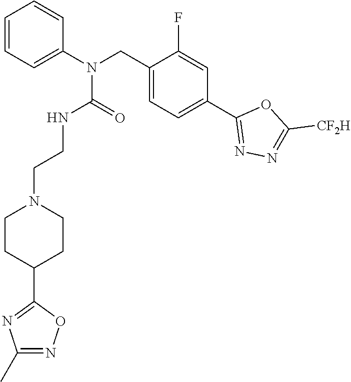 [SMILES: Cc1noc(C2CCN(CCNC(=O)N(Cc3ccc(-c4nnc(C(F)F)o4)cc3F)c3ccccc3)CC2)n1] | 434 | 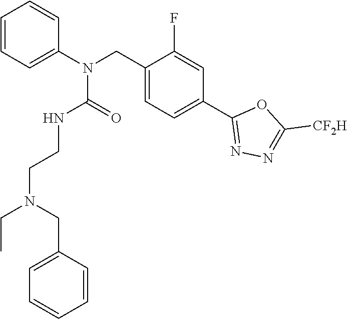 [SMILES: CCN(CCNC(=O)N(Cc1ccc(-c2nnc(C(F)F)o2)cc1F)c1ccccc1)Cc1ccccc1] |
| 435 | 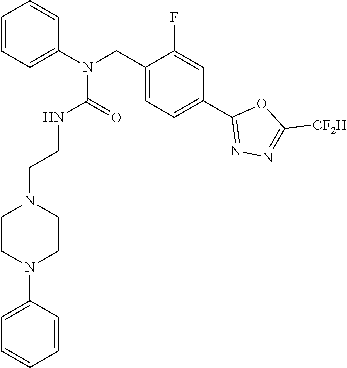 [SMILES: O=C(NCCN1CCN(c2ccccc2)CC1)N(Cc1ccc(-c2nnc(C(F)F)o2)cc1F)c1ccccc1] | 436 | 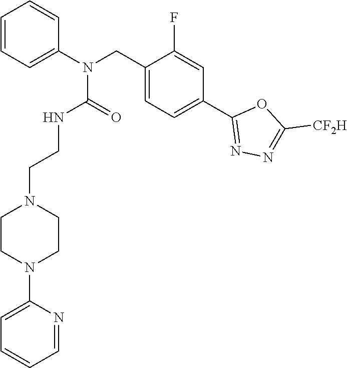 [SMILES: O=C(NCCN1CCN(c2ccccn2)CC1)N(Cc1ccc(-c2nnc(C(F)F)o2)cc1F)c1ccccc1] |
| 437 | 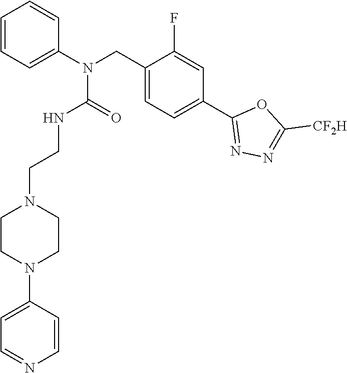 [SMILES: O=C(NCCN1CCN(c2ccncc2)CC1)N(Cc1ccc(-c2nnc(C(F)F)o2)cc1F)c1ccccc1] | 438 | 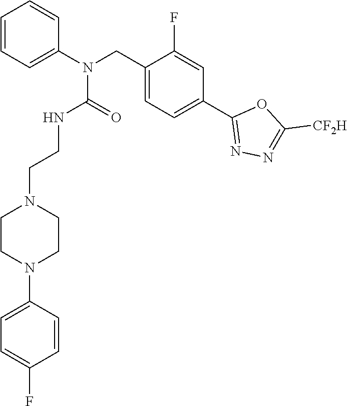 [SMILES: O=C(NCCN1CCN(c2ccc(F)cc2)CC1)N(Cc1ccc(-c2nnc(C(F)F)o2)cc1F)c1ccccc1] |
| 439 | 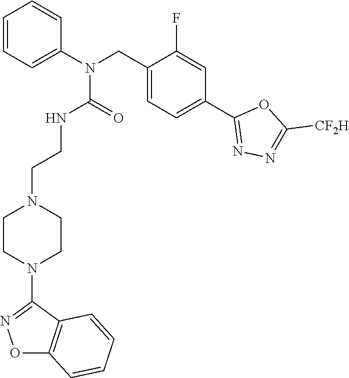 [SMILES: O=C(NCCN1CCN(c2noc3ccccc23)CC1)N(Cc1ccc(-c2nnc(C(F)F)o2)cc1F)c1ccccc1] | 440 | 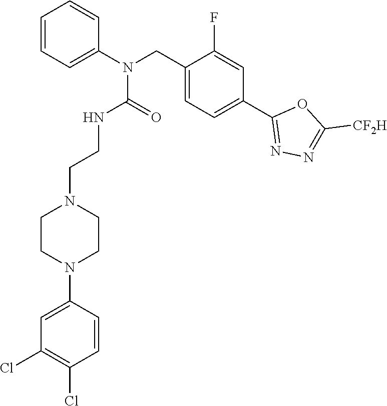 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 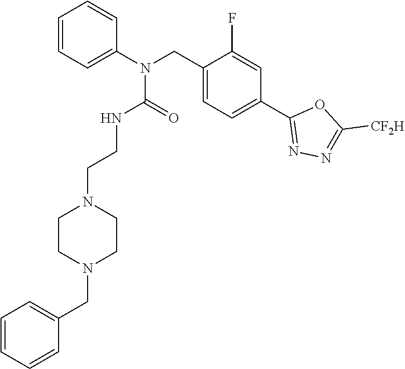 [SMILES: O=C(NCCN1CCN(Cc2ccccc2)CC1)N(Cc1ccc(-c2nnc(C(F)F)o2)cc1F)c1ccccc1] | 442 | 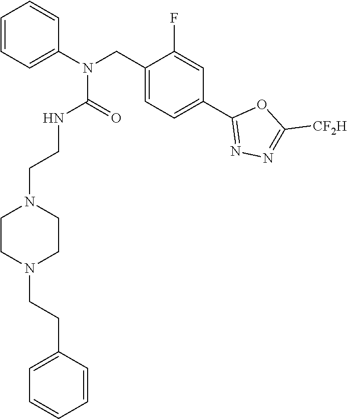 [SMILES: O=C(NCCN1CCN(CCc2ccccc2)CC1)N(Cc1ccc(-c2nnc(C(F)F)o2)cc1F)c1ccccc1] |
| 443 | 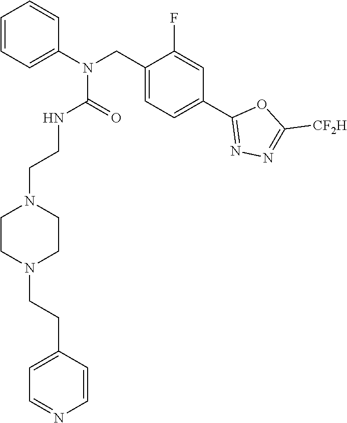 [SMILES: O=C(NCCN1CCN(CCc2ccncc2)CC1)N(Cc1ccc(-c2nnc(C(F)F)o2)cc1F)c1ccccc1] | 444 | 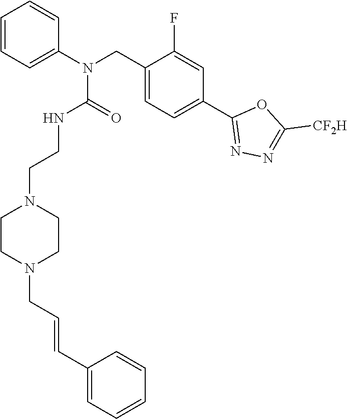 [SMILES: O=C(NCCN1CCN(C/C=C/c2ccccc2)CC1)N(Cc1ccc(-c2nnc(C(F)F)o2)cc1F)c1ccccc1] |
| 445 | 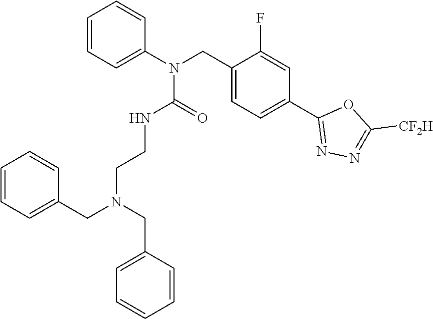 [SMILES: O=C(NCCN(Cc1ccccc1)Cc1ccccc1)N(Cc1ccc(-c2nnc(C(F)F)o2)cc1F)c1ccccc1] | 446 | 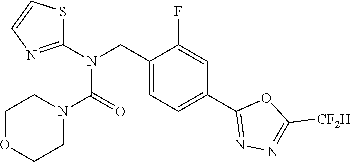 [SMILES: O=C(N1CCOCC1)N(Cc1ccc(-c2nnc(C(F)F)o2)cc1F)c1nccs1] |
| 447 | 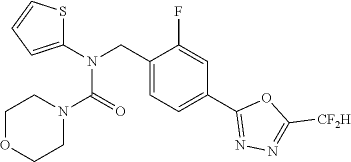 [SMILES: O=C(N1CCOCC1)N(Cc1ccc(-c2nnc(C(F)F)o2)cc1F)c1cccs1] | 448 | 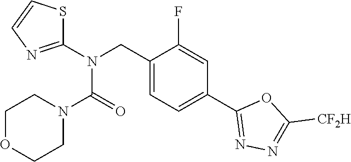 [SMILES: O=C(N1CCOCC1)N(Cc1ccc(-c2nnc(C(F)F)o2)cc1F)c1nccs1] |
| 449 | 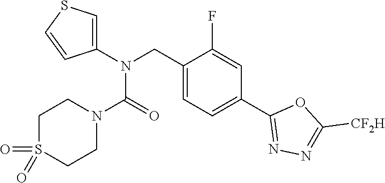 [SMILES: O=C(N1CCS(=O)(=O)CC1)N(Cc1ccc(-c2nnc(C(F)F)o2)cc1F)c1ccsc1] | 450 | 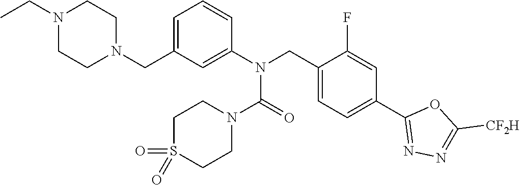 [SMILES: CCN1CCN(Cc2cccc(N(Cc3ccc(-c4nnc(C(F)F)o4)cc3F)C(=O)N3CCS(=O)(=O)CC3)c2)CC1] |

In example embodiment of the present disclosure, the pharmaceutical composition including a compound of the above table, optical isomers thereof or pharmaceutically acceptable salts thereof as an effective ingredient may prevent or treat idiopathic pulmonary fibrosis.

In the present disclosure, the compound represented by the above formula I may be a compound described in a following table.

| Com- | |
|---|---|
| pound | Structure |
| 40 | 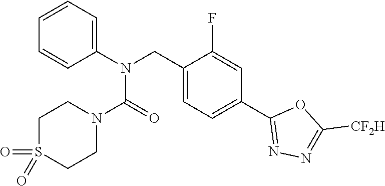 [SMILES: O=C(N1CCS(=O)(=O)CC1)N(Cc1ccc(-c2nnc(C(F)F)o2)cc1F)c1ccccc1] |
| 43 | 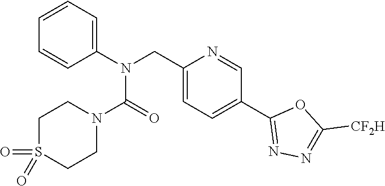 [SMILES: O=C(N1CCS(=O)(=O)CC1)N(Cc1ccc(-c2nnc(C(F)F)o2)cn1)c1ccccc1] |
| 239 | 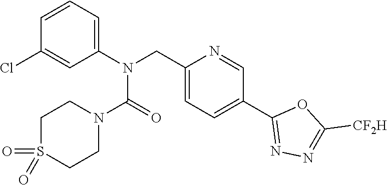 [SMILES: O=C(N1CCS(=O)(=O)CC1)N(Cc1ccc(-c2nnc(C(F)F)o2)cn1)c1cccc(Cl)c1] |
| 285 | 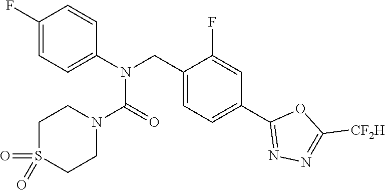 [SMILES: O=C(N1CCS(=O)(=O)CC1)N(Cc1ccc(-c2nnc(C(F)F)o2)cc1F)c1ccc(F)cc1] |
| 295 | 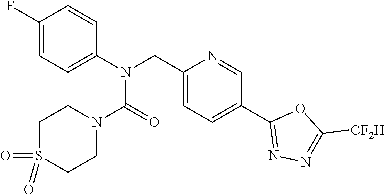 [SMILES: O=C(N1CCS(=O)(=O)CC1)N(Cc1ccc(-c2nnc(C(F)F)o2)cn1)c1ccc(F)cc1] |
| 296 | 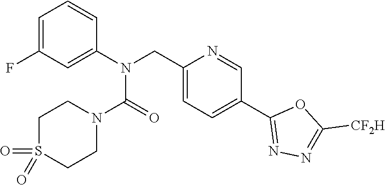 [SMILES: O=C(N1CCS(=O)(=O)CC1)N(Cc1ccc(-c2nnc(C(F)F)o2)cn1)c1cccc(F)c1] |

In example embodiment of the present disclosure, the pharmaceutical composition including a compound of the above table, optical isomers thereof or pharmaceutically acceptable salts thereof as an effective ingredient may prevent or treat idiopathic pulmonary fibrosis.

In the present disclosure, the compound represented by the above formula I may be prepared by a method disclosed in Korean Unexamined Patent Application Publication No. 10-2017-0017792, but is not limited thereto.

In the present disclosure, the compound represented by the above formula I may contain at least one asymmetric carbon, and thus may be present as an enantiomer mixture including a racemic mixture, a single enantiomer (optical isomer), a mixture of diastereomers, and a single diastereomer. Such isomer may be separated by split according to the related art, for example, column chromatography, HPLC or the like. Alternatively, the isomer may be stereospecifically synthesized by using a known array of optically pure starting materials and/or reagents. Particularly, said isomer may be an optical isomer (enantiomer).

In the present disclosure, the term “pharmaceutically acceptable” may refer to the one which is physiologically acceptable and does not conventionally cause gastrointestinal disturbance, an allergic response such as dizziness or other responses similar thereto, when being administered to an individual.

The pharmaceutically acceptable salts according to the embodiments of the present disclosure may be prepared by a conventional method known to those skilled in the art.

The pharmaceutically acceptable salts according to the embodiment of the present disclosure may include, for example, inorganic ion salts prepared from calcium, potassium, sodium, magnesium, etc.; inorganic acid salts prepared from hydrochloric acid, nitric acid, phosphoric acid, bromic acid, iodic acid, perchloric acid, sulfuric acid, hydroiodic acid, etc.; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, etc.; sulfonic acid salts prepared from methanesulfonic acid, ethanesulfonic acid, benzenesulfonic acid, p-toluenesulfonic acid, naphthalene sulfonic acid, etc.; amino acid salts prepared from glycine, arginine, lysine, etc.; amine salts prepared from trimethylamine, triethylamine, ammonia, pyridine, picoline, etc.; and the like, but are not limited thereto. In the embodiments of the present disclosure, salts may include hydrochloric acid, trifluoroacetic acid, citric acid, bromic acid, maleic acid, phosphoric acid, sulfuric acid, tartaric acid or a mixture thereof.

In the present disclosure, the term “idiopathic pulmonary fibrosis (IPF)” may refer to a disease in which the lung parenchyma becomes fibrous due to an abnormal tissue repair mechanism after damage to alveolar epithelial cells for unknown reasons.

In the present disclosure, the term “prevention” may refer to all the acts, which inhibit or delay the occurrence of a disease by administering the compound of formula I of the present disclosure, optical isomers thereof or pharmaceutically acceptable salts thereof.

In the present disclosure, the term “treatment” may refer to all the acts, by which a symptom of an individual likely to develop or suffering from a disease gets better or takes a favorable turn by administering the compound of formula I of the present disclosure, optical isomers thereof or pharmaceutically acceptable salts thereof.

The compound represented by formula I of the present disclosure, optical isomers thereof or pharmaceutically acceptable salts thereof may be advantageously used in preventing or treating idiopathic pulmonary fibrosis.

A pharmaceutical composition including the compound represented by formula I of the present disclosure, optical isomers thereof, or pharmaceutically acceptable salts thereof as an effective ingredient may be advantageously used in preventing or treating idiopathic pulmonary fibrosis.

In this regard, in one specific embodiment of the present disclosure, it was confirmed that the compound represented by formula I of the present disclosure, optical isomers thereof or pharmaceutically acceptable salts thereof inhibit the expression of TGF-β1-induced fibrotic proteins, FN-EDA and proCOL1A1 (FIG. 1).

In addition, it was confirmed that the compound represented by formula I of the present disclosure, optical isomers thereof or pharmaceutically acceptable salts thereof inhibit the expression of fibrotic proteins, COL1A1 and αSMA, which are increased in mice with BLM-induced pulmonary fibrosis (FIG. 2). Furthermore, it was confirmed that the compound represented by formula I of the present disclosure, optical isomers thereof or pharmaceutically acceptable salts thereof decrease an increased Ashcroft score and improve a decreased physical activity in mice with BLM-induced pulmonary fibrosis (FIG. 3).

The compound represented by formula I of the present disclosure, optical isomers thereof or pharmaceutically acceptable salts thereof may show an effect of preventing or treating idiopathic pulmonary fibrosis at a level that is similar to or substantially the same as or superior to a conventionally known drug for preventing or treating idiopathic pulmonary fibrosis.

A pharmaceutical composition including the compound represented by formula I of the present disclosure, optical isomers thereof, or pharmaceutically acceptable salts thereof as an effective ingredient may show an effect of preventing or treating idiopathic pulmonary fibrosis at a level that is similar to or substantially the same as or superior to a conventionally known drug for preventing or treating idiopathic pulmonary fibrosis.

The pharmaceutical composition according to the embodiments of the present disclosure may further include at least one pharmaceutically acceptable carrier, in addition to the compound represented by the above formula I, optical isomers thereof or pharmaceutically acceptable salts thereof. The pharmaceutically acceptable carrier may be the one which is conventionally used in the art, specifically including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidine, cellulose, water, syrup, methylcellulose, methyl hydroxybenzoate, propyl hydroxybenzoate, talc, magnesium stearate, or mineral oil. The pharmaceutical composition according to the embodiments of the present disclosure may further include lubricants, humectants, sweetening agents, flavoring agents, emulsifiers, suspending agents, preservatives, dispersing agents, stabilizing agents, etc., in addition to the above ingredients. In addition, the pharmaceutical composition according to the embodiments of the present disclosure may be formulated into an oral dosage form such as a tablet, powder, granule, pill, capsule, suspension, emulsion, liquid for internal use, oiling agent, syrup, etc., as well as a form of external application, suppository or sterile solution for injection, by using pharmaceutically acceptable carriers and excipients and thus may be prepared in a unit dose form or prepared by being inserted into a multi-dose container. Such preparations may be prepared according to a conventional method used for formulation in the art or a method disclosed in Remington's Pharmaceutical Science (19^{th} ed., 1995), and may be formulated into various preparations depending on each disease or ingredient.

A non-limiting example of preparations for oral administration using the pharmaceutical composition of the present disclosure may include tablets, troches, lozenges, water-soluble suspensions, oil suspensions, prepared powders, granules, emulsions, hard capsules, soft capsules, syrups, elixirs or the like. To formulate the pharmaceutical composition according to the embodiments of the present disclosure into preparation for oral administration, the followings may be used: binders such as lactose, saccharose, sorbitol, mannitol, starch, amylopectin, cellulose, gelatin or the like; excipients such as dicalcium phosphate, etc.; disintegrants such as maize starch, sweet potato starch or the like; lubricants such as magnesium stearate, calcium stearate, sodium stearyl fumarate, polyethylene glycol wax, or the like; etc., in which sweetening agents, flavoring agents, syrups, etc. may also be used. Furthermore, in the case of the capsules, liquid carriers such as fatty oil, etc. may be further used in addition to the above-mentioned materials.

A non-limiting example of parenteral preparations using the pharmaceutical composition according to the embodiments of the present disclosure may include injectable solutions, suppositories, powders for respiratory inhalation, aerosols for spray, ointments, powders for application, oils, creams, etc. To formulate the pharmaceutical composition according to the embodiments of the present disclosure into preparation for parenteral administration, the following may be used: sterilized aqueous solutions, non-aqueous solvents, suspensions, emulsions, freeze-dried preparations, external preparations, etc. As said non-aqueous solvents and suspensions, the following may be used, but without limitation thereto: propylene glycol, polyethylene glycol, vegetable oils such as olive oil, injectable esters such as ethyl oleate, etc.

The pharmaceutical composition according to the embodiments of the present disclosure may be subjected to oral administration or parenteral administration according to a targeted method, preferably oral administration, but is not limited thereto.

A daily dosage of the compound represented by formula I of the present disclosure, optical isomers thereof or pharmaceutically acceptable salts thereof may be particularly about 0.1 to about 10,000 mg/kg, about 1 to about 8,000 mg/kg, about 5 to about 6,000 mg/kg, or about 10 to about 4,000 mg/kg, and more particularly about 50 to about 2,000 mg/kg, but is not limited thereto and may be also administered once a day or several times a day by dividing the daily dosage of the compound.

A pharmaceutically effective dose and an effective dosage of the pharmaceutical composition according to the embodiments of the present disclosure may vary depending on a method for formulating the pharmaceutical composition, an administration mode, an administration time, an administration route, and/or the like, and may be diversified according to various factors including a type and degree of reaction to be achieved by administration of the pharmaceutical composition, a type of an individual for administration, the individual's age, weight, general health condition, disease symptom or severity, gender, diet and excretion, ingredients of other drug compositions to be used for the corresponding individual at the same time or different times, etc., as well as other similar factors well known in a pharmaceutical field, and those skilled in the art may easily determine and prescribe an effective dosage for the intended treatment.

The pharmaceutical composition according to the embodiments of the present disclosure may be administered once a day or several times a day. The pharmaceutical composition according to the embodiments of the present disclosure may be administered as an individual therapeutic agent or in combination with other therapeutic agents, and may be administered sequentially or simultaneously with a conventional therapeutic agent. Considering all the above factors, the pharmaceutical composition according to the embodiments of the present disclosure may be administered in such an amount that a maximum effect may be achieved by a minimum amount without a side effect, and such amount may be easily determined by those skilled in the art to which the present disclosure pertains.

The pharmaceutical composition according to the embodiments of the present disclosure may show an excellent effect even when solely used, but may be further used in combination with various methods such as hormone therapy, drug treatment, etc. to increase therapeutic efficiency.

The present disclosure may provide a method for preventing or treating idiopathic pulmonary fibrosis, including administering a compound represented by the above formula I, optical isomers thereof or pharmaceutically acceptable salts thereof into an individual.

Said terms “idiopathic pulmonary fibrosis,” “prevention” and “treatment” may be the same as described above.

In the present disclosure, the term “administration” may refer to introducing a predetermined substance into an individual by an appropriate method.

In the present disclosure, the term “individual” may refer to all the animals such as rats, mice, livestock, etc., including humans, who have developed or are likely to develop idiopathic pulmonary fibrosis, and may be particularly mammals including humans, but is not limited thereto.

The method for preventing or treating idiopathic pulmonary fibrosis according to the embodiments of the present disclosure may include administering a therapeutically effective amount of the compound represented by the above formula I, optical isomers thereof or pharmaceutically acceptable salts thereof.

In the present disclosure, the term “therapeutically effective amount” may refer to an amount enough to treat a disease at a reasonable risk/benefit ratio applicable to medical treatment and not to cause a side effect, and may be determined by those skilled in the art according to factors including a patient's gender, age, weight, health condition, a type of disease, severity, the activity of a drug, sensitivity to a drug, an administration method, an administration time, an administration route, an excretion rate, a treatment period, a drug combined or concurrently used, as well as other factors well known in a pharmaceutical field. It is preferable to differently apply a particular therapeutically effective amount for a certain patient depending on various factors including a type and degree of reaction to be achieved therefrom, a particular composition including a presence of other preparations used in some cases, a patient's age, weight, general health condition, gender and diet, an administration time, an administration route, a secretion rate of the composition, a treatment period and a drug used together with the particular composition or simultaneously therewith, as well as other similar factors well known in a pharmaceutical field.

The method for preventing or treating idiopathic pulmonary fibrosis according to the embodiments of the present disclosure may include not only dealing with the disease per se before expression of its symptoms, but also inhibiting or avoiding such symptoms by administering the compound represented by the above formula I, isomers thereof or pharmaceutically acceptable salts thereof. In managing the disease, a preventive or therapeutic dose of a certain active ingredient may vary depending on the characteristics and severity of the disease or conditions, and a route in which the active ingredient is administered. A dose and a frequency thereof may vary depending on an individual patient's age, weight and reactions. A suitable dose and usage may be easily selected by those skilled in the art, naturally considering such factors. In addition, the method for preventing or treating idiopathic pulmonary fibrosis according to the embodiments of the present disclosure may further include administering a therapeutically effective amount of an additional active agent, which helps prevent or treat the disease, along with the compound represented by the above formula I, optical isomers thereof or pharmaceutically acceptable salts thereof, and the additional active agent may show a synergy effect or an additive effect together with the compound represented by the above formula I, optical isomers thereof or pharmaceutically acceptable salts thereof.

The present disclosure may provide a use of the compound represented by the above formula I, optical isomers thereof or pharmaceutically acceptable salts thereof for preventing or treating idiopathic pulmonary fibrosis.

The present disclosure may provide a use of the compound represented by the above formula I, optical isomers thereof or pharmaceutically acceptable salts thereof in preparing a medicament for preventing or treating idiopathic pulmonary fibrosis.

Said terms “idiopathic pulmonary fibrosis,” “prevention” and “treatment” may be the same as described above.

For the preparation of the medicament, the compound represented by the above formula I, optical isomers thereof or pharmaceutically acceptable salts thereof may be mixed with pharmaceutically acceptable adjuvants, diluents, carriers, etc., and may be prepared into a complex preparation together with other active agents, thus providing a synergy action.

Matters mentioned in the pharmaceutical composition, treatment method and use of the present disclosure are applied the same, if not contradictory to each other.

### Advantageous Effects

The compound represented by formula I of the present disclosure, optical isomers thereof or pharmaceutically acceptable salts thereof and the pharmaceutical composition including the same as an effective ingredient may be advantageously used in preventing or treating idiopathic pulmonary fibrosis.

### BRIEF DESCRIPTION OF THE DRAWINGS

FIG. 1 is a graph showing an expression level of fibrotic proteins (FN-EDA and proCOL1A1) in each group. (* p<0.05, ** p<0.01, *** p<0.001)

FIG. 2 is a graph showing an expression level of fibrotic proteins (COL1A1 and α-SMA) in each group. (** p<0.01, *** p<0.001)

FIG. 3 is a graph showing an Ashcroft score and treadmill test evaluation results (physical activity) of each group. (###p<0.001, * p<0.05)

### MODE FOR INVENTION

The present disclosure will be described in detail with reference to Examples hereinafter. However, the Examples are only for the purpose of illustrating the present disclosure and it is obvious to those skilled in the art that the scope of the present disclosure is not limited to the Examples disclosed hereinafter.

### Synthesis Example 1. Synthesis of Compound 40, N-(4-(5-(difluoromethyl)-1,3,4-oxadiazol-2-yl)-2-fluorobenzyl)-N-phenylthiomorpholine-4-carboxamide 1,1-dioxide

### [Step 1] Synthesis of N-phenylthiomorpholine-4-carboxamide 1,1-dioxide

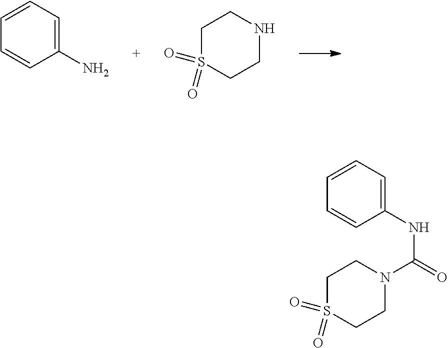 [SMILES: Nc1ccccc1 ; O=S1(=O)CCNCC1 ; O=C(Nc1ccccc1)N1CCS(=O)(=O)CC1]

Triphosgene (4.780 g, 16.107 mmol) was added to a solution of aniline (3.000 g, 32.213 mmol) and N,N-diisopropylethylamine (33.439 mL, 193.278 mmol) dissolved in dichloromethane (100 mL) at 0° C., and stirred at the same temperature. Thiomorpholine 1,1-dioxide (4.790 g, 35.434 mmol) was added to the reaction mixture, and stirred at the room temperature for additional 16 hr. Then, water was poured to the reaction mixture, followed by extraction with ethyl acetate. The organic layer was washed with saturated aqueous sodium chloride solution, dried (anhydrous MgSO₄), filtered, and concentrated in vacuo. The concentrate was purified and concentrated by column chromatography (SiO₂, 40 g cartridge; methanol/dichloromethane=2%) to give the title compound as yellow solid (1.325 g, 16.2%).

### [Step 2] Synthesis of Methyl 4-((1,1-dioxido-N-phenylthiomorpholine-4-carboxamido)methyl)-3-fluorobenzoate

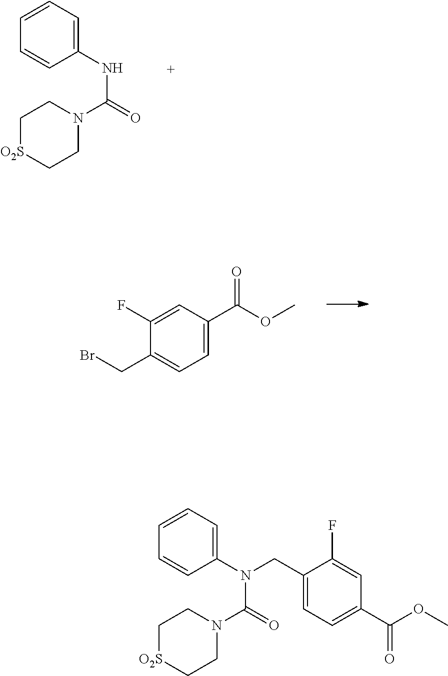 [SMILES: O=C(Nc1ccccc1)N1CCS(=O)(=O)CC1 ; COC(=O)c1ccc(CBr)c(F)c1 ; COC(=O)c1ccc(CN(C(=O)N2CCS(=O)(=O)CC2)c2ccccc2)c(F)c1]

Methyl 4-(bromomethyl)-3-fluorobenzoate (1.020 g, 4.129 mmol) is added to a solution of N-phenylthiomorpholine-4-carboxamide 1,1-dioxide (1.000 g, 3.932 mmol) prepared in Step 1 and sodium hydride (60.00%, 0.189 g, 4.719 mmol) dissolved in N,N-dimethylformamide (30 mL) at 0° C., and stirred at the room temperature for 18 hr. Then, saturated aqueous sodium bicarbonate solution was poured to the reaction mixture, followed by extraction with ethyl acetate. The organic layer was washed with saturated aqueous sodium chloride solution, dried (anhydrous MgSO₄), filtered, and concentrated in vacuo. The concentrate was purified and concentrated by column chromatography (SiO₂, 40 g cartridge; ethyl acetate/hexane=0% to 50%) to give the title compound as white solid (1.240 g, 75.0%).

### [Step 3] Synthesis of N-(2-fluoro-4-(hydrazinecarbonyl)benzyl)-N-phenylthiomorpholine-4-carboxamide 1,1-dioxide

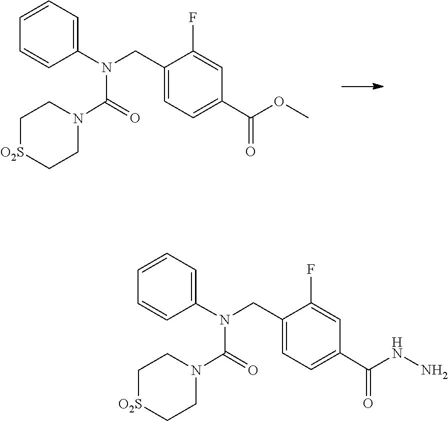 [SMILES: COC(=O)c1ccc(CN(C(=O)N2CCS(=O)(=O)CC2)c2ccccc2)c(F)c1 ; NNC(=O)c1ccc(CN(C(=O)N2CCS(=O)(=O)CC2)c2ccccc2)c(F)c1]

A solution of methyl 4-((1,1-dioxido-N-phenylthiomorpholine-4-carboxamido)methyl)-3-fluorobenzoate (1.240 g, 2.949 mmol) prepared in Step 2 and hydrazine monohydrate (2.786 mL, 58.983 mmol) dissolved in ethanol (15 mL) at 120° C. was stirred for 1 hr, and cooled down to the room temperature to terminate the reaction. The reaction mixture was concentrated in vacuo to remove the solvent, and saturated aqueous sodium bicarbonate solution was poured to the concentrate, followed by extraction with dichloromethane. The bi-phasic mixture was passed through a plastic filter to remove solid residues and aqueous layer, and the organic layer collected was concentrated in vacuo. The crude title compound was used without further purification (1.240 g, 100.0%, white solid).

### [Step 4] Synthesis of N-(4-(2-(2,2-difluoroacetyl)hydrazine-1-carbonyl)-2-fluorobenzyl)-N-phenylthiomorpholine-4-carboxamide 1,1-dioxide

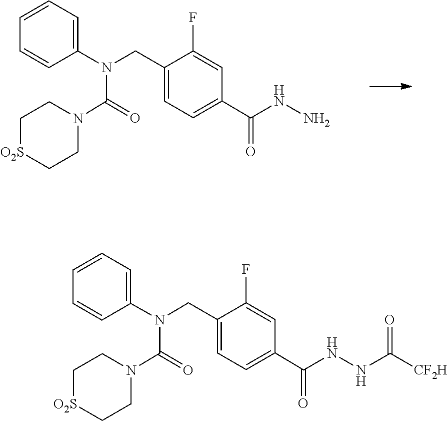 [SMILES: NNC(=O)c1ccc(CN(C(=O)N2CCS(=O)(=O)CC2)c2ccccc2)c(F)c1 ; O=C(NNC(=O)C(F)F)c1ccc(CN(C(=O)N2CCS(=O)(=O)CC2)c2ccccc2)c(F)c1]

A solution of N-(2-fluoro-4-(hydrazinecarbonyl)benzyl)-N-phenylthiomorpholine-4-carboxamide 1,1-dioxide (0.615 g, 1.463 mmol) prepared in Step 3, triethylamine (0.304 mL, 2.194 mmol) and difluoroacetic anhydride (0.164 mL, 1.316 mmol) dissolved in dichloromethane (10 mL) at the room temperature was stirred at the same temperature for 18 hr. Then, saturated aqueous sodium bicarbonate solution was poured to the reaction mixture, followed by extraction with ethyl acetate. The organic layer was washed with saturated aqueous sodium chloride solution, dried (anhydrous MgSO₄), filtered, and concentrated in vacuo. The concentrate was purified and concentrated by column chromatography (SiO₂, 24 g cartridge; methanol/dichloromethane=0% to 3%) to give the title compound as white solid (0.462 g, 63.4%).

### [Step 5] Synthesis of Compound 40

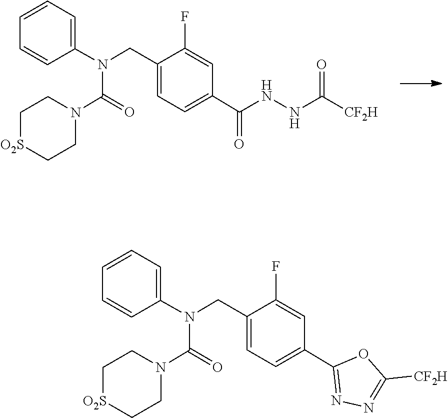 [SMILES: O=C(NNC(=O)C(F)F)c1ccc(CN(C(=O)N2CCS(=O)(=O)CC2)c2ccccc2)c(F)c1 ; O=C(N1CCS(=O)(=O)CC1)N(Cc1ccc(-c2nnc(C(F)F)o2)cc1F)c1ccccc1]

N-(4-(2-(2,2-difluoroacetyl) hydrazine-1-carbonyl)-2-fluorobenzyl)-N-phenylthiomorpholine-4-carboxamide 1,1-dioxide (0.462 g, 0.927 mmol) prepared in Step 4 and 1-methoxy-N-triethylammoniosulfonyl-methanimidate (burgess reagent, 0.331 g, 1.390 mmol) were mixed in tetrahydrofuran (10 mL), and heated at 150° C. for 30 min under the microwaves, and then cooled down to the room temperature to terminate the reaction. Then, saturated aqueous sodium bicarbonate solution was poured to the reaction mixture, followed by extraction with dichloromethane. The bi-phasic mixture was passed through a plastic filter to remove solid residues and aqueous layer, and the organic layer collected was concentrated in vacuo. The concentrate was purified and concentrated by column chromatography (SiO₂, 12 g cartridge; ethyl acetate/hexane=0% to 50%) to give the title compound as white solid (0.337 g, 75.7%).

¹H NMR (400 MHz, CDCl₃) δ 7.87-7.85 (m, 1H), 7.75-7.72 (m, 1H), 7.67-7.64 (m, 1H), 7.38-7.34 (m, 2H), 7.25-7.20 (m, 1H), 7.13-7.10 (m, 2H), 7.03-6.77 (m, 1H), 4.92 (s, 2H), 3.71-3.67 (m, 4H), 2.77-2.74 (m, 4H); LRMS (ES) m/z 481.1 (M⁺+1).

### Synthesis Example 2. Synthesis of Compound 43, N-((5-(5-(difluoromethyl)-1,3,4-oxadiazol-2-yl)pyridin-2-yl)methyl)-N-phenylthiomorpholine-4-carboxamide 1,1-dioxide

### [Step 1] Synthesis of Methyl 6-((1,1-dioxido-N-phenylthiomorpholine-4-carboxamido)methyl)nicotinate

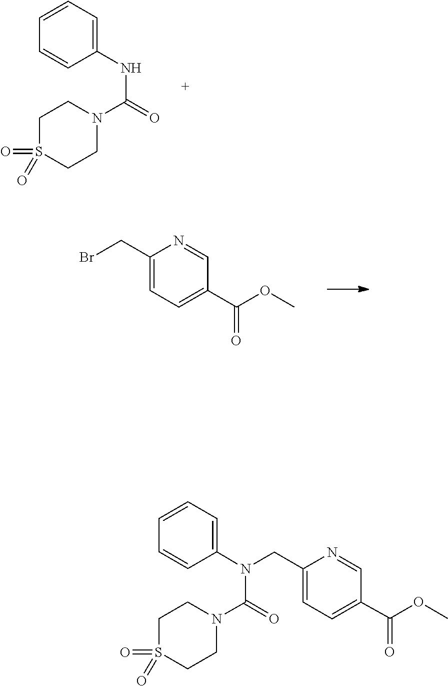 [SMILES: O=C(Nc1ccccc1)N1CCS(=O)(=O)CC1 ; COC(=O)c1ccc(CBr)nc1 ; COC(=O)c1ccc(CN(C(=O)N2CCS(=O)(=O)CC2)c2ccccc2)nc1]

A solution of N-phenylthiomorpholine-4-carboxamide 1,1-dioxide (1.000 g, 3.932 mmol) prepared in Step 1 of Synthesis Example 1 and sodium hydride (60.00%, 0.157 g, 3.932 mmol) dissolved in N,N-dimethylformamide (10 mL) was stirred at 0° C. for 1 hr. Methyl 4-(bromomethyl)-3-fluorobenzoate (0.905 g, 3.932 mmol) was added to the solution, and stirred at the room temperature for additional 2 hr. The reaction mixture was concentrated in vacuo to remove the solvent, and water was poured to the concentrate, followed by extraction with ethyl acetate. The organic layer was washed with saturated aqueous sodium chloride solution, dried (anhydrous MgSO₄), filtered, and concentrated in vacuo. The crude product was crystallized at the room temperature using methanol (20 mL) and filtered. The resulting precipitates were washed by methanol, and dried to give the title compound as brown solid (0.816 g, 51.4%).

### [Step 2] Synthesis of N-((5-(hydrazinecarbonyl)pyridin-2-yl)methyl)-N-phenylthiomorpholine-4-carboxamide 1,1-dioxide

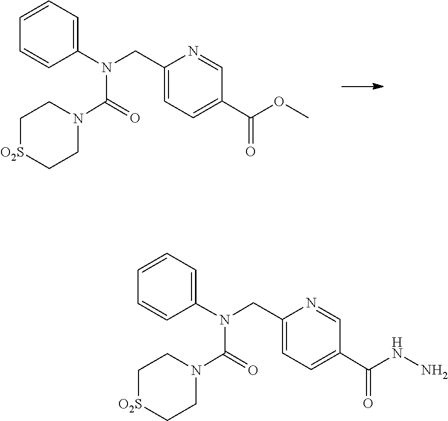 [SMILES: COC(=O)c1ccc(CN(C(=O)N2CCS(=O)(=O)CC2)c2ccccc2)nc1 ; NNC(=O)c1ccc(CN(C(=O)N2CCS(=O)(=O)CC2)c2ccccc2)nc1]

Methyl 6-((1,1-dioxido-N-phenylthiomorpholine-4-carboxamido)methyl)nicotinate (0.816 g, 2.023 mmol) prepared in Step 1 and hydrazine monohydrate (1.910 mL, 40.451 mmol) were mixed in ethanol (10 mL) at the room temperature, and heated at 100° C. under the microwaves for 1 hr, and then cooled down to the room temperature to terminate the reaction. The reaction mixture was concentrated in vacuo to remove the solvent. The crude product was crystallized at the room temperature using dichloromethane (20 mL) and filtered. The resulting precipitates were washed by dichloromethane, and dried to give the title compound as light brown solid (0.560 g, 68.6%).

### [Step 3] Synthesis of N-((5-(2-(2,2-difluoroacetyl) hydrazine-1-carbonyl)pyridin-2-yl)methyl)-N-phenylthiomorpholine-4-carboxamide 1,1-dioxide

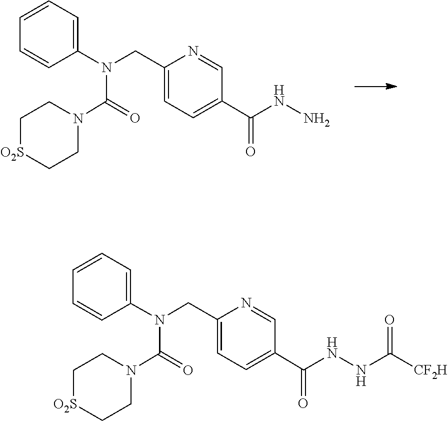 [SMILES: NNC(=O)c1ccc(CN(C(=O)N2CCS(=O)(=O)CC2)c2ccccc2)nc1 ; O=C(NNC(=O)C(F)F)c1ccc(CN(C(=O)N2CCS(=O)(=O)CC2)c2ccccc2)nc1]

Difluoroacetic anhydride (0.087 mL, 0.580 mmol) was added to a solution of N-((5-(hydrazinecarbonyl)pyridin-2-yl)methyl)-N-phenylthiomorpholine-4-carboxamide 1,1-dioxide (0.260 g, 0.644 mmol) prepared in Step 2 and triethylamine (0.178 mL, 1.289 mmol) dissolved in dichloromethane (2 mL) at the room temperature, and stirred at the same temperature for 16 hr. Then, water was poured to the reaction mixture, followed by extraction with dichloromethane. The bi-phasic mixture was passed through a plastic filter to remove solid residues and aqueous layer, and the organic layer collected was concentrated in vacuo. The concentrate was purified and concentrated by column chromatography (SiO₂, 4 g cartridge; methanol/dichloromethane=0% to 5%) to give the title compound as white foam (0.156 g, 50.3%).

### [Step 4] Synthesis of Compound 43

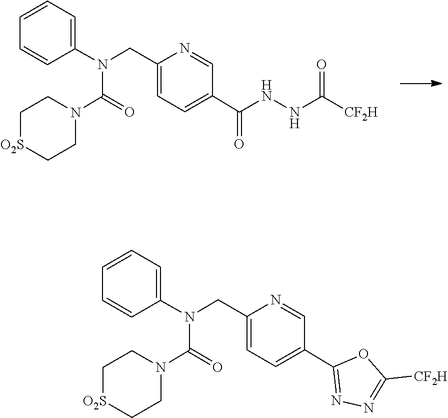 [SMILES: O=C(NNC(=O)C(F)F)c1ccc(CN(C(=O)N2CCS(=O)(=O)CC2)c2ccccc2)nc1 ; O=C(N1CCS(=O)(=O)CC1)N(Cc1ccc(-c2nnc(C(F)F)o2)cn1)c1ccccc1]

N-((5-(2-(2,2-difluoroacetyl) hydrazine-1-carbonyl)pyridin-2-yl)methyl)-N-phenylthiomorpholine-4-carboxamide 1,1-dioxide (0.156 g, 0.324 mmol) prepared in Step 3 and 1-methoxy-N-triethylammoniosulfonyl-methanimidate (Burgess reagent, 0.116 g, 0.486 mmol) were mixed in tetrahydrofuran (2 mL), and heated at 150° C. for 30 min under the microwaves, and then cooled down to the room temperature to terminate the reaction. Then, water was poured to the reaction mixture, followed by extraction with dichloromethane. The bi-phasic mixture was passed through a plastic filter to remove solid residues and aqueous layer, and the organic layer collected was concentrated in vacuo. The concentrate was purified and concentrated by column chromatography (SiO₂, 4 g cartridge; methanol/dichloromethane=3%) to give the title compound as colorless oil (0.078 g, 51.9%).

¹H NMR (400 MHZ, CDCl₃) δ 9.23 (d, 1H, J=2.2 Hz), 8.38 (dd, 1H, J=8.2, 2.2 Hz), 7.54 (d, 1H, J=8.2 Hz), 7.41-7.31 (m, 2H), 7.19 (ddd, 3H, J=6.4, 3.0, 1.6 Hz), 6.94 (m, 1H), 5.10 (s, 2H), 3.72 (dd, 4H, J=6.9, 3.7 Hz), 2.97-2.90 (m, 4H); LRMS (ES) m/z 464.2 (M⁺+1).

### Synthesis Example 3. Synthesis of Compound 232, N-(3-chloro-4-fluorophenyl)-N-((5-(5-(difluoromethyl)-1,3,4-oxadiazol-2-yl)pyridin-2-yl)methyl)morpholine-4-carboxamide

### [Step 1] Synthesis of N-(3-chloro-4-fluorophenyl) morpholine-4-carboxamide

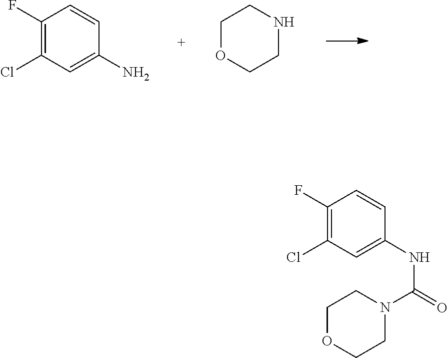 [SMILES: Nc1ccc(F)c(Cl)c1 ; C1COCCN1 ; O=C(Nc1ccc(F)c(Cl)c1)N1CCOCC1]

Morpholine (0.311 mL, 3.607 mmol) was added to a solution of 3-chloro-4-fluoroaniline (0.500 g, 3.435 mmol), 1,1′-carbonyldiimidazole (0.613 g, 3.779 mmol) and triethylamine (0.575 mL, 4.122 mmol) dissolved in acetonitrile (10 mL) at the room temperature, and stirred at the same temperature for 18 hr. Then, water was poured to the reaction mixture, followed by extraction with ethyl acetate. The organic layer was washed with saturated aqueous sodium chloride solution, dried (anhydrous MgSO₄), filtered, and concentrated in vacuo. The concentrate was purified and concentrated by column chromatography (SiO₂, 12 g cartridge; methanol/dichloromethane=0% to 5%) to give the title compound as purple solid (0.200 g, 22.5%).

[Step 2] Synthesis of Methyl 6-((N-(3-chloro-4-fluorophenyl) morpholine-4-carboxamido)methyl)nicotinate

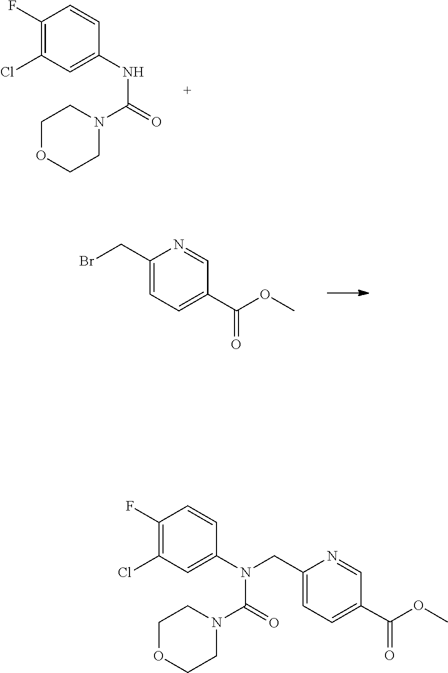 [SMILES: O=C(Nc1ccc(F)c(Cl)c1)N1CCOCC1 ; COC(=O)c1ccc(CBr)nc1 ; COC(=O)c1ccc(CN(C(=O)N2CCOCC2)c2ccc(F)c(Cl)c2)nc1]

Sodium hydride (60.00%, 0.037 g, 0.928 mmol) was added to a solution of N-(3-chloro-4-fluorophenyl) morpholine-4-carboxamide (0.200 g, 0.773 mmol) prepared in Step 1 dissolved in N,N-dimethylformide (5 mL) at 0° C., and stirred at the same temperature. Methyl 6-(bromomethyl) nicotinate (0.196 g, 0.850 mmol) was added to the reaction mixture, and stirred at the room temperature for additional 3 hr. Then, water was poured to the reaction mixture, followed by extraction with ethyl acetate. The organic layer was washed with saturated aqueous sodium chloride solution, dried (anhydrous MgSO₄), filtered, and concentrated in vacuo. The concentrate was purified and concentrated by column chromatography (SiO₂, 4 g cartridge; ethyl acetate/hexane=0% to 70%) to give the title compound as brown oil (0.110 g, 34.9%).

### [Step 3] Synthesis of N-(3-chloro-4-fluorophenyl)-N-((5-(hydrazinecarbonyl)pyridin-2-yl)methyl)morpholine-4-carboxamide

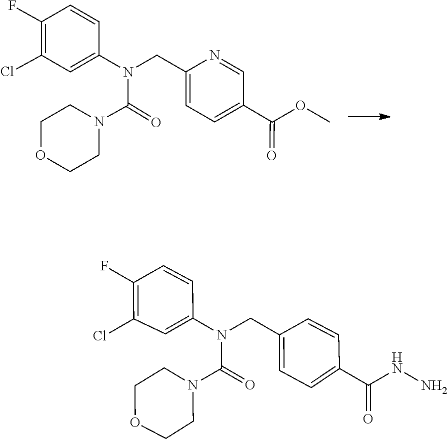 [SMILES: COC(=O)c1ccc(CN(C(=O)N2CCOCC2)c2ccc(F)c(Cl)c2)nc1 ; NNC(=O)c1ccc(CN(C(=O)N2CCOCC2)c2ccc(F)c(Cl)c2)cc1]

Methyl 6-((N-(3-chloro-4-fluorophenyl) morpholine-4-carboxamido)methyl)nicotinate (0.110 g, 0.270 mmol) prepared in Step 2 and hydrazine monohydrate (0.262 mL, 5.394 mmol) were mixed at the room temperature in ethanol (5 mL), and heated at reflux for 18 hr, and then cooled down to the room temperature. Then, water was poured to the reaction mixture, followed by extraction with ethyl acetate. The organic layer was washed with saturated aqueous sodium chloride solution, dried (anhydrous MgSO₄), filtered, and concentrated in vacuo. The crude title compound was used without further purification (0.110 g, 100.0%, light yellow solid).

### [Step 4] Synthesis of Compound 232

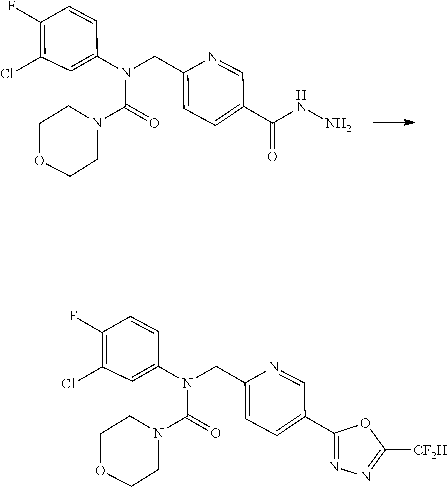 [SMILES: NNC(=O)c1ccc(CN(C(=O)N2CCOCC2)c2ccc(F)c(Cl)c2)nc1 ; O=C(N1CCOCC1)N(Cc1ccc(-c2nnc(C(F)F)o2)cn1)c1ccc(F)c(Cl)c1]

2,2-difluoroacetic anhydride (0.059 mL, 0.539 mmol) was added to a solution of N-(3-chloro-4-fluorophenyl)-N-((5-(hydrazinecarbonyl)pyridin-2-yl)methyl)morpholine-4-carboxamide (0.110 g, 0.270 mmol) prepared in Step 3 and N,N-diisopropylethylamine (0.070 mL, 0.405 mmol) dissolved in dichloromethane (3 mL) at 0° C., and stirred at the room temperature for 16 hr. Then, saturated aqueous sodium bicarbonate solution was poured to the reaction mixture, followed by extraction with dichloromethane. The bi-phasic mixture was passed through a plastic filter to remove the solid residues and aqueous layer, and the organic layer collected was concentrated in vacuo. The concentrate was purified and concentrated by column chromatography (SiO₂, 4 g cartridge; methanol/dichloromethane=0% to 5%) to give the title compound as yellow solid (0.057 g, 45.2%).

¹H NMR (400 MHz, CDCl₃) δ 9.24-9.24 (m, 1H), 8.36 (dd, 1H, J=8.2, 2.2 Hz), 7.59 (dd, 1H, J=8.2, 0.8 Hz), 7.30-7.28 (m, 1H), 7.10-7.08 (m, 2H), 6.93 (t, 1H, J=51.6 Hz), 5.05 (s, 2H), 3.54-3.52 (m, 4H), 3.27-3.26 (m, 4H); LRMS (ES) m/z 468.2 (M⁺+1).

### Synthesis Example 4. Synthesis of Compound 285, N-(4-(5-(difluoromethyl)-1,3,4-oxadiazol-2-yl)-2-fluorobenzyl)-N-(4-fluorophenyl)thiomorpholine-4-carboxamide 1,1-dioxide

### [Step 1] Synthesis of N-(4-fluorophenyl)thiomorpholine-4-carboxamide 1,1-dioxide

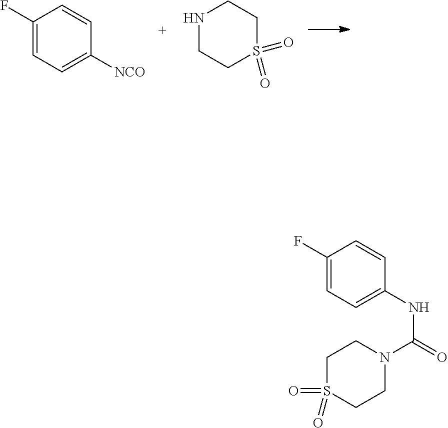

A solution of 1-fluoro-4-isocyanatobenzene (0.500 g, 3.647 mmol) dissolved in diethylether (10 mL) was mixed with thiomorpholine 1,1-dioxide (0.493 g, 3.647 mmol) at 0° C., and stirred at the same temperature for 1 hr. The reaction mixture was stirred at the room temperature for additional 4 hr. The precipitates were collected by filtration, washed by diethylether, and dried to give the title compound as white solid (0.920 g, 92.7%).

### [Step 2] Synthesis of Methyl 3-fluoro-4-((N-(4-fluorophenyl)-1,1-dioxidothiomorpholine-4-carboxamido)methyl)benzoate

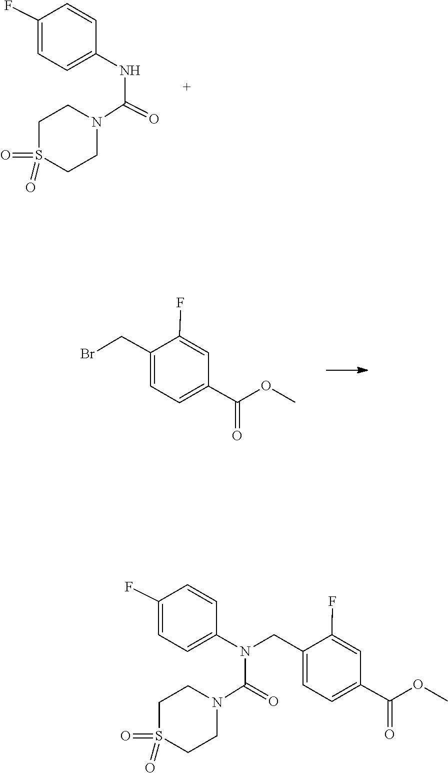 [SMILES: O=C(Nc1ccc(F)cc1)N1CCS(=O)(=O)CC1 ; COC(=O)c1ccc(CBr)c(F)c1 ; COC(=O)c1ccc(CN(C(=O)N2CCS(=O)(=O)CC2)c2ccc(F)cc2)c(F)c1]

A solution of N-(4-fluorophenyl)thiomorpholine-4-carboxamide 1,1-dioxide (0.300 g, 1.102 mmol) prepared in Step 1 and sodium hydride (60.00%, 0.048 g, 1.212 mmol) dissolved in N,N-dimethylformamide (5 mL) was stirred at 0° C. for 2 hr. Methyl 4-(bromomethyl)-3-fluorobenzoate (0.299 g, 1.212 mmol) was added to the solution, and stirred at the room temperature for additional 17 hr. The reaction was terminated by adding water (2 mL) to the reaction mixture at the room temperature and stirring for 10 min. Then, water was poured to the reaction mixture, followed by extraction with dichloromethane. The bi-phasic mixture was passed through a plastic filter to remove the solid residues and aqueous layer, and the organic layer collected was concentrated in vacuo. The crude product was crystallized at the room temperature using dichloromethane (3 mL) and filtered. The resulting precipitates were washed by dichloromethane, and dried to give the title compound as white solid (0.212 g, 43.9%).

### [Step 3] Synthesis of N-(2-fluoro-4-(hydrazinecarbonyl)benzyl)-N-(4-fluorophenyl)thiomorpholine-4-carboxamide 1,1-dioxide

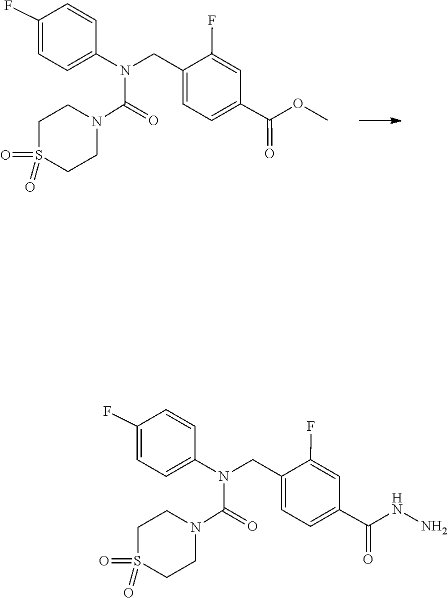 [SMILES: COC(=O)c1ccc(CN(C(=O)N2CCS(=O)(=O)CC2)c2ccc(F)cc2)c(F)c1 ; NNC(=O)c1ccc(CN(C(=O)N2CCS(=O)(=O)CC2)c2ccc(F)cc2)c(F)c1]

Methyl 3-fluoro-4-((N-(4-fluorophenyl)-1,1-dioxidothiomorpholine-4-carboxamido)methyl)benzoate (0.212 g, 0.484 mmol) prepared in Step 2 and hydrazine monohydrate (0.470 mL, 9.670 mmol) was mixed in ethanol (4 mL) at the room temperature, and heated at 120° C. under the microwaves for 1 hr, and then cooled down to the room temperature to terminate the reaction. The reaction mixture was concentrated in vacuo to remove the solvent. Then, water was poured to the reaction mixture, followed by extraction with dichloromethane. The bi-phasic mixture was passed through a plastic filter to remove the solid residues and aqueous layer, and the organic layer collected was concentrated in vacuo. TDiethylether (5 mL) and ethyl acetate (1 mL) were added to the concentrate and stirred. The resulting precipitates were collected by filtration, washed by hexane, and dried to give the title compound as white solid (0.179 g, 84.4%).

### [Step 4] Synthesis of Compound 285

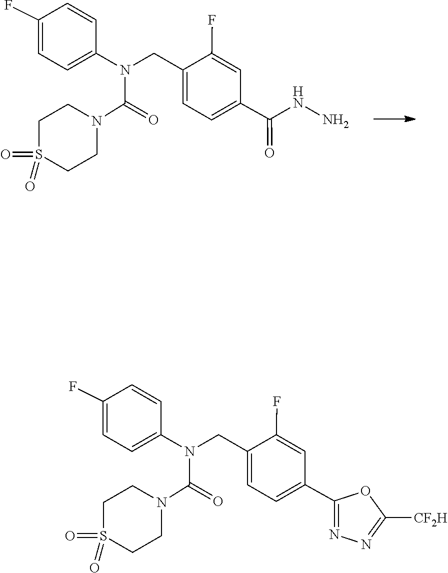 [SMILES: NNC(=O)c1ccc(CN(C(=O)N2CCS(=O)(=O)CC2)c2ccc(F)cc2)c(F)c1 ; O=C(N1CCS(=O)(=O)CC1)N(Cc1ccc(-c2nnc(C(F)F)o2)cc1F)c1ccc(F)cc1]

2,2-difluoroacetic anhydride (0.028 mL, 0.228 mmol) was added to a solution of N-(2-fluoro-4-(hydrazinecarbonyl)benzyl)-N-(4-fluorophenyl)thiomorpholine-4-carboxamide 1,1-dioxide (0.100 g, 0.228 mmol) prepared in Step 3 and triethylamine (0.095 mL, 0.684 mmol) dissolved in dichloromethane (4 mL) at the room temperature, and stirred at the same temperature for 17 hr. Then, saturated aqueous sodium bicarbonate solution was poured to the reaction mixture, followed by extraction with dichloromethane. The bi-phasic mixture was passed through a plastic filter to remove the solid residues and aqueous layer, and the organic layer collected was concentrated in vacuo. The concentrate was purified and concentrated by column chromatography (SiO₂, 4 g cartridge; ethyl acetate/hexane=20% to 50%) to give the title compound as white solid (0.053 g, 46.6%).

¹H NMR (400 MHZ, CDCl₃) δ 7.90 (dd, 1H, J=8.0, 1.6 Hz), 7.77 (dd, 1H, J=10.1, 1.6 Hz), 7.69 (t, 1H, J=7.6 Hz), 7.14-6.81 (m, 5H), 4.90 (s, 2H), 3.74-3.71 (m, 4H), 2.85-2.82 (m, 4H); LRMS (ES) m/z 499.3 (M⁺+1).

### Synthesis Example 5. Synthesis of Compound 295, N-((5-(5-(difluoromethyl)-1,3,4-oxadiazol-2-yl)pyridin-2-yl)methyl)-N-(4-fluorophenyl)thiomorpholine-4-carboxamide 1,1-dioxide

### [Step 1] Synthesis of Methyl 6-((N-(4-fluorophenyl)-1,1-dioxidothiomorpholine-4-carboxamido)methyl)nicotinate

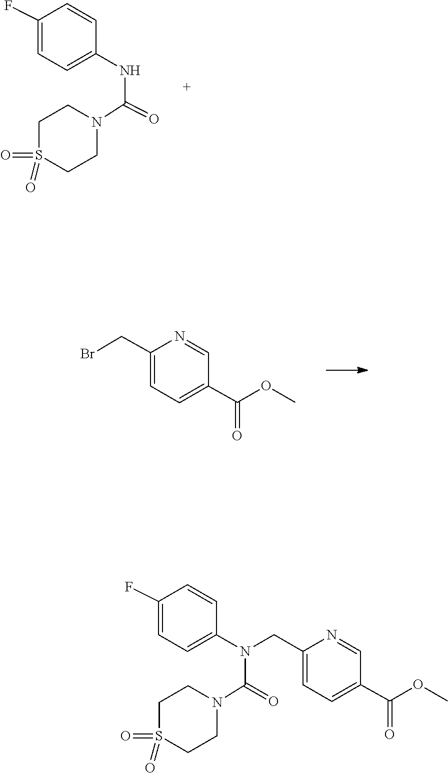 [SMILES: O=C(Nc1ccc(F)cc1)N1CCS(=O)(=O)CC1 ; COC(=O)c1ccc(CBr)nc1 ; COC(=O)c1ccc(CN(C(=O)N2CCS(=O)(=O)CC2)c2ccc(F)cc2)nc1]

A solution of N-(4-fluorophenyl)thiomorpholine-4-carboxamide 1,1-dioxide (0.500 g, 1.836 mmol) prepared in Step 1 of Synthesis Example 4 and sodium hydride (60.00%, 0.081 g, 2.020 mmol) dissolved in N,N-dimethylformamide (10 mL) was stirred at 0° C. for 30 min. Methyl 6-(bromomethyl) nicotinate (0.465 g, 2.020 mmol) was added to the solution, and stirred at the room temperature for additional 5 hr. The reaction was terminated by adding water (5 mL) to the reaction mixture at the room temperature and stirring for 10 min. Then, water was poured to the reaction mixture, followed by extraction with ethyl acetate. The organic layer was washed with saturated aqueous sodium chloride solution, dried (anhydrous MgSO₄), filtered, and concentrated in vacuo. The crude title compound was used without further purification (0.450 g, 58.1%, brown solid).

### [Step 2] Synthesis of N-(4-fluorophenyl)-N-((5-(hydrazinecarbonyl)pyridin-2-yl)methyl)thiomorpholine-4-carboxamide 1,1-dioxide

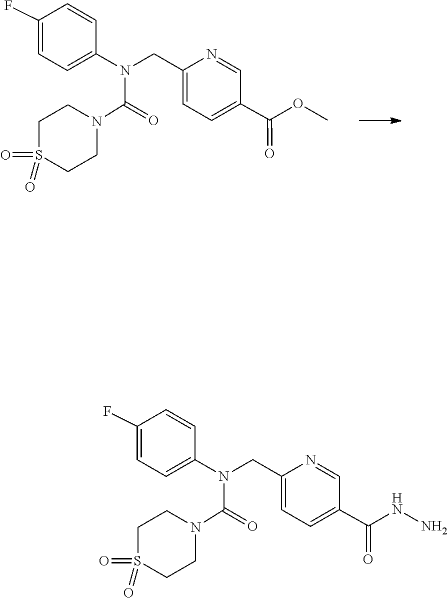 [SMILES: COC(=O)c1ccc(CN(C(=O)N2CCS(=O)(=O)CC2)c2ccc(F)cc2)nc1 ; NNC(=O)c1ccc(CN(C(=O)N2CCS(=O)(=O)CC2)c2ccc(F)cc2)nc1]

A solution of methyl 6-((N-(4-fluorophenyl)-1,1-dioxidothiomorpholine-4-carboxamido)methyl)nicotinate (0.150 g, 0.356 mmol) prepared in Step 1 and hydrazine monohydrate (0.346 mL, 7.118 mmol) dissolved in ethanol (5 mL) at the room temperature was stirred at 100° C. for 17 hr, and then cooled down to the room temperature to terminate the reaction. The precipitates were collected by filtration, washed by ethanol, and dried to give the title compound as pale yellow solid (0.111 g, 74.0%).

### [Step 3] Synthesis of N-((5-(2-(2,2-difluoroacetyl) hydrazine-1-carbonyl)pyridin-2-yl)methyl)-N-(4-fluorophenyl)thiomorpholine-4-carboxamide 1,1-dioxide

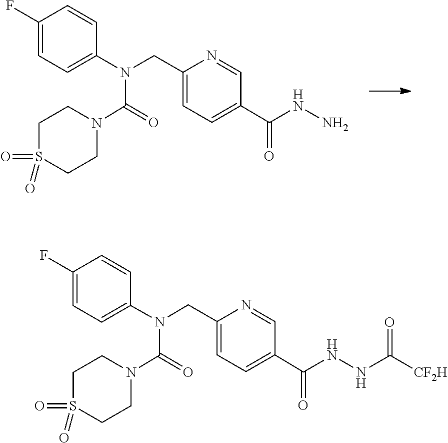 [SMILES: NNC(=O)c1ccc(CN(C(=O)N2CCS(=O)(=O)CC2)c2ccc(F)cc2)nc1 ; O=C(NNC(=O)C(F)F)c1ccc(CN(C(=O)N2CCS(=O)(=O)CC2)c2ccc(F)cc2)nc1]

2,2-difluoroacetic anhydride (0.065 mL, 0.527 mmol) was added to a solution of N-(4-fluorophenyl)-N-((5-(hydrazinecarbonyl)pyridin-2-yl)methyl)thiomorpholine-4-carboxamide 1,1-dioxide (0.111 g, 0.263 mmol) prepared in Step 2 and triethylamine (0.110 mL, 0.790 mmol) dissolved in dichloromethane (5 mL) at the room temperature, and stirred at the same temperature for 1 hr. Then, water was poured to the reaction mixture, followed by extraction with dichloromethane. The bi-phasic mixture was passed through a plastic filter to remove the solid residues and aqueous layer, and the organic layer collected was concentrated in vacuo. The crude title compound was used without further purification (0.082 g, 62.3%, yellow solid).

### [Step 4] Synthesis of Compound 295

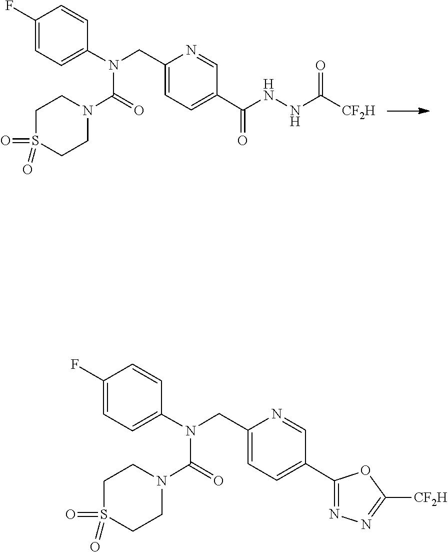 [SMILES: O=C(NNC(=O)C(F)F)c1ccc(CN(C(=O)N2CCS(=O)(=O)CC2)c2ccc(F)cc2)nc1 ; O=C(N1CCS(=O)(=O)CC1)N(Cc1ccc(-c2nnc(C(F)F)o2)cn1)c1ccc(F)cc1]

A solution of N-((5-(2-(2,2-difluoroacetyl) hydrazine-1-carbonyl)pyridin-2-yl)methyl)-N-(4-fluorophenyl)thiomorpholine-4-carboxamide 1,1-dioxide (0.082 g, 0.164 mmol) prepared in Step 3 and 1-methoxy-N-triethylammoniosulfonyl-methanimidate (Burgess reagent, 0.117 g, 0.493 mmol) dissolved in tetrahydrofuran (5 mL) at the room temperature was stirred at 70° C. for 5 hr, and then cooled down to the room temperature to terminate the reaction. The reaction mixture was passed through paper filter to remove solids, and the filtrate was concentrated in vacuo to remove the solvent. The concentrate was purified and concentrated by column chromatography (SiO₂, 4 g cartridge; methanol/dichloromethane=0% to 10%) to give the title compound as white solid (0.015 g, 19.0%).

¹H NMR (400 MHZ, CDCl₃) δ 9.27 (d, 1H, J=1.6 Hz), 8.43 (dd, 1H, J=8.2, 2.2 Hz), 7.58 (d, 2H, J=8.2 Hz), 7.25-7.21 (m, 2H), 7.10-6.84 (m, 3H), 5.08 (s, 2H), 3.73 (t, 4H, J=5.1 Hz), 2.98 (t, 4H, J=5.2 Hz); LRMS (ES) m/z 482.1 (M⁺+1).

### Synthesis Example 6. Synthesis of Compound 296, N-((5-(5-(difluoromethyl)-1,3,4-oxadiazol-2-yl)pyridin-2-yl)methyl)-N-(3-fluorophenyl)thiomorpholine-4-carboxamide 1,1-dioxide

### [Step 1] Synthesis of N-(3-fluorophenyl)thiomorpholine-4-carboxamide 1,1-dioxide

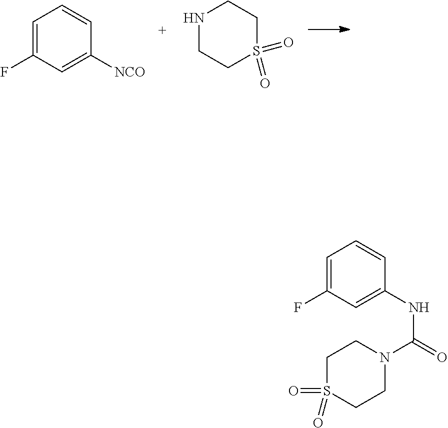

Thiomorpholine 1,1-dioxide (0.493 g, 3.647 mmol) was added to a solution of 1-fluoro-3-isocyanatobenzene (0.500 g. 3.647 mmol) dissolved in diethyl ether (10 mL) at 0° C., and stirred at the same temperature for 1 hr. The reaction mixture was stirred at room temperature for an additional 4 hr. The precipitates were collected by filtration, washed by diethyl ether, and dried to give the title compound as white solid (0.870 g, 87.6%).

### [Step 2] Synthesis of Methyl 6-((N-(3-fluorophenyl)-1,1-dioxidothiomorpholine-4-carboxamido)methyl)nicotinate

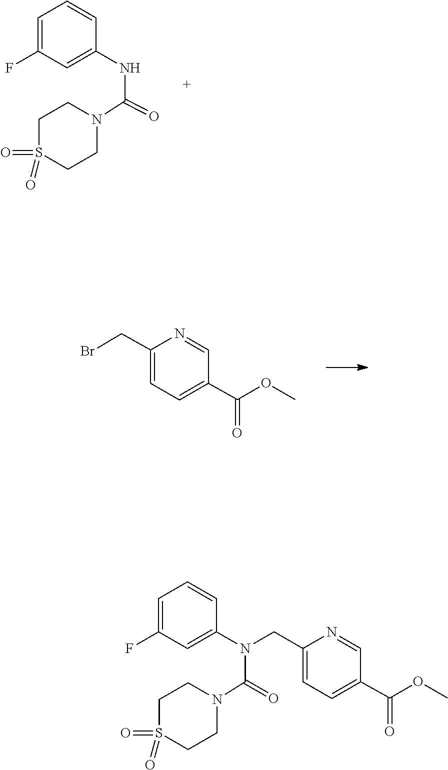 [SMILES: O=C(Nc1cccc(F)c1)N1CCS(=O)(=O)CC1 ; COC(=O)c1ccc(CBr)nc1 ; COC(=O)c1ccc(CN(C(=O)N2CCS(=O)(=O)CC2)c2cccc(F)c2)nc1]

A solution of N-(3-fluorophenyl)thiomorpholine-4-carboxamide 1,1-dioxide (0.500 g, 1.836 mmol) prepared in Step 1 and sodium hydride (60.00%, 0.081 g, 2.020 mmol) dissolved in N,N-dimethylformamide (10 mL) was stirred at 0° C. for 30 min. Methyl 6-(bromomethyl) nicotinate (0.465 g, 2.020 mmol) was added to the solution, and stirred at the room temperature for additional 5 hr. The reaction was terminated by adding water (5 mL) to the reaction mixture at the room temperature and stirring for 10 min. Then, water was poured to the reaction mixture, followed by extraction with ethyl acetate. The organic layer was washed with saturated aqueous sodium chloride solution, dried (anhydrous MgSO₄), filtered, and concentrated in vacuo. The crude title compound was used without further purification (0.450 g, 58.1%, brown solid).

### [Step 3] Synthesis of N-(3-fluorophenyl)-N-((5-(hydrazinecarbonyl)pyridin-2-yl)methyl)thiomorpholine-4-carboxamide 1,1-dioxide

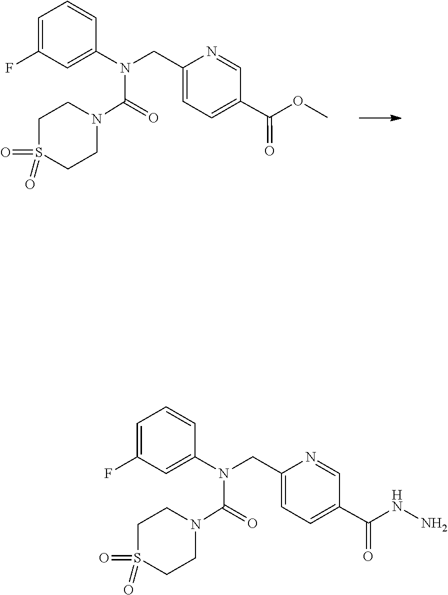 [SMILES: COC(=O)c1ccc(CN(C(=O)N2CCS(=O)(=O)CC2)c2cccc(F)c2)nc1 ; NNC(=O)c1ccc(CN(C(=O)N2CCS(=O)(=O)CC2)c2cccc(F)c2)nc1]

A solution of methyl 6-((N-(3-fluorophenyl)-1,1-dioxidothiomorpholine-4-carboxamido)methyl)nicotinate (0.150 g, 0.356 mmol) prepared in Step 2 and hydrazine monohydrate (0.346 mL, 7.118 mmol) dissolved in ethanol (5 mL) was stirred at 100° C. for 17 hr, and then cooled down to the room temperature to terminate the reaction. The precipitates were collected by filtration, washed by ethanol, and dried to give the title compound as pale yellow solid (0.113 g, 75.3%).

### [Step 4] Synthesis of N-((5-(2-(2,2-difluoroacetyl) hydrazine-1-carbonyl)pyridin-2-yl)methyl)-N-(3-fluorophenyl)thiomorpholine-4-carboxamide 1,1-dioxide

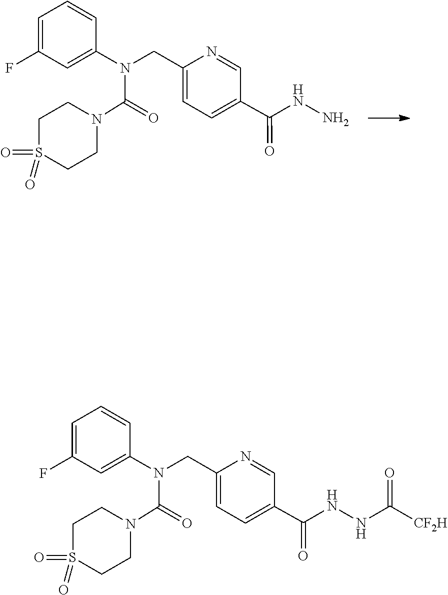 [SMILES: NNC(=O)c1ccc(CN(C(=O)N2CCS(=O)(=O)CC2)c2cccc(F)c2)nc1 ; O=C(NNC(=O)C(F)F)c1ccc(CN(C(=O)N2CCS(=O)(=O)CC2)c2cccc(F)c2)nc1]

2,2-difluoroacetic anhydride (0.067 mL, 0.536 mmol) was added to a solution of N-(3-fluorophenyl)-N-((5-(hydrazinecarbonyl)pyridin-2-yl)methyl)thiomorpholine-4-carboxamide 1,1-dioxide (0.113 g, 0.268 mmol) prepared in Step 3 and triethylamine (0.112 mL, 0.804 mmol) dissolved in dichloromethane (5 mL) at the room temperature, and stirred at the same temperature for 1 hr. Then, water was poured to the reaction mixture, followed by extraction with dichloromethane. The bi-phasic mixture was passed through a plastic filter to remove the solid residues and aqueous layer, and the organic layer collected was concentrated in vacuo. The crude title compound was used without further purification (0.090 g, 67.2%, yellow solid).

### [Step 5] Synthesis of Compound 296

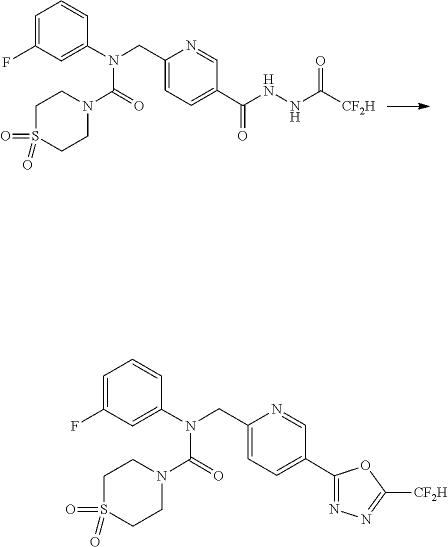 [SMILES: O=C(NNC(=O)C(F)F)c1ccc(CN(C(=O)N2CCS(=O)(=O)CC2)c2cccc(F)c2)nc1 ; O=C(N1CCS(=O)(=O)CC1)N(Cc1ccc(-c2nnc(C(F)F)o2)cn1)c1cccc(F)c1]

A solution of N-((5-(2-(2,2-difluoroacetyl) hydrazine-1-carbonyl)pyridin-2-yl)methyl)-N-(3-fluorophenyl)thiomorpholine-4-carboxamide 1,1-dioxide (0.090 g, 0.180 mmol) prepared in Step 4 and 1-methoxy-N-triethylammoniosulfonyl-methanimidate (Burgess reagent, 0.129 g, 0.541 mmol) dissolved in tetrahydrofuran (5 mL) was stirred at 70° C. for 5 hr, and then cooled down to the room temperature to terminate the reaction. The reaction mixture was passed through paper filter to remove solids, and the filtrate was concentrated in vacuo to remove the solvent. The concentrate was purified and concentrated by column chromatography (SiO₂, 4 g cartridge; methanol/dichloromethane=0% to 10%) to give the title compound as white solid (0.044 g, 50.7%).

¹H NMR (400 MHZ, CDCl₃) δ 9.28 (d, 1H, J=1.6 Hz), 8.46 (dd, 1H, J=8.2, 2.2 Hz), 7.58 (d, 1H, J=8.2 Hz), 7.37-7.32 (m, 1H), 7.10-6.92 (m, 4H), 5.14 (s, 2H), 3.76 (t, 4H, J=5.1 Hz), 3.03 (t, 4H, J=5.2 Hz); LRMS (ES) m/z 482.3 (M⁺+1).

### <Example 1> Effect of Inhibiting Expression of Fibrotic Proteins

In order to confirm an effect of the compound according to the present disclosure on preventing or treating idiopathic pulmonary fibrosis, an expression of fibrotic proteins was analyzed.

A549 cells, a pulmonary alveolar epithelial cell line, were seeded at 1×10⁵ cells/well in a 6-well plate and cultured in a CO₂ incubator (37° C., 5% CO₂) for 24 hours. After that, for serum starvation, a culture medium was replaced with a RPMI1690 (1% FBS, 1% P/S) culture medium and cultured in a CO₂ incubator (37° C., 5% CO₂) for 24 hours. After the serum starvation, 10 μM of test substances (compounds 40, 43, 239, 285, 295 and 296) were treated with 5 ng/ml of TGF-β1 and cultured in a CO₂ incubator (37° C., 5% CO₂) for 48 hours. A culture medium containing 0.1% DMSO was added to the control group. The expression of fibrotic proteins (FN-EDA, proCOL1A1) in the completely cultured cells was compared by using western blot.

Cells were lysed in 100 μL of RIPA buffer (containing proteinase & phosphatase inhibitors) and incubated on ice for 30 minutes. The resulting product was centrifuged at 13,000 g, 4° C. for 20 minutes. The supernatant was separated and quantified by using a BCA protein assay kit. Then, a NuPAGE sample reducing agent and a NuPAGE LDS sample buffer (4×) were added to prepare a sample at a concentration of 0.5 μg/μL. The prepared sample was boiled at 100° C. for five minutes to denaturate the proteins. 5 μg of protein was separated at 120 V after loading on NuPAGE Novex 4-12% Bis-Tris gel and transferred to a nitrocellulose (NC) membrane through an iBlot 2 dry blotting system. After that, the membrane was blocked with a blocking solution (EzBlock Chemi:distilled water=1:4) at room temperature for 30 minutes. The membrane was reacted overnight with a primary antibody at 4° C., and then washed three times with 1×TBST for 10 minutes each. Then, the resulting product was reacted with a horseradish peroxidase (HRP)-linked secondary antibody at room temperature for one hour, and washed three times with 1×TBST for 10 minutes each. The primary and secondary antibodies were diluted in a solution of a blocking solution and 1×TBST mixed in a ratio of 1:4. After that, the proteins were visualized by using Amersham™ ECL Select™ western blotting detection reagent and ChemiDoc™ MP imaging system. The observed proteins were quantified by using Image Lab 5.0 software, and then corrected by using a β-actin level.

One-way ANOVA (post hoc: Dunnett's multiple comparison test) was performed by using GraphPad Prism 9.3.0 software. All data were expressed as mean±SEM, and P<0.05 was considered statistically significant.

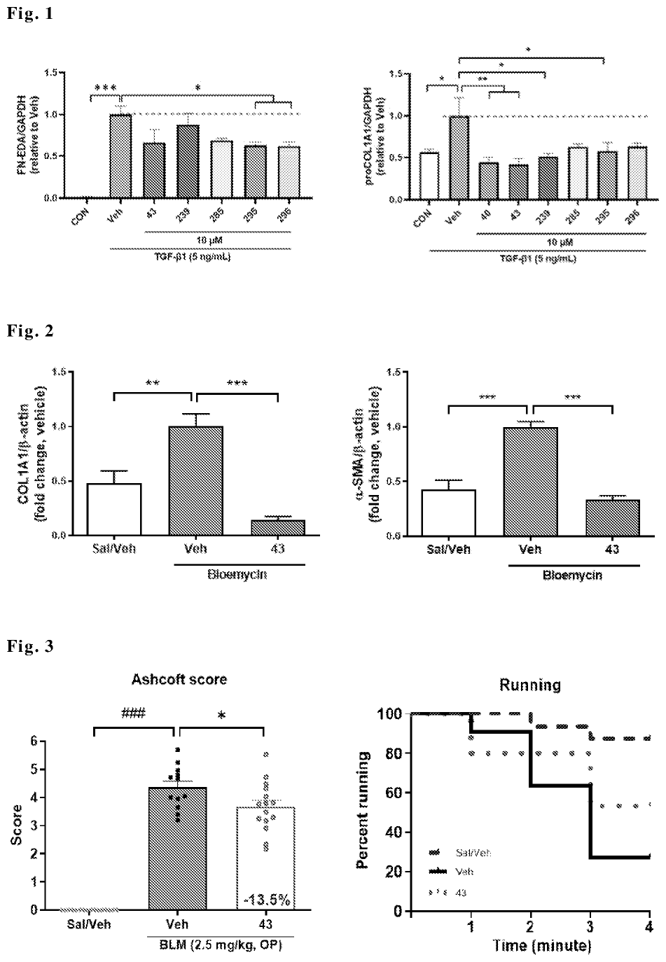

As a result, as shown in FIG. 1, it was confirmed that the expression of FN-EDA and proCOL1A1 induced by TGF-β1 is remarkably inhibited when treated with the compound of the present disclosure.

Thus, it could be seen that the compound of the present disclosure shows an effect of inhibiting an expression of fibrotic proteins, and thus may be advantageously used in preventing or treating idiopathic pulmonary fibrosis.

### <Example 2> Preventive or Therapeutic Effect in Animal Model with BLM-Induced Pulmonary Fibrosis

In order to confirm an effect of the compound according to the present disclosure on preventing or treating idiopathic pulmonary fibrosis, an expression of fibrotic proteins was analyzed in an animal model with BLM-induced pulmonary fibrosis.

An eight-week-old C57BL/6 mouse was induced to develop pulmonary fibrosis by intra-airway single administration with bleomycin (BLM, 0.3U/mouse). In the case of a prevention model, administration of the test substances was performed from the day after BLM administration and repeated twice a day for three weeks, and in the case of a treatment model, the administration of the test substances was performed from the 7th day of BLM administration and repeated twice a day for two weeks. The mice with BLM-induced pulmonary fibrosis were divided into groups as shown in table 1 below according to an administered substance [vehicle (Veh) or compound 43 (43)], a route of administration [oral administration (PO)], and an administration interval [twice daily (BID)].

TABLE 1

| | Admin- | | | | |
|---|---|---|---|---|---|
| | istered | Adminis- | Adminis- | Number of animals | |
| | dose | tration | tration | Prevention | Treatment |
| Group | (mg/kg) | route | cycle | model | model |
| Saline/Veh¹⁾ | — | PO | BID | 10 | 16 |
| BLM/Veh | — | PO | BID | 15 | 12 |
| BLM/43 | 30 | PO | BID | 15 | 15 |

¹⁾0.5% methyl cellulose

On the 21st day, the exercise capacity of the animals was compared using a treadmill test. For the treadmill test, an evaluation was performed on the 3rd day after a two-day training period. On the 1st day of training, each animal was trained for 0-1 min at 5 rpm and 1-2 min at 10 rpm for a total of 2 min. On the 2nd day of training, each animal was trained for 0-1 min at 5 rpm, 1-2 min at 10 rpm, and 2-3 min at 15 rpm, for a total of 3 min. On the 3rd day, the treadmill was set to 0-1 min at 5 rpm, 1-2 min at 10 rpm, 2-3 min at 15 rpm, and 3-4 min at 20 rpm for a total of 4 min and the evaluation was performed. At the time of evaluation, if an animal dropped out of the treadmill 10 times or more due to a decrease in vitality, it was judged that the evaluation was not possible and thus was excluded.

On the 21st day, the animals were anesthetized and exsanguinated, and the lungs were removed and the expression of fibrotic proteins (COL1A1, α-SMA) was compared using western blot and histological analysis.

For Western blot, lung tissues were lysed in RIPA buffer (containing proteinase & phosphatase inhibitors) and incubated on ice for 30 minutes. The resulting product was centrifuged at 13,000 g, 4° C. for 20 minutes. The supernatant was separated and quantified by using a BCA protein assay kit. Then, a NuPAGE sample reducing agent and a NuPAGE LDS sample buffer (4×) were added to prepare a sample at a concentration of 0.5 μg/μL. The prepared sample was boiled at 100° C. for five minutes to denaturate the proteins. 5 μg of protein was separated at 120 V after loading on NuPAGE Novex 4-12% Bis-Tris gel and transferred to a nitrocellulose (NC) membrane through an iBlot 2 dry blotting system. After that, the membrane was blocked with a blocking solution (EzBlock Chemi:distilled water=1:4) at room temperature for 30 minutes. The membrane was reacted overnight with a primary antibody at 4° C., and then washed three times with 1×TBST for 10 minutes each. Then, the resulting product was reacted with a horseradish peroxidase (HRP)-linked secondary antibody at room temperature for one hour, and washed three times with 1×TBST for 10 minutes each. The primary and secondary antibodies were diluted in a solution of a blocking solution and 1×TBST mixed in a ratio of 1:4. After that, the proteins were visualized by using Amersham™ ECL Select™ western blotting detection reagent and ChemiDoc™ MP imaging system. The observed proteins were quantified by using Image Lab 5.0 software, and then corrected by using a β-actin level.

For histological analysis, a lung tissue fixed in 10% neutral buffered formalin solution was cut, dehydrated and paraffin-infiltrated, and then transversely embedded in paraffin to make a block. The paraffin block was cut into 3 μm to prepare tissue sections, and hematoxylin and eosin (H&E) staining was performed for morphological evaluation of the tissue. The degree of fibrosis in lung tissue was semi-quantitatively determined based on a modified Ashcroft scale system (Biotechniques. 2008; 44 (4): 507-11, 514-7. Table 2).

One-tail unpaired t test (post hoc: Mann-Whitney) or one-way ANOVA (post hoc: Dunnett's multiple comparison test) was performed by using GraphPad Prism 9.3.0 software. All data were expressed as mean±SEM, and P<0.05 was considered statistically significant.

As a result, as shown in FIG. 2, it was confirmed that the expression of COL1A1 and α-SMA is remarkably inhibited in the mouse lung tissue with BLM-induced pulmonary fibrosis of the prevention model when treated with the compound of the present disclosure.

In addition, as shown in FIG. 3, it was confirmed that the Ashcroft score is remarkably decreased and physical activity is improved in the mouse with BLM-induced pulmonary fibrosis of the treatment model when treated with the compound of the present disclosure.

Thus, it could be seen that the compound of the present disclosure shows an effect of inhibiting an expression of fibrotic proteins, decreasing pulmonary fibrosis and improving exercise capacity and thus may be advantageously used in preventing or treating idiopathic pulmonary fibrosis.

The present disclosure provides a pharmaceutical composition, a method, and a use as follow:
Item 1. A pharmaceutical composition for preventing or treating idiopathic pulmonary fibrosis, comprising a compound represented by the above-mentioned formula I, optical isomers thereof or pharmaceutically acceptable salts thereof as an effective ingredient.
Item 2. The pharmaceutical composition of item 1, wherein the compound represented by the above-mentioned formula I is at least one selected from the group consisting of the above-mentioned compounds 1 to 450 which is described in the above-mentioned Table.
Item 3. The pharmaceutical composition of item 1 or 2, wherein the compound represented by the above-mentioned formula I is at least one selected from the group consisting of compound 40, compound 43, compound 239, compound 285, compound 295 and compound 296 which is described in the above-mentioned Table.
Item 4. A method for preventing or treating idiopathic pulmonary fibrosis, including administering the compound represented by formula I, optical isomers thereof or pharmaceutically acceptable salts thereof described in any one of items 1 to 3 into an individual.
Item 5. A use of the compound represented by formula I, optical isomers thereof or pharmaceutically acceptable salts thereof described in any one of items 1 to 3 for preventing or treating idiopathic pulmonary fibrosis.
Item 6. A use of the compound represented by formula I, optical isomers thereof or pharmaceutically acceptable salts thereof described in any one of items 1 to 3 in preparing a medicament for preventing or treating idiopathic pulmonary fibrosis.
Item 7. The pharmaceutical composition according to any one of items 1 to 3, wherein the pharmaceutical composition is orally administered.
Item 8. The method according to item 4, or the use according to item 5 or 6, wherein the compound represented by formula I, optical isomers thereof or pharmaceutically acceptable salts thereof described in any one of items 1 to 3 is orally administered.

While specific portions of the present disclosure have been described in detail above, it is apparent to those skilled in the art that such detailed descriptions are set forth to illustrate exemplary embodiments only, but are not construed to limit the scope of the present disclosure. Thus, it should be understood that the substantial scope of the present disclosure is defined by the accompanying claims and equivalents thereto.

1. A pharmaceutical composition for preventing or treating idiopathic pulmonary fibrosis, comprising a compound represented by a following formula I, optical isomers thereof or pharmaceutically acceptable salts thereof as an effective ingredient: 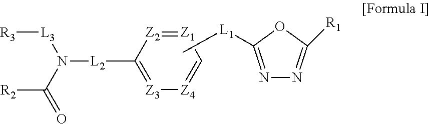
in formula I,
wherein L₁, L₂ and L₃ are each independently a bond or —(C₁-C₂ alkylene)-;
R₁ is —CX₂H or —CX₃;
R₂ is —NR^{A}R^{B}, —OR^{C}, 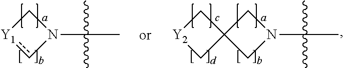
{wherein at least one H of 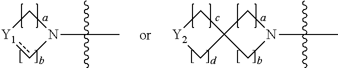
may be each independently substituted with —X, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be each independently substituted with —X, —OH, —CF₃ or —CF₂H]};
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl, 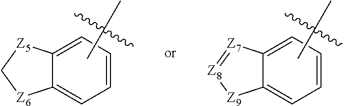
{wherein, at least one H of —(C₁-C₄ alkyl) may be each independently substituted with —X or —OH,
at least one H of -aryl or -heteroaryl may be each independently substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), -aryl, heteroaryl, 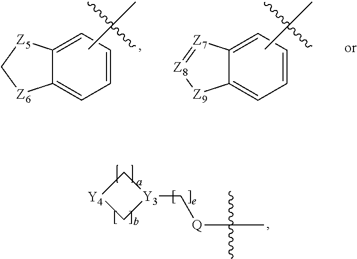
[wherein, at least one H of 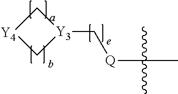
may be each independently substituted with —X, —(C₁-C₄ alkyl), —NR^{D}R^{E}, —CF₃ or —CF₂H],
at least one H of —(C₃-C₇ cycloalkyl), —(C₂-C₆ cycloheteroalkyl), adamantyl, 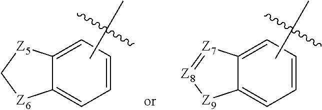
may be each independently substituted with —X, —OH or (C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ are each independently N or CR^{Z}, {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently —CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ alkyl)-heteroaryl, —(C₃-C₇ cycloalkyl), —(C₂-C₆ heterocycloalkyl) or 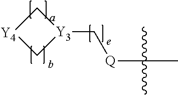
{wherein, at least one H of the —(C₁-C₄ alkyl), —(C₁-C₄ alkyl)-OH or —(C₁-C₄ alkyl)-NR^{D}R^{E} may be each independently substituted with —X,
at least one H of the -aryl, —(C₁-C₄ alkyl)-aryl, -heteroaryl, —(C₁-C₄ alkyl)-heteroaryl, (C₃-C₇ cycloalkyl) or —(C₂-C₆ heterocycloalkyl) may be each independently substituted with —X, —OH, —O(C₁-C₄ alkyl), —(C₁-C₄ alkyl), —CF₃, —CF₂H or —CN,
at least one H of 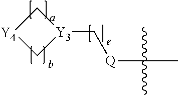
may be each independently substituted with —X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl)};
R^{C} is —(C₁-C₄ alkyl), -aryl, —(C₁-C₄ alkyl)-aryl, -heteroaryl or —(C₁-C₄ alkyl)-heteroaryl {wherein, at least one H of —(C₁-C₄ alkyl) may be each independently substituted with —X or —OH, at least one H of -aryl, —(C₁-C₄ alkyl)-aryl, -heteroaryl or —(C₁-C₄ alkyl)-heteroaryl may be each independently substituted with —X, —OH, —CF₃ or —CF₂H};
R^{D} and R^{E} are each independently —H, —(C₁-C₄ alkyl), -aryl or —(C₁-C₄ alkyl)-aryl {wherein, at least one H of —(C₁-C₄ alkyl) may be each independently substituted with —X or —OH, at least one H of -aryl or —(C₁-C₄ alkyl)-aryl may be each independently substituted with —X, —OH, —CF₃ or —CF₂H};
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein, at least one H of —(C₁-C₄ alkyl), —(C₁-C₄ alkyl)-OH, —(C₁-C₄ alkyl)-O—(C₁-C₄ alkyl), —C(═O)—(C₁-C₄alkyl), —C(═O)—O(C₁-C₄ alkyl), —(C₁-C₄ alkyl)-C(═O)—O(C₁-C₄ alkyl), (C₁-C₄ alkyl)-NR^{D}R^{E} or —S(═O)₂—(C₁-C₄ alkyl) may be each independently substituted with —X,
at least one H of -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —C₂-C₆ heterocycloalkyl or —(C₁-C₄ alkyl)-C(═O)—(C₂-C₆ heterocycloalkyl) may be each independently substituted with —X, —OH, —CF₃ or —CF₂H};
R^{G} is —H or —(C₁-C₄ alkyl);
Q is —O— or a bond; [symbol]
is a single bond or double bond {provided that, when [symbol] is a double bond, then Y₁ is ═CH—};
a to e are each independently an integer of 0, 1, 2, 3 or 4 {provided that, a and b may not be simultaneously 0, and c and d may not be simultaneously 0};
X is each independently F, Cl, Br or I.

2. The pharmaceutical composition according to claim 1, wherein in the compound represented by the above formula I,
L₁, L₂ and L₃ are each independently a bond or —(C₁-C₂ alkylene)-;
R₁ is —CX₂H or —CX₃;
R₂ is —NR^{A}R^{B}, —OR^{C}, 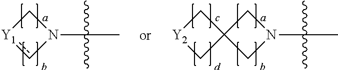
{wherein at least one of H of 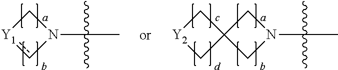
may be each independently substituted with —X, —OH, —NR^{D}R^{E} or —(C₁-C₄ alkyl)};
R₃ is —(C₁-C₄ alkyl), —(C₃-C₇ cycloalkyl), -aryl, -heteroaryl, -adamantyl, 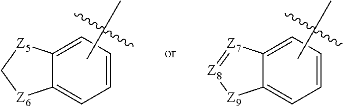
{wherein at least one H of -aryl or -heteroaryl may be each independently substituted with —X, —O(C₁-C₄ alkyl), —OCF₃, —O-aryl, —NR^{D}R^{E}, —(C₁-C₄ alkyl), —CF₃, —S(═O)₂—(C₁-C₄ alkyl), -aryl, -heteroaryl, 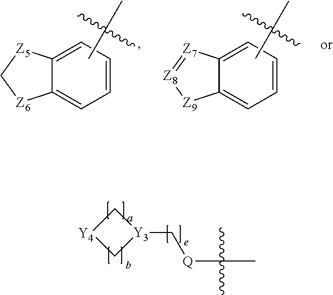
[wherein, at least one H of 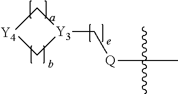
may be each independently substituted with —NR^{D}R^{E} or —(C₁-C₄ alkyl)],
at least one H of 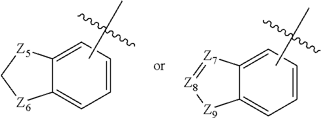
may be each independently substituted with —(C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ is each independently N or CR^{Z} {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, —(C₃-C₇ cycloalkyl) or 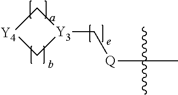
{wherein, at least one H of 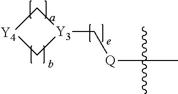
may be each independently substituted with —X, —(C₁-C₄ alkyl), —CF₃, —(C₂-C₆ heterocycloalkyl), —(C₁-C₄ alkyl)-aryl, -heteroaryl or -heteroaryl-(C₁-C₄ alkyl)};
R^{C} is —(C₁-C₄ alkyl) or -aryl;
R^{D} and R^{E} are each independently —H, —(C₁-C₄ alkyl) or —(C₁-C₄ alkyl)-aryl;
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein at least one H of —(C₁-C₄ alkyl) or —C(═O)—O(C₁-C₄ alkyl) may be each independently substituted with —X,
at least one H of -aryl may be each independently substituted with —X};
R^{G} is —(C₁-C₄ alkyl);
Q is —O— or a bond; [symbol]
is a single bond or a double bond {provided that, when [symbol] is a double bond, then Y₁ is —CH—};
a to e are each independently an integer of 0, 1, 2, 3 or 4 {provided that, a and b may not be simultaneously 0, and c and d may not be simultaneously 0};
X is each independently F, Cl, Br or I.

3. The pharmaceutical composition according to claim 1, wherein the compound represented by the above formula I is the compound represented by a following formula Ia: 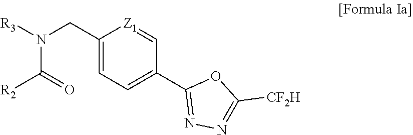
in formula Ia,
R₂ is 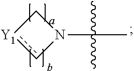
R₃ is -aryl {wherein at least one H of -aryl may be each independently substituted with —X};
Y₁ is —S(═O)₂—;
Z₁ is N or CR^{Z} {wherein R^{Z} is —X};
a and b are each independently an integer of 0, 1, 2, 3 or 4 {wherein a and b may not be simultaneously 0};
X is each independently F, Cl, Br or I.

4. The pharmaceutical composition according to claim 3, wherein in the compound represented by the above formula Ia,
R₂ is 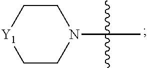
R₃ is -phenyl {wherein at least one H of -phenyl may be each independently substituted with —F or —Cl};
Y₁ is —S(═O)—;
Z₁ is N or CF.

5. The pharmaceutical composition according to claim 1, wherein the compound represented by the above formula I is a compound described in a following table:

| Compound | Structure |
|---|---|
| 1 | 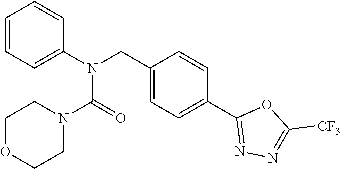 [SMILES: O=C(N1CCOCC1)N(Cc1ccc(-c2nnc(C(F)(F)F)o2)cc1)c1ccccc1] |
| 2 | 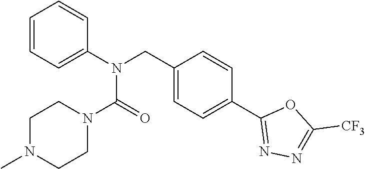 [SMILES: CN1CCN(C(=O)N(Cc2ccc(-c3nnc(C(F)(F)F)o3)cc2)c2ccccc2)CC1] |
| 3 | 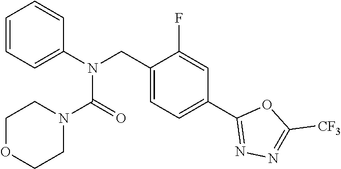 [SMILES: O=C(N1CCOCC1)N(Cc1ccc(-c2nnc(C(F)(F)F)o2)cc1F)c1ccccc1] |
| 4 | 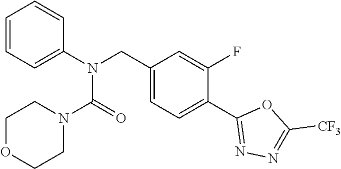 [SMILES: O=C(N1CCOCC1)N(Cc1ccc(-c2nnc(C(F)(F)F)o2)c(F)c1)c1ccccc1] |
| 5 | 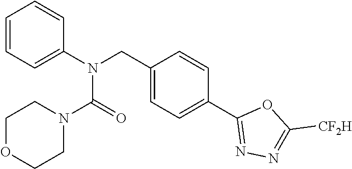 [SMILES: O=C(N1CCOCC1)N(Cc1ccc(-c2nnc(C(F)F)o2)cc1)c1ccccc1] |
| 6 | 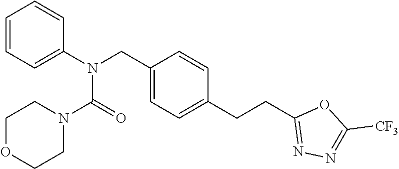 [SMILES: O=C(N1CCOCC1)N(Cc1ccc(CCc2nnc(C(F)(F)F)o2)cc1)c1ccccc1] |
| 7 | 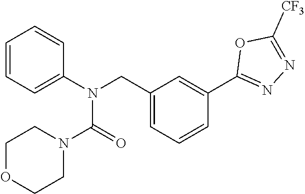 [SMILES: O=C(N1CCOCC1)N(Cc1cccc(-c2nnc(C(F)(F)F)o2)c1)c1ccccc1] |
| 8 | 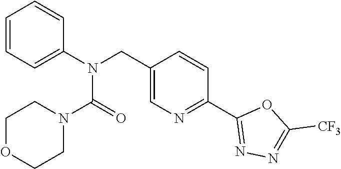 [SMILES: O=C(N1CCOCC1)N(Cc1ccc(-c2nnc(C(F)(F)F)o2)nc1)c1ccccc1] |
| 9 | 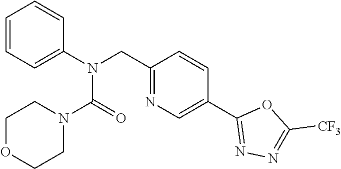 [SMILES: O=C(N1CCOCC1)N(Cc1ccc(-c2nnc(C(F)(F)F)o2)cn1)c1ccccc1] |
| 10 | 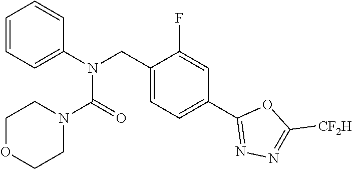 [SMILES: O=C(N1CCOCC1)N(Cc1ccc(-c2nnc(C(F)F)o2)cc1F)c1ccccc1] |
| 11 | 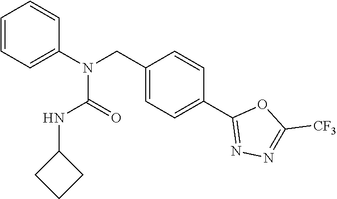 [SMILES: O=C(NC1CCC1)N(Cc1ccc(-c2nnc(C(F)(F)F)o2)cc1)c1ccccc1] |
| 12 | 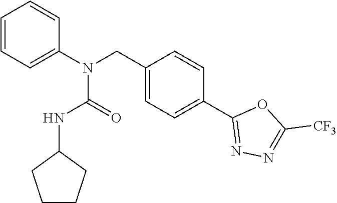 [SMILES: O=C(NC1CCCC1)N(Cc1ccc(-c2nnc(C(F)(F)F)o2)cc1)c1ccccc1] |
| 13 | 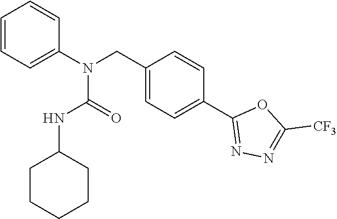 [SMILES: O=C(NC1CCCCC1)N(Cc1ccc(-c2nnc(C(F)(F)F)o2)cc1)c1ccccc1] |
| 14 | 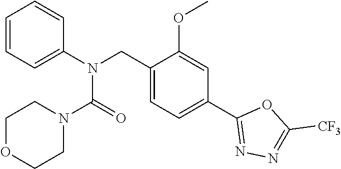 [SMILES: COc1cc(-c2nnc(C(F)(F)F)o2)ccc1CN(C(=O)N1CCOCC1)c1ccccc1] |
| 15 | 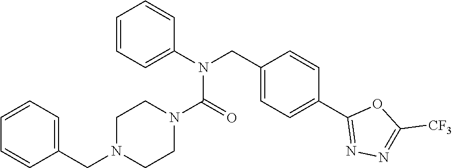 [SMILES: O=C(N1CCN(Cc2ccccc2)CC1)N(Cc1ccc(-c2nnc(C(F)(F)F)o2)cc1)c1ccccc1] |
| 16 | 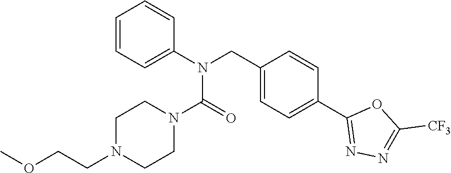 [SMILES: COCCN1CCN(C(=O)N(Cc2ccc(-c3nnc(C(F)(F)F)o3)cc2)c2ccccc2)CC1] |
| 17 | 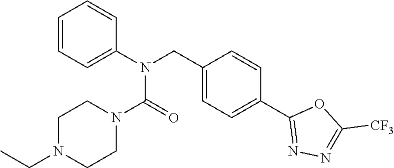 [SMILES: CCN1CCN(C(=O)N(Cc2ccc(-c3nnc(C(F)(F)F)o3)cc2)c2ccccc2)CC1] |
| 18 | 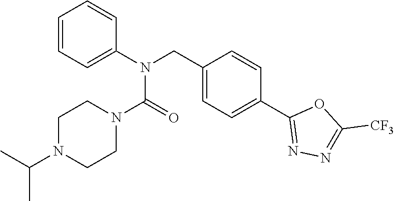 [SMILES: CC(C)N1CCN(C(=O)N(Cc2ccc(-c3nnc(C(F)(F)F)o3)cc2)c2ccccc2)CC1] |
| 19 | 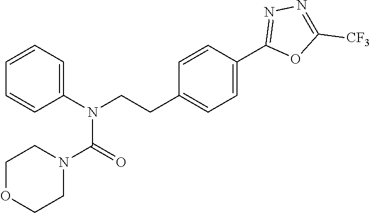 [SMILES: O=C(N1CCOCC1)N(CCc1ccc(-c2nnc(C(F)(F)F)o2)cc1)c1ccccc1] |
| 20 | 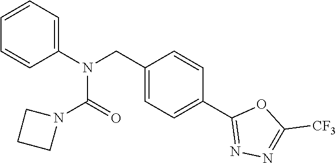 [SMILES: O=C(N1CCC1)N(Cc1ccc(-c2nnc(C(F)(F)F)o2)cc1)c1ccccc1] |
| 21 | 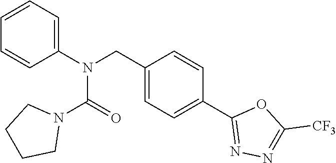 [SMILES: O=C(N1CCCC1)N(Cc1ccc(-c2nnc(C(F)(F)F)o2)cc1)c1ccccc1] |
| 22 | 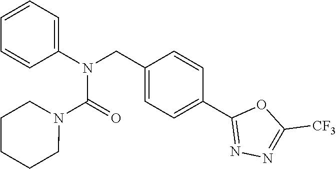 [SMILES: O=C(N1CCCCC1)N(Cc1ccc(-c2nnc(C(F)(F)F)o2)cc1)c1ccccc1] |
| 23 | 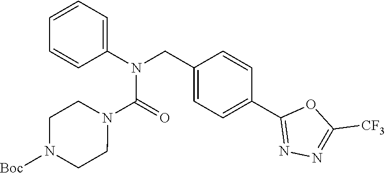 [SMILES: CC(C)(C)OC(=O)N1CCN(C(=O)N(Cc2ccc(-c3nnc(C(F)(F)F)o3)cc2)c2ccccc2)CC1] |
| 24 | 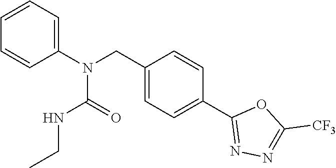 [SMILES: CCNC(=O)N(Cc1ccc(-c2nnc(C(F)(F)F)o2)cc1)c1ccccc1] |
| 25 | 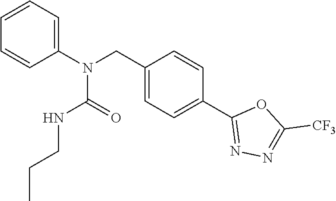 [SMILES: CCCNC(=O)N(Cc1ccc(-c2nnc(C(F)(F)F)o2)cc1)c1ccccc1] |
| 26 | 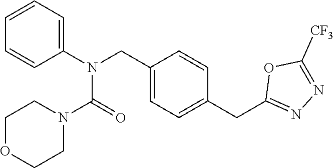 [SMILES: O=C(N1CCOCC1)N(Cc1ccc(Cc2nnc(C(F)(F)F)o2)cc1)c1ccccc1] |
| 27 | 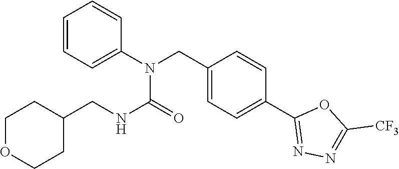 [SMILES: O=C(NCC1CCOCC1)N(Cc1ccc(-c2nnc(C(F)(F)F)o2)cc1)c1ccccc1] |
| 28 | 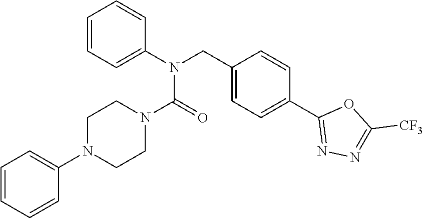 [SMILES: O=C(N1CCN(c2ccccc2)CC1)N(Cc1ccc(-c2nnc(C(F)(F)F)o2)cc1)c1ccccc1] |
| 29 | 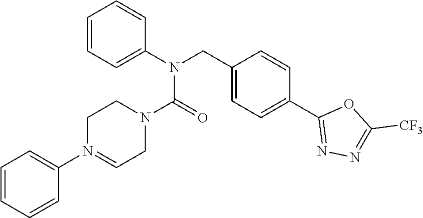 |
| 30 | 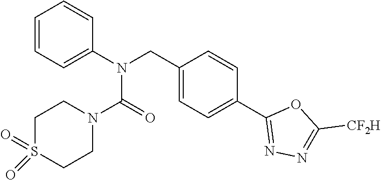 [SMILES: O=C(N1CCS(=O)(=O)CC1)N(Cc1ccc(-c2nnc(C(F)F)o2)cc1)c1ccccc1] |
| 31 | 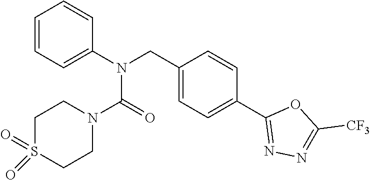 [SMILES: O=C(N1CCS(=O)(=O)CC1)N(Cc1ccc(-c2nnc(C(F)(F)F)o2)cc1)c1ccccc1] |
| 32 | 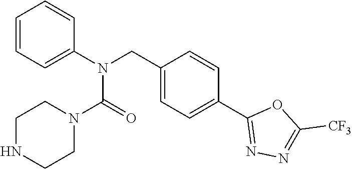 [SMILES: O=C(N1CCNCC1)N(Cc1ccc(-c2nnc(C(F)(F)F)o2)cc1)c1ccccc1] |
| 33 | 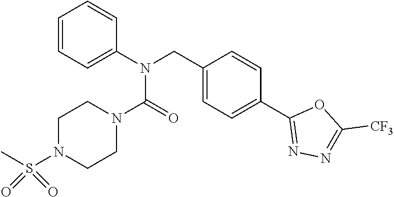 [SMILES: CS(=O)(=O)N1CCN(C(=O)N(Cc2ccc(-c3nnc(C(F)(F)F)o3)cc2)c2ccccc2)CC1] |
| 34 | 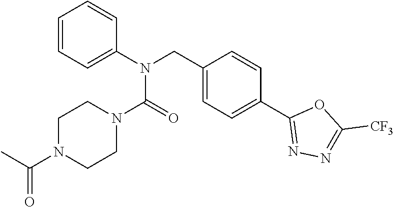 [SMILES: CC(=O)N1CCN(C(=O)N(Cc2ccc(-c3nnc(C(F)(F)F)o3)cc2)c2ccccc2)CC1] |
| 35 | 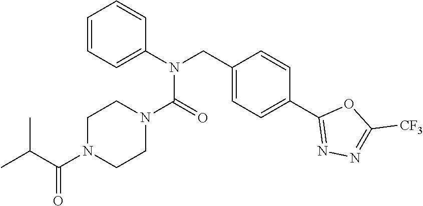 [SMILES: CC(C)C(=O)N1CCN(C(=O)N(Cc2ccc(-c3nnc(C(F)(F)F)o3)cc2)c2ccccc2)CC1] |
| 36 | 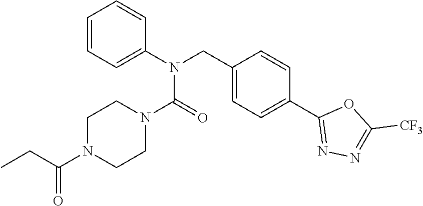 [SMILES: CCC(=O)N1CCN(C(=O)N(Cc2ccc(-c3nnc(C(F)(F)F)o3)cc2)c2ccccc2)CC1] |
| 37 | 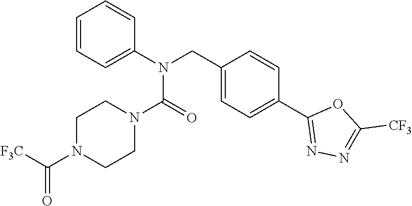 [SMILES: O=C(N1CCN(C(=O)C(F)(F)F)CC1)N(Cc1ccc(-c2nnc(C(F)(F)F)o2)cc1)c1ccccc1] |
| 38 | 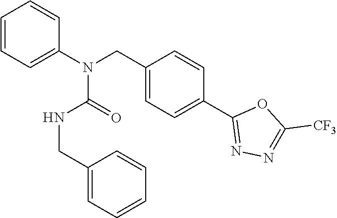 [SMILES: O=C(NCc1ccccc1)N(Cc1ccc(-c2nnc(C(F)(F)F)o2)cc1)c1ccccc1] |
| 39 | 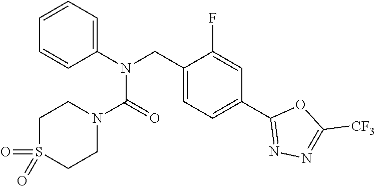 [SMILES: O=C(N1CCS(=O)(=O)CC1)N(Cc1ccc(-c2nnc(C(F)(F)F)o2)cc1F)c1ccccc1] |
| 40 | 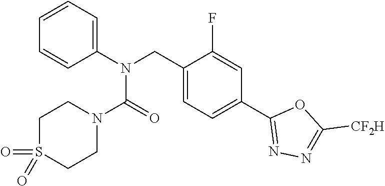 [SMILES: O=C(N1CCS(=O)(=O)CC1)N(Cc1ccc(-c2nnc(C(F)F)o2)cc1F)c1ccccc1] |
| 41 | 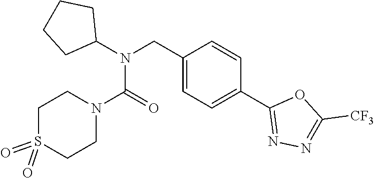 [SMILES: O=C(N1CCS(=O)(=O)CC1)N(Cc1ccc(-c2nnc(C(F)(F)F)o2)cc1)C1CCCC1] |
| 42 | 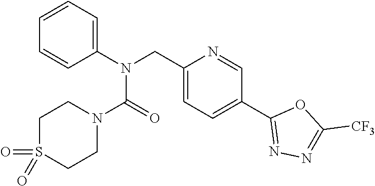 [SMILES: O=C(N1CCS(=O)(=O)CC1)N(Cc1ccc(-c2nnc(C(F)(F)F)o2)cn1)c1ccccc1] |
| 43 | 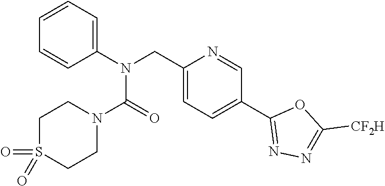 [SMILES: O=C(N1CCS(=O)(=O)CC1)N(Cc1ccc(-c2nnc(C(F)F)o2)cn1)c1ccccc1] |
| 44 | 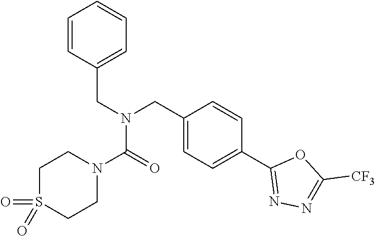 [SMILES: O=C(N1CCS(=O)(=O)CC1)N(Cc1ccccc1)Cc1ccc(-c2nnc(C(F)(F)F)o2)cc1] |
| 45 | 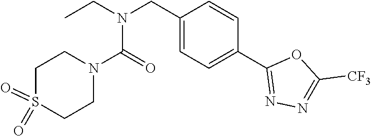 [SMILES: CCN(Cc1ccc(-c2nnc(C(F)(F)F)o2)cc1)C(=O)N1CCS(=O)(=O)CC1] |
| 46 | 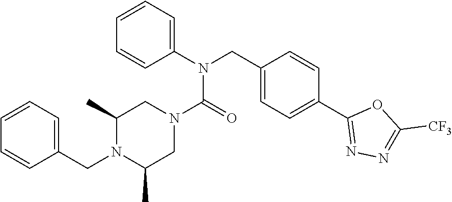 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 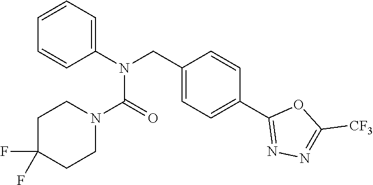 [SMILES: O=C(N1CCC(F)(F)CC1)N(Cc1ccc(-c2nnc(C(F)(F)F)o2)cc1)c1ccccc1] |
| 48 | 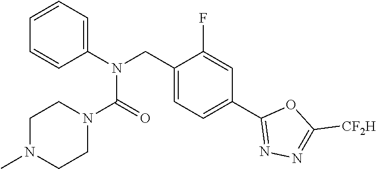 [SMILES: CN1CCN(C(=O)N(Cc2ccc(-c3nnc(C(F)F)o3)cc2F)c2ccccc2)CC1] |
| 49 | 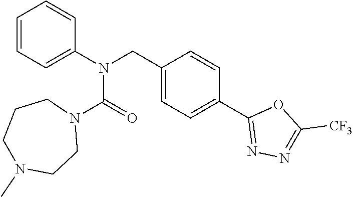 [SMILES: CN1CCCN(C(=O)N(Cc2ccc(-c3nnc(C(F)(F)F)o3)cc2)c2ccccc2)CC1] |
| 50 | 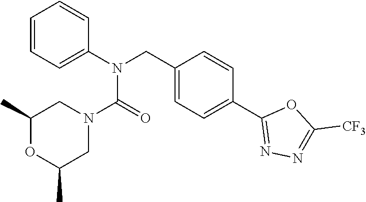 [SMILES: C[C@@H]1CN(C(=O)N(Cc2ccc(-c3nnc(C(F)(F)F)o3)cc2)c2ccccc2)C[C@H](C)O1] |
| 51 | 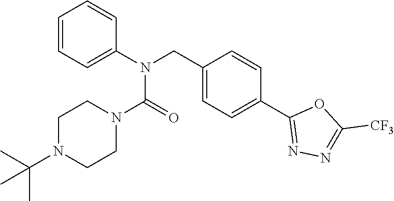 [SMILES: CC(C)(C)N1CCN(C(=O)N(Cc2ccc(-c3nnc(C(F)(F)F)o3)cc2)c2ccccc2)CC1] |
| 52 | 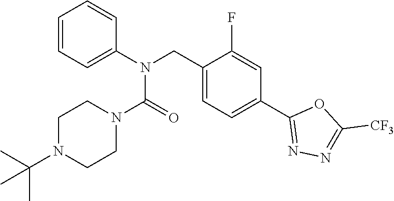 [SMILES: CC(C)(C)N1CCN(C(=O)N(Cc2ccc(-c3nnc(C(F)(F)F)o3)cc2F)c2ccccc2)CC1] |
| 53 | 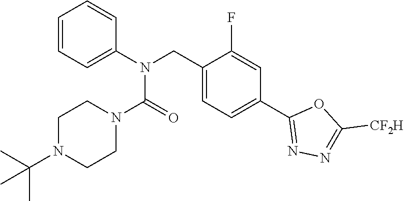 [SMILES: CC(C)(C)N1CCN(C(=O)N(Cc2ccc(-c3nnc(C(F)F)o3)cc2F)c2ccccc2)CC1] |
| 54 | 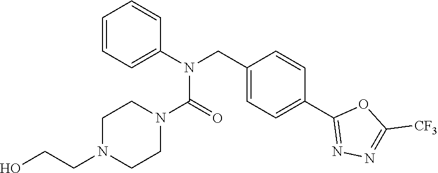 [SMILES: O=C(N1CCN(CCO)CC1)N(Cc1ccc(-c2nnc(C(F)(F)F)o2)cc1)c1ccccc1] |
| 55 | 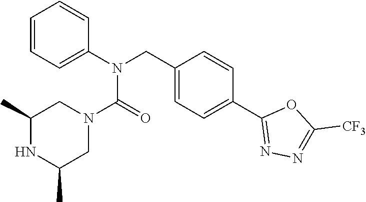 [SMILES: C[C@@H]1CN(C(=O)N(Cc2ccc(-c3nnc(C(F)(F)F)o3)cc2)c2ccccc2)C[C@H](C)N1] |
| 56 | 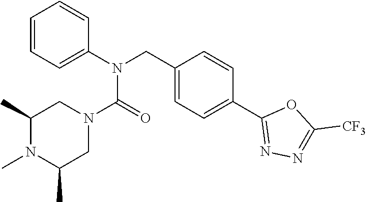 [SMILES: C[C@@H]1CN(C(=O)N(Cc2ccc(-c3nnc(C(F)(F)F)o3)cc2)c2ccccc2)C[C@H](C)N1C] |
| 57 | 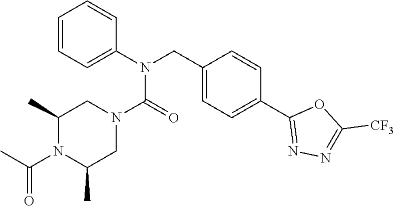 [SMILES: CC(=O)N1[C@H](C)CN(C(=O)N(Cc2ccc(-c3nnc(C(F)(F)F)o3)cc2)c2ccccc2)C[C@@H]1C] |
| 58 | 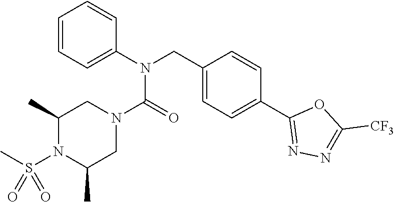 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 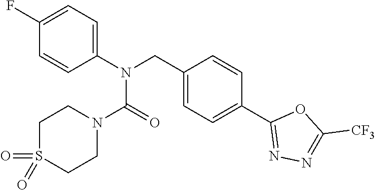 [SMILES: O=C(N1CCS(=O)(=O)CC1)N(Cc1ccc(-c2nnc(C(F)(F)F)o2)cc1)c1ccc(F)cc1] |
| 60 | 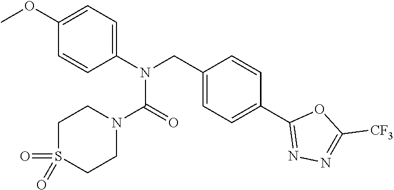 [SMILES: COc1ccc(N(Cc2ccc(-c3nnc(C(F)(F)F)o3)cc2)C(=O)N2CCS(=O)(=O)CC2)cc1] |
| 61 | 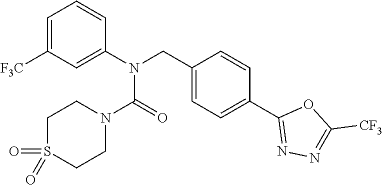 [SMILES: O=C(N1CCS(=O)(=O)CC1)N(Cc1ccc(-c2nnc(C(F)(F)F)o2)cc1)c1cccc(C(F)(F)F)c1] |
| 62 | 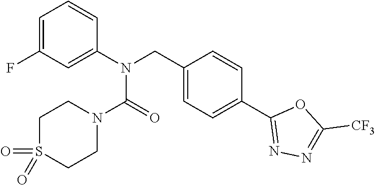 [SMILES: O=C(N1CCS(=O)(=O)CC1)N(Cc1ccc(-c2nnc(C(F)(F)F)o2)cc1)c1cccc(F)c1] |
| 63 | 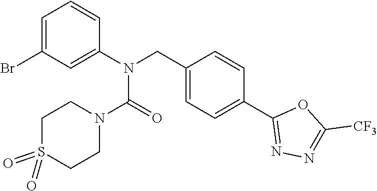 [SMILES: O=C(N1CCS(=O)(=O)CC1)N(Cc1ccc(-c2nnc(C(F)(F)F)o2)cc1)c1cccc(Br)c1] |
| 64 | 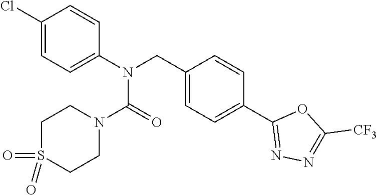 [SMILES: O=C(N1CCS(=O)(=O)CC1)N(Cc1ccc(-c2nnc(C(F)(F)F)o2)cc1)c1ccc(Cl)cc1] |
| 65 | 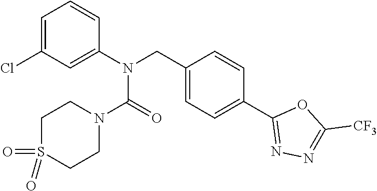 [SMILES: O=C(N1CCS(=O)(=O)CC1)N(Cc1ccc(-c2nnc(C(F)(F)F)o2)cc1)c1cccc(Cl)c1] |
| 66 | 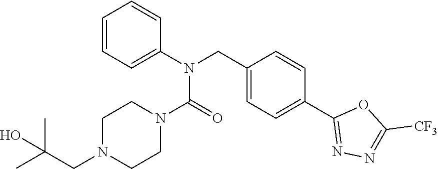 [SMILES: CC(C)(O)CN1CCN(C(=O)N(Cc2ccc(-c3nnc(C(F)(F)F)o3)cc2)c2ccccc2)CC1] |
| 67 | 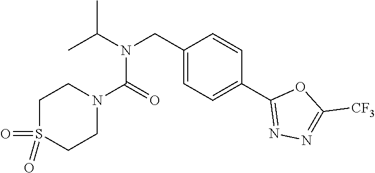 [SMILES: CC(C)N(Cc1ccc(-c2nnc(C(F)(F)F)o2)cc1)C(=O)N1CCS(=O)(=O)CC1] |
| 68 | 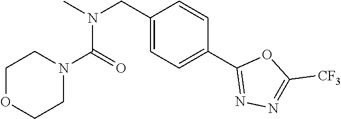 [SMILES: CN(Cc1ccc(-c2nnc(C(F)(F)F)o2)cc1)C(=O)N1CCOCC1] |
| 69 | 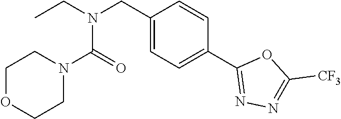 [SMILES: CCN(Cc1ccc(-c2nnc(C(F)(F)F)o2)cc1)C(=O)N1CCOCC1] |
| 70 | 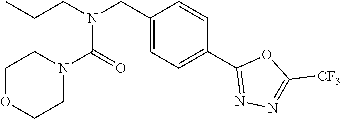 [SMILES: CCCN(Cc1ccc(-c2nnc(C(F)(F)F)o2)cc1)C(=O)N1CCOCC1] |
| 71 | 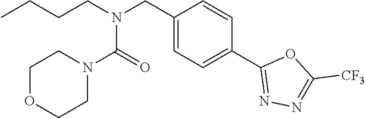 [SMILES: CCCCN(Cc1ccc(-c2nnc(C(F)(F)F)o2)cc1)C(=O)N1CCOCC1] |
| 72 | 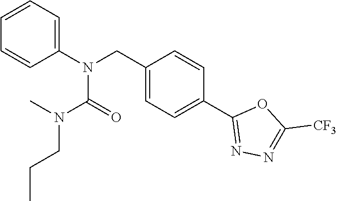 [SMILES: CCCN(C)C(=O)N(Cc1ccc(-c2nnc(C(F)(F)F)o2)cc1)c1ccccc1] |
| 73 | 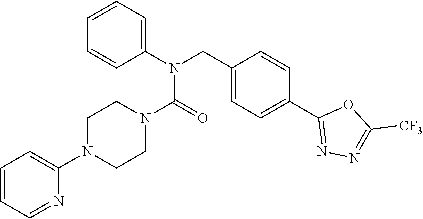 [SMILES: O=C(N1CCN(c2ccccn2)CC1)N(Cc1ccc(-c2nnc(C(F)(F)F)o2)cc1)c1ccccc1] |
| 74 | 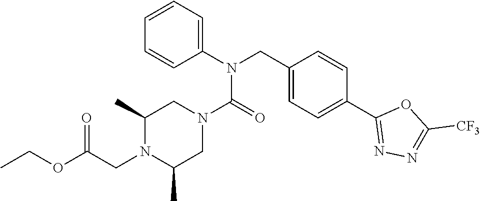 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 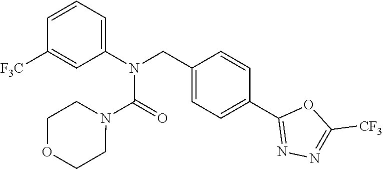 [SMILES: O=C(N1CCOCC1)N(Cc1ccc(-c2nnc(C(F)(F)F)o2)cc1)c1cccc(C(F)(F)F)c1] |
| 76 | 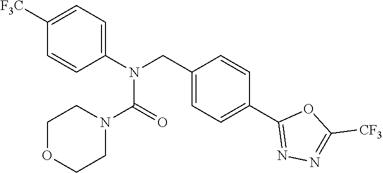 [SMILES: O=C(N1CCOCC1)N(Cc1ccc(-c2nnc(C(F)(F)F)o2)cc1)c1ccc(C(F)(F)F)cc1] |
| 77 | 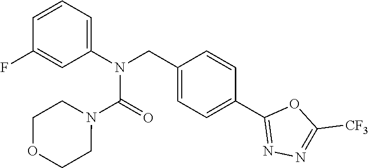 [SMILES: O=C(N1CCOCC1)N(Cc1ccc(-c2nnc(C(F)(F)F)o2)cc1)c1cccc(F)c1] |
| 78 | 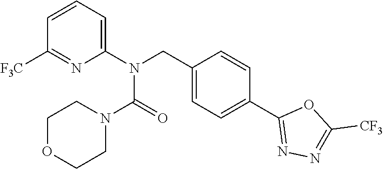 [SMILES: O=C(N1CCOCC1)N(Cc1ccc(-c2nnc(C(F)(F)F)o2)cc1)c1cccc(C(F)(F)F)n1] |
| 79 | 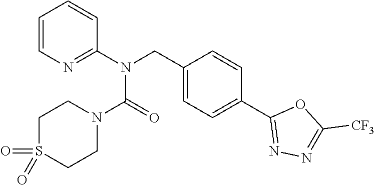 [SMILES: O=C(N1CCS(=O)(=O)CC1)N(Cc1ccc(-c2nnc(C(F)(F)F)o2)cc1)c1ccccn1] |
| 80 | 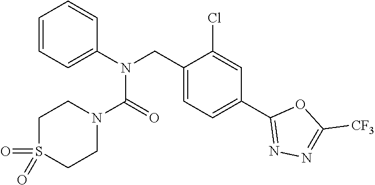 [SMILES: O=C(N1CCS(=O)(=O)CC1)N(Cc1ccc(-c2nnc(C(F)(F)F)o2)cc1Cl)c1ccccc1] |
| 81 | 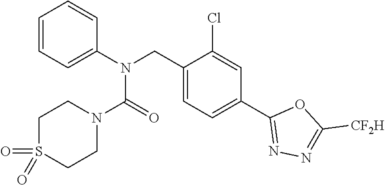 [SMILES: O=C(N1CCS(=O)(=O)CC1)N(Cc1ccc(-c2nnc(C(F)F)o2)cc1Cl)c1ccccc1] |
| 82 | 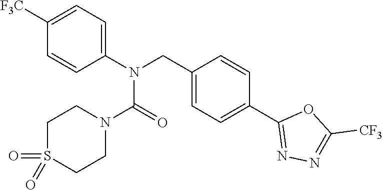 [SMILES: O=C(N1CCS(=O)(=O)CC1)N(Cc1ccc(-c2nnc(C(F)(F)F)o2)cc1)c1ccc(C(F)(F)F)cc1] |
| 83 | 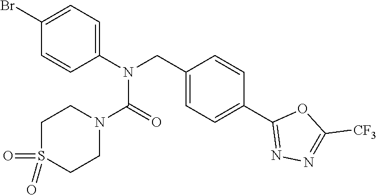 [SMILES: O=C(N1CCS(=O)(=O)CC1)N(Cc1ccc(-c2nnc(C(F)(F)F)o2)cc1)c1ccc(Br)cc1] |
| 84 | 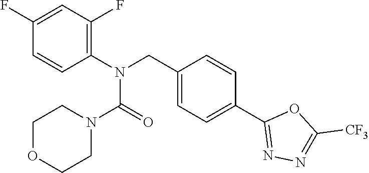 [SMILES: O=C(N1CCOCC1)N(Cc1ccc(-c2nnc(C(F)(F)F)o2)cc1)c1ccc(F)cc1F] |
| 85 | 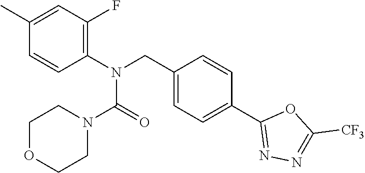 [SMILES: Cc1ccc(N(Cc2ccc(-c3nnc(C(F)(F)F)o3)cc2)C(=O)N2CCOCC2)c(F)c1] |
| 86 | 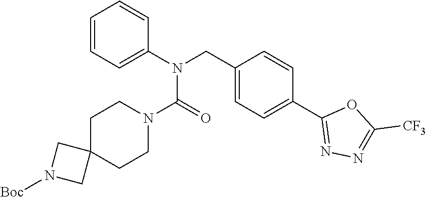 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 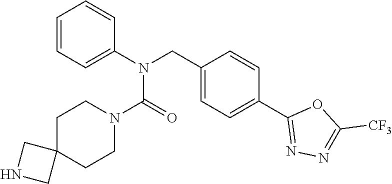 [SMILES: O=C(N1CCC2(CC1)CNC2)N(Cc1ccc(-c2nnc(C(F)(F)F)o2)cc1)c1ccccc1] |
| 88 | 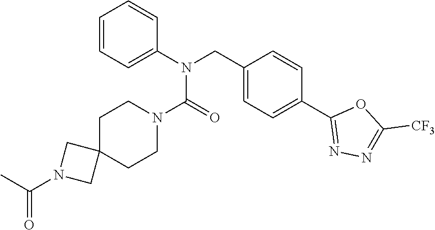 [SMILES: CC(=O)N1CC2(CCN(C(=O)N(Cc3ccc(-c4nnc(C(F)(F)F)o4)cc3)c3ccccc3)CC2)C1] |
| 89 | 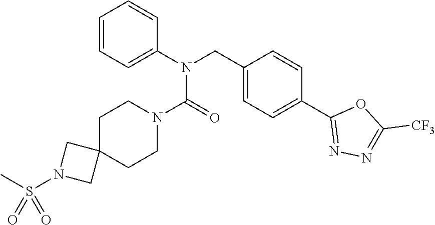 [SMILES: CS(=O)(=O)N1CC2(CCN(C(=O)N(Cc3ccc(-c4nnc(C(F)(F)F)o4)cc3)c3ccccc3)CC2)C1] |
| 90 | 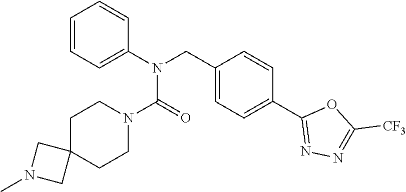 [SMILES: CN1CC2(CCN(C(=O)N(Cc3ccc(-c4nnc(C(F)(F)F)o4)cc3)c3ccccc3)CC2)C1] |
| 91 | 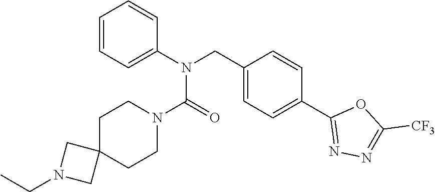 [SMILES: CCN1CC2(CCN(C(=O)N(Cc3ccc(-c4nnc(C(F)(F)F)o4)cc3)c3ccccc3)CC2)C1] |
| 92 | 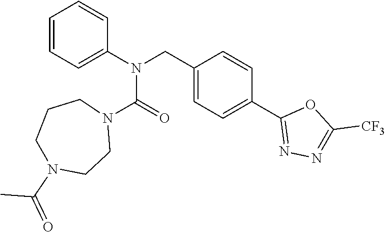 [SMILES: CC(=O)N1CCCN(C(=O)N(Cc2ccc(-c3nnc(C(F)(F)F)o3)cc2)c2ccccc2)CC1] |
| 93 | 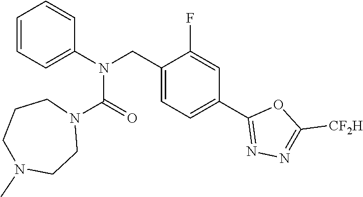 [SMILES: CN1CCCN(C(=O)N(Cc2ccc(-c3nnc(C(F)F)o3)cc2F)c2ccccc2)CC1] |
| 94 | 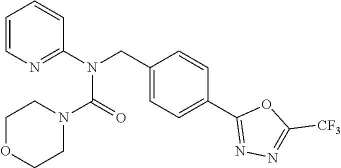 [SMILES: O=C(N1CCOCC1)N(Cc1ccc(-c2nnc(C(F)(F)F)o2)cc1)c1ccccn1] |
| 95 | 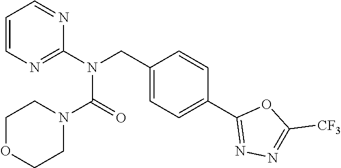 [SMILES: O=C(N1CCOCC1)N(Cc1ccc(-c2nnc(C(F)(F)F)o2)cc1)c1ncccn1] |
| 96 | 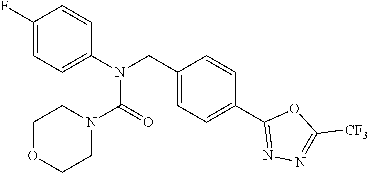 [SMILES: O=C(N1CCOCC1)N(Cc1ccc(-c2nnc(C(F)(F)F)o2)cc1)c1ccc(F)cc1] |
| 97 | 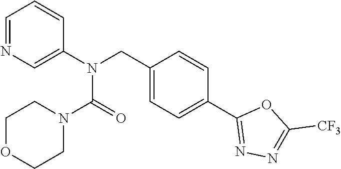 [SMILES: O=C(N1CCOCC1)N(Cc1ccc(-c2nnc(C(F)(F)F)o2)cc1)c1cccnc1] |
| 98 | 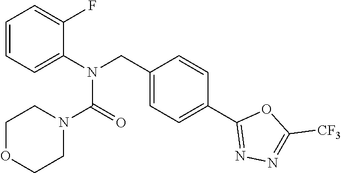 [SMILES: O=C(N1CCOCC1)N(Cc1ccc(-c2nnc(C(F)(F)F)o2)cc1)c1ccccc1F] |
| 99 | 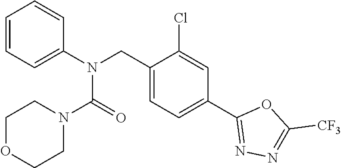 [SMILES: O=C(N1CCOCC1)N(Cc1ccc(-c2nnc(C(F)(F)F)o2)cc1Cl)c1ccccc1] |
| 100 | 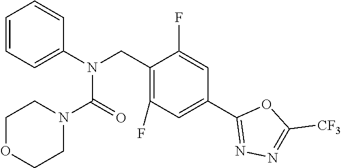 [SMILES: O=C(N1CCOCC1)N(Cc1c(F)cc(-c2nnc(C(F)(F)F)o2)cc1F)c1ccccc1] |
| 101 | 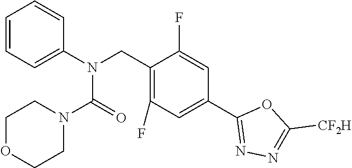 [SMILES: O=C(N1CCOCC1)N(Cc1c(F)cc(-c2nnc(C(F)F)o2)cc1F)c1ccccc1] |
| 102 | 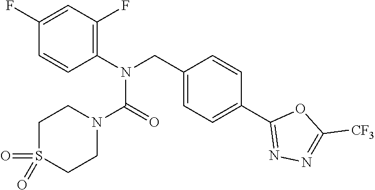 [SMILES: O=C(N1CCS(=O)(=O)CC1)N(Cc1ccc(-c2nnc(C(F)(F)F)o2)cc1)c1ccc(F)cc1F] |
| 103 | 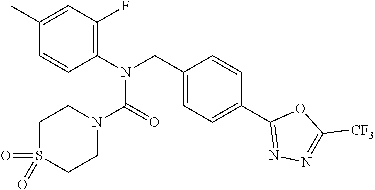 [SMILES: Cc1ccc(N(Cc2ccc(-c3nnc(C(F)(F)F)o3)cc2)C(=O)N2CCS(=O)(=O)CC2)c(F)c1] |
| 104 | 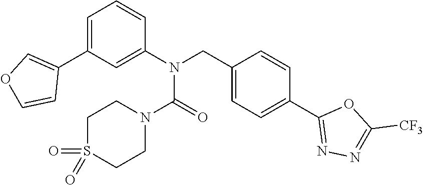 [SMILES: O=C(N1CCS(=O)(=O)CC1)N(Cc1ccc(-c2nnc(C(F)(F)F)o2)cc1)c1cccc(-c2ccoc2)c1] |
| 105 | 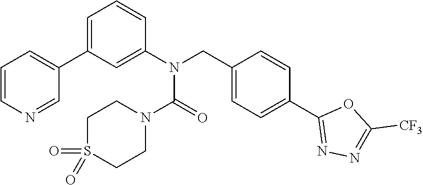 [SMILES: O=C(N1CCS(=O)(=O)CC1)N(Cc1ccc(-c2nnc(C(F)(F)F)o2)cc1)c1cccc(-c2cccnc2)c1] |
| 106 | 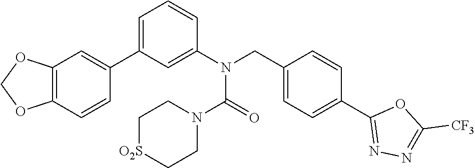 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 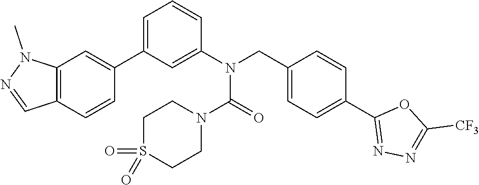 [SMILES: Cn1ncc2ccc(-c3cccc(N(Cc4ccc(-c5nnc(C(F)(F)F)o5)cc4)C(=O)N4CCS(=O)(=O)CC4)c3)cc21] |
| 108 | 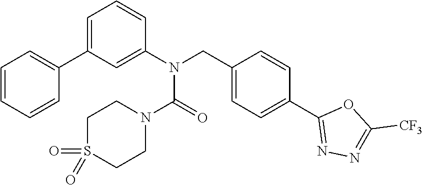 [SMILES: O=C(N1CCS(=O)(=O)CC1)N(Cc1ccc(-c2nnc(C(F)(F)F)o2)cc1)c1cccc(-c2ccccc2)c1] |
| 109 | 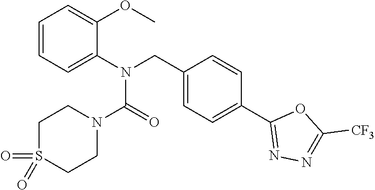 [SMILES: COc1ccccc1N(Cc1ccc(-c2nnc(C(F)(F)F)o2)cc1)C(=O)N1CCS(=O)(=O)CC1] |
| 110 | 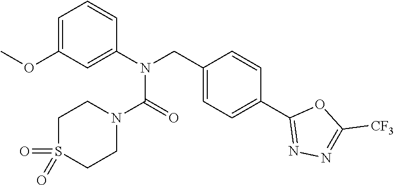 [SMILES: COc1cccc(N(Cc2ccc(-c3nnc(C(F)(F)F)o3)cc2)C(=O)N2CCS(=O)(=O)CC2)c1] |
| 111 | 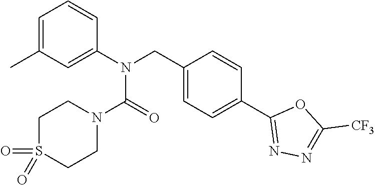 [SMILES: Cc1cccc(N(Cc2ccc(-c3nnc(C(F)(F)F)o3)cc2)C(=O)N2CCS(=O)(=O)CC2)c1] |
| 112 | 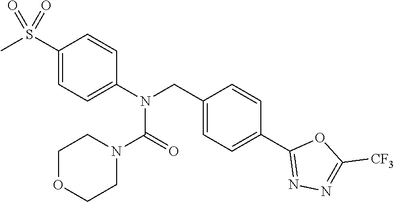 [SMILES: CS(=O)(=O)c1ccc(N(Cc2ccc(-c3nnc(C(F)(F)F)o3)cc2)C(=O)N2CCOCC2)cc1] |
| 113 | 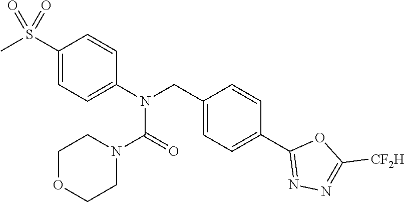 [SMILES: CS(=O)(=O)c1ccc(N(Cc2ccc(-c3nnc(C(F)F)o3)cc2)C(=O)N2CCOCC2)cc1] |
| 114 | 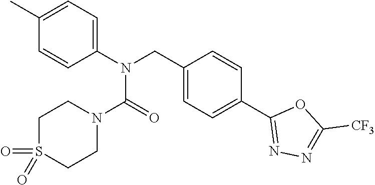 [SMILES: Cc1ccc(N(Cc2ccc(-c3nnc(C(F)(F)F)o3)cc2)C(=O)N2CCS(=O)(=O)CC2)cc1] |
| 115 | 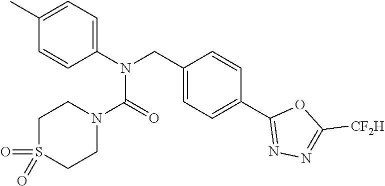 [SMILES: Cc1ccc(N(Cc2ccc(-c3nnc(C(F)F)o3)cc2)C(=O)N2CCS(=O)(=O)CC2)cc1] |
| 116 | 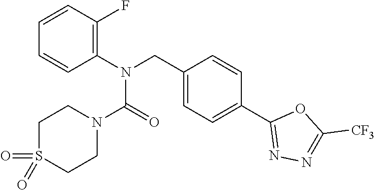 [SMILES: O=C(N1CCS(=O)(=O)CC1)N(Cc1ccc(-c2nnc(C(F)(F)F)o2)cc1)c1ccccc1F] |
| 117 | 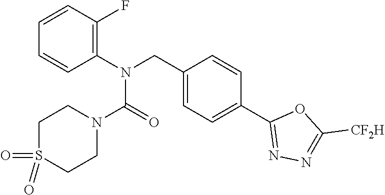 [SMILES: O=C(N1CCS(=O)(=O)CC1)N(Cc1ccc(-c2nnc(C(F)F)o2)cc1)c1ccccc1F] |
| 118 | 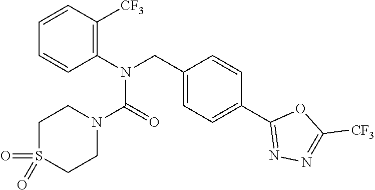 [SMILES: O=C(N1CCS(=O)(=O)CC1)N(Cc1ccc(-c2nnc(C(F)(F)F)o2)cc1)c1ccccc1C(F)(F)F] |
| 119 | 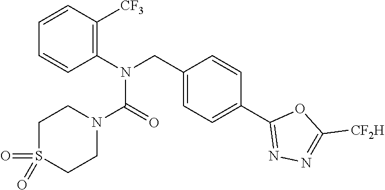 [SMILES: O=C(N1CCS(=O)(=O)CC1)N(Cc1ccc(-c2nnc(C(F)F)o2)cc1)c1ccccc1C(F)(F)F] |
| 120 | 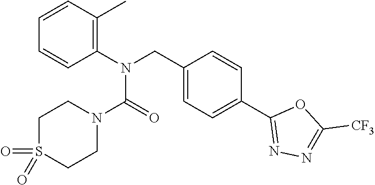 [SMILES: Cc1ccccc1N(Cc1ccc(-c2nnc(C(F)(F)F)o2)cc1)C(=O)N1CCS(=O)(=O)CC1] |
| 121 | 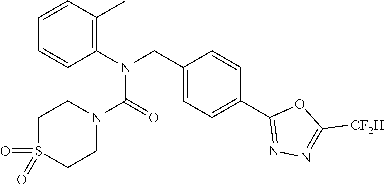 [SMILES: Cc1ccccc1N(Cc1ccc(-c2nnc(C(F)F)o2)cc1)C(=O)N1CCS(=O)(=O)CC1] |
| 122 | 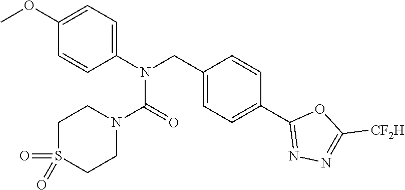 [SMILES: COc1ccc(N(Cc2ccc(-c3nnc(C(F)F)o3)cc2)C(=O)N2CCS(=O)(=O)CC2)cc1] |
| 123 | 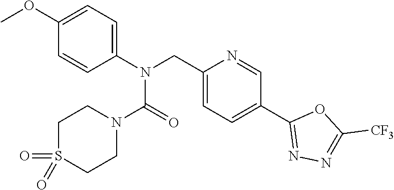 [SMILES: COc1ccc(N(Cc2ccc(-c3nnc(C(F)(F)F)o3)cn2)C(=O)N2CCS(=O)(=O)CC2)cc1] |
| 124 | 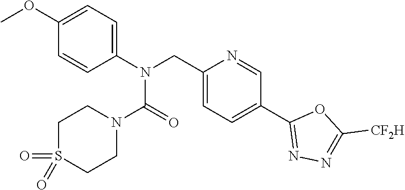 [SMILES: COc1ccc(N(Cc2ccc(-c3nnc(C(F)F)o3)cn2)C(=O)N2CCS(=O)(=O)CC2)cc1] |
| 125 | 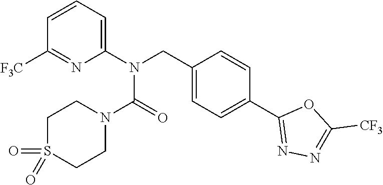 [SMILES: O=C(N1CCS(=O)(=O)CC1)N(Cc1ccc(-c2nnc(C(F)(F)F)o2)cc1)c1cccc(C(F)(F)F)n1] |
| 126 | 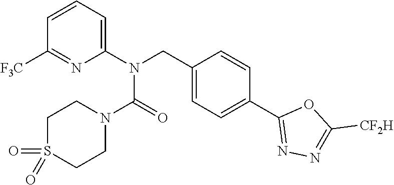 [SMILES: O=C(N1CCS(=O)(=O)CC1)N(Cc1ccc(-c2nnc(C(F)F)o2)cc1)c1cccc(C(F)(F)F)n1] |
| 127 | 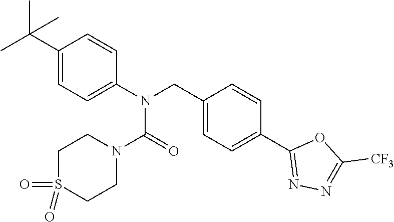 [SMILES: CC(C)(C)c1ccc(N(Cc2ccc(-c3nnc(C(F)(F)F)o3)cc2)C(=O)N2CCS(=O)(=O)CC2)cc1] |
| 128 | 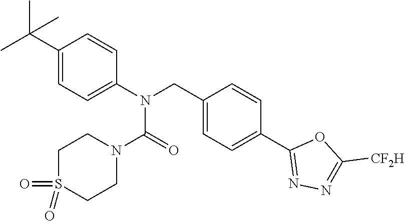 [SMILES: CC(C)(C)c1ccc(N(Cc2ccc(-c3nnc(C(F)F)o3)cc2)C(=O)N2CCS(=O)(=O)CC2)cc1] |
| 129 | 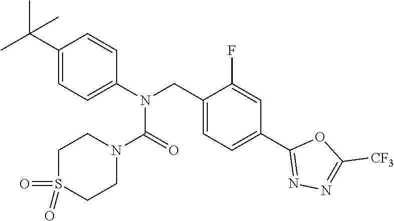 [SMILES: CC(C)(C)c1ccc(N(Cc2ccc(-c3nnc(C(F)(F)F)o3)cc2F)C(=O)N2CCS(=O)(=O)CC2)cc1] |
| 130 | 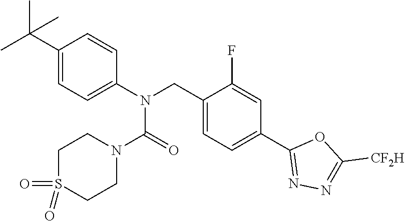 [SMILES: CC(C)(C)c1ccc(N(Cc2ccc(-c3nnc(C(F)F)o3)cc2F)C(=O)N2CCS(=O)(=O)CC2)cc1] |
| 131 | 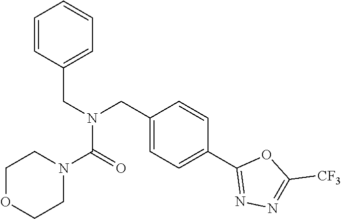 [SMILES: O=C(N1CCOCC1)N(Cc1ccccc1)Cc1ccc(-c2nnc(C(F)(F)F)o2)cc1] |
| 132 | 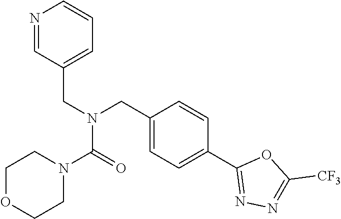 [SMILES: O=C(N1CCOCC1)N(Cc1ccc(-c2nnc(C(F)(F)F)o2)cc1)Cc1cccnc1] |
| 133 | 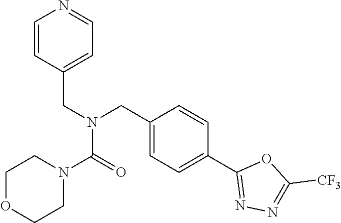 [SMILES: O=C(N1CCOCC1)N(Cc1ccncc1)Cc1ccc(-c2nnc(C(F)(F)F)o2)cc1] |
| 134 | 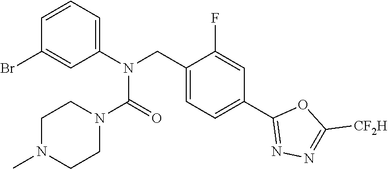 [SMILES: CN1CCN(C(=O)N(Cc2ccc(-c3nnc(C(F)F)o3)cc2F)c2cccc(Br)c2)CC1] |
| 135 | 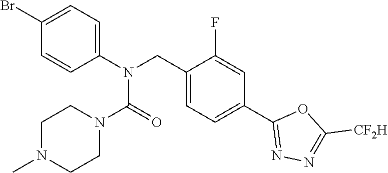 [SMILES: CN1CCN(C(=O)N(Cc2ccc(-c3nnc(C(F)F)o3)cc2F)c2ccc(Br)cc2)CC1] |
| 136 | 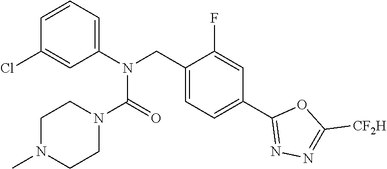 [SMILES: CN1CCN(C(=O)N(Cc2ccc(-c3nnc(C(F)F)o3)cc2F)c2cccc(Cl)c2)CC1] |
| 137 | 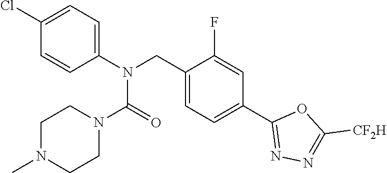 [SMILES: CN1CCN(C(=O)N(Cc2ccc(-c3nnc(C(F)F)o3)cc2F)c2ccc(Cl)cc2)CC1] |
| 138 | 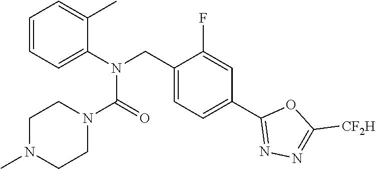 [SMILES: Cc1ccccc1N(Cc1ccc(-c2nnc(C(F)F)o2)cc1F)C(=O)N1CCN(C)CC1] |
| 139 | 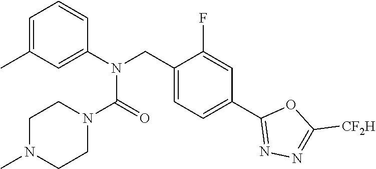 [SMILES: Cc1cccc(N(Cc2ccc(-c3nnc(C(F)F)o3)cc2F)C(=O)N2CCN(C)CC2)c1] |
| 140 | 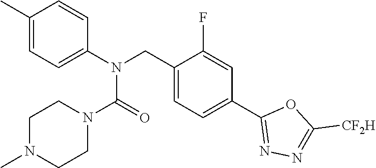 [SMILES: Cc1ccc(N(Cc2ccc(-c3nnc(C(F)F)o3)cc2F)C(=O)N2CCN(C)CC2)cc1] |
| 141 | 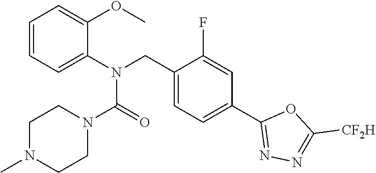 [SMILES: COc1ccccc1N(Cc1ccc(-c2nnc(C(F)F)o2)cc1F)C(=O)N1CCN(C)CC1] |
| 142 | 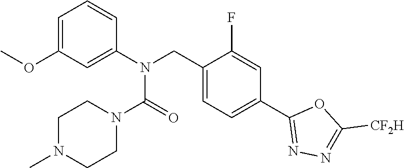 [SMILES: COc1cccc(N(Cc2ccc(-c3nnc(C(F)F)o3)cc2F)C(=O)N2CCN(C)CC2)c1] |
| 143 | 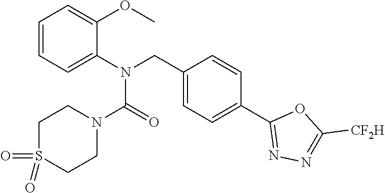 [SMILES: COc1ccccc1N(Cc1ccc(-c2nnc(C(F)F)o2)cc1)C(=O)N1CCS(=O)(=O)CC1] |
| 144 | 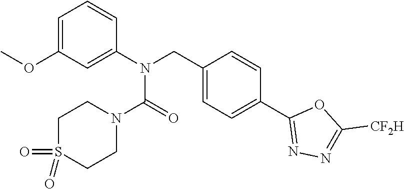 [SMILES: COc1cccc(N(Cc2ccc(-c3nnc(C(F)F)o3)cc2)C(=O)N2CCS(=O)(=O)CC2)c1] |
| 145 | 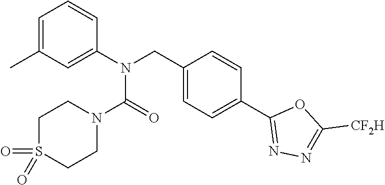 [SMILES: Cc1cccc(N(Cc2ccc(-c3nnc(C(F)F)o3)cc2)C(=O)N2CCS(=O)(=O)CC2)c1] |
| 146 | 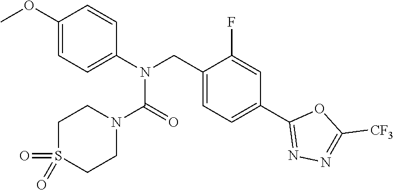 [SMILES: COc1ccc(N(Cc2ccc(-c3nnc(C(F)(F)F)o3)cc2F)C(=O)N2CCS(=O)(=O)CC2)cc1] |
| 147 | 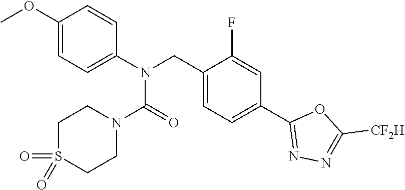 [SMILES: COc1ccc(N(Cc2ccc(-c3nnc(C(F)F)o3)cc2F)C(=O)N2CCS(=O)(=O)CC2)cc1] |
| 148 | 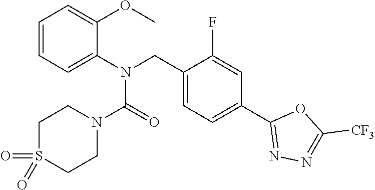 [SMILES: COc1ccccc1N(Cc1ccc(-c2nnc(C(F)(F)F)o2)cc1F)C(=O)N1CCS(=O)(=O)CC1] |
| 149 | 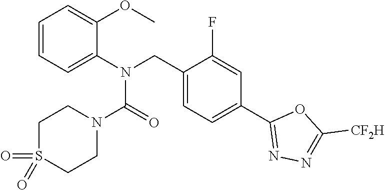 [SMILES: COc1ccccc1N(Cc1ccc(-c2nnc(C(F)F)o2)cc1F)C(=O)N1CCS(=O)(=O)CC1] |
| 150 | 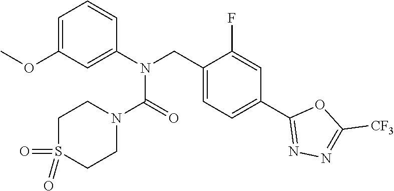 [SMILES: COc1cccc(N(Cc2ccc(-c3nnc(C(F)(F)F)o3)cc2F)C(=O)N2CCS(=O)(=O)CC2)c1] |
| 151 | 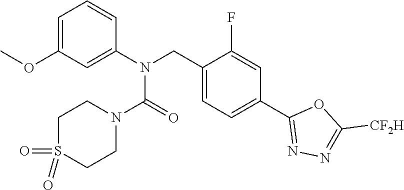 [SMILES: COc1cccc(N(Cc2ccc(-c3nnc(C(F)F)o3)cc2F)C(=O)N2CCS(=O)(=O)CC2)c1] |
| 152 | 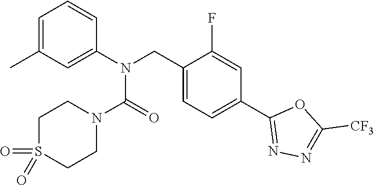 [SMILES: Cc1cccc(N(Cc2ccc(-c3nnc(C(F)(F)F)o3)cc2F)C(=O)N2CCS(=O)(=O)CC2)c1] |
| 153 | 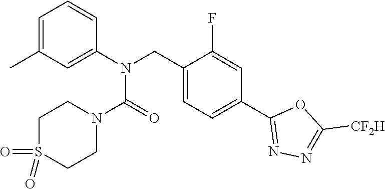 [SMILES: Cc1cccc(N(Cc2ccc(-c3nnc(C(F)F)o3)cc2F)C(=O)N2CCS(=O)(=O)CC2)c1] |
| 154 | 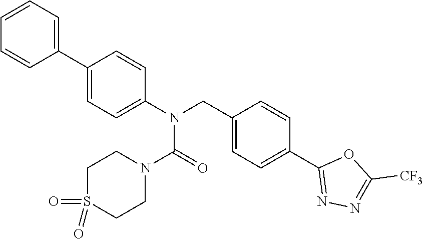 [SMILES: O=C(N1CCS(=O)(=O)CC1)N(Cc1ccc(-c2nnc(C(F)(F)F)o2)cc1)c1ccc(-c2ccccc2)cc1] |
| 155 | 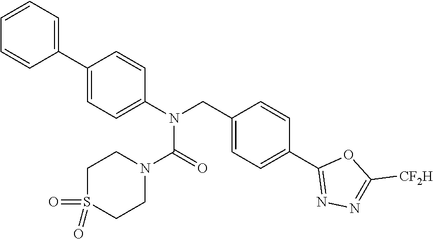 [SMILES: O=C(N1CCS(=O)(=O)CC1)N(Cc1ccc(-c2nnc(C(F)F)o2)cc1)c1ccc(-c2ccccc2)cc1] |
| 156 | 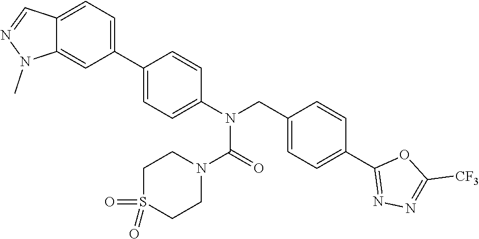 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 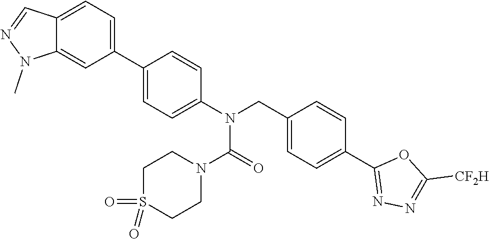 [SMILES: Cn1ncc2ccc(-c3ccc(N(Cc4ccc(-c5nnc(C(F)F)o5)cc4)C(=O)N4CCS(=O)(=O)CC4)cc3)cc21] |
| 158 | 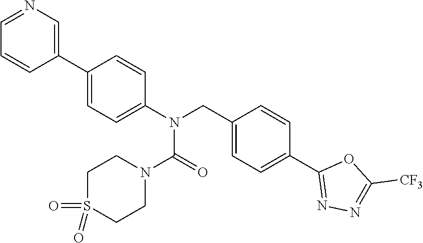 [SMILES: O=C(N1CCS(=O)(=O)CC1)N(Cc1ccc(-c2nnc(C(F)(F)F)o2)cc1)c1ccc(-c2cccnc2)cc1] |
| 159 | 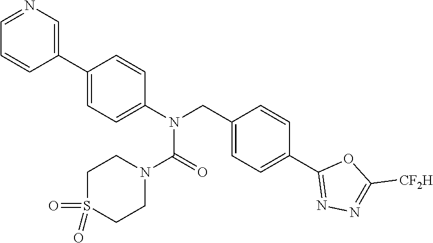 [SMILES: O=C(N1CCS(=O)(=O)CC1)N(Cc1ccc(-c2nnc(C(F)F)o2)cc1)c1ccc(-c2cccnc2)cc1] |
| 160 | 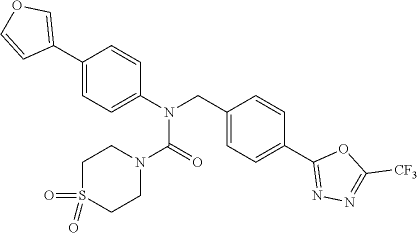 [SMILES: O=C(N1CCS(=O)(=O)CC1)N(Cc1ccc(-c2nnc(C(F)(F)F)o2)cc1)c1ccc(-c2ccoc2)cc1] |
| 161 | 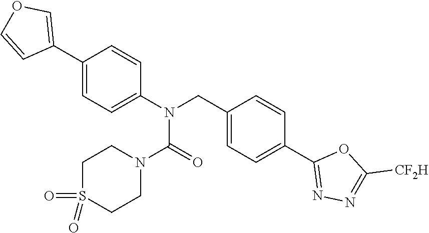 [SMILES: O=C(N1CCS(=O)(=O)CC1)N(Cc1ccc(-c2nnc(C(F)F)o2)cc1)c1ccc(-c2ccoc2)cc1] |
| 162 | 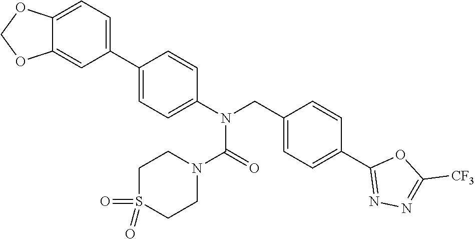 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 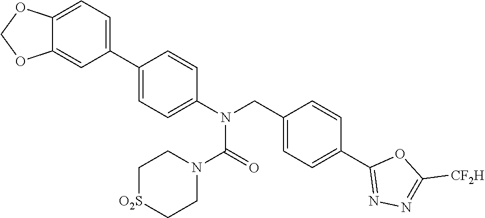 [SMILES: O=C(N1CCS(=O)(=O)CC1)N(Cc1ccc(-c2nnc(C(F)F)o2)cc1)c1ccc(-c2ccc3c(c2)OCO3)cc1] |
| 164 | 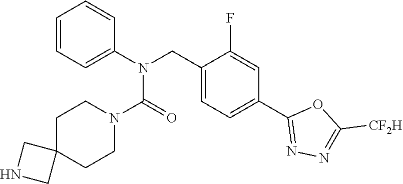 [SMILES: O=C(N1CCC2(CC1)CNC2)N(Cc1ccc(-c2nnc(C(F)F)o2)cc1F)c1ccccc1] |
| 165 | 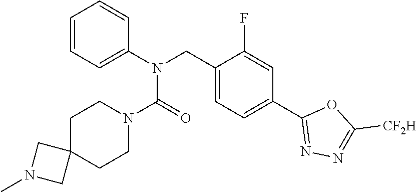 [SMILES: CN1CC2(CCN(C(=O)N(Cc3ccc(-c4nnc(C(F)F)o4)cc3F)c3ccccc3)CC2)C1] |
| 166 | 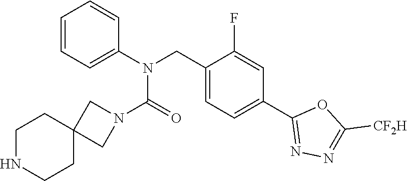 [SMILES: O=C(N1CC2(CCNCC2)C1)N(Cc1ccc(-c2nnc(C(F)F)o2)cc1F)c1ccccc1] |
| 167 | 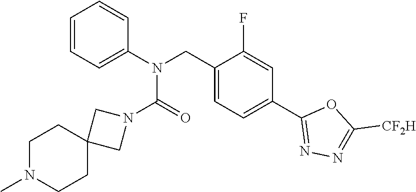 [SMILES: CN1CCC2(CC1)CN(C(=O)N(Cc1ccc(-c3nnc(C(F)F)o3)cc1F)c1ccccc1)C2] |
| 168 | 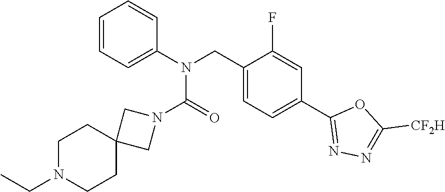 [SMILES: CCN1CCC2(CC1)CN(C(=O)N(Cc1ccc(-c3nnc(C(F)F)o3)cc1F)c1ccccc1)C2] |
| 169 | 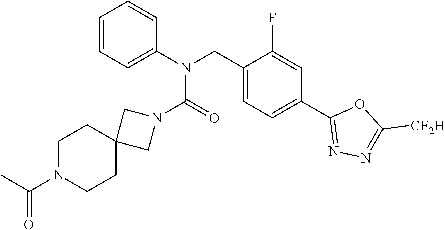 [SMILES: CC(=O)N1CCC2(CC1)CN(C(=O)N(Cc1ccc(-c3nnc(C(F)F)o3)cc1F)c1ccccc1)C2] |
| 170 | 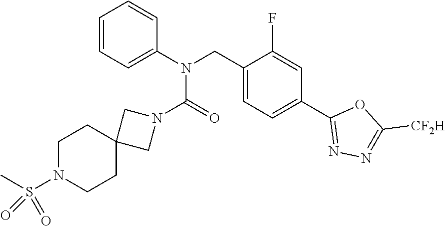 [SMILES: CS(=O)(=O)N1CCC2(CC1)CN(C(=O)N(Cc1ccc(-c3nnc(C(F)F)o3)cc1F)c1ccccc1)C2] |
| 171 | 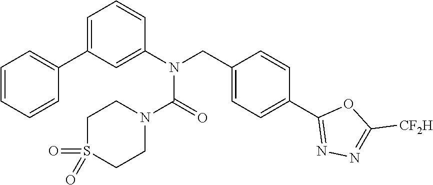 [SMILES: O=C(N1CCS(=O)(=O)CC1)N(Cc1ccc(-c2nnc(C(F)F)o2)cc1)c1cccc(-c2ccccc2)c1] |
| 172 | 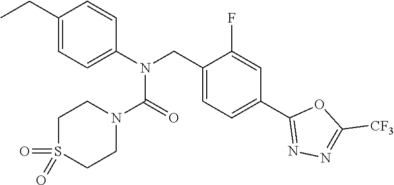 [SMILES: CCc1ccc(N(Cc2ccc(-c3nnc(C(F)(F)F)o3)cc2F)C(=O)N2CCS(=O)(=O)CC2)cc1] |
| 173 | 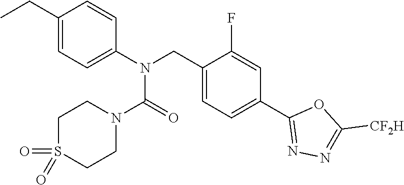 [SMILES: CCc1ccc(N(Cc2ccc(-c3nnc(C(F)F)o3)cc2F)C(=O)N2CCS(=O)(=O)CC2)cc1] |
| 174 | 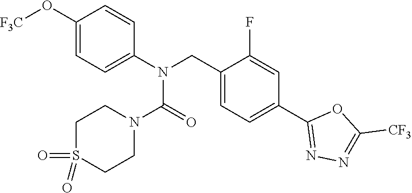 [SMILES: O=C(N1CCS(=O)(=O)CC1)N(Cc1ccc(-c2nnc(C(F)(F)F)o2)cc1F)c1ccc(OC(F)(F)F)cc1] |
| 175 | 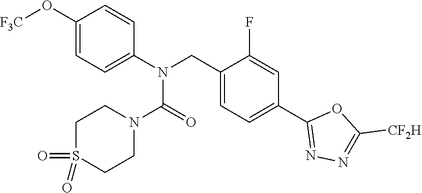 [SMILES: O=C(N1CCS(=O)(=O)CC1)N(Cc1ccc(-c2nnc(C(F)F)o2)cc1F)c1ccc(OC(F)(F)F)cc1] |
| 176 | 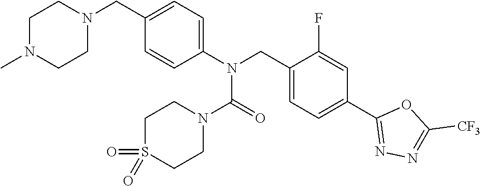 [SMILES: CN1CCN(Cc2ccc(N(Cc3ccc(-c4nnc(C(F)(F)F)o4)cc3F)C(=O)N3CCS(=O)(=O)CC3)cc2)CC1] |
| 177 | 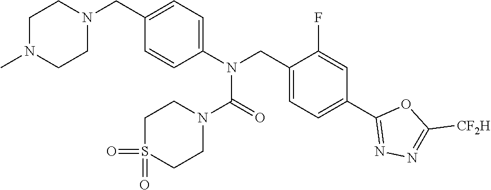 [SMILES: CN1CCN(Cc2ccc(N(Cc3ccc(-c4nnc(C(F)F)o4)cc3F)C(=O)N3CCS(=O)(=O)CC3)cc2)CC1] |
| 178 | 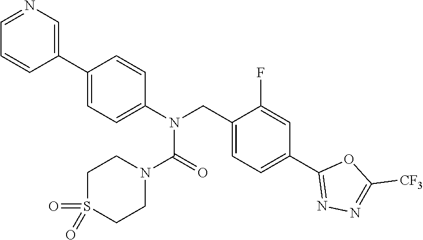 [SMILES: O=C(N1CCS(=O)(=O)CC1)N(Cc1ccc(-c2nnc(C(F)(F)F)o2)cc1F)c1ccc(-c2cccnc2)cc1] |
| 179 | 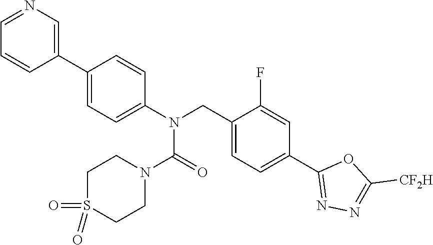 [SMILES: O=C(N1CCS(=O)(=O)CC1)N(Cc1ccc(-c2nnc(C(F)F)o2)cc1F)c1ccc(-c2cccnc2)cc1] |
| 180 | 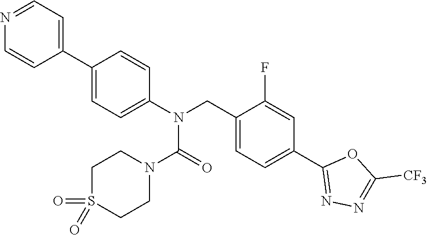 [SMILES: O=C(N1CCS(=O)(=O)CC1)N(Cc1ccc(-c2nnc(C(F)(F)F)o2)cc1F)c1ccc(-c2ccncc2)cc1] |
| 181 | 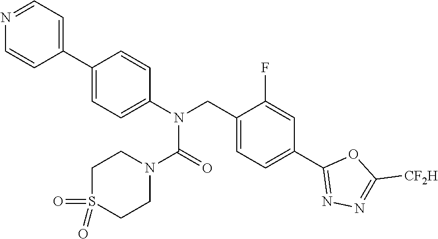 [SMILES: O=C(N1CCS(=O)(=O)CC1)N(Cc1ccc(-c2nnc(C(F)F)o2)cc1F)c1ccc(-c2ccncc2)cc1] |
| 182 | 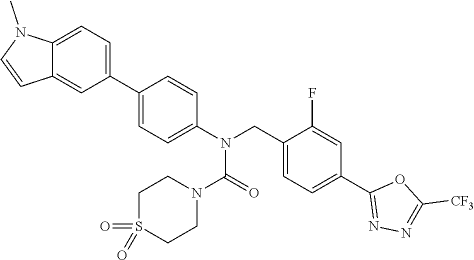 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 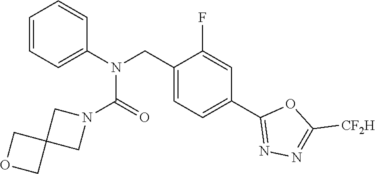 [SMILES: O=C(N1CC2(COC2)C1)N(Cc1ccc(-c2nnc(C(F)F)o2)cc1F)c1ccccc1] |
| 184 | 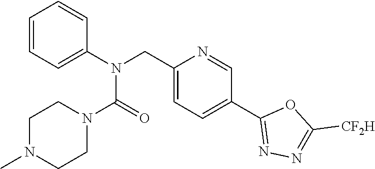 [SMILES: CN1CCN(C(=O)N(Cc2ccc(-c3nnc(C(F)F)o3)cn2)c2ccccc2)CC1] |
| 185 | 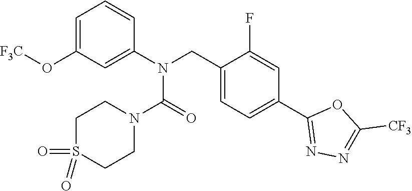 [SMILES: O=C(N1CCS(=O)(=O)CC1)N(Cc1ccc(-c2nnc(C(F)(F)F)o2)cc1F)c1cccc(OC(F)(F)F)c1] |
| 186 | 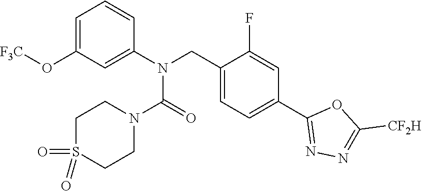 [SMILES: O=C(N1CCS(=O)(=O)CC1)N(Cc1ccc(-c2nnc(C(F)F)o2)cc1F)c1cccc(OC(F)(F)F)c1] |
| 187 | 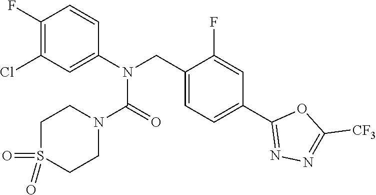 [SMILES: O=C(N1CCS(=O)(=O)CC1)N(Cc1ccc(-c2nnc(C(F)(F)F)o2)cc1F)c1ccc(F)c(Cl)c1] |
| 188 | 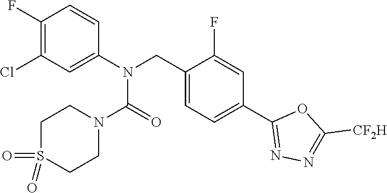 [SMILES: O=C(N1CCS(=O)(=O)CC1)N(Cc1ccc(-c2nnc(C(F)F)o2)cc1F)c1ccc(F)c(Cl)c1] |
| 189 | 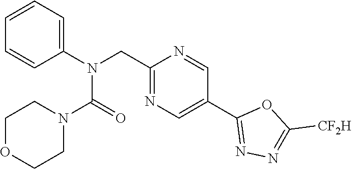 [SMILES: O=C(N1CCOCC1)N(Cc1ncc(-c2nnc(C(F)F)o2)cn1)c1ccccc1] |
| 190 | 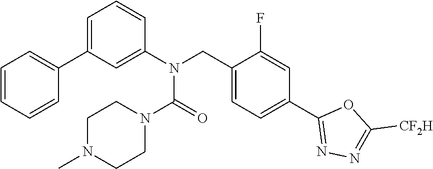 [SMILES: CN1CCN(C(=O)N(Cc2ccc(-c3nnc(C(F)F)o3)cc2F)c2cccc(-c3ccccc3)c2)CC1] |
| 191 | 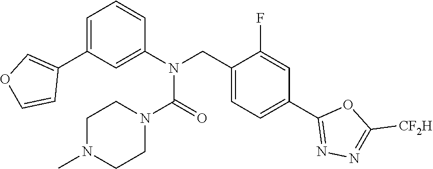 [SMILES: CN1CCN(C(=O)N(Cc2ccc(-c3nnc(C(F)F)o3)cc2F)c2cccc(-c3ccoc3)c2)CC1] |
| 192 | 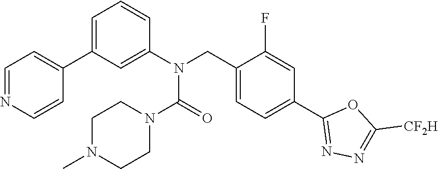 [SMILES: CN1CCN(C(=O)N(Cc2ccc(-c3nnc(C(F)F)o3)cc2F)c2cccc(-c3ccncc3)c2)CC1] |
| 193 | 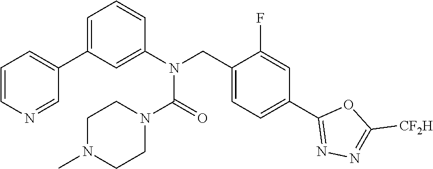 [SMILES: CN1CCN(C(=O)N(Cc2ccc(-c3nnc(C(F)F)o3)cc2F)c2cccc(-c3cccnc3)c2)CC1] |
| 194 | 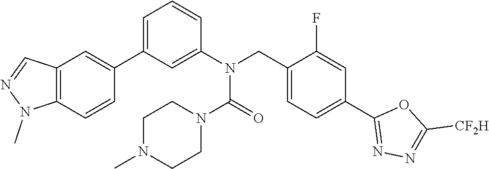 [SMILES: CN1CCN(C(=O)N(Cc2ccc(-c3nnc(C(F)F)o3)cc2F)c2cccc(-c3ccc4c(cnn4C)c3)c2)CC1] |
| 195 | 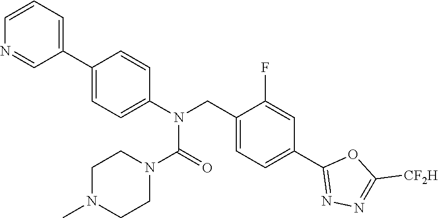 [SMILES: CN1CCN(C(=O)N(Cc2ccc(-c3nnc(C(F)F)o3)cc2F)c2ccc(-c3cccnc3)cc2)CC1] |
| 196 | 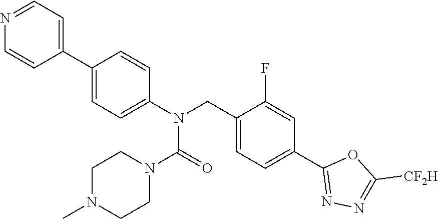 [SMILES: CN1CCN(C(=O)N(Cc2ccc(-c3nnc(C(F)F)o3)cc2F)c2ccc(-c3ccncc3)cc2)CC1] |
| 197 | 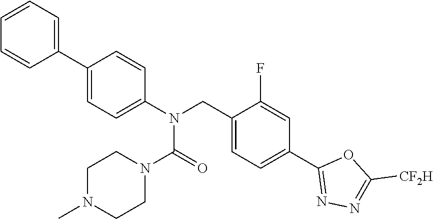 [SMILES: CN1CCN(C(=O)N(Cc2ccc(-c3nnc(C(F)F)o3)cc2F)c2ccc(-c3ccccc3)cc2)CC1] |
| 198 | 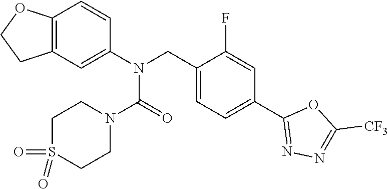 [SMILES: O=C(N1CCS(=O)(=O)CC1)N(Cc1ccc(-c2nnc(C(F)(F)F)o2)cc1F)c1ccc2c(c1)CCO2] |
| 199 | 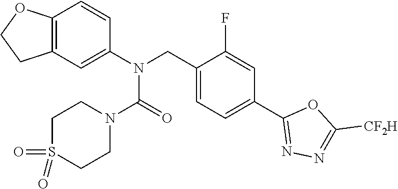 [SMILES: O=C(N1CCS(=O)(=O)CC1)N(Cc1ccc(-c2nnc(C(F)F)o2)cc1F)c1ccc2c(c1)CCO2] |
| 200 | 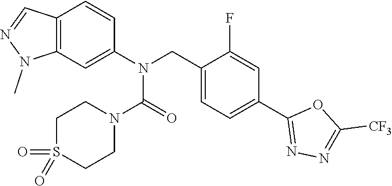 [SMILES: Cn1ncc2ccc(N(Cc3ccc(-c4nnc(C(F)(F)F)o4)cc3F)C(=O)N3CCS(=O)(=O)CC3)cc21] |
| 201 | 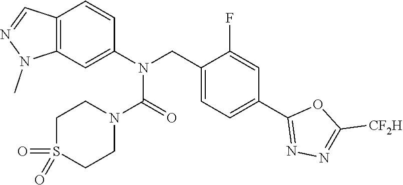 [SMILES: Cn1ncc2ccc(N(Cc3ccc(-c4nnc(C(F)F)o4)cc3F)C(=O)N3CCS(=O)(=O)CC3)cc21] |
| 202 | 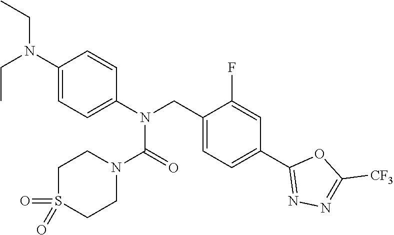 [SMILES: CCN(CC)c1ccc(N(Cc2ccc(-c3nnc(C(F)(F)F)o3)cc2F)C(=O)N2CCS(=O)(=O)CC2)cc1] |
| 203 | 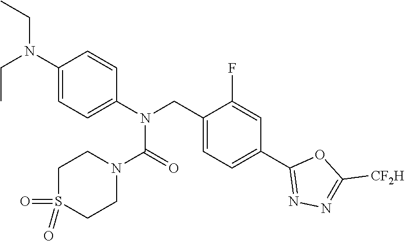 [SMILES: CCN(CC)c1ccc(N(Cc2ccc(-c3nnc(C(F)F)o3)cc2F)C(=O)N2CCS(=O)(=O)CC2)cc1] |
| 204 | 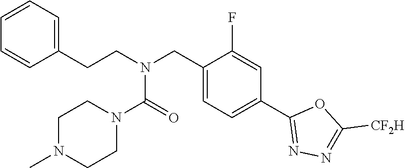 [SMILES: CN1CCN(C(=O)N(CCc2ccccc2)Cc2ccc(-c3nnc(C(F)F)o3)cc2F)CC1] |
| 205 | 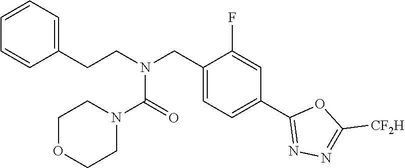 [SMILES: O=C(N1CCOCC1)N(CCc1ccccc1)Cc1ccc(-c2nnc(C(F)F)o2)cc1F] |
| 206 | 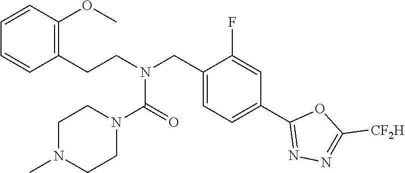 [SMILES: COc1ccccc1CCN(Cc1ccc(-c2nnc(C(F)F)o2)cc1F)C(=O)N1CCN(C)CC1] |
| 207 | 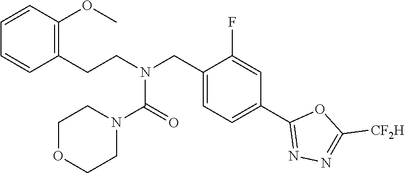 [SMILES: COc1ccccc1CCN(Cc1ccc(-c2nnc(C(F)F)o2)cc1F)C(=O)N1CCOCC1] |
| 208 | 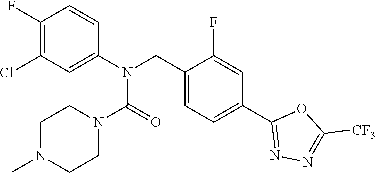 [SMILES: CN1CCN(C(=O)N(Cc2ccc(-c3nnc(C(F)(F)F)o3)cc2F)c2ccc(F)c(Cl)c2)CC1] |
| 209 | 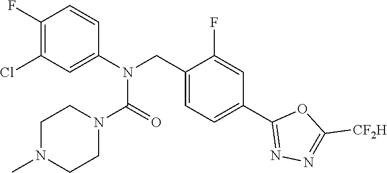 [SMILES: CN1CCN(C(=O)N(Cc2ccc(-c3nnc(C(F)F)o3)cc2F)c2ccc(F)c(Cl)c2)CC1] |
| 210 | 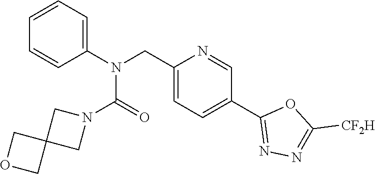 [SMILES: O=C(N1CC2(COC2)C1)N(Cc1ccc(-c2nnc(C(F)F)o2)cn1)c1ccccc1] |
| 211 | 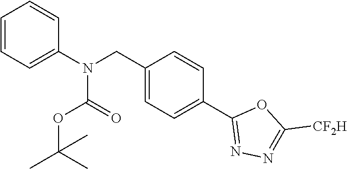 [SMILES: CC(C)(C)OC(=O)N(Cc1ccc(-c2nnc(C(F)F)o2)cc1)c1ccccc1] |
| 212 | 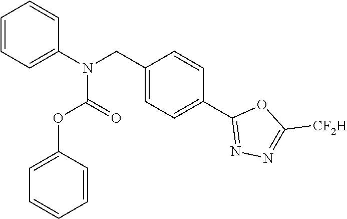 [SMILES: O=C(Oc1ccccc1)N(Cc1ccc(-c2nnc(C(F)F)o2)cc1)c1ccccc1] |
| 213 | 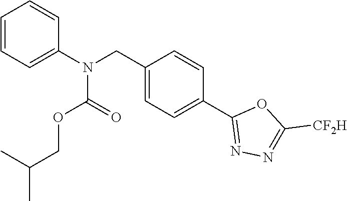 [SMILES: CC(C)COC(=O)N(Cc1ccc(-c2nnc(C(F)F)o2)cc1)c1ccccc1] |
| 214 | 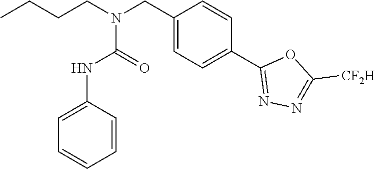 [SMILES: CCCCN(Cc1ccc(-c2nnc(C(F)F)o2)cc1)C(=O)Nc1ccccc1] |
| 215 | 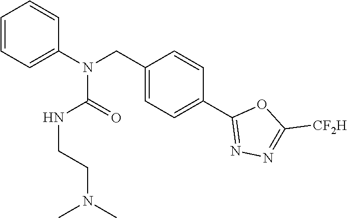 [SMILES: CN(C)CCNC(=O)N(Cc1ccc(-c2nnc(C(F)F)o2)cc1)c1ccccc1] |
| 216 | 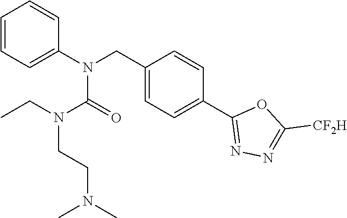 [SMILES: CCN(CCN(C)C)C(=O)N(Cc1ccc(-c2nnc(C(F)F)o2)cc1)c1ccccc1] |
| 217 | 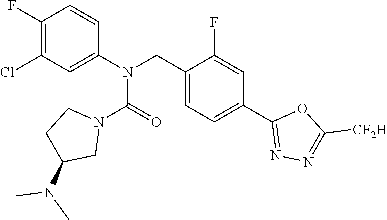 [SMILES: CN(C)[C@H]1CCN(C(=O)N(Cc2ccc(-c3nnc(C(F)F)o3)cc2F)c2ccc(F)c(Cl)c2)C1] |
| 218 | 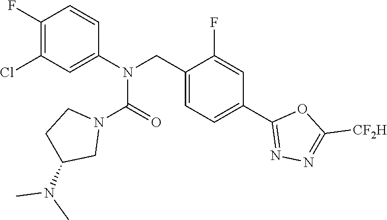 [SMILES: CN(C)[C@@H]1CCN(C(=O)N(Cc2ccc(-c3nnc(C(F)F)o3)cc2F)c2ccc(F)c(Cl)c2)C1] |
| 219 | 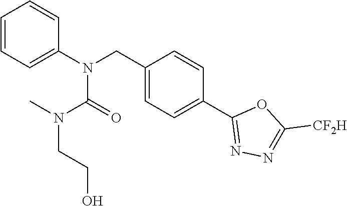 [SMILES: CN(CCO)C(=O)N(Cc1ccc(-c2nnc(C(F)F)o2)cc1)c1ccccc1] |
| 220 | 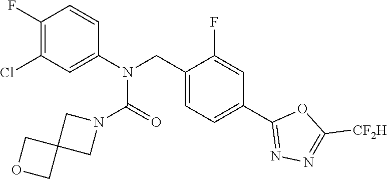 [SMILES: O=C(N1CC2(COC2)C1)N(Cc1ccc(-c2nnc(C(F)F)o2)cc1F)c1ccc(F)c(Cl)c1] |
| 221 | 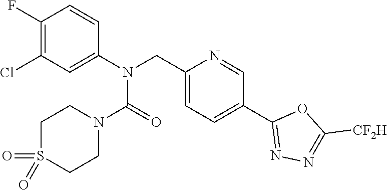 [SMILES: O=C(N1CCS(=O)(=O)CC1)N(Cc1ccc(-c2nnc(C(F)F)o2)cn1)c1ccc(F)c(Cl)c1] |
| 222 | 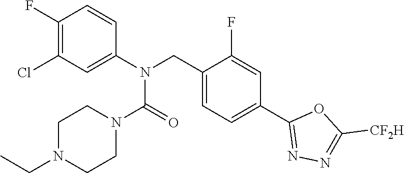 [SMILES: CCN1CCN(C(=O)N(Cc2ccc(-c3nnc(C(F)F)o3)cc2F)c2ccc(F)c(Cl)c2)CC1] |
| 223 | 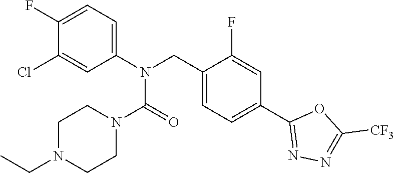 [SMILES: CCN1CCN(C(=O)N(Cc2ccc(-c3nnc(C(F)(F)F)o3)cc2F)c2ccc(F)c(Cl)c2)CC1] |
| 224 | 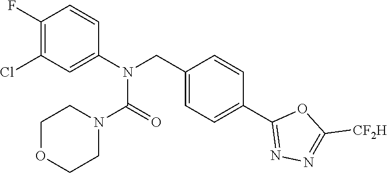 [SMILES: O=C(N1CCOCC1)N(Cc1ccc(-c2nnc(C(F)F)o2)cc1)c1ccc(F)c(Cl)c1] |
| 225 | 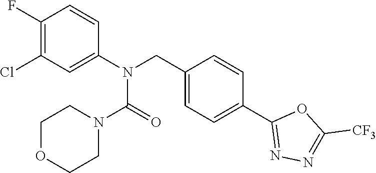 [SMILES: O=C(N1CCOCC1)N(Cc1ccc(-c2nnc(C(F)(F)F)o2)cc1)c1ccc(F)c(Cl)c1] |
| 226 | 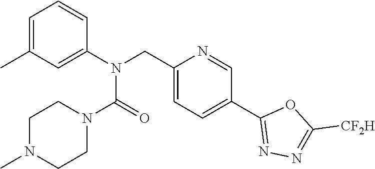 [SMILES: Cc1cccc(N(Cc2ccc(-c3nnc(C(F)F)o3)cn2)C(=O)N2CCN(C)CC2)c1] |
| 227 | 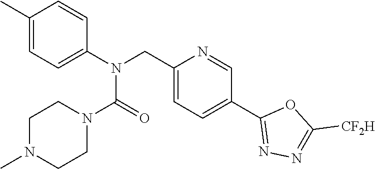 [SMILES: Cc1ccc(N(Cc2ccc(-c3nnc(C(F)F)o3)cn2)C(=O)N2CCN(C)CC2)cc1] |
| 228 | 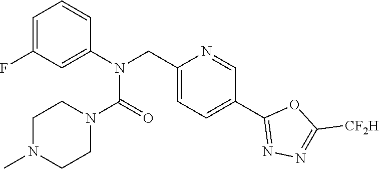 [SMILES: CN1CCN(C(=O)N(Cc2ccc(-c3nnc(C(F)F)o3)cn2)c2cccc(F)c2)CC1] |
| 229 | 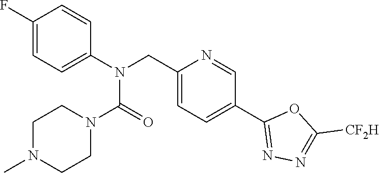 [SMILES: CN1CCN(C(=O)N(Cc2ccc(-c3nnc(C(F)F)o3)cn2)c2ccc(F)cc2)CC1] |
| 230 | 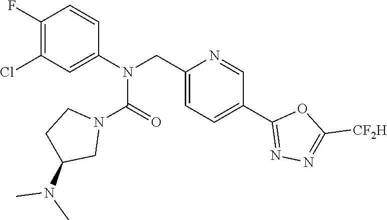 [SMILES: CN(C)[C@H]1CCN(C(=O)N(Cc2ccc(-c3nnc(C(F)F)o3)cn2)c2ccc(F)c(Cl)c2)C1] |
| 231 | 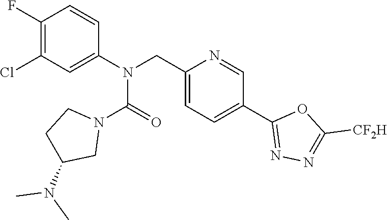 [SMILES: CN(C)[C@@H]1CCN(C(=O)N(Cc2ccc(-c3nnc(C(F)F)o3)cn2)c2ccc(F)c(Cl)c2)C1] |
| 232 | 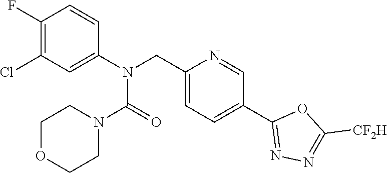 [SMILES: O=C(N1CCOCC1)N(Cc1ccc(-c2nnc(C(F)F)o2)cn1)c1ccc(F)c(Cl)c1] |
| 233 | 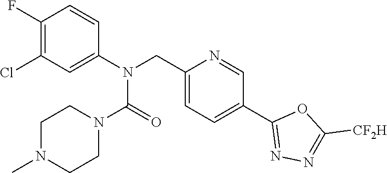 [SMILES: CN1CCN(C(=O)N(Cc2ccc(-c3nnc(C(F)F)o3)cn2)c2ccc(F)c(Cl)c2)CC1] |
| 234 | 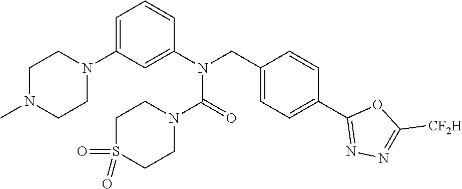 [SMILES: CN1CCN(c2cccc(N(Cc3ccc(-c4nnc(C(F)F)o4)cc3)C(=O)N3CCS(=O)(=O)CC3)c2)CC1] |
| 235 | 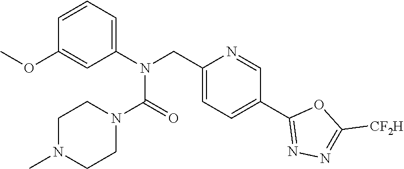 [SMILES: COc1cccc(N(Cc2ccc(-c3nnc(C(F)F)o3)cn2)C(=O)N2CCN(C)CC2)c1] |
| 236 | 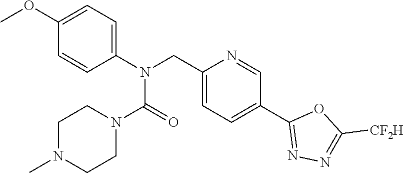 [SMILES: COc1ccc(N(Cc2ccc(-c3nnc(C(F)F)o3)cn2)C(=O)N2CCN(C)CC2)cc1] |
| 237 | 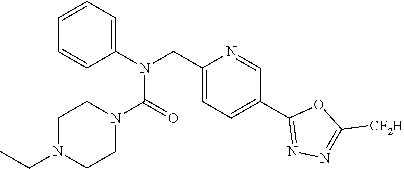 [SMILES: CCN1CCN(C(=O)N(Cc2ccc(-c3nnc(C(F)F)o3)cn2)c2ccccc2)CC1] |
| 238 | 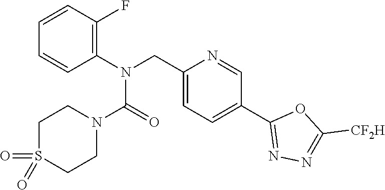 [SMILES: O=C(N1CCS(=O)(=O)CC1)N(Cc1ccc(-c2nnc(C(F)F)o2)cn1)c1ccccc1F] |
| 239 | 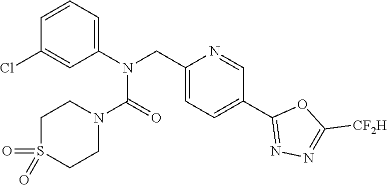 [SMILES: O=C(N1CCS(=O)(=O)CC1)N(Cc1ccc(-c2nnc(C(F)F)o2)cn1)c1cccc(Cl)c1] |
| 240 | 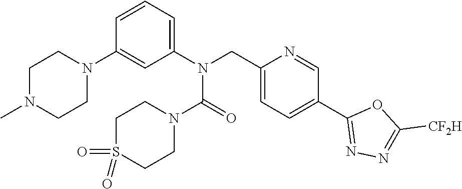 [SMILES: CN1CCN(c2cccc(N(Cc3ccc(-c4nnc(C(F)F)o4)cn3)C(=O)N3CCS(=O)(=O)CC3)c2)CC1] |
| 241 | 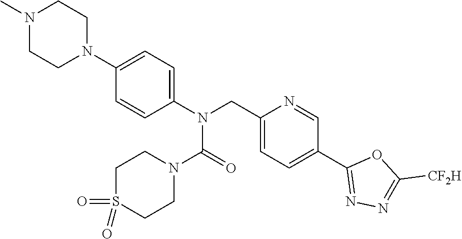 [SMILES: CN1CCN(c2ccc(N(Cc3ccc(-c4nnc(C(F)F)o4)cn3)C(=O)N3CCS(=O)(=O)CC3)cc2)CC1] |
| 242 | 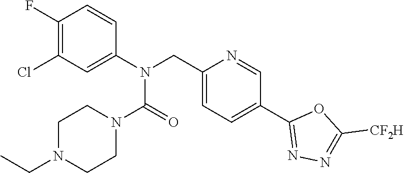 [SMILES: CCN1CCN(C(=O)N(Cc2ccc(-c3nnc(C(F)F)o3)cn2)c2ccc(F)c(Cl)c2)CC1] |
| 243 | 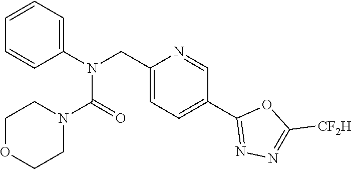 [SMILES: O=C(N1CCOCC1)N(Cc1ccc(-c2nnc(C(F)F)o2)cn1)c1ccccc1] |
| 244 | 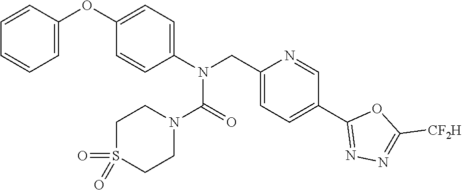 [SMILES: O=C(N1CCS(=O)(=O)CC1)N(Cc1ccc(-c2nnc(C(F)F)o2)cn1)c1ccc(Oc2ccccc2)cc1] |
| 245 | 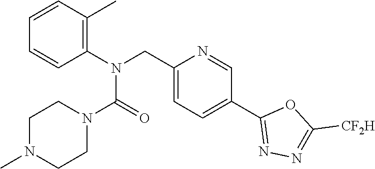 [SMILES: Cc1ccccc1N(Cc1ccc(-c2nnc(C(F)F)o2)cn1)C(=O)N1CCN(C)CC1] |
| 246 | 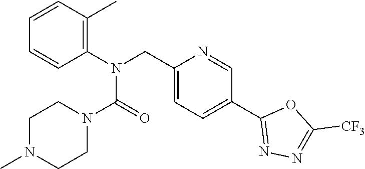 [SMILES: Cc1ccccc1N(Cc1ccc(-c2nnc(C(F)(F)F)o2)cn1)C(=O)N1CCN(C)CC1] |
| 247 | 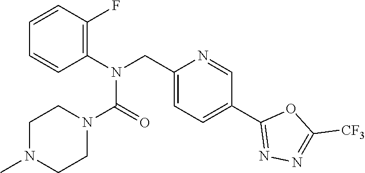 [SMILES: CN1CCN(C(=O)N(Cc2ccc(-c3nnc(C(F)(F)F)o3)cn2)c2ccccc2F)CC1] |
| 248 | 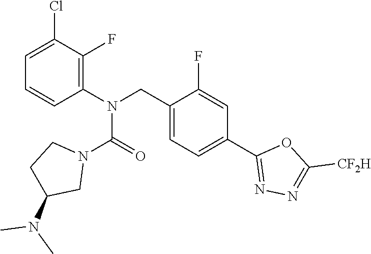 [SMILES: CN(C)[C@H]1CCN(C(=O)N(Cc2ccc(-c3nnc(C(F)F)o3)cc2F)c2cccc(Cl)c2F)C1] |
| 249 | 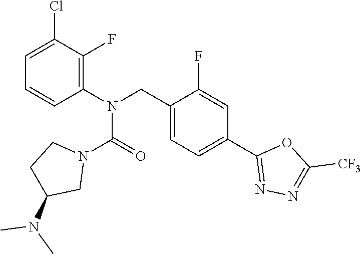 [SMILES: CN(C)[C@H]1CCN(C(=O)N(Cc2ccc(-c3nnc(C(F)(F)F)o3)cc2F)c2cccc(Cl)c2F)C1] |
| 250 | 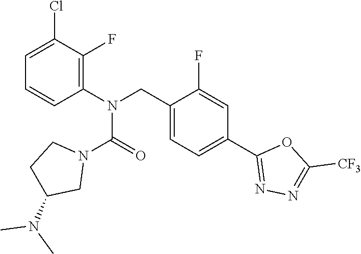 [SMILES: CN(C)[C@@H]1CCN(C(=O)N(Cc2ccc(-c3nnc(C(F)(F)F)o3)cc2F)c2cccc(Cl)c2F)C1] |
| 251 | 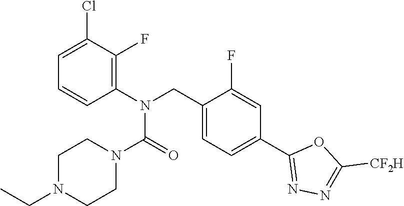 [SMILES: CCN1CCN(C(=O)N(Cc2ccc(-c3nnc(C(F)F)o3)cc2F)c2cccc(Cl)c2F)CC1] |
| 252 | 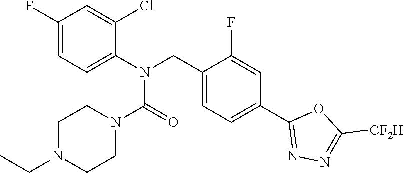 [SMILES: CCN1CCN(C(=O)N(Cc2ccc(-c3nnc(C(F)F)o3)cc2F)c2ccc(F)cc2Cl)CC1] |
| 253 | 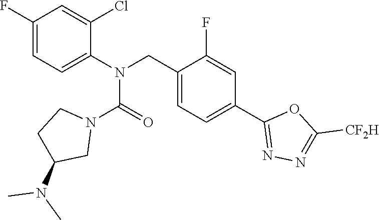 [SMILES: CN(C)[C@H]1CCN(C(=O)N(Cc2ccc(-c3nnc(C(F)F)o3)cc2F)c2ccc(F)cc2Cl)C1] |
| 254 | 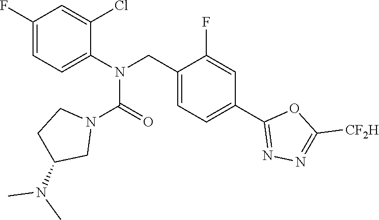 [SMILES: CN(C)[C@@H]1CCN(C(=O)N(Cc2ccc(-c3nnc(C(F)F)o3)cc2F)c2ccc(F)cc2Cl)C1] |
| 255 | 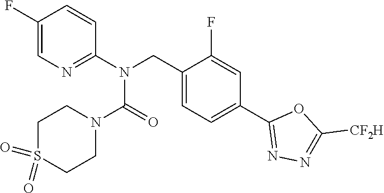 [SMILES: O=C(N1CCS(=O)(=O)CC1)N(Cc1ccc(-c2nnc(C(F)F)o2)cc1F)c1ccc(F)cn1] |
| 256 | 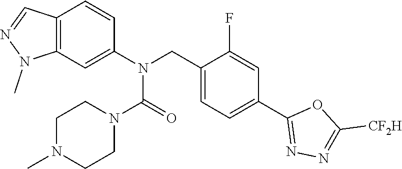 [SMILES: CN1CCN(C(=O)N(Cc2ccc(-c3nnc(C(F)F)o3)cc2F)c2ccc3cnn(C)c3c2)CC1] |
| 257 | 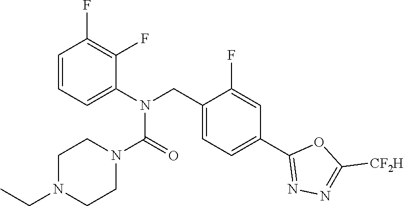 [SMILES: CCN1CCN(C(=O)N(Cc2ccc(-c3nnc(C(F)F)o3)cc2F)c2cccc(F)c2F)CC1] |
| 258 | 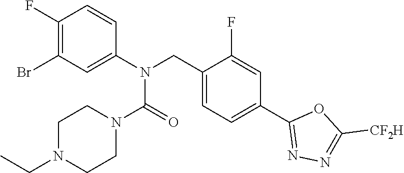 [SMILES: CCN1CCN(C(=O)N(Cc2ccc(-c3nnc(C(F)F)o3)cc2F)c2ccc(F)c(Br)c2)CC1] |
| 259 | 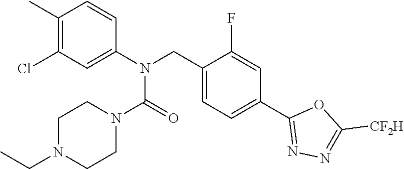 [SMILES: CCN1CCN(C(=O)N(Cc2ccc(-c3nnc(C(F)F)o3)cc2F)c2ccc(C)c(Cl)c2)CC1] |
| 260 | 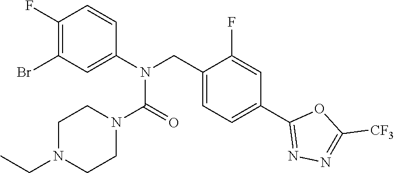 [SMILES: CCN1CCN(C(=O)N(Cc2ccc(-c3nnc(C(F)(F)F)o3)cc2F)c2ccc(F)c(Br)c2)CC1] |
| 261 | 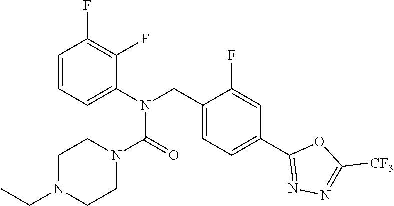 [SMILES: CCN1CCN(C(=O)N(Cc2ccc(-c3nnc(C(F)(F)F)o3)cc2F)c2cccc(F)c2F)CC1] |
| 262 | 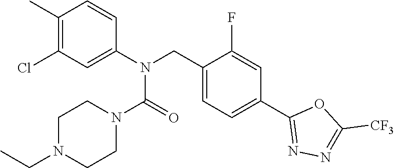 [SMILES: CCN1CCN(C(=O)N(Cc2ccc(-c3nnc(C(F)(F)F)o3)cc2F)c2ccc(C)c(Cl)c2)CC1] |
| 263 | 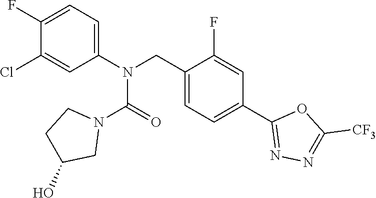 [SMILES: O=C(N1CC[C@@H](O)C1)N(Cc1ccc(-c2nnc(C(F)(F)F)o2)cc1F)c1ccc(F)c(Cl)c1] |
| 264 | 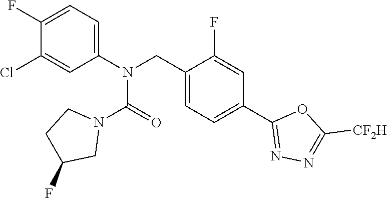 [SMILES: O=C(N1CC[C@H](F)C1)N(Cc1ccc(-c2nnc(C(F)F)o2)cc1F)c1ccc(F)c(Cl)c1] |
| 265 | 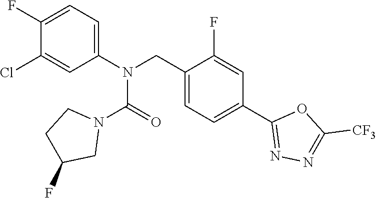 [SMILES: O=C(N1CC[C@H](F)C1)N(Cc1ccc(-c2nnc(C(F)(F)F)o2)cc1F)c1ccc(F)c(Cl)c1] |
| 266 | 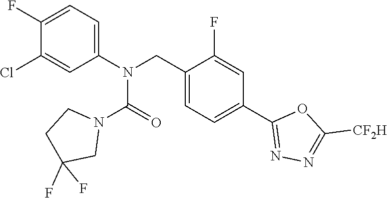 [SMILES: O=C(N1CCC(F)(F)C1)N(Cc1ccc(-c2nnc(C(F)F)o2)cc1F)c1ccc(F)c(Cl)c1] |
| 267 | 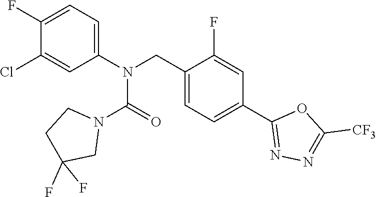 [SMILES: O=C(N1CCC(F)(F)C1)N(Cc1ccc(-c2nnc(C(F)(F)F)o2)cc1F)c1ccc(F)c(Cl)c1] |
| 268 | 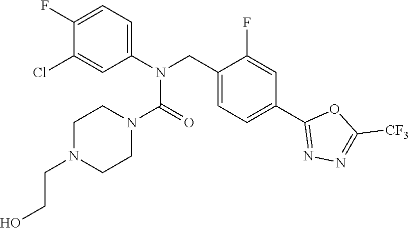 [SMILES: O=C(N1CCN(CCO)CC1)N(Cc1ccc(-c2nnc(C(F)(F)F)o2)cc1F)c1ccc(F)c(Cl)c1] |
| 269 | 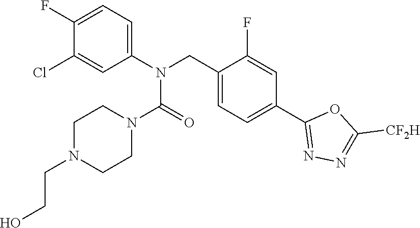 [SMILES: O=C(N1CCN(CCO)CC1)N(Cc1ccc(-c2nnc(C(F)F)o2)cc1F)c1ccc(F)c(Cl)c1] |
| 270 | 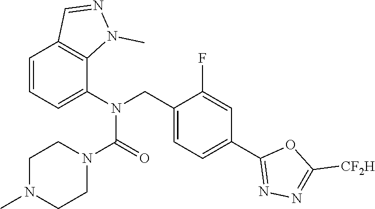 [SMILES: CN1CCN(C(=O)N(Cc2ccc(-c3nnc(C(F)F)o3)cc2F)c2cccc3cnn(C)c23)CC1] |
| 271 | 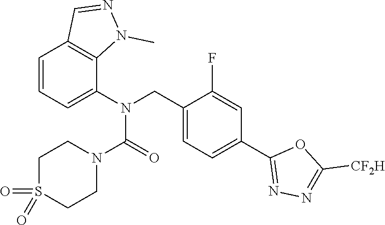 [SMILES: Cn1ncc2cccc(N(Cc3ccc(-c4nnc(C(F)F)o4)cc3F)C(=O)N3CCS(=O)(=O)CC3)c21] |
| 272 | 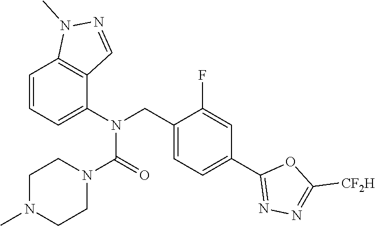 [SMILES: CN1CCN(C(=O)N(Cc2ccc(-c3nnc(C(F)F)o3)cc2F)c2cccc3c2cnn3C)CC1] |
| 273 | 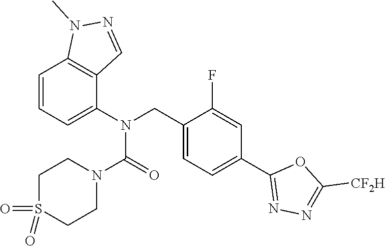 [SMILES: Cn1ncc2c(N(Cc3ccc(-c4nnc(C(F)F)o4)cc3F)C(=O)N3CCS(=O)(=O)CC3)cccc21] |
| 274 | 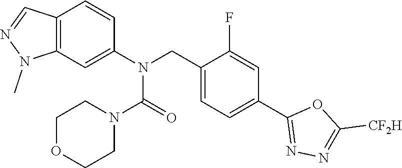 |
| 275 | 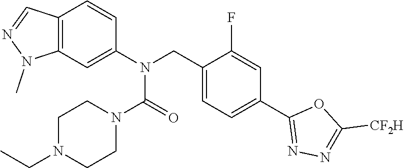 [SMILES: CCN1CCN(C(=O)N(Cc2ccc(-c3nnc(C(F)F)o3)cc2F)c2ccc3cnn(C)c3c2)CC1] |
| 276 | 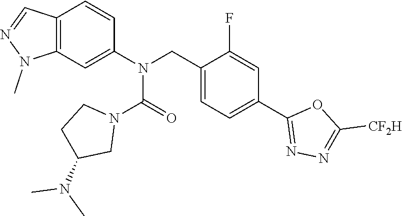 [SMILES: CN(C)[C@@H]1CCN(C(=O)N(Cc2ccc(-c3nnc(C(F)F)o3)cc2F)c2ccc3cnn(C)c3c2)C1] |
| 277 | 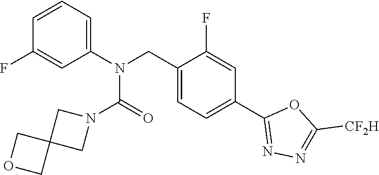 [SMILES: O=C(N1CC2(COC2)C1)N(Cc1ccc(-c2nnc(C(F)F)o2)cc1F)c1cccc(F)c1] |
| 278 | 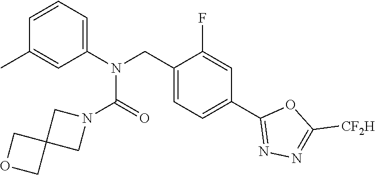 [SMILES: Cc1cccc(N(Cc2ccc(-c3nnc(C(F)F)o3)cc2F)C(=O)N2CC3(COC3)C2)c1] |
| 279 | 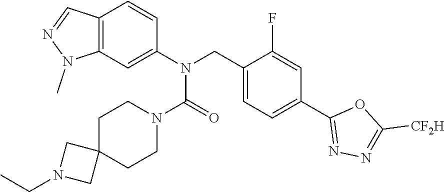 [SMILES: CCN1CC2(CCN(C(=O)N(Cc3ccc(-c4nnc(C(F)F)o4)cc3F)c3ccc4cnn(C)c4c3)CC2)C1] |
| 280 | 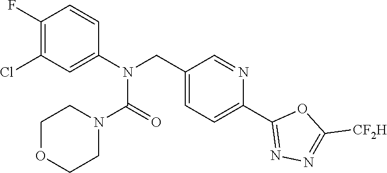 [SMILES: O=C(N1CCOCC1)N(Cc1ccc(-c2nnc(C(F)F)o2)nc1)c1ccc(F)c(Cl)c1] |
| 281 | 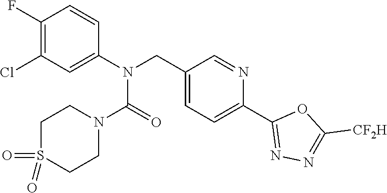 [SMILES: O=C(N1CCS(=O)(=O)CC1)N(Cc1ccc(-c2nnc(C(F)F)o2)nc1)c1ccc(F)c(Cl)c1] |
| 282 | 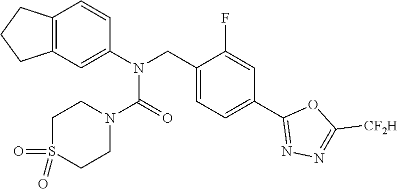 [SMILES: O=C(N1CCS(=O)(=O)CC1)N(Cc1ccc(-c2nnc(C(F)F)o2)cc1F)c1ccc2c(c1)CCC2] |
| 283 | 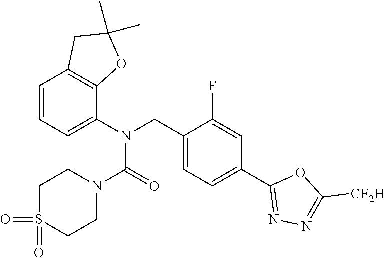 [SMILES: CC1(C)Cc2cccc(N(Cc3ccc(-c4nnc(C(F)F)o4)cc3F)C(=O)N3CCS(=O)(=O)CC3)c2O1] |
| 284 | 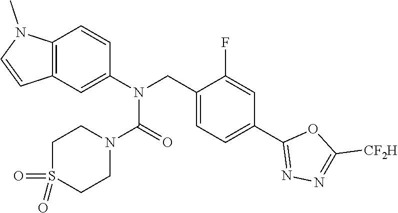 [SMILES: Cn1ccc2cc(N(Cc3ccc(-c4nnc(C(F)F)o4)cc3F)C(=O)N3CCS(=O)(=O)CC3)ccc21] |
| 285 | 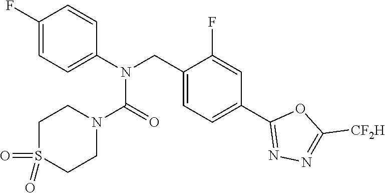 [SMILES: O=C(N1CCS(=O)(=O)CC1)N(Cc1ccc(-c2nnc(C(F)F)o2)cc1F)c1ccc(F)cc1] |
| 286 | 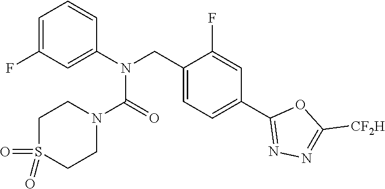 [SMILES: O=C(N1CCS(=O)(=O)CC1)N(Cc1ccc(-c2nnc(C(F)F)o2)cc1F)c1cccc(F)c1] |
| 287 | 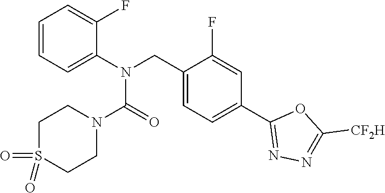 [SMILES: O=C(N1CCS(=O)(=O)CC1)N(Cc1ccc(-c2nnc(C(F)F)o2)cc1F)c1ccccc1F] |
| 288 | 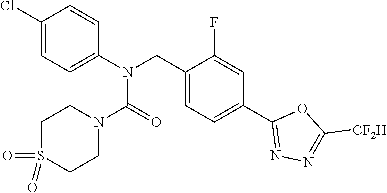 [SMILES: O=C(N1CCS(=O)(=O)CC1)N(Cc1ccc(-c2nnc(C(F)F)o2)cc1F)c1ccc(Cl)cc1] |
| 289 | 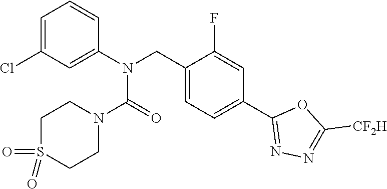 [SMILES: O=C(N1CCS(=O)(=O)CC1)N(Cc1ccc(-c2nnc(C(F)F)o2)cc1F)c1cccc(Cl)c1] |
| 290 | 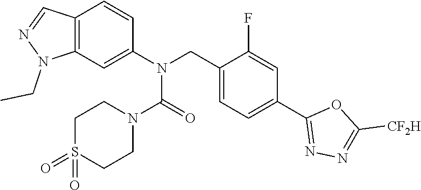 [SMILES: CCn1ncc2ccc(N(Cc3ccc(-c4nnc(C(F)F)o4)cc3F)C(=O)N3CCS(=O)(=O)CC3)cc21] |
| 291 | 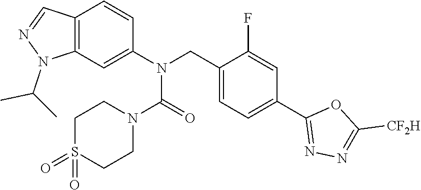 [SMILES: CC(C)n1ncc2ccc(N(Cc3ccc(-c4nnc(C(F)F)o4)cc3F)C(=O)N3CCS(=O)(=O)CC3)cc21] |
| 292 | 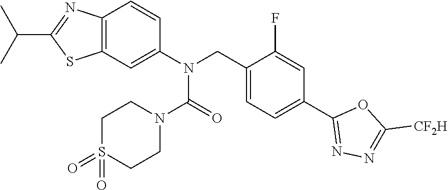 [SMILES: CC(C)c1nc2ccc(N(Cc3ccc(-c4nnc(C(F)F)o4)cc3F)C(=O)N3CCS(=O)(=O)CC3)cc2s1] |
| 293 | 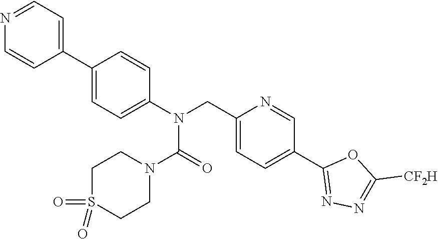 [SMILES: O=C(N1CCS(=O)(=O)CC1)N(Cc1ccc(-c2nnc(C(F)F)o2)cn1)c1ccc(-c2ccncc2)cc1] |
| 294 | 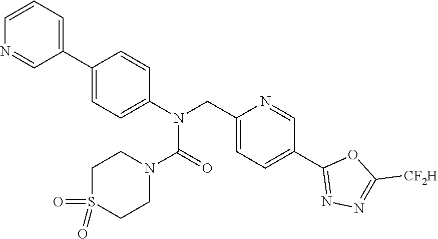 [SMILES: O=C(N1CCS(=O)(=O)CC1)N(Cc1ccc(-c2nnc(C(F)F)o2)cn1)c1ccc(-c2cccnc2)cc1] |
| 295 | 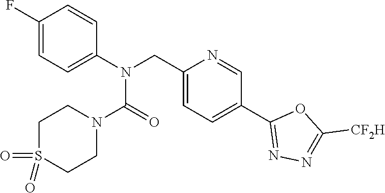 [SMILES: O=C(N1CCS(=O)(=O)CC1)N(Cc1ccc(-c2nnc(C(F)F)o2)cn1)c1ccc(F)cc1] |
| 296 | 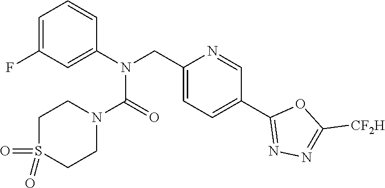 [SMILES: O=C(N1CCS(=O)(=O)CC1)N(Cc1ccc(-c2nnc(C(F)F)o2)cn1)c1cccc(F)c1] |
| 297 | 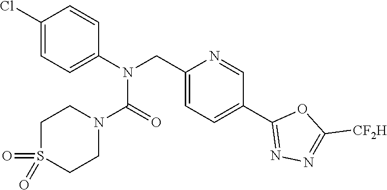 [SMILES: O=C(N1CCS(=O)(=O)CC1)N(Cc1ccc(-c2nnc(C(F)F)o2)cn1)c1ccc(Cl)cc1] |
| 298 | 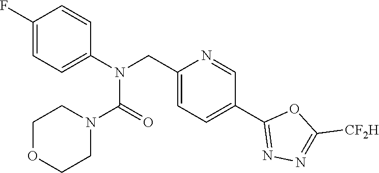 [SMILES: O=C(N1CCOCC1)N(Cc1ccc(-c2nnc(C(F)F)o2)cn1)c1ccc(F)cc1] |
| 299 | 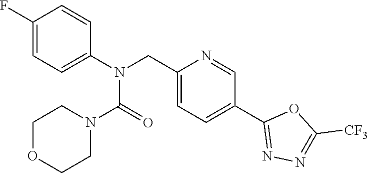 [SMILES: O=C(N1CCOCC1)N(Cc1ccc(-c2nnc(C(F)(F)F)o2)cn1)c1ccc(F)cc1] |
| 300 | 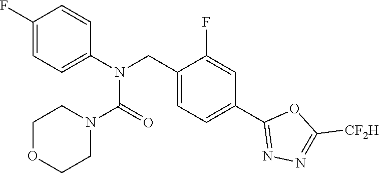 [SMILES: O=C(N1CCOCC1)N(Cc1ccc(-c2nnc(C(F)F)o2)cc1F)c1ccc(F)cc1] |
| 301 | 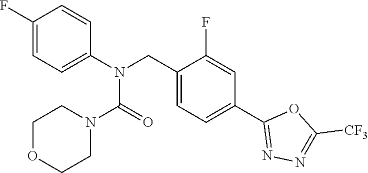 [SMILES: O=C(N1CCOCC1)N(Cc1ccc(-c2nnc(C(F)(F)F)o2)cc1F)c1ccc(F)cc1] |
| 302 | 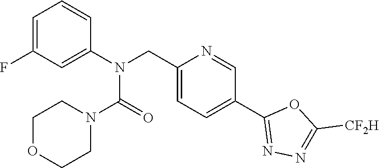 [SMILES: O=C(N1CCOCC1)N(Cc1ccc(-c2nnc(C(F)F)o2)cn1)c1cccc(F)c1] |
| 303 | 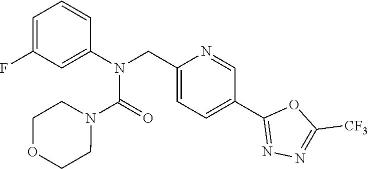 [SMILES: O=C(N1CCOCC1)N(Cc1ccc(-c2nnc(C(F)(F)F)o2)cn1)c1cccc(F)c1] |
| 304 | 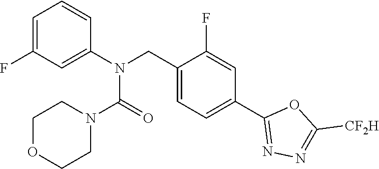 [SMILES: O=C(N1CCOCC1)N(Cc1ccc(-c2nnc(C(F)F)o2)cc1F)c1cccc(F)c1] |
| 305 | 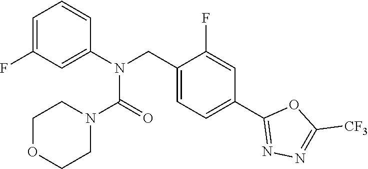 [SMILES: O=C(N1CCOCC1)N(Cc1ccc(-c2nnc(C(F)(F)F)o2)cc1F)c1cccc(F)c1] |
| 306 | 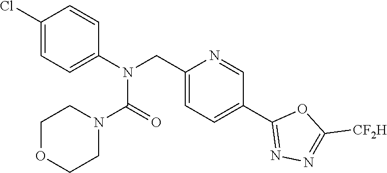 [SMILES: O=C(N1CCOCC1)N(Cc1ccc(-c2nnc(C(F)F)o2)cn1)c1ccc(Cl)cc1] |
| 307 | 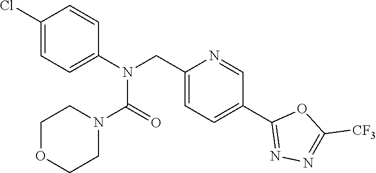 [SMILES: O=C(N1CCOCC1)N(Cc1ccc(-c2nnc(C(F)(F)F)o2)cn1)c1ccc(Cl)cc1] |
| 308 | 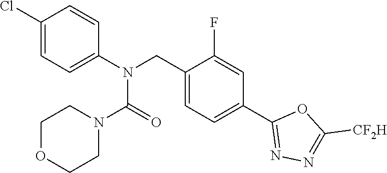 [SMILES: O=C(N1CCOCC1)N(Cc1ccc(-c2nnc(C(F)F)o2)cc1F)c1ccc(Cl)cc1] |
| 309 | 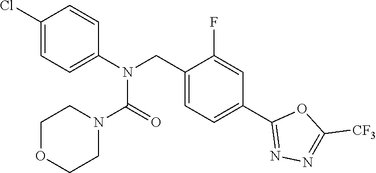 [SMILES: O=C(N1CCOCC1)N(Cc1ccc(-c2nnc(C(F)(F)F)o2)cc1F)c1ccc(Cl)cc1] |
| 310 | 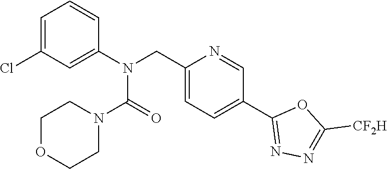 [SMILES: O=C(N1CCOCC1)N(Cc1ccc(-c2nnc(C(F)F)o2)cn1)c1cccc(Cl)c1] |
| 311 | 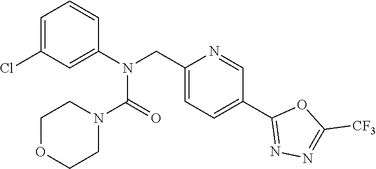 [SMILES: O=C(N1CCOCC1)N(Cc1ccc(-c2nnc(C(F)(F)F)o2)cn1)c1cccc(Cl)c1] |
| 312 | 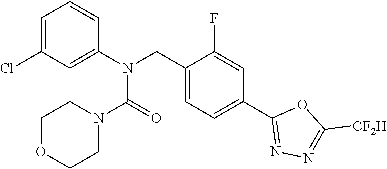 [SMILES: O=C(N1CCOCC1)N(Cc1ccc(-c2nnc(C(F)F)o2)cc1F)c1cccc(Cl)c1] |
| 313 | 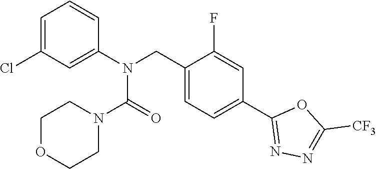 [SMILES: O=C(N1CCOCC1)N(Cc1ccc(-c2nnc(C(F)(F)F)o2)cc1F)c1cccc(Cl)c1] |
| 314 | 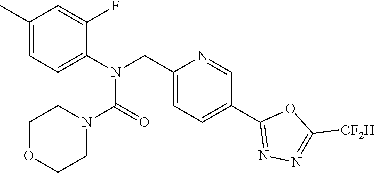 [SMILES: Cc1ccc(N(Cc2ccc(-c3nnc(C(F)F)o3)cn2)C(=O)N2CCOCC2)c(F)c1] |
| 315 | 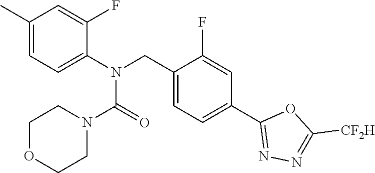 [SMILES: Cc1ccc(N(Cc2ccc(-c3nnc(C(F)F)o3)cc2F)C(=O)N2CCOCC2)c(F)c1] |
| 316 | 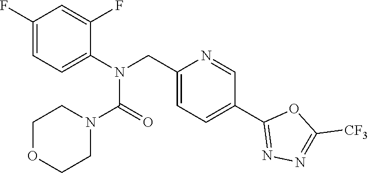 [SMILES: O=C(N1CCOCC1)N(Cc1ccc(-c2nnc(C(F)(F)F)o2)cn1)c1ccc(F)cc1F] |
| 317 | 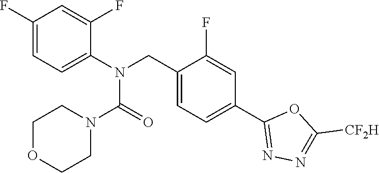 [SMILES: O=C(N1CCOCC1)N(Cc1ccc(-c2nnc(C(F)F)o2)cc1F)c1ccc(F)cc1F] |
| 318 | 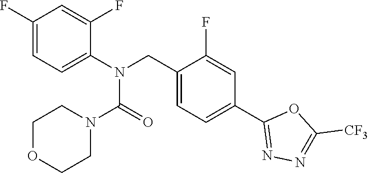 [SMILES: O=C(N1CCOCC1)N(Cc1ccc(-c2nnc(C(F)(F)F)o2)cc1F)c1ccc(F)cc1F] |
| 319 | 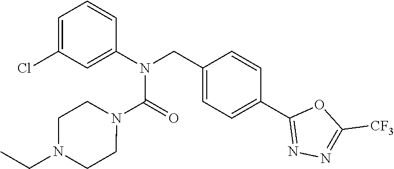 [SMILES: CCN1CCN(C(=O)N(Cc2ccc(-c3nnc(C(F)(F)F)o3)cc2)c2cccc(Cl)c2)CC1] |
| 320 | 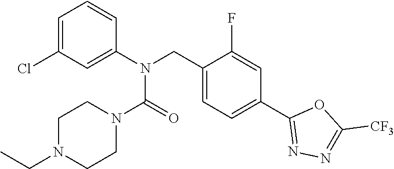 [SMILES: CCN1CCN(C(=O)N(Cc2ccc(-c3nnc(C(F)(F)F)o3)cc2F)c2cccc(Cl)c2)CC1] |
| 321 | 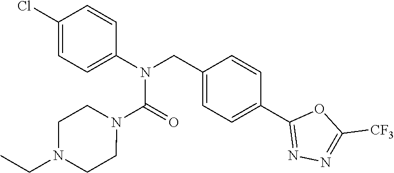 [SMILES: CCN1CCN(C(=O)N(Cc2ccc(-c3nnc(C(F)(F)F)o3)cc2)c2ccc(Cl)cc2)CC1] |
| 322 | 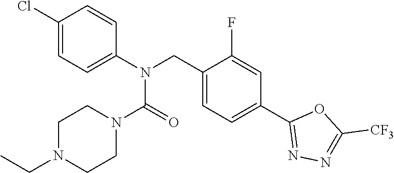 [SMILES: CCN1CCN(C(=O)N(Cc2ccc(-c3nnc(C(F)(F)F)o3)cc2F)c2ccc(Cl)cc2)CC1] |
| 323 | 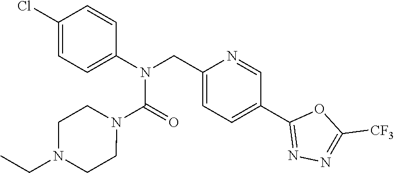 [SMILES: CCN1CCN(C(=O)N(Cc2ccc(-c3nnc(C(F)(F)F)o3)cn2)c2ccc(Cl)cc2)CC1] |
| 324 | 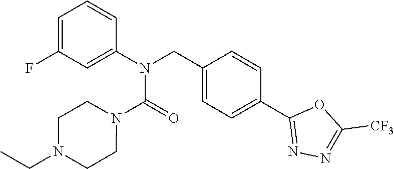 [SMILES: CCN1CCN(C(=O)N(Cc2ccc(-c3nnc(C(F)(F)F)o3)cc2)c2cccc(F)c2)CC1] |
| 325 | 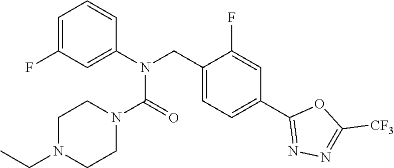 [SMILES: CCN1CCN(C(=O)N(Cc2ccc(-c3nnc(C(F)(F)F)o3)cc2F)c2cccc(F)c2)CC1] |
| 326 | 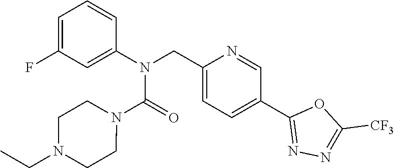 [SMILES: CCN1CCN(C(=O)N(Cc2ccc(-c3nnc(C(F)(F)F)o3)cn2)c2cccc(F)c2)CC1] |
| 327 | 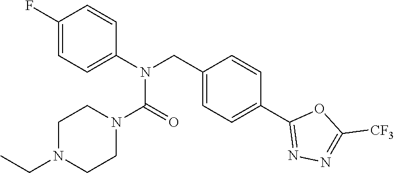 [SMILES: CCN1CCN(C(=O)N(Cc2ccc(-c3nnc(C(F)(F)F)o3)cc2)c2ccc(F)cc2)CC1] |
| 328 | 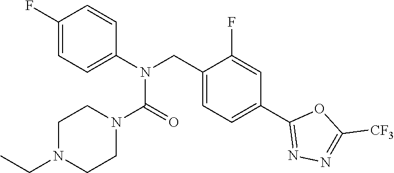 [SMILES: CCN1CCN(C(=O)N(Cc2ccc(-c3nnc(C(F)(F)F)o3)cc2F)c2ccc(F)cc2)CC1] |
| 329 | 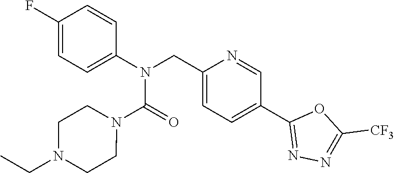 [SMILES: CCN1CCN(C(=O)N(Cc2ccc(-c3nnc(C(F)(F)F)o3)cn2)c2ccc(F)cc2)CC1] |
| 330 | 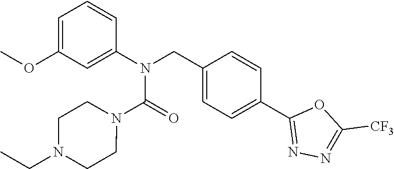 [SMILES: CCN1CCN(C(=O)N(Cc2ccc(-c3nnc(C(F)(F)F)o3)cc2)c2cccc(OC)c2)CC1] |
| 331 | 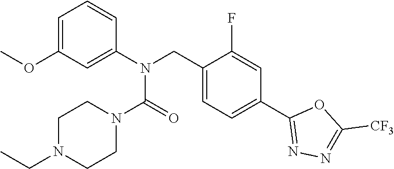 [SMILES: CCN1CCN(C(=O)N(Cc2ccc(-c3nnc(C(F)(F)F)o3)cc2F)c2cccc(OC)c2)CC1] |
| 332 | 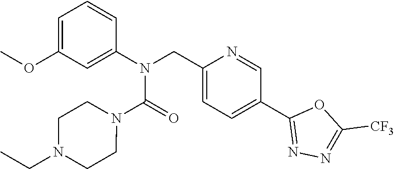 [SMILES: CCN1CCN(C(=O)N(Cc2ccc(-c3nnc(C(F)(F)F)o3)cn2)c2cccc(OC)c2)CC1] |
| 333 | 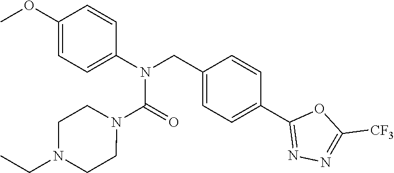 [SMILES: CCN1CCN(C(=O)N(Cc2ccc(-c3nnc(C(F)(F)F)o3)cc2)c2ccc(OC)cc2)CC1] |
| 334 | 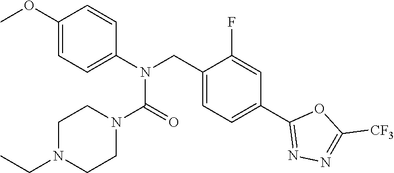 [SMILES: CCN1CCN(C(=O)N(Cc2ccc(-c3nnc(C(F)(F)F)o3)cc2F)c2ccc(OC)cc2)CC1] |
| 335 | 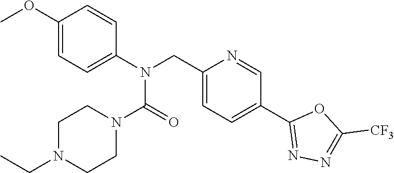 [SMILES: CCN1CCN(C(=O)N(Cc2ccc(-c3nnc(C(F)(F)F)o3)cn2)c2ccc(OC)cc2)CC1] |
| 336 | 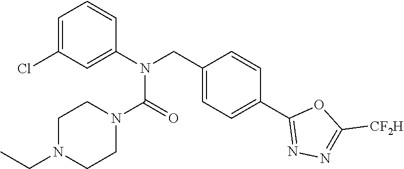 [SMILES: CCN1CCN(C(=O)N(Cc2ccc(-c3nnc(C(F)F)o3)cc2)c2cccc(Cl)c2)CC1] |
| 337 | 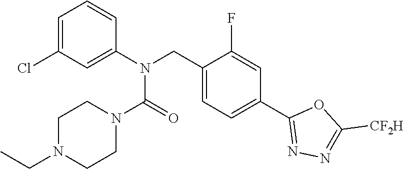 [SMILES: CCN1CCN(C(=O)N(Cc2ccc(-c3nnc(C(F)F)o3)cc2F)c2cccc(Cl)c2)CC1] |
| 338 | 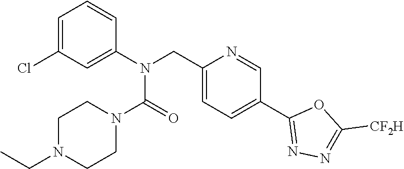 [SMILES: CCN1CCN(C(=O)N(Cc2ccc(-c3nnc(C(F)F)o3)cn2)c2cccc(Cl)c2)CC1] |
| 339 | 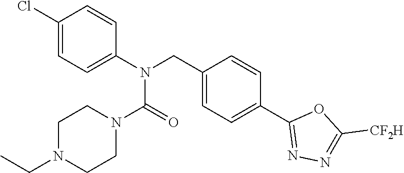 [SMILES: CCN1CCN(C(=O)N(Cc2ccc(-c3nnc(C(F)F)o3)cc2)c2ccc(Cl)cc2)CC1] |
| 340 | 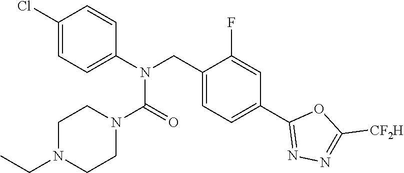 [SMILES: CCN1CCN(C(=O)N(Cc2ccc(-c3nnc(C(F)F)o3)cc2F)c2ccc(Cl)cc2)CC1] |
| 341 | 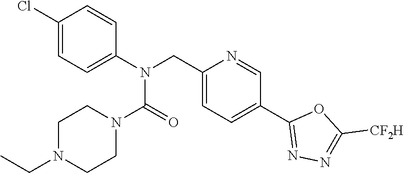 [SMILES: CCN1CCN(C(=O)N(Cc2ccc(-c3nnc(C(F)F)o3)cn2)c2ccc(Cl)cc2)CC1] |
| 342 | 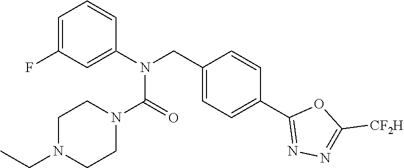 [SMILES: CCN1CCN(C(=O)N(Cc2ccc(-c3nnc(C(F)F)o3)cc2)c2cccc(F)c2)CC1] |
| 343 | 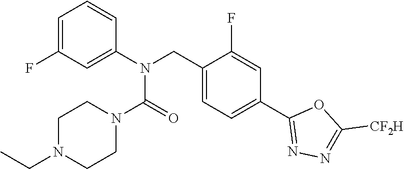 [SMILES: CCN1CCN(C(=O)N(Cc2ccc(-c3nnc(C(F)F)o3)cc2F)c2cccc(F)c2)CC1] |
| 344 | 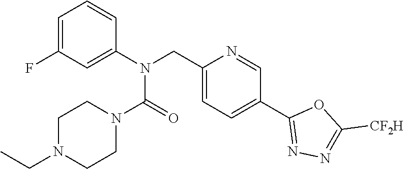 [SMILES: CCN1CCN(C(=O)N(Cc2ccc(-c3nnc(C(F)F)o3)cn2)c2cccc(F)c2)CC1] |
| 345 | 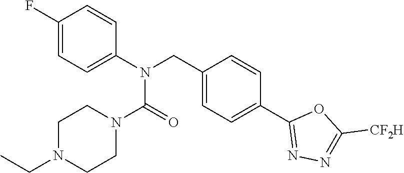 [SMILES: CCN1CCN(C(=O)N(Cc2ccc(-c3nnc(C(F)F)o3)cc2)c2ccc(F)cc2)CC1] |
| 346 | 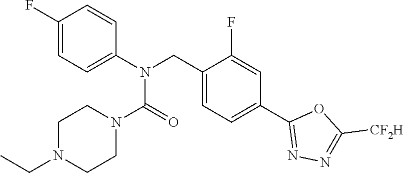 [SMILES: CCN1CCN(C(=O)N(Cc2ccc(-c3nnc(C(F)F)o3)cc2F)c2ccc(F)cc2)CC1] |
| 347 | 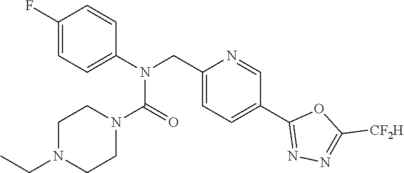 [SMILES: CCN1CCN(C(=O)N(Cc2ccc(-c3nnc(C(F)F)o3)cn2)c2ccc(F)cc2)CC1] |
| 348 | 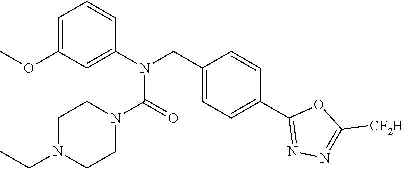 [SMILES: CCN1CCN(C(=O)N(Cc2ccc(-c3nnc(C(F)F)o3)cc2)c2cccc(OC)c2)CC1] |
| 349 | 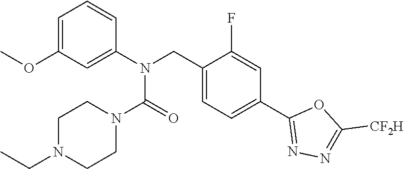 [SMILES: CCN1CCN(C(=O)N(Cc2ccc(-c3nnc(C(F)F)o3)cc2F)c2cccc(OC)c2)CC1] |
| 350 | 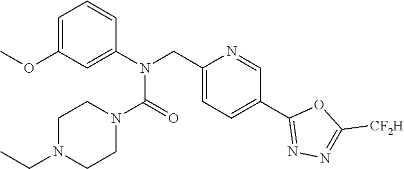 [SMILES: CCN1CCN(C(=O)N(Cc2ccc(-c3nnc(C(F)F)o3)cn2)c2cccc(OC)c2)CC1] |
| 351 | 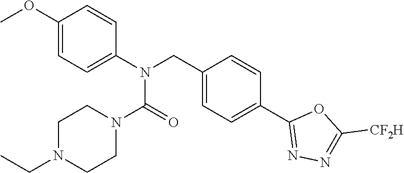 [SMILES: CCN1CCN(C(=O)N(Cc2ccc(-c3nnc(C(F)F)o3)cc2)c2ccc(OC)cc2)CC1] |
| 352 | 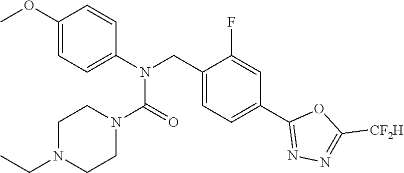 [SMILES: CCN1CCN(C(=O)N(Cc2ccc(-c3nnc(C(F)F)o3)cc2F)c2ccc(OC)cc2)CC1] |
| 353 | 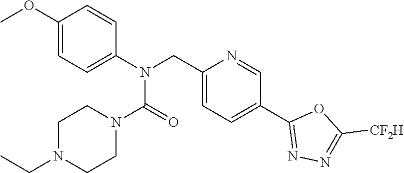 [SMILES: CCN1CCN(C(=O)N(Cc2ccc(-c3nnc(C(F)F)o3)cn2)c2ccc(OC)cc2)CC1] |
| 354 | 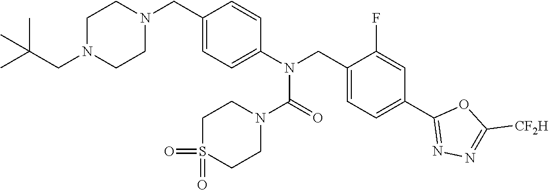 [SMILES: CC(C)(C)CN1CCN(Cc2ccc(N(Cc3ccc(-c4nnc(C(F)F)o4)cc3F)C(=O)N3CCS(=O)(=O)CC3)cc2)CC1] |
| 355 | 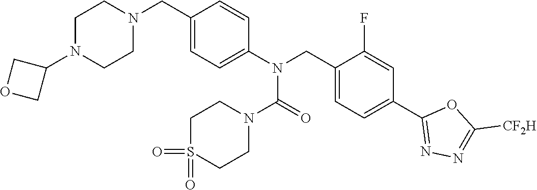 [SMILES: O=C(N1CCS(=O)(=O)CC1)N(Cc1ccc(-c2nnc(C(F)F)o2)cc1F)c1ccc(CN2CCN(C3COC3)CC2)cc1] |
| 356 | 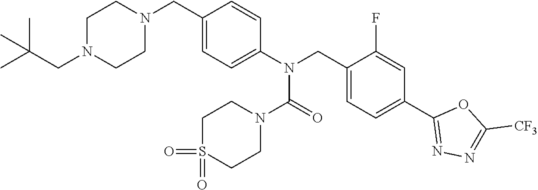 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 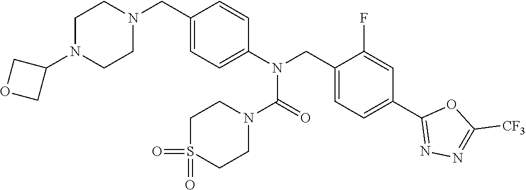 [SMILES: O=C(N1CCS(=O)(=O)CC1)N(Cc1ccc(-c2nnc(C(F)(F)F)o2)cc1F)c1ccc(CN2CCN(C3COC3)CC2)cc1] |
| 358 | 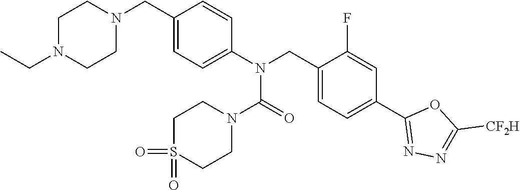 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 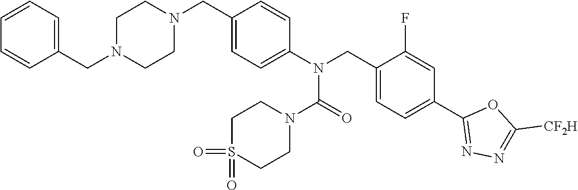 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] |
| 360 | 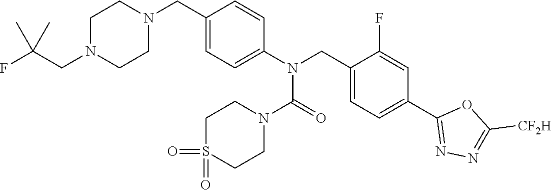 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 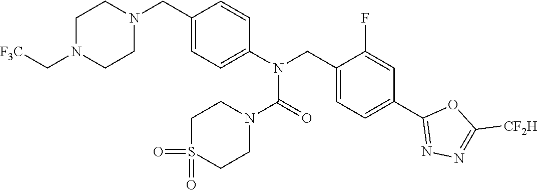 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] |
| 362 | 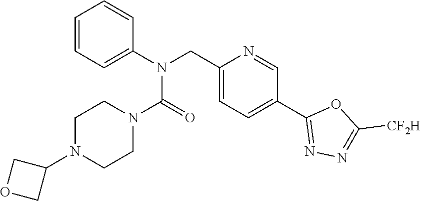 [SMILES: O=C(N1CCN(C2COC2)CC1)N(Cc1ccc(-c2nnc(C(F)F)o2)cn1)c1ccccc1] |
| 363 | 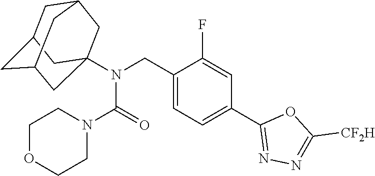 [SMILES: O=C(N1CCOCC1)N(Cc1ccc(-c2nnc(C(F)F)o2)cc1F)C12CC3CC(CC(C3)C1)C2] |
| 364 | 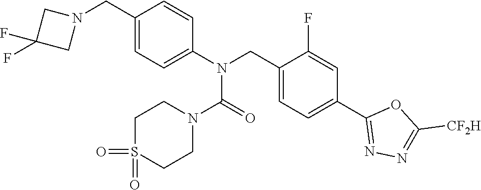 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 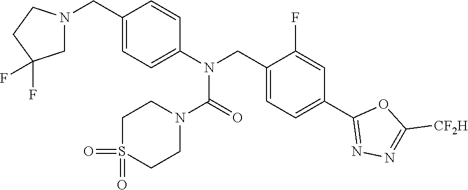 [SMILES: O=C(N1CCS(=O)(=O)CC1)N(Cc1ccc(-c2nnc(C(F)F)o2)cc1F)c1ccc(CN2CCC(F)(F)C2)cc1] |
| 366 | 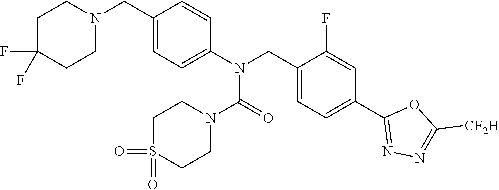 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 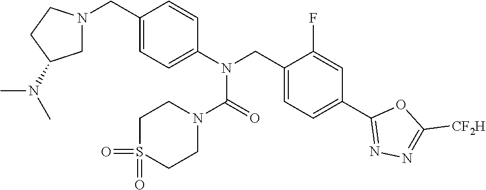 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] |
| 368 | 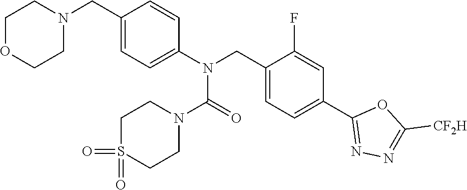 [SMILES: O=C(N1CCS(=O)(=O)CC1)N(Cc1ccc(-c2nnc(C(F)F)o2)cc1F)c1ccc(CN2CCOCC2)cc1] |
| 369 | 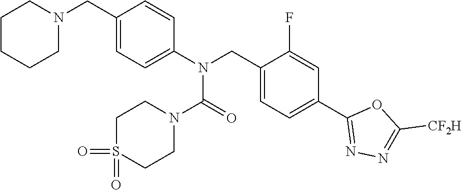 [SMILES: O=C(N1CCS(=O)(=O)CC1)N(Cc1ccc(-c2nnc(C(F)F)o2)cc1F)c1ccc(CN2CCCCC2)cc1] |
| 370 | 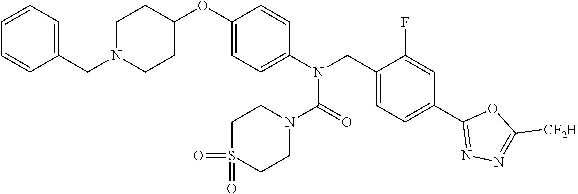 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 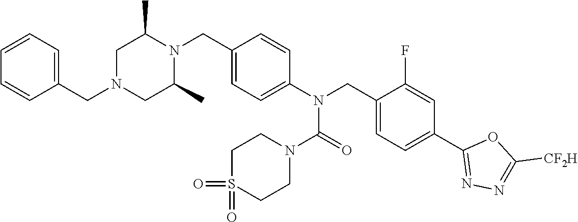 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] |
| 372 | 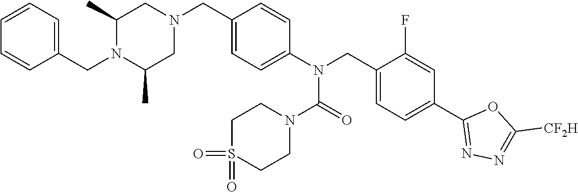 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 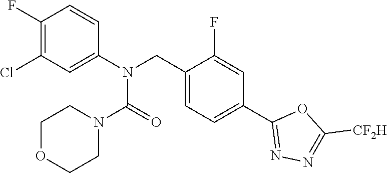 [SMILES: O=C(N1CCOCC1)N(Cc1ccc(-c2nnc(C(F)F)o2)cc1F)c1ccc(F)c(Cl)c1] |
| 374 | 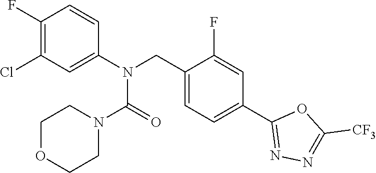 [SMILES: O=C(N1CCOCC1)N(Cc1ccc(-c2nnc(C(F)(F)F)o2)cc1F)c1ccc(F)c(Cl)c1] |
| 375 | 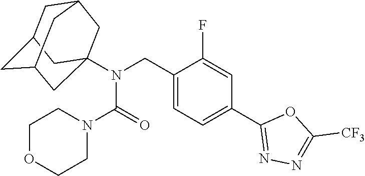 [SMILES: O=C(N1CCOCC1)N(Cc1ccc(-c2nnc(C(F)(F)F)o2)cc1F)C12CC3CC(CC(C3)C1)C2] |
| 376 | 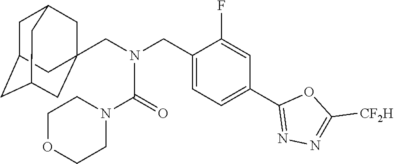 [SMILES: O=C(N1CCOCC1)N(Cc1ccc(-c2nnc(C(F)F)o2)cc1F)CC12CC3CC(CC(C3)C1)C2] |
| 377 | 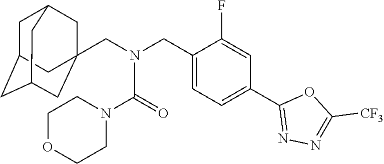 [SMILES: O=C(N1CCOCC1)N(Cc1ccc(-c2nnc(C(F)(F)F)o2)cc1F)CC12CC3CC(CC(C3)C1)C2] |
| 378 | 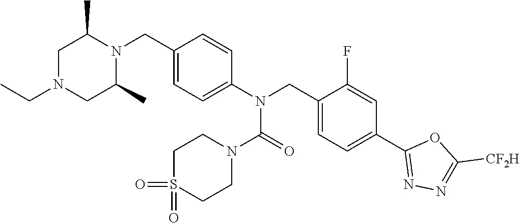 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 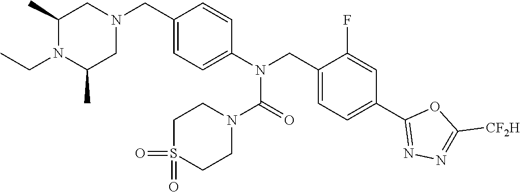 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] |
| 380 | 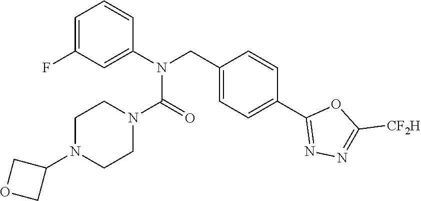 [SMILES: O=C(N1CCN(C2COC2)CC1)N(Cc1ccc(-c2nnc(C(F)F)o2)cc1)c1cccc(F)c1] |
| 381 | 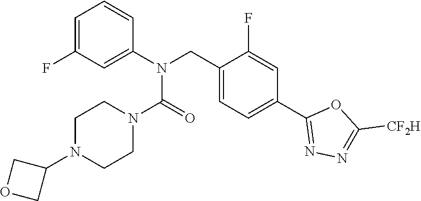 [SMILES: O=C(N1CCN(C2COC2)CC1)N(Cc1ccc(-c2nnc(C(F)F)o2)cc1F)c1cccc(F)c1] |
| 382 | 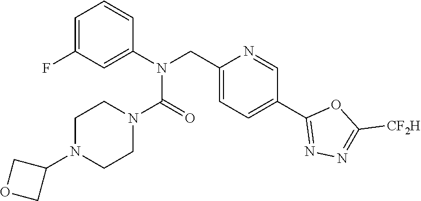 [SMILES: O=C(N1CCN(C2COC2)CC1)N(Cc1ccc(-c2nnc(C(F)F)o2)cn1)c1cccc(F)c1] |
| 383 | 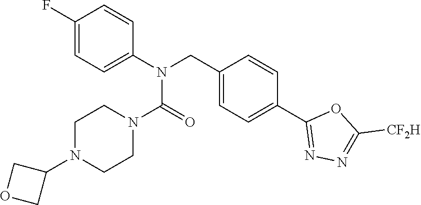 [SMILES: O=C(N1CCN(C2COC2)CC1)N(Cc1ccc(-c2nnc(C(F)F)o2)cc1)c1ccc(F)cc1] |
| 384 | 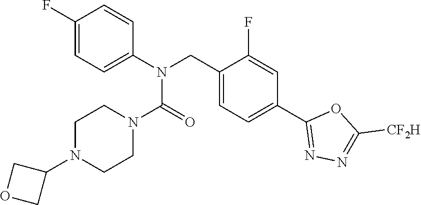 [SMILES: O=C(N1CCN(C2COC2)CC1)N(Cc1ccc(-c2nnc(C(F)F)o2)cc1F)c1ccc(F)cc1] |
| 385 | 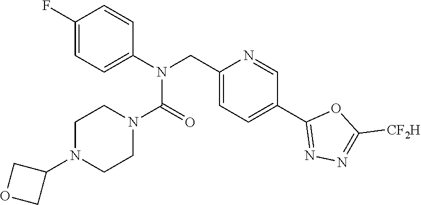 [SMILES: O=C(N1CCN(C2COC2)CC1)N(Cc1ccc(-c2nnc(C(F)F)o2)cn1)c1ccc(F)cc1] |
| 386 | 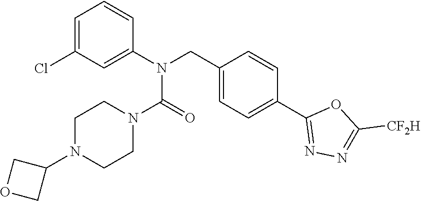 [SMILES: O=C(N1CCN(C2COC2)CC1)N(Cc1ccc(-c2nnc(C(F)F)o2)cc1)c1cccc(Cl)c1] |
| 387 | 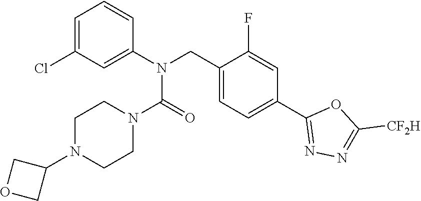 [SMILES: O=C(N1CCN(C2COC2)CC1)N(Cc1ccc(-c2nnc(C(F)F)o2)cc1F)c1cccc(Cl)c1] |
| 388 | 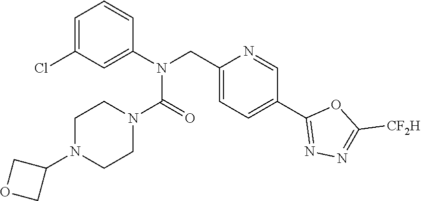 [SMILES: O=C(N1CCN(C2COC2)CC1)N(Cc1ccc(-c2nnc(C(F)F)o2)cn1)c1cccc(Cl)c1] |
| 389 | 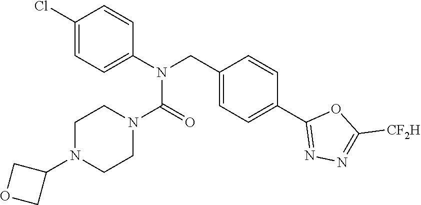 [SMILES: O=C(N1CCN(C2COC2)CC1)N(Cc1ccc(-c2nnc(C(F)F)o2)cc1)c1ccc(Cl)cc1] |
| 390 | 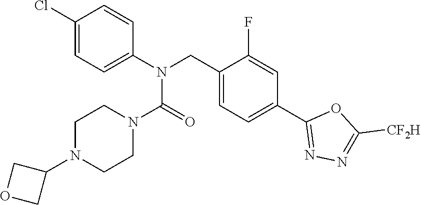 [SMILES: O=C(N1CCN(C2COC2)CC1)N(Cc1ccc(-c2nnc(C(F)F)o2)cc1F)c1ccc(Cl)cc1] |
| 391 | 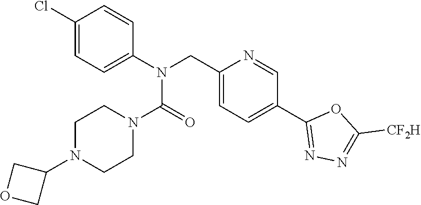 [SMILES: O=C(N1CCN(C2COC2)CC1)N(Cc1ccc(-c2nnc(C(F)F)o2)cn1)c1ccc(Cl)cc1] |
| 392 | 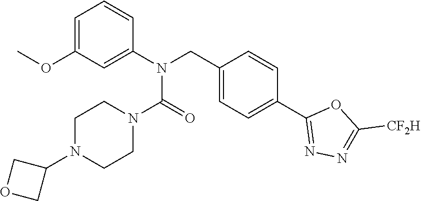 [SMILES: COc1cccc(N(Cc2ccc(-c3nnc(C(F)F)o3)cc2)C(=O)N2CCN(C3COC3)CC2)c1] |
| 393 | 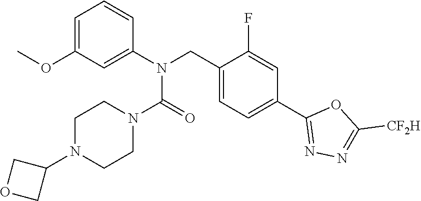 [SMILES: COc1cccc(N(Cc2ccc(-c3nnc(C(F)F)o3)cc2F)C(=O)N2CCN(C3COC3)CC2)c1] |
| 394 | 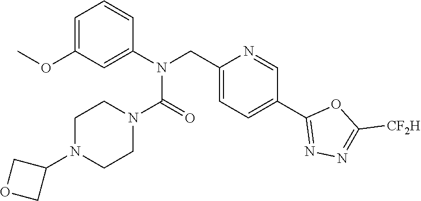 [SMILES: COc1cccc(N(Cc2ccc(-c3nnc(C(F)F)o3)cn2)C(=O)N2CCN(C3COC3)CC2)c1] |
| 395 | 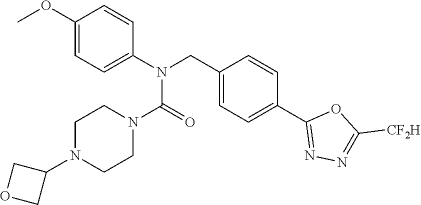 [SMILES: COc1ccc(N(Cc2ccc(-c3nnc(C(F)F)o3)cc2)C(=O)N2CCN(C3COC3)CC2)cc1] |
| 396 | 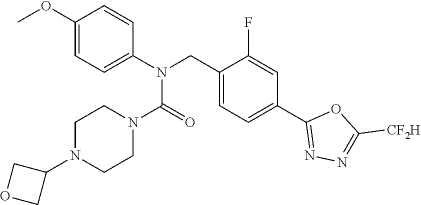 [SMILES: COc1ccc(N(Cc2ccc(-c3nnc(C(F)F)o3)cc2F)C(=O)N2CCN(C3COC3)CC2)cc1] |
| 397 | 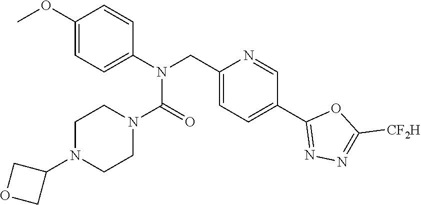 [SMILES: COc1ccc(N(Cc2ccc(-c3nnc(C(F)F)o3)cn2)C(=O)N2CCN(C3COC3)CC2)cc1] |
| 398 | 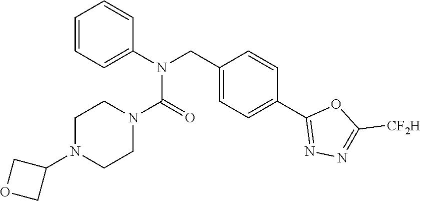 [SMILES: O=C(N1CCN(C2COC2)CC1)N(Cc1ccc(-c2nnc(C(F)F)o2)cc1)c1ccccc1] |
| 399 | 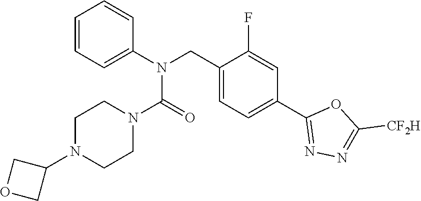 [SMILES: O=C(N1CCN(C2COC2)CC1)N(Cc1ccc(-c2nnc(C(F)F)o2)cc1F)c1ccccc1] |
| 400 | 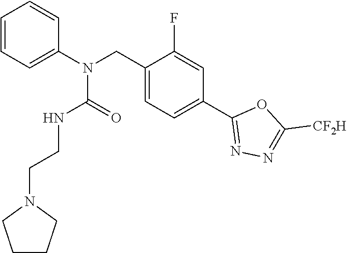 [SMILES: O=C(NCCN1CCCC1)N(Cc1ccc(-c2nnc(C(F)F)o2)cc1F)c1ccccc1] |
| 401 | 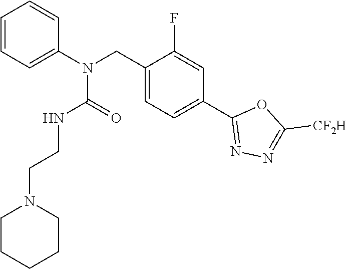 [SMILES: O=C(NCCN1CCCCC1)N(Cc1ccc(-c2nnc(C(F)F)o2)cc1F)c1ccccc1] |
| 402 | 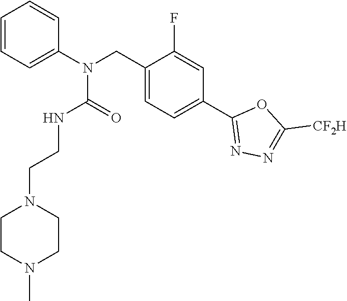 [SMILES: CN1CCN(CCNC(=O)N(Cc2ccc(-c3nnc(C(F)F)o3)cc2F)c2ccccc2)CC1] |
| 403 | 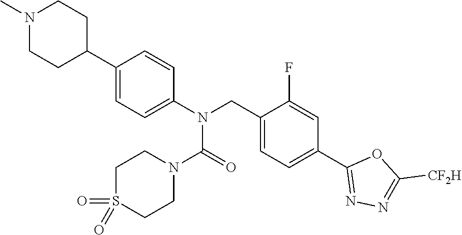 [SMILES: CN1CCC(c2ccc(N(Cc3ccc(-c4nnc(C(F)F)o4)cc3F)C(=O)N3CCS(=O)(=O)CC3)cc2)CC1] |
| 404 | 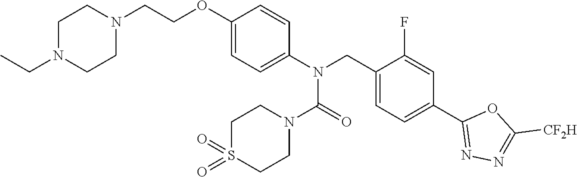 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 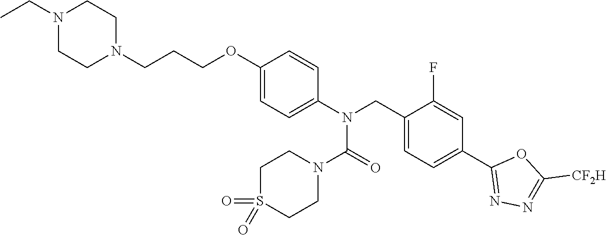 [SMILES: CCN1CCN(CCCOc2ccc(N(Cc3ccc(-c4nnc(C(F)F)o4)cc3F)C(=O)N3CCS(=O)(=O)CC3)cc2)CC1] |
| 406 | 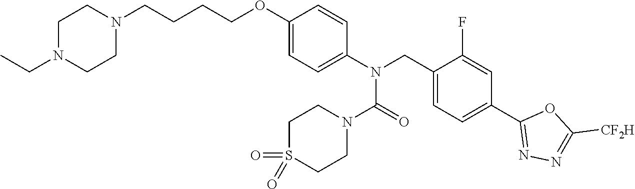 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 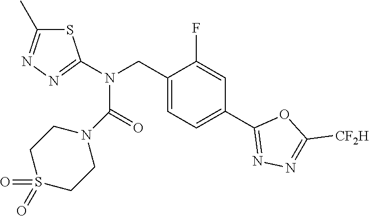 [SMILES: Cc1nnc(N(Cc2ccc(-c3nnc(C(F)F)o3)cc2F)C(=O)N2CCS(=O)(=O)CC2)s1] |
| 408 | 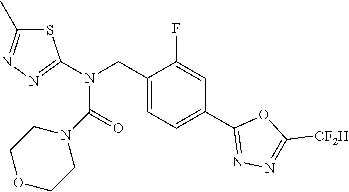 [SMILES: Cc1nnc(N(Cc2ccc(-c3nnc(C(F)F)o3)cc2F)C(=O)N2CCOCC2)s1] |
| 409 | 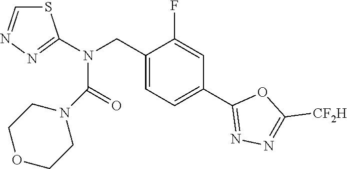 [SMILES: O=C(N1CCOCC1)N(Cc1ccc(-c2nnc(C(F)F)o2)cc1F)c1nncs1] |
| 410 | 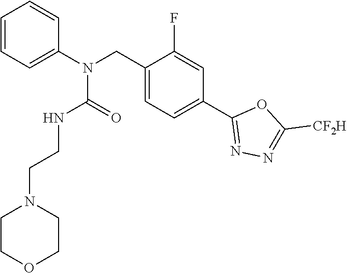 [SMILES: O=C(NCCN1CCOCC1)N(Cc1ccc(-c2nnc(C(F)F)o2)cc1F)c1ccccc1] |
| 411 | 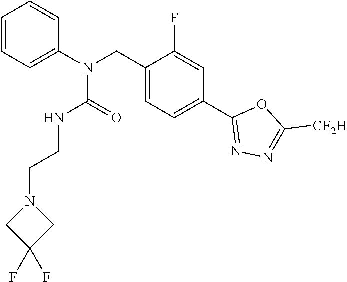 [SMILES: O=C(NCCN1CC(F)(F)C1)N(Cc1ccc(-c2nnc(C(F)F)o2)cc1F)c1ccccc1] |
| 412 | 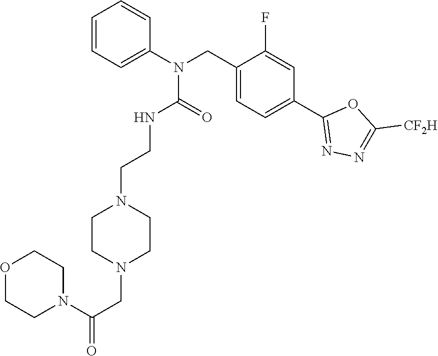 [SMILES: O=C(CN1CCN(CCNC(=O)N(Cc2ccc(-c3nnc(C(F)F)o3)cc2F)c2ccccc2)CC1)N1CCOCC1] |
| 413 | 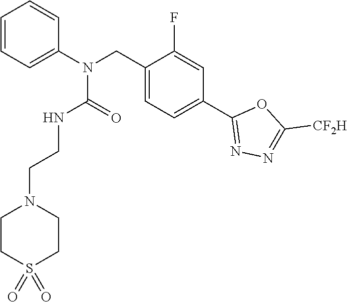 [SMILES: O=C(NCCN1CCS(=O)(=O)CC1)N(Cc1ccc(-c2nnc(C(F)F)o2)cc1F)c1ccccc1] |
| 414 | 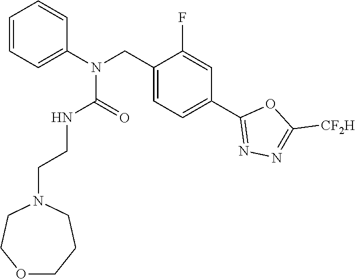 [SMILES: O=C(NCCN1CCCOCC1)N(Cc1ccc(-c2nnc(C(F)F)o2)cc1F)c1ccccc1] |
| 415 | 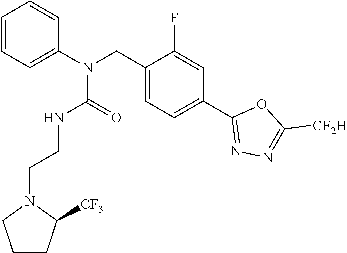 [SMILES: O=C(NCCN1CCC[C@@H]1C(F)(F)F)N(Cc1ccc(-c2nnc(C(F)F)o2)cc1F)c1ccccc1] |
| 416 | 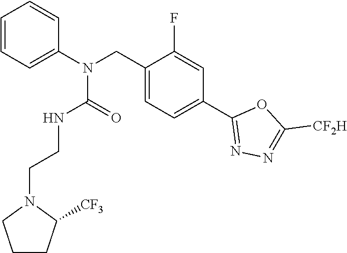 [SMILES: O=C(NCCN1CCC[C@H]1C(F)(F)F)N(Cc1ccc(-c2nnc(C(F)F)o2)cc1F)c1ccccc1] |
| 417 | 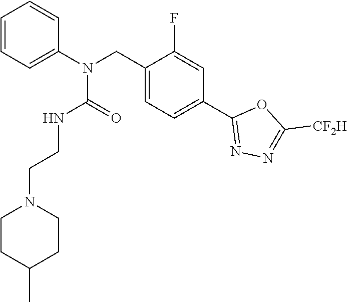 [SMILES: CC1CCN(CCNC(=O)N(Cc2ccc(-c3nnc(C(F)F)o3)cc2F)c2ccccc2)CC1] |
| 418 | 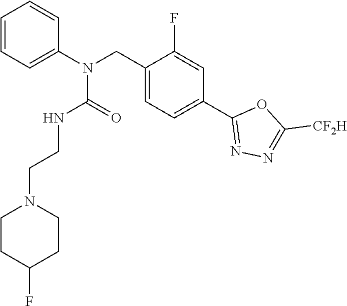 [SMILES: O=C(NCCN1CCC(F)CC1)N(Cc1ccc(-c2nnc(C(F)F)o2)cc1F)c1ccccc1] |
| 419 | 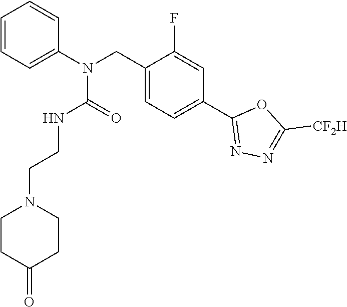 [SMILES: O=C1CCN(CCNC(=O)N(Cc2ccc(-c3nnc(C(F)F)o3)cc2F)c2ccccc2)CC1] |
| 420 | 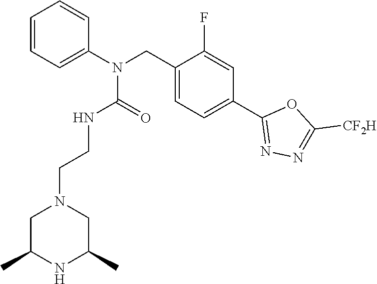 [SMILES: C[C@@H]1CN(CCNC(=O)N(Cc2ccc(-c3nnc(C(F)F)o3)cc2F)c2ccccc2)C[C@H](C)N1] |
| 421 | 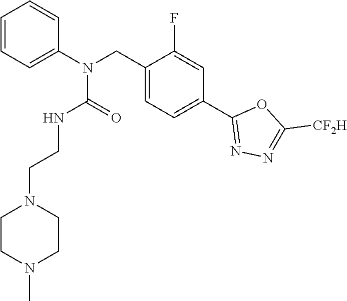 [SMILES: CN1CCN(CCNC(=O)N(Cc2ccc(-c3nnc(C(F)F)o3)cc2F)c2ccccc2)CC1] |
| 422 | 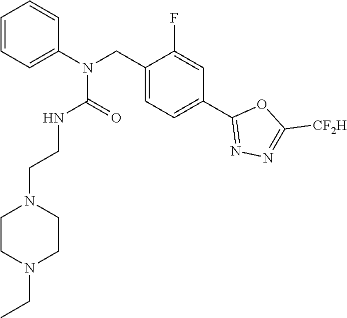 [SMILES: CCN1CCN(CCNC(=O)N(Cc2ccc(-c3nnc(C(F)F)o3)cc2F)c2ccccc2)CC1] |
| 423 | 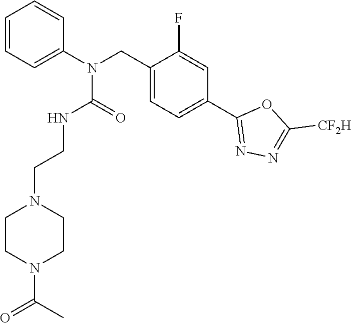 [SMILES: CC(=O)N1CCN(CCNC(=O)N(Cc2ccc(-c3nnc(C(F)F)o3)cc2F)c2ccccc2)CC1] |
| 424 | 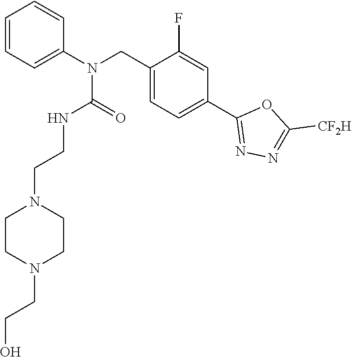 [SMILES: O=C(NCCN1CCN(CCO)CC1)N(Cc1ccc(-c2nnc(C(F)F)o2)cc1F)c1ccccc1] |
| 425 | 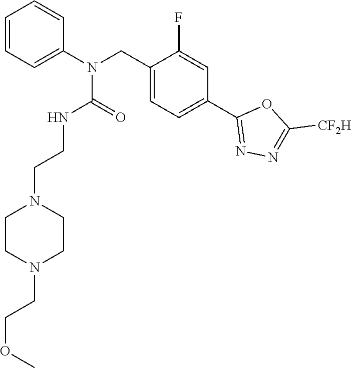 [SMILES: COCCN1CCN(CCNC(=O)N(Cc2ccc(-c3nnc(C(F)F)o3)cc2F)c2ccccc2)CC1] |
| 426 | 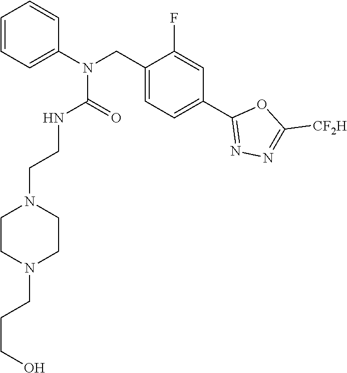 [SMILES: O=C(NCCN1CCN(CCCO)CC1)N(Cc1ccc(-c2nnc(C(F)F)o2)cc1F)c1ccccc1] |
| 427 | 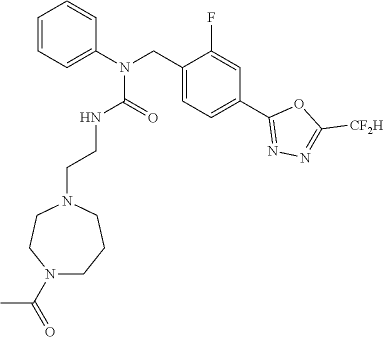 [SMILES: CC(=O)N1CCCN(CCNC(=O)N(Cc2ccc(-c3nnc(C(F)F)o3)cc2F)c2ccccc2)CC1] |
| 428 | 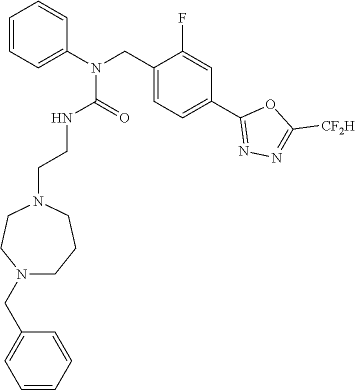 [SMILES: O=C(NCCN1CCCN(Cc2ccccc2)CC1)N(Cc1ccc(-c2nnc(C(F)F)o2)cc1F)c1ccccc1] |
| 429 | 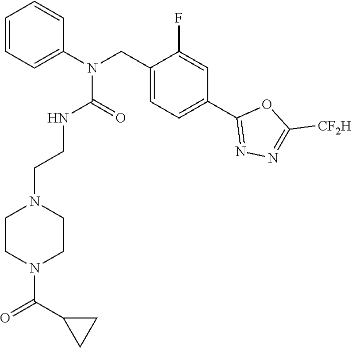 [SMILES: O=C(C1CC1)N1CCN(CCNC(=O)N(Cc2ccc(-c3nnc(C(F)F)o3)cc2F)c2ccccc2)CC1] |
| 430 | 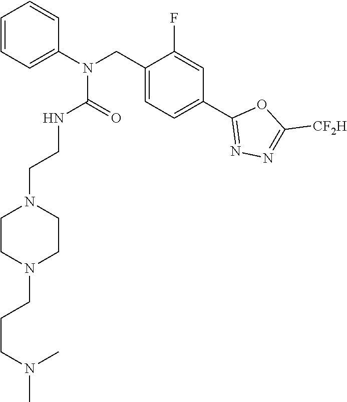 [SMILES: CN(C)CCCN1CCN(CCNC(=O)N(Cc2ccc(-c3nnc(C(F)F)o3)cc2F)c2ccccc2)CC1] |
| 431 | 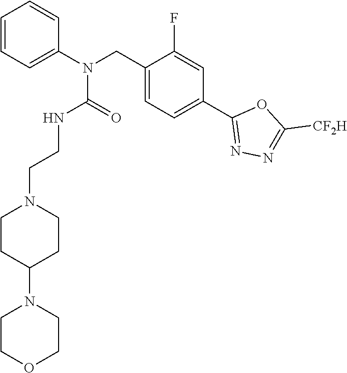 [SMILES: O=C(NCCN1CCC(N2CCOCC2)CC1)N(Cc1ccc(-c2nnc(C(F)F)o2)cc1F)c1ccccc1] |
| 432 | 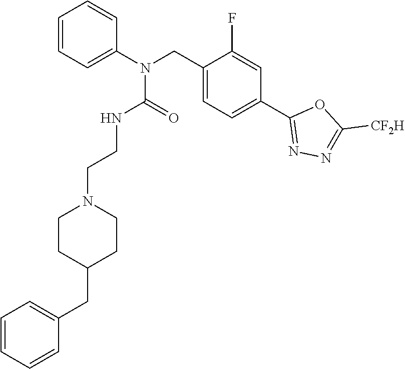 [SMILES: O=C(NCCN1CCC(Cc2ccccc2)CC1)N(Cc1ccc(-c2nnc(C(F)F)o2)cc1F)c1ccccc1] |
| 433 | 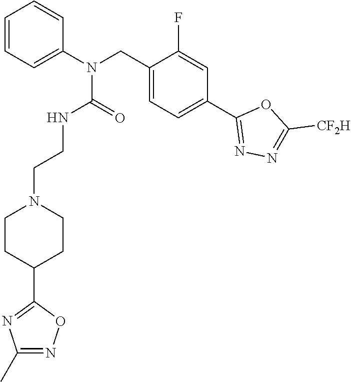 [SMILES: Cc1noc(C2CCN(CCNC(=O)N(Cc3ccc(-c4nnc(C(F)F)o4)cc3F)c3ccccc3)CC2)n1] |
| 434 | 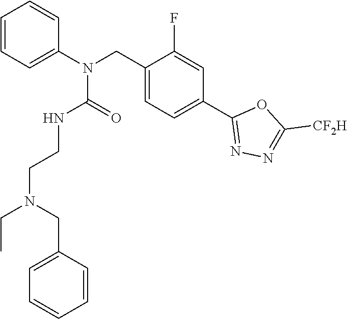 [SMILES: CCN(CCNC(=O)N(Cc1ccc(-c2nnc(C(F)F)o2)cc1F)c1ccccc1)Cc1ccccc1] |
| 435 | 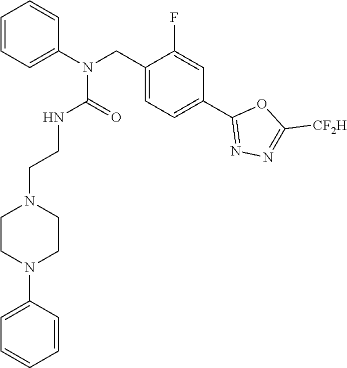 [SMILES: O=C(NCCN1CCN(c2ccccc2)CC1)N(Cc1ccc(-c2nnc(C(F)F)o2)cc1F)c1ccccc1] |
| 436 | 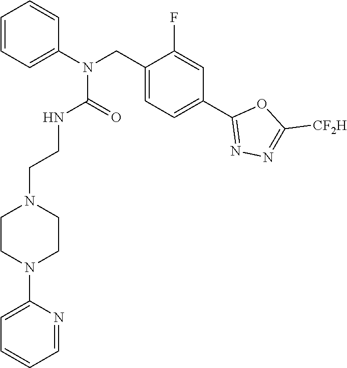 [SMILES: O=C(NCCN1CCN(c2ccccn2)CC1)N(Cc1ccc(-c2nnc(C(F)F)o2)cc1F)c1ccccc1] |
| 437 | 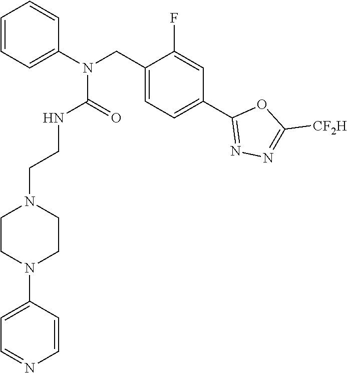 [SMILES: O=C(NCCN1CCN(c2ccncc2)CC1)N(Cc1ccc(-c2nnc(C(F)F)o2)cc1F)c1ccccc1] |
| 438 | 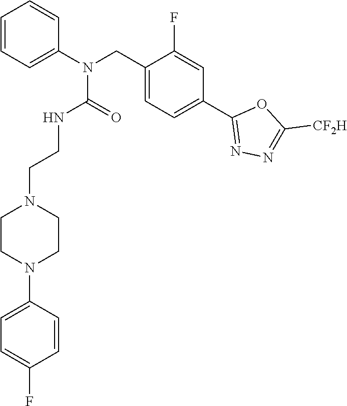 [SMILES: O=C(NCCN1CCN(c2ccc(F)cc2)CC1)N(Cc1ccc(-c2nnc(C(F)F)o2)cc1F)c1ccccc1] |
| 439 | 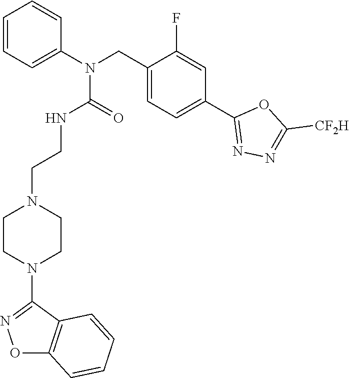 [SMILES: O=C(NCCN1CCN(c2noc3ccccc23)CC1)N(Cc1ccc(-c2nnc(C(F)F)o2)cc1F)c1ccccc1] |
| 440 | 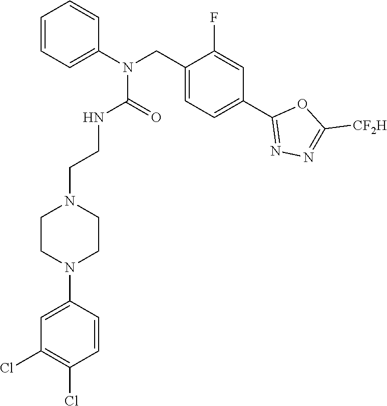 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 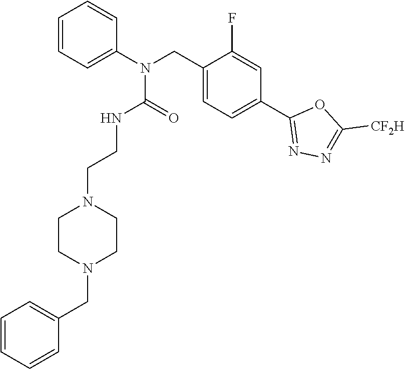 [SMILES: O=C(NCCN1CCN(Cc2ccccc2)CC1)N(Cc1ccc(-c2nnc(C(F)F)o2)cc1F)c1ccccc1] |
| 442 | 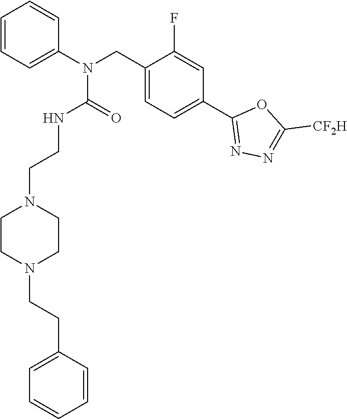 [SMILES: O=C(NCCN1CCN(CCc2ccccc2)CC1)N(Cc1ccc(-c2nnc(C(F)F)o2)cc1F)c1ccccc1] |
| 443 | 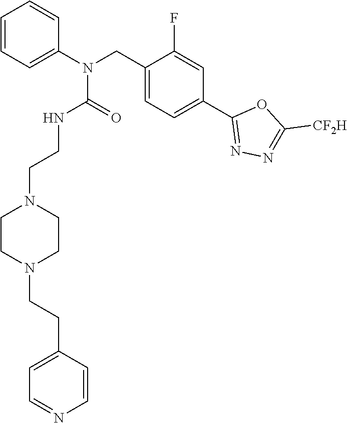 [SMILES: O=C(NCCN1CCN(CCc2ccncc2)CC1)N(Cc1ccc(-c2nnc(C(F)F)o2)cc1F)c1ccccc1] |
| 444 | 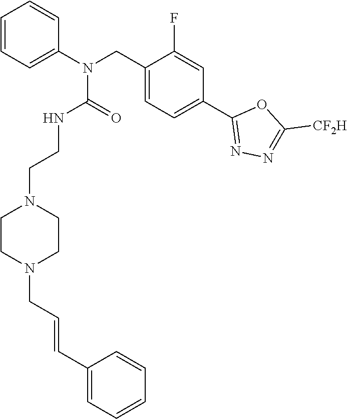 [SMILES: O=C(NCCN1CCN(C/C=C/c2ccccc2)CC1)N(Cc1ccc(-c2nnc(C(F)F)o2)cc1F)c1ccccc1] |
| 445 | 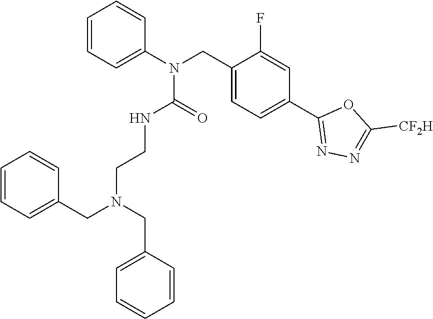 [SMILES: O=C(NCCN(Cc1ccccc1)Cc1ccccc1)N(Cc1ccc(-c2nnc(C(F)F)o2)cc1F)c1ccccc1] |
| 446 | 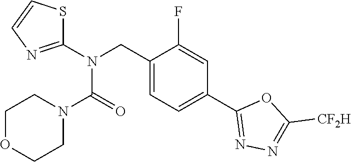 [SMILES: O=C(N1CCOCC1)N(Cc1ccc(-c2nnc(C(F)F)o2)cc1F)c1nccs1] |
| 447 | 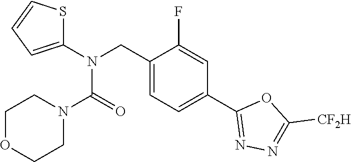 [SMILES: O=C(N1CCOCC1)N(Cc1ccc(-c2nnc(C(F)F)o2)cc1F)c1cccs1] |
| 448 | 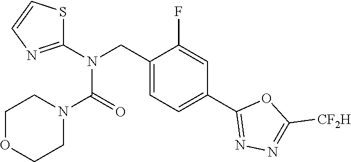 [SMILES: O=C(N1CCOCC1)N(Cc1ccc(-c2nnc(C(F)F)o2)cc1F)c1nccs1] |
| 449 | 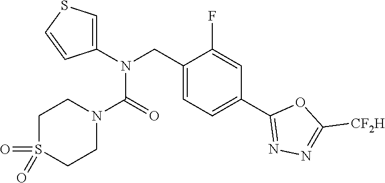 [SMILES: O=C(N1CCS(=O)(=O)CC1)N(Cc1ccc(-c2nnc(C(F)F)o2)cc1F)c1ccsc1] |
| 450 | 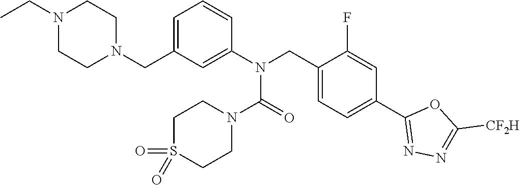 [SMILES: CCN1CCN(Cc2cccc(N(Cc3ccc(-c4nnc(C(F)F)o4)cc3F)C(=O)N3CCS(=O)(=O)CC3)c2)CC1] |

6. The pharmaceutical composition according to claim 1, wherein the compound represented by the above formula I is a compound described in a following table:

| Com- | |
|---|---|
| pound | Structure |
| 40 | 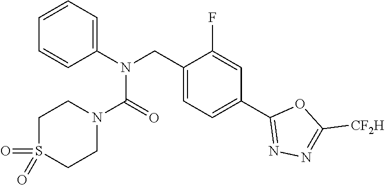 [SMILES: O=C(N1CCS(=O)(=O)CC1)N(Cc1ccc(-c2nnc(C(F)F)o2)cc1F)c1ccccc1] |
| 43 | 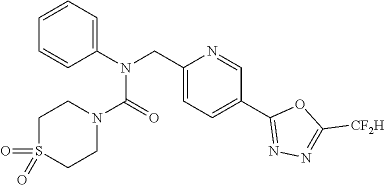 [SMILES: O=C(N1CCS(=O)(=O)CC1)N(Cc1ccc(-c2nnc(C(F)F)o2)cn1)c1ccccc1] |
| 239 | 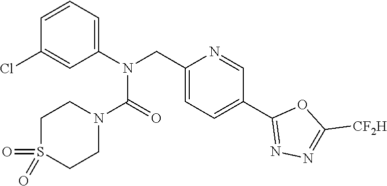 [SMILES: O=C(N1CCS(=O)(=O)CC1)N(Cc1ccc(-c2nnc(C(F)F)o2)cn1)c1cccc(Cl)c1] |
| 285 | 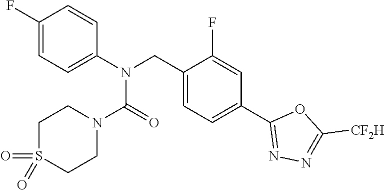 [SMILES: O=C(N1CCS(=O)(=O)CC1)N(Cc1ccc(-c2nnc(C(F)F)o2)cc1F)c1ccc(F)cc1] |
| 295 | 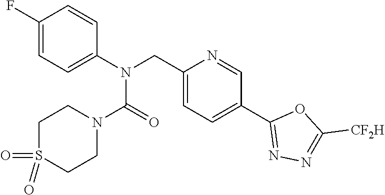 [SMILES: O=C(N1CCS(=O)(=O)CC1)N(Cc1ccc(-c2nnc(C(F)F)o2)cn1)c1ccc(F)cc1] |
| 296 | 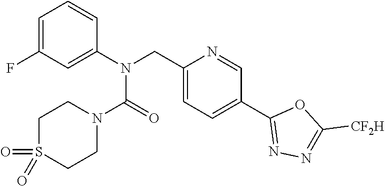 [SMILES: O=C(N1CCS(=O)(=O)CC1)N(Cc1ccc(-c2nnc(C(F)F)o2)cn1)c1cccc(F)c1] |

7. The pharmaceutical composition according to claim 1, wherein the pharmaceutical composition is orally administered.

8. A method for preventing or treating idiopathic pulmonary fibrosis, comprising administering a compound represented by formula I, optical isomers thereof or pharmaceutically acceptable salts thereof into an individual, wherein the compound represented by formula I is the same as defined in claim 1.

9. The method according to claim 8, wherein the compound represented by formula I is a compound described in a following table:

| Com- | |
|---|---|
| pound | Structure |
| 40 | 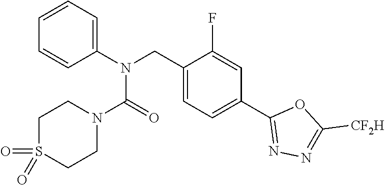 [SMILES: O=C(N1CCS(=O)(=O)CC1)N(Cc1ccc(-c2nnc(C(F)F)o2)cc1F)c1ccccc1] |
| 43 | 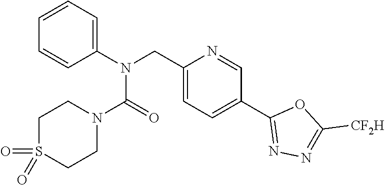 [SMILES: O=C(N1CCS(=O)(=O)CC1)N(Cc1ccc(-c2nnc(C(F)F)o2)cn1)c1ccccc1] |
| 239 | 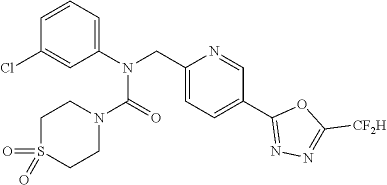 [SMILES: O=C(N1CCS(=O)(=O)CC1)N(Cc1ccc(-c2nnc(C(F)F)o2)cn1)c1cccc(Cl)c1] |
| 285 | 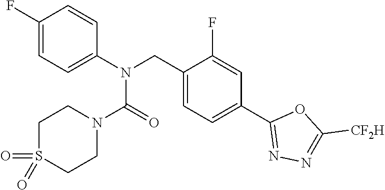 [SMILES: O=C(N1CCS(=O)(=O)CC1)N(Cc1ccc(-c2nnc(C(F)F)o2)cc1F)c1ccc(F)cc1] |
| 295 | 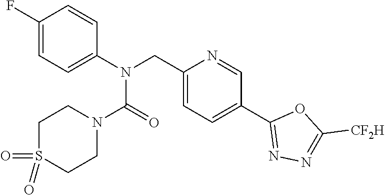 [SMILES: O=C(N1CCS(=O)(=O)CC1)N(Cc1ccc(-c2nnc(C(F)F)o2)cn1)c1ccc(F)cc1] |
| 296 | 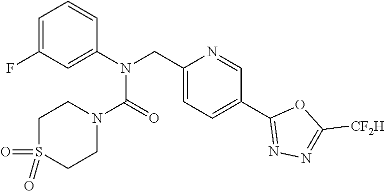 [SMILES: O=C(N1CCS(=O)(=O)CC1)N(Cc1ccc(-c2nnc(C(F)F)o2)cn1)c1cccc(F)c1] |

10. A use of a compound represented by formula I, optical isomers thereof or pharmaceutically acceptable salts thereof for preventing or treating idiopathic pulmonary fibrosis, wherein the compound represented by formula I is the same as defined in claim 1.

11. The use according to claim 10, wherein the compound represented by formula I is a compound described in a following table:

| Com- | |
|---|---|
| pound | Structure |
| 40 | 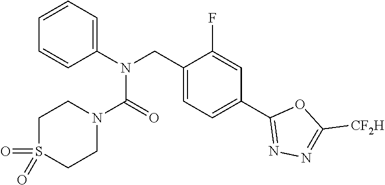 [SMILES: O=C(N1CCS(=O)(=O)CC1)N(Cc1ccc(-c2nnc(C(F)F)o2)cc1F)c1ccccc1] |
| 43 | 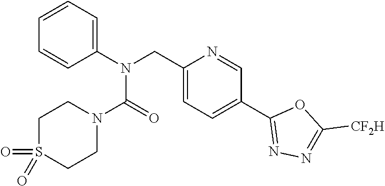 [SMILES: O=C(N1CCS(=O)(=O)CC1)N(Cc1ccc(-c2nnc(C(F)F)o2)cn1)c1ccccc1] |
| 239 | 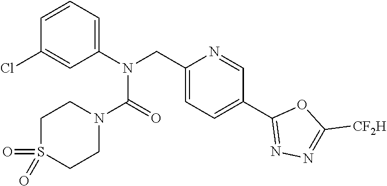 [SMILES: O=C(N1CCS(=O)(=O)CC1)N(Cc1ccc(-c2nnc(C(F)F)o2)cn1)c1cccc(Cl)c1] |
| 285 | 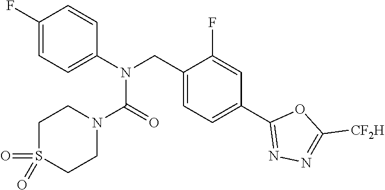 [SMILES: O=C(N1CCS(=O)(=O)CC1)N(Cc1ccc(-c2nnc(C(F)F)o2)cc1F)c1ccc(F)cc1] |
| 295 | 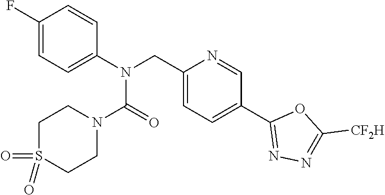 [SMILES: O=C(N1CCS(=O)(=O)CC1)N(Cc1ccc(-c2nnc(C(F)F)o2)cn1)c1ccc(F)cc1] |
| 296 | 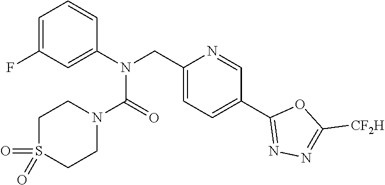 [SMILES: O=C(N1CCS(=O)(=O)CC1)N(Cc1ccc(-c2nnc(C(F)F)o2)cn1)c1cccc(F)c1] |

12. A use of a compound represented by formula I, optical isomers thereof or pharmaceutically acceptable salts thereof in preparing a medicament for preventing or treating idiopathic pulmonary fibrosis, wherein the compound represented by formula I is the same as defined in claim 1.

13. The use according to claim 12, wherein the compound represented by formula I is a compound described in a following table:

| Com- | |
|---|---|
| pound | Structure |
| 40 | 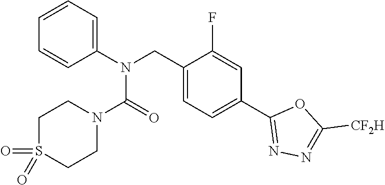 [SMILES: O=C(N1CCS(=O)(=O)CC1)N(Cc1ccc(-c2nnc(C(F)F)o2)cc1F)c1ccccc1] |
| 43 | 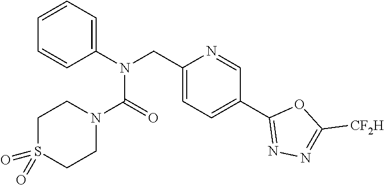 [SMILES: O=C(N1CCS(=O)(=O)CC1)N(Cc1ccc(-c2nnc(C(F)F)o2)cn1)c1ccccc1] |
| 239 | 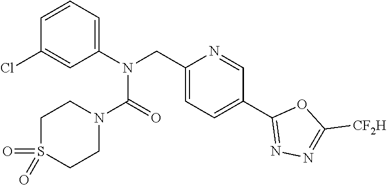 [SMILES: O=C(N1CCS(=O)(=O)CC1)N(Cc1ccc(-c2nnc(C(F)F)o2)cn1)c1cccc(Cl)c1] |
| 285 | 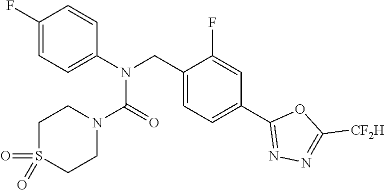 [SMILES: O=C(N1CCS(=O)(=O)CC1)N(Cc1ccc(-c2nnc(C(F)F)o2)cc1F)c1ccc(F)cc1] |
| 295 | 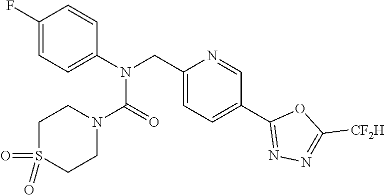 [SMILES: O=C(N1CCS(=O)(=O)CC1)N(Cc1ccc(-c2nnc(C(F)F)o2)cn1)c1ccc(F)cc1] |
| 296 | 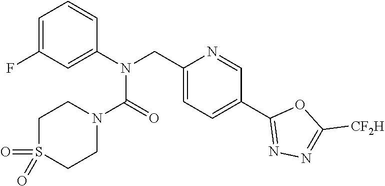 [SMILES: O=C(N1CCS(=O)(=O)CC1)N(Cc1ccc(-c2nnc(C(F)F)o2)cn1)c1cccc(F)c1] |
